SEQUENCE LISTING (215 sequences)

SEQ ID NO: 1 - protein, 120 aa, synthetic construct
EVQLQESGPSLVKPSQTLSLTCSVTGDSITSGYWNWIRKFPGNKLEYMGYISYSGSTYYNPSLKSRISITRDTSKNQYSLHLNSVTAEDTATFYCARYYGDNYVGAMDYWGQGTSVTVSS

SEQ ID NO: 2 - protein, 112 aa, synthetic construct
DIVMTQAAFSNPVTLGISASISCRSSKSLLHSNGITYLYWYLQKPGQSPQLLIYQMSNLASGVPDRFSSSGSGTDFTLRISRVEAEDVGVYYCAQNLELPWTFGGGTKLEIK

SEQ ID NO: 3 - protein, 120 aa, synthetic construct
QVQLQESGPGLVKPSQTLSLTCTVSGDSITSGYWSWIRQHPGKGLEWIGYISYSGSTYYNPSLKSRVTISVDTSKNQFSLKLSSVTAADTAVYYCARYYGDNYVGAMDYWGQGTLVTVSS

SEQ ID NO: 4 - protein, 120 aa, synthetic construct
QVQLQESGPGLVKPSQTLSLTCTVSGDSITSGYWNWIRQHPGKGLEYMGYISYSGSTYYNPSLKSRVTISVDTSKNQFSLKLSSVTAADTAVYYCARYYGDNYVGAMDYWGQGTLVTVSS

SEQ ID NO: 5 - protein, 120 aa, synthetic construct
QVQLQESGPGLVKPSQTLSLTCTVSGDSITSGYWNWIRQHPGKGLEYMGYISYSGSTYYNPSLKSRVTISVDTSKNQFSLKLSSVTAADTATFYCARYYGDNYVGAMDYWGQGTLVTVSS

SEQ ID NO: 6 - protein, 112 aa, synthetic construct
DIVMTQSPLSLPVTPGEPASISCRSSKSLLHSNGITYLDWYLQKPGQSPQLLIYQMSNRASGVPDRFSGSGSGTDFTLKISRVEAEDVGVYYCAQNLELPWTFGGGTKVEIK

SEQ ID NO: 7 - protein, 112 aa, synthetic construct
DIVMTQSPLSLPVTPGEPASISCRSSKSLLHSNGITYLYWYLQKPGQSPQLLIYQMSNLASGVPDRFSGSGSGTDFTLKISRVEAEDVGVYYCAQNLELPWTFGGGTKVEIK

SEQ ID NO: 8 - protein, 112 aa, synthetic construct
DIVLTQSPSSLAVSLGERATINCKSSKSLLHSNGITYLAWYQQKPGQPPKLLIYQMSNRESGVPDRFSGSGSGTDFTLTISSLQAEDVAVYYCAQNLELPWTFGGGTKVEIK

SEQ ID NO: 9 - protein, 112 aa, synthetic construct
DIVLTQSPSSLAVSLGERATINCRSSKSLLHSNGITYLYWYQQKPGQPPKLLIYQMSNLASGVPDRFSGSGSGTDFTLTISSLQAEDVAVYYCAQNLELPWTFGGGTKVEIK

SEQ ID NO: 10 - protein, 112 aa, synthetic construct
DIVMTQSPLSLPVTPGEPASISCRSSKSLLHSQGITYLYWYLQKPGQSPQLLIYQMSNLASGVPDRFSGSGSGTDFTLKISRVEAEDVGVYYCAQNLELPWTFGGGTKVEIK

SEQ ID NO: 11 - protein, 112 aa, synthetic construct
DIVMTQSPLSLPVTPGEPASISCRSSKSLLHSNKITYLYWYLQKPGQSPQLLIYQMSNLASGVPDRFSGSGSGTDFTLKISRVEAEDVGVYYCAQNLELPWTFGGGTKVEIK

SEQ ID NO: 12 - protein, 112 aa, synthetic construct
DIVMTQSPLSLPVTPGEPASISCRSSKSLLHSNRITYLYWYLQKPGQSPQLLIYQMSNLASGVPDRFSGSGSGTDFTLKISRVEAEDVGVYYCAQNLELPWTFGGGTKVEIK

SEQ ID NO: 13 - protein, 112 aa, synthetic construct
DIVMTQSPLSLPVTPGEPASISCRSSKSLLHSDGITYLYWYLQKPGQSPQLLIYQMSNLASGVPDRFSGSGSGTDFTLKISRVEAEDVGVYYCAQNLELPWTFGGGTKVEIK

SEQ ID NO: 14 - protein, 120 aa, synthetic construct
QVQLQQSGAELVKPGASVKLSCKASGYTFTNYDINWVRQRPEQGLEWIGWIFPGDGSTKYNEKFKGKATLTTDKSSSTAYMQLSRLTSEDSAVFFCATELVKDYYAMDYWGQGTSVTVSS

SEQ ID NO: 15 - protein, 107 aa, synthetic construct
DIVMTQSQKFMSTSVGDRVSVTCKASQNVGTNVAWYQQKPGQSPKALIYSASYRYSGVPDRFTGSGSGTDFTLTISNVQSEDLAEYFCQQYNSYPYTFGGGTKLEIK

SEQ ID NO: 16 - protein, 107 aa, synthetic construct
DIVMTQSQKFMSTSVGDRVSVTCKASQNVGTNVAWYQQKPGQSPKTLIYSASYRYSGVPDRFTGSGSGTDFTLTISNVQSEDLAEYFCQQYYNYPYTFGGGTKLEIK

SEQ ID NO: 17 - protein, 107 aa, synthetic construct
DIVMTQSQKFMSTSVGDRVSVTCKASQNVGTNVAWYQQKPGQSPKALIYSASYRYSGVPDRFTGSGSGTDFTLTISNVQSEDLAEYFCQQYNSYPLTFGAGTKLELK

SEQ ID NO: 18 - protein, 120 aa, synthetic construct
QVQLVQSGAEVKKPGASVKVSCKASGYTFTNYDMHWVRQAPGQGLEWMGWIFPGDGSTNYAQKFQGRVTMTRDTSISTAYMELSRLRSDDTAVYYCATELVKDYYAMDYWGQGTLVTVSS

SEQ ID NO: 19 - protein, 120 aa, synthetic construct
QVQLVQSGAEVKKPGASVKVSCKASGYTFTNYDINWVRQAPGQGLEWIGWIFPGDGSTKYNEKFKGRVTMTRDTSISTAYMELSRLRSDDTAVYYCATELVKDYYAMDYWGQGTLVTVSS

SEQ ID NO: 20 - protein, 120 aa, synthetic construct
QVQLVQSGAEVKKPGASVKVSCKASGYTFTNYDINWVRQAPGQGLEWIGWIFPGDGSTKYNEKFKGRVTMTRDTSISTAYMELSRLRSDDTAVFFCATELVKDYYAMDYWGQGTLVTVSS

SEQ ID NO: 21 - protein, 107 aa, synthetic construct
DIQMTQSPSSLSASVGDRVTITCRASQNVGTNLAWFQQKPGKAPKSLIYSASYLQSGVPSRFSGSGSGTDFTLTISSLQPEDFATYYCQQYNSYPYTFGQGTKLEIK

SEQ ID NO: 22 - protein, 107 aa, synthetic construct
DIQMTQSPSSLSASVGDRVTITCKASQNVGTNVAWFQQKPGKAPKALIYSASYRYSGVPSRFSGSGSGTDFTLTISSLQPEDFATYYCQQYNSYPYTFGQGTKLEIK

SEQ ID NO: 23 - protein, 107 aa, synthetic construct
DIQMTQSPSSLSASVGDRVTITCRASQNVGTNLAWFQQKPGKAPKSLIYSASYLQSGVPSRFSGSGSGTDFTLTISSLQPEDFATYYCQQYNSYPLTFGQGTKLEIK

SEQ ID NO: 24 - protein, 107 aa, synthetic construct
DIQMTQSPSSLSASVGDRVTITCKASQNVGTNVAWFQQKPGKAPKALIYSASYRYSGVPSRFSGSGSGTDFTLTISSLQPEDFATYYCQQYNSYPLTFGQGTKLEIK

SEQ ID NO: 25 - protein, 120 aa, synthetic construct
QVQLVQSGAEVKKPGASVKVSCKASGYTFTNYDINWVRQAPGQGLEWIGWIFPGEGSTKYNEKFKGRVTMTRDTSISTAYMELSRLRSDDTAVYYCATELVKDYYAMDYWGQGTLVTVSS

SEQ ID NO: 26 - protein, 120 aa, synthetic construct
QVQLVQSGAEVKKPGASVKVSCKASGYTFTNYDINWVRQAPGQGLEWIGWIFPGDVSTKYNEKFKGRVTMTRDTSISTAYMELSRLRSDDTAVYYCATELVKDYYAMDYWGQGTLVTVSS

SEQ ID NO: 27 - protein, 116 aa, synthetic construct
EVQLQQSGPELVKPGASVKMSCKASGYTFTSYVMHWVKQKPGQGLEWIGYINPYNDGTKYNEKFKGKATLTSDKSSSTAYMELSSLTSEDSAIYYCASEGNYEDYWGQGTLVTVSA

SEQ ID NO: 28 - protein, 116 aa, synthetic construct
QVRLQQSGPELVKPGASVRISCKASGHTFTSYYIYWVKQRPGQGLEWIGWIYFGNINLKYNEKFKGKATLTADKSSSTAYIQLSSLTSEDSAVYFCASEDGPGAYWGQGTLVTVSA

SEQ ID NO: 29 - protein, 117 aa, synthetic construct
EVQLQQSGPELVKPGASMKISCKTSGYTFSENTIYWVKQRQGKSLEWIGGINPKNGGTTYNPEFKGKATLTVDKSSSTAYMDLRSLTSDDSAVYYCAIYDSDYFDYWGQGTTLTVSS

SEQ ID NO: 30 - protein, 107 aa, synthetic construct
DIVMTQSQKFMSTSVGDRVIVTCKASQNVGTNVAWYQQKPGQSPKALIYSASFRYGGVPDRFTGIGSGTEFTLTIRNVQSEDLAVYFCQQYNSYPITFGGGTKLEIK

SEQ ID NO: 31 - protein, 121 aa, synthetic construct
EVKLVESGGGLVLPGGSLRLSCATSGFTFTDYYMSWVRQPPGKALEWLGFIGNRPNGYTTEYNASVKGRFTISRDNSQSILYLQMNTLRAEDSATYYCARGSTMITWFAYWGQGTLVTVSA

SEQ ID NO: 32 - protein, 112 aa, synthetic construct
DVLMTQSPLSLPVSLGDQASISCRSSQSIVHSNGDTYFDWYLQKPGQSPKLLIYKVSNRFSGVPDRFSGSGSGTDFTLKISRVEAEDLGVYYCFQGSLLPPAFGGGTKLEIK

SEQ ID NO: 33 - protein, 107 aa, synthetic construct
DVLMTQSPKFMSTSVGDRVSVTCKASQNVGTNVAWYRQKQGQSPKALIYSASYRYSGVPDRFTGSGSGTDFTLTISNVQSEDLADYFCQQYNSYPLTFGAGTKLEIK

SEQ ID NO: 34 - protein, 120 aa, synthetic construct
QVQLQQSAAELARPGASVKMSCEASGYTFTTYTMYWVKQRPGQGLEWIGYINPGSGYTEYNQKFKDKTTLTADKSSSTAYMQLSSLTSEDSAVYYCATEEVRRGDYFDYWGQGTTLTVSS

SEQ ID NO: 35 - protein, 107 aa, synthetic construct
DIVMTQSQKFMSTSVGNRVSVTCKASQNVGTNVAWYQQKPGQSPKALIYSASYRYSGVPDRFTGSGSGTDFTLTISNVQSEDLADYFCQQYNSFPFTFGSGTKLEIK

SEQ ID NO: 36 - protein, 119 aa, synthetic construct
EVQLQQSGAELVKPGASVKLSCTASGFNIKDTYIYWVRQRPEQGLEWIGRIDPANGNTKYDPKVQGKATITADTSSNTAYLQLSSLTFEDTAVYYCALEHGYYDVMDYWGQGTSVTVSS

SEQ ID NO: 37 - protein, 107 aa, synthetic construct
DIVMTQSQKFMSTSVGDRVSVTCKASQNVDTNVAWYQKKPGQSPKALIYSASYRYSGVPDRFTGSGSGTDFTLTISNVQSEDLAEYFCQQYNSYPYTFGGGTKLEIK

SEQ ID NO: 38 - protein, 116 aa, synthetic construct
EIQLQQSGPELVKPGASVHISCKSSGYAFTSYNIYWVKQSHGKSLEWIGHIDPDNGGIIYNQKFRGKATLTVDKSSSTAYMHLNSLTSEDSAVYYCAREDGNYDYWGQGTTLTVSS

SEQ ID NO: 39 - protein, 107 aa, synthetic construct
DIVMTQSPKFMSTSVGDRVSVTCKASQNVGTNVAWYRQKQGQSPKALIYSASYRYSGVPDRFTGSGSGTDFTLTISNVQSEDLADYFCQQYNSYPLTFGAGTKLELK

SEQ ID NO: 40 - protein, 116 aa, synthetic construct
EVQLQQSGPELVKPGASVKISCKTSGYTFTESTMYWVKQSHGKSLEWIGHINPNNGGTSYNQKFRGKATLTVDKSSSTAYMELRSLTSEDSAVYYCAMELFYFDYWGQGTTLTVSS

SEQ ID NO: 41 - protein, 107 aa, synthetic construct
DIVMTQSQKFMSTSVGDRVSVTCKASQNVGTNVAWYQQKPGQSPKALIYSASYRYSGVPDRFTGSGSGTDFTLTISNVQSEDLAEYFCQQYNISPYTFGGGTKLEIK

SEQ ID NO: 42 - protein, 122 aa, synthetic construct
QAYLQQSGAELVRSGASVKMSCKASGYTFTSFSMHWIKQTPGQGLEWIGYIYPGNGYTNYNQKFKGKATLTADTSSNTAYMQISSLTSEDSAVYFCGRDYGHTDYDYAMDYWGQGTSVTVSS

SEQ ID NO: 43 - protein, 107 aa, synthetic construct
DIVMTQSQKFMSTSVGDRVSVSCKASQNVGTNVAWYQQKPGRSPKALIYSASYRYSGVPDRFTGSGSGTDFTLTISNVQSEDLAEYFCQQYNTYPYTFGGGTKLEIK

SEQ ID NO: 44 - protein, 121 aa, synthetic construct
EVQLQQSGAELVRSGASVKLSCTASGFNIKDQFMHWVNQRPEQGLEWIGWIDPQNGNTEYAPKFQGKATMTADTSSNTAYLQLSSLTSEDTAVYYCNAEKYVNYETWFAYWGQGTLVTVSA

SEQ ID NO: 45 - protein, 107 aa, synthetic construct
DIVMTQSQKFMSTSVGDRVSVTCKASQNVGTNVAWYQQKPGQSPKALIYSASYRYSGVPDRFTGSGSGTDFTLTISNVQSEDLAEYFCQQYNTYPFTFGGGTKLEIK

SEQ ID NO: 46 - protein, 116 aa, synthetic construct
QVQLQQSGPELVRPGASVKMSCKASGYTFTNYWMYWVKQRPGQGLEWIGMIDPSNSETRLNQKFKDKATLNVDKSSNTAYMQLSSLTSEDSAVYYCASELGRYAYWGQGTLVTVSA

SEQ ID NO: 47 - protein, 107 aa, synthetic construct
DIVMTQSQNFMSSSVGDRVSVTCKASQNVDTNVAWYQQKPGQSPKALIYSASYRYSGVPDRFTGSGSGTDFTLTISNVQSEDLAEYFCQQYNSYPFTFGSGTKLEIK

SEQ ID NO: 48 - protein, 122 aa, synthetic construct
QVQLQQPGAELVRPGASVKLSCKASGYSFTSYWMYWVKQRPGQGLEWIGMIHPSDSETRLNQKFKDKATLTVDKSSSTAFMQLSSPTSEDSAVYYCASEIYYDYDNGWFAYWGQGTLVTVSA

SEQ ID NO: 49 - protein, 107 aa, synthetic construct
DIVMTQSQKFMSTLVGDRVSVTCKASQNVDTNVAWYQQKPGQSPKALIYSASYRYSGVPDRFTGSGSGTDFTLTISNVQSEDLAEYFCQQYNSHPYTFGGGTKLEIK

SEQ ID NO: 50 - protein, 116 aa, synthetic construct
QVQLQQSGAELVKPGASVKLSCKAFGYTFTNYDIHWVRQRPEQGLEWIGWIFPGDGSTKYNEKFKGKATLTTDKSSSTAYMQLSRLTSEDSAVYFCASELGREDYWGQGTTLTVSS

SEQ ID NO: 51 - protein, 116 aa, synthetic construct
QVQLQQSGPELVRPGASVKMSCKASGYTFTNYWIYWVKQRPGQGLEWIGMIDPSNTEARLHQRFKDKATLNVDKSSNTAYMQLSSLTSEDSAVYYCAIEYGNSDYWGQGTTLTVSS

SEQ ID NO: 52 - protein, 107 aa, synthetic construct
DIVMTQSQKFMSTSVGDRVSVTCKASQNVGTNVAWYQQKPGQSPKALIYSASYRYSGVPDRFTGSGSGTDFTLTINNVQSEDLAEYFCQQYNSYPYTFGGGTKLEIK

SEQ ID NO: 53 - protein, 120 aa, synthetic construct
EVHLQQSGAELVKPGASVKLSCTPSGFNIKDTYIHWVNQRPEQGLEWIGGIDPANVNPKYDPKFQAKATITADTSSNTAYLQLSSLTSEDTAVYYCGIAYYRYDVWFAYWGQGTLVTVSA

SEQ ID NO: 54 - protein, 107 aa, synthetic construct
DIVMTQSQKFMSTSVGDRVSVTCKASQNVNTNVVWYQQKPGQSPKALIYSASYRYSGVPDRFTGSGSGTDFTLTISNVQSENLGKYFCQQYNSYPYTFGGGTKLEIK

SEQ ID NO: 55-56 - skipped (2 entries)

SEQ ID NO: 57 - protein, 112 aa, synthetic construct
DIVMTQSPLSLPVTPGEPASISCRSSQSIVHSNGDTYLDWYLQKPGQSPQLLIYKVSNRASGVPDRFSGSGSGTDFTLKISRVEAEDVGVYYCFQGSLLPPAFGGGTKVEIK

SEQ ID NO: 58 - protein, 121 aa, synthetic construct
QVQLVESGGGLVKPGGSLRLSCAASGFTFTDYYMSWIRQAPGKGLEWVSYIGNRPNGYTTYYADSVKGRFTISRDNAKNSLYLQMNSLRAEDTAVYYCARGSTMITWFAYWGQGTTVTVSS

SEQ ID NO: 59 - protein, 121 aa, synthetic construct
QVQLVESGGGLVKPGGSLRLSCAASGFTFTDYYMSWIRQAPGKGLEWLGFIGNRPNGYTTEYNASVKGRFTISRDNAKNSLYLQMNSLRAEDTAVYYCARGSTMITWFAYWGQGTTVTVSS

SEQ ID NO: 60 - protein, 121 aa, synthetic construct
QVQLVESGGGLVKPGGSLRLSCAASGFTFTDYYMSWIRQAPGKGLEWLGFIGNRPNGYTTEYQASVKGRFTISRDNAKNSLYLQMNSLRAEDTAVYYCARGSTMITWFAYWGQGTTVTVSS

SEQ ID NO: 61 - protein, 10 aa, synthetic construct
  VARIANT 10: note = N or S
GDSITSGYWX

SEQ ID NO: 62 - protein, 10 aa, synthetic construct
GDSITSGYWN

SEQ ID NO: 63 - protein, 10 aa, synthetic construct
GDSITSGYWS

SEQ ID NO: 64 - protein, 16 aa, synthetic construct
YISYSGSTYYNPSLKS

SEQ ID NO: 65 - protein, 14 aa, synthetic construct
ARYYGDNYVGAMDY

SEQ ID NO: 66 - protein, 16 aa, synthetic construct
  VARIANT 1: note = R or K
  VARIANT 10: note = N, Q or D
  VARIANT 11: note = G, K or R
  VARIANT 16: note = Y, D or A
XSSKSLLHSXXITYLX

SEQ ID NO: 67 - protein, 16 aa, synthetic construct
RSSKSLLHSNGITYLY

SEQ ID NO: 68 - protein, 16 aa, synthetic construct
RSSKSLLHSNGITYLD

SEQ ID NO: 69 - protein, 16 aa, synthetic construct
KSSKSLLHSNGITYLA

SEQ ID NO: 70 - protein, 16 aa, synthetic construct
RSSKSLLHSQGITYLY

SEQ ID NO: 71 - protein, 16 aa, synthetic construct
RSSKSLLHSNKITYLY

SEQ ID NO: 72 - protein, 16 aa, synthetic construct
RSSKSLLHSNRITYLY

SEQ ID NO: 73 - protein, 16 aa, synthetic construct
RSSKSLLHSDGITYLY

SEQ ID NO: 74 - protein, 7 aa, synthetic construct
  VARIANT 5: note = L or R
  VARIANT 6: note = A or E
QMSNXXS

SEQ ID NO: 75 - protein, 7 aa, synthetic construct
QMSNLAS

SEQ ID NO: 76 - protein, 7 aa, synthetic construct
QMSNRAS

SEQ ID NO: 77 - protein, 7 aa, synthetic construct
QMSNRES

SEQ ID NO: 78 - protein, 9 aa, synthetic construct
AQNLELPWT

SEQ ID NO: 79 - protein, 10 aa, synthetic construct
  VARIANT 9: note = I or M
  VARIANT 10: note = N or H
GYTFTNYDXX

SEQ ID NO: 80 - protein, 10 aa, synthetic construct
GYTFTNYDIN

SEQ ID NO: 81 - protein, 10 aa, synthetic construct
GYTFTNYDMH

SEQ ID NO: 82 - protein, 17 aa, synthetic construct
  VARIANT 6: note = D or E
  VARIANT 7: note = G or V
  VARIANT 10: note = K or N
  VARIANT 12: note = N or A
  VARIANT 13: note = E or Q
  VARIANT 16: note = K or Q
WIFPGXXSTXYXXKFXG

SEQ ID NO: 83 - protein, 17 aa, synthetic construct
WIFPGDGSTKYNEKFKG

SEQ ID NO: 84 - protein, 17 aa, synthetic construct
WIFPGDGSTNYAQKFQG

SEQ ID NO: 85 - protein, 17 aa, synthetic construct
WIFPGEGSTKYNEKFKG

SEQ ID NO: 86 - protein, 17 aa, synthetic construct
WIFPGDVSTKYNEKFKG

SEQ ID NO: 87 - protein, 13 aa, synthetic construct
ATELVKDYYAMDY

SEQ ID NO: 88 - protein, 11 aa, synthetic construct
  VARIANT 1: note = K or R
  VARIANT 10: note = V or L
XASQNVGTNXA

SEQ ID NO: 89 - protein, 11 aa, synthetic construct
KASQNVGTNVA

SEQ ID NO: 90 - protein, 11 aa, synthetic construct
RASQNVGTNLA

SEQ ID NO: 91 - protein, 7 aa, synthetic construct
  VARIANT 5: note = R or L
  VARIANT 6: note = Y or Q
SASYXXS

SEQ ID NO: 92 - protein, 7 aa, synthetic construct
SASYRYS

SEQ ID NO: 93 - protein, 7 aa, synthetic construct
SASYLQS

SEQ ID NO: 94 - protein, 9 aa, synthetic construct
  VARIANT 4: note = N or Y
  VARIANT 5: note = S or N
  VARIANT 8: note = Y or L
QQYXXYPXT

SEQ ID NO: 95 - protein, 9 aa, synthetic construct
QQYNSYPYT

SEQ ID NO: 96 - protein, 9 aa, synthetic construct
QQYYNYPYT

SEQ ID NO: 97 - protein, 9 aa, synthetic construct
QQYNSYPLT

SEQ ID NO: 98 - protein, 10 aa, synthetic construct
GFTFTDYYMS

SEQ ID NO: 99 - protein, 19 aa, synthetic construct
  VARIANT 1: note = F or Y
  VARIANT 12: note = E or Y
  VARIANT 14: note = N, A, or Q
  VARIANT 15: note = A or D
XIGNRPNGYTTXYXXSVKG

SEQ ID NO: 100 - skipped

SEQ ID NO: 101 - protein, 19 aa, synthetic construct
FIGNRPNGYTTEYNASVKG

SEQ ID NO: 102 - protein, 19 aa, synthetic construct
YIGNRPNGYTTYYADSVKG

SEQ ID NO: 103 - protein, 19 aa, synthetic construct
FIGNRPNGYTTEYQASVKG

SEQ ID NO: 104 - protein, 12 aa, synthetic construct
ARGSTMITWFAY

SEQ ID NO: 105 - protein, 16 aa, synthetic construct
RSSQSIVHSNGDTYFD

SEQ ID NO: 106 - protein, 16 aa, synthetic construct
RSSQSIVHSNGDTYLD

SEQ ID NO: 107 - protein, 7 aa, synthetic construct
KVSNRFS

SEQ ID NO: 108 - protein, 7 aa, synthetic construct
KVSNRAS

SEQ ID NO: 109 - protein, 9 aa, synthetic construct
FQGSLLPPA

SEQ ID NO: 110 - protein, 10 aa, synthetic construct
GYTFTTYTMY

SEQ ID NO: 111 - protein, 17 aa, synthetic construct
YINPGSGYTEYNQKFKD

SEQ ID NO: 112 - protein, 13 aa, synthetic construct
ATEEVRRGDYFDY

SEQ ID NO: 113 - protein, 9 aa, synthetic construct
QQYNSFPFT

SEQ ID NO: 114 - protein, 10 aa, synthetic construct
GFNIKDTYIY

SEQ ID NO: 115 - protein, 17 aa, synthetic construct
RIDPANGNTKYDPKVQG

SEQ ID NO: 116 - protein, 12 aa, synthetic construct
ALEHGYYDVMDY

SEQ ID NO: 117 - protein, 11 aa, synthetic construct
KASQNVDTNVA

SEQ ID NO: 118 - protein, 10 aa, synthetic construct
GYAFTSYNIY

SEQ ID NO: 119 - protein, 17 aa, synthetic construct
HIDPDNGGIIYNQKFRG

SEQ ID NO: 120 - protein, 9 aa, synthetic construct
AREDGNYDY

SEQ ID NO: 121 - protein, 10 aa, synthetic construct
GYTFTESTMY

SEQ ID NO: 122 - protein, 17 aa, synthetic construct
HINPNNGGTSYNQKFRG

SEQ ID NO: 123 - protein, 9 aa, synthetic construct
AMELFYFDY

SEQ ID NO: 124 - protein, 9 aa, synthetic construct
QQYNISPYT

SEQ ID NO: 125 - protein, 10 aa, synthetic construct
GYTFTSFSMH

SEQ ID NO: 126 - protein, 17 aa, synthetic construct
YIYPGNGYTNYNQKFKG

SEQ ID NO: 127 - protein, 15 aa, synthetic construct
GRDYGHTDYDYAMDY

SEQ ID NO: 128 - protein, 9 aa, synthetic construct
QQYNTYPYT

SEQ ID NO: 129 - protein, 10 aa, synthetic construct
GFNIKDQFMH

SEQ ID NO: 130 - protein, 17 aa, synthetic construct
WIDPQNGNTEYAPKFQG

SEQ ID NO: 131 - protein, 14 aa, synthetic construct
NAEKYVNYETWFAY

SEQ ID NO: 132 - protein, 9 aa, synthetic construct
QQYNTYPFT

SEQ ID NO: 133 - protein, 10 aa, synthetic construct
GYTFTNYWMY

SEQ ID NO: 134 - protein, 17 aa, synthetic construct
MIDPSNSETRLNQKFKD

SEQ ID NO: 135 - protein, 9 aa, synthetic construct
ASELGRYAY

SEQ ID NO: 136 - protein, 9 aa, synthetic construct
QQYNSYPFT

SEQ ID NO: 137 - protein, 10 aa, synthetic construct
GYSFTSYWMY

SEQ ID NO: 138 - protein, 17 aa, synthetic construct
MIHPSDSETRLNQKFKD

SEQ ID NO: 139 - protein, 15 aa, synthetic construct
ASEIYYDYDNGWFAY

SEQ ID NO: 140 - protein, 9 aa, synthetic construct
QQYNSHPYT

SEQ ID NO: 141 - protein, 10 aa, synthetic construct
GYTFTNYDIH

SEQ ID NO: 142 - protein, 9 aa, synthetic construct
ASELGREDY

SEQ ID NO: 143 - protein, 10 aa, synthetic construct
GYTFTNYWIY

SEQ ID NO: 144 - protein, 17 aa, synthetic construct
MIDPSNTEARLHQRFKD

SEQ ID NO: 145 - protein, 9 aa, synthetic construct
AIEYGNSDY

SEQ ID NO: 146 - protein, 10 aa, synthetic construct
GFNIKDTYIH

SEQ ID NO: 147 - protein, 17 aa, synthetic construct
GIDPANVNPKYDPKFQA

SEQ ID NO: 148 - protein, 13 aa, synthetic construct
GIAYYRYDVWFAY

SEQ ID NO: 149 - protein, 11 aa, synthetic construct
KASQNVNTNVV

SEQ ID NO: 150 - protein, 10 aa, synthetic construct
GYTFTSYVMH

SEQ ID NO: 151 - protein, 17 aa, synthetic construct
YINPYNDGTKYNEKFKG

SEQ ID NO: 152 - protein, 9 aa, synthetic construct
ASEGNYEDY

SEQ ID NO: 153 - protein, 10 aa, synthetic construct
GHTFTSYYIY

SEQ ID NO: 154 - protein, 17 aa, synthetic construct
WIYFGNINLKYNEKFKG

SEQ ID NO: 155 - protein, 9 aa, synthetic construct
ASEDGPGAY

SEQ ID NO: 156 - protein, 10 aa, synthetic construct
GYTFSENTIY

SEQ ID NO: 157 - protein, 17 aa, synthetic construct
GINPKNGGTTYNPEFKG

SEQ ID NO: 158 - protein, 10 aa, synthetic construct
AIYDSDYFDY

SEQ ID NO: 159 - protein, 7 aa, synthetic construct
SASFRYG

SEQ ID NO: 160 - protein, 9 aa, synthetic construct
QQYNSYPIT

SEQ ID NO: 161 - protein, 14 aa, synthetic construct
ARYYGDNYVGALDY

SEQ ID NO: 162 - protein, 14 aa, synthetic construct
ARYYGDNYVGAQDY

SEQ ID NO: 163 - protein, 14 aa, synthetic construct
ARYYGDNYVGAIDY

SEQ ID NO: 164 - protein, 14 aa, synthetic construct
ARYYGDNYVGAADY

SEQ ID NO: 165 - protein, 14 aa, synthetic construct
  VARIANT 12: note = M, L, Q, I, or A
ARYYGDNYVGAXDY

SEQ ID NO: 166 - protein, 120 aa, synthetic construct
QVQLQESGPGLVKPSQTLSLTCTVSGDSITSGYWNWIRQHPGKGLEYMGYISYSGSTYYNPSLKSRVTISVDTSKNQFSLKLSSVTAADTAVYYCARYYGDNYVGALDYWGQGTLVTVSS

SEQ ID NO: 167 - protein, 119 aa, synthetic construct
QVQLQESGPGLVKPSQTLSLTCTVSGDSITSGYWNWIRQHPGKGLEYMGYISYSGSTYYNPSLKSRVTISVDTSKNQFSLKLSSVTAADTAVYYCARYYGDNYVGAQDYWGQGTLVTVS

SEQ ID NO: 168 - protein, 120 aa, synthetic construct
QVQLQESGPGLVKPSQTLSLTCTVSGDSITSGYWNWIRQHPGKGLEYMGYISYSGSTYYNPSLKSRVTISVDTSKNQFSLKLSSVTAADTAVYYCARYYGDNYVGAIDYWGQGTLVTVSS

SEQ ID NO: 169 - protein, 120 aa, synthetic construct
QVQLQESGPGLVKPSQTLSLTCTVSGDSITSGYWNWIRQHPGKGLEYMGYISYSGSTYYNPSLKSRVTISVDTSKNQFSLKLSSVTAADTAVYYCARYYGDNYVGAADYWGQGTLVTVSS

SEQ ID NO: 170 - protein, 107 aa, synthetic construct
DIQMTQSPSSLSASVGDRVTITCKASQNVGTNVAWYQQKPGKAPKALIYSASYRYSGVPSRFSGSGSGTDFTLTISSLQPEDFATYYCQQYNSYPLTFGQGTKLEIK

SEQ ID NO: 171 - protein, 117 aa, synthetic construct
QVQLVQSGAEVKKPGASVKVSCKASGYTFSENTIYWVRQAPGQGLEWIGGINPKNGGTTYNPEFKGRVTMTTDTSTSTAYMELRSLRSDDTAVYYCAIYDSDYFDYWGQGTLVTVSS

SEQ ID NO: 172 - protein, 119 aa, synthetic construct
QVQLVQSGAEVKKPGASVKVSCKASGFNIKDTYINWVRQATGQGLEWMGWIDPANGNTGYAQKFQGRVTITRNTSISTAYMELSSLRSEDTAVYYCALEHGYYDVMDYWGQGTLVTVSS

SEQ ID NO: 173 - protein, 119 aa, synthetic construct
QVQLVQSGAEVKKPGASVKVSCKASGFNIKDTYIYWVRQATGQGLEWIGRIDPANGNTKYDPKVQGRVTITRNTSISTAYMELSSLRSEDTAVYYCALEHGYYDVMDYWGQGTLVTVSS

SEQ ID NO: 174 - protein, 10 aa, synthetic construct
GFNIKDTYIN

SEQ ID NO: 175 - protein, 17 aa, synthetic construct
WIDPANGNTGYAQKFQG

SEQ ID NO: 176 - protein, 116 aa, synthetic construct
QVQLVQSGAEVKKPGASVKVSCKASGYTFTESTMHWVRQAPGQGLEWMGRINPNNGGTNYAQKFQGRVTMTRDTSISTAYMELSRLRSDDTAVYYCAMELFYFDYWGQGTLVTVSS

SEQ ID NO: 177 - protein, 116 aa, synthetic construct
QVQLVQSGAEVKKPGASVKVSCKASGYTFTESTMYWVRQAPGQGLEWIGHINPNNGGTSYNQKFRGRVTMTRDTSISTAYMELSRLRSDDTAVYYCAMELFYFDYWGQGTLVTVSS

SEQ ID NO: 178 - protein, 107 aa, synthetic construct
DIQMTQSPSSLSASVGDRVTITCKASQNVGTNVAWFQQKPGKAPKALIYSASYRYSGVPSRFSGSGSGTDFTLTISSLQPEDFATYYCQQYNISPYTFGQGTKLEIK

SEQ ID NO: 179 - protein, 107 aa, synthetic construct
DIQMTQSPSSLSASVGDRVTITCKASQNVGTNVAWFQQKPGKAPKALIYSASYRYSGVPSRFSGSGSGTDFTLTISSLQPEDFATYYCQQYQISPYTFGQGTKLEIK

SEQ ID NO: 180 - protein, 116 aa, synthetic construct
QVQLVQSGAEVKKPGASVKVSCKASGYTFTESTMYWVRQAPGQGLEWIGHINPNQGGTSYNQKFRGRVTMTRDTSISTAYMELSRLRSDDTAVYYCAMELFYFDYWGQGTLVTVSS

SEQ ID NO: 181 - protein, 10 aa, synthetic construct
  VARIANT 10: note = Y or H
GYTFTESTMX

SEQ ID NO: 182 - protein, 10 aa, synthetic construct
GYTFTESTMH

SEQ ID NO: 183 - protein, 17 aa, synthetic construct
  VARIANT 1: note = H or R
  VARIANT 6: note = N or Q
  VARIANT 10: note = N or S
  VARIANT 12: note = N or A
  VARIANT 16: note = R or Q
XINPNXGGTXYXQKFXG

SEQ ID NO: 184 - protein, 17 aa, synthetic construct
RINPNNGGTNYAQKFQG

SEQ ID NO: 185 - protein, 17 aa, synthetic construct
HINPNQGGTSYNQKFRG

SEQ ID NO: 186 - protein, 9 aa, synthetic construct
  VARIANT 4: note = N or Q
  VARIANT 5: note = I or S
  VARIANT 6: note = S or Y
  VARIANT 8: note = Y or L
QQYXXXPXT

SEQ ID NO: 187 - protein, 9 aa, synthetic construct
QQYQISPYT

SEQ ID NO: 188 - protein, 481 aa, synthetic construct
QVQLQESGPGLVKPSQTLSLTCTVSGDSITSGYWNWIRQHPGKGLEYMGYISYSGSTYYNPSLKSRVTISVDTSKNQFSLKLSSVTAADTAVYYCARYYGDNYVGAMDYWGQGTLVTVSSGGGGSGGGGSGGGGSGGGGSDIVMTQSPLSLPVTPGEPASISCRSSKSLLHSNGITYLYWYLQKPGQSPQLLIYQMSNLASGVPDRFSGSGSGTDFTLKISRVEAEDVGVYYCAQNLELPWTFGGGTKVEIKGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 189 - protein, 738 aa, synthetic construct
QVQLQESGPGLVKPSQTLSLTCTVSGDSITSGYWNWIRQHPGKGLEYMGYISYSGSTYYNPSLKSRVTISVDTSKNQFSLKLSSVTAADTAVYYCARYYGDNYVGAMDYWGQGTLVTVSSGGGGSGGGGSGGGGSGGGGSDIVMTQSPLSLPVTPGEPASISCRSSKSLLHSNGITYLYWYLQKPGQSPQLLIYQMSNLASGVPDRFSGSGSGTDFTLKISRVEAEDVGVYYCAQNLELPWTFGGGTKVEIKGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSQVQLQESGPGLVKPSQTLSLTCTVSGDSITSGYWNWIRQHPGKGLEYMGYISYSGSTYYNPSLKSRVTISVDTSKNQFSLKLSSVTAADTAVYYCARYYGDNYVGAMDYWGQGTLVTVSSGGGGSGGGGSGGGGSGGGGSDIVMTQSPLSLPVTPGEPASISCRSSKSLLHSNGITYLYWYLQKPGQSPQLLIYQMSNLASGVPDRFSGSGSGTDFTLKISRVEAEDVGVYYCAQNLELPWTFGGGTKVEIK

SEQ ID NO: 190 - protein, 738 aa, synthetic construct
QVQLQESGPGLVKPSQTLSLTCTVSGDSITSGYWNWIRQHPGKGLEYMGYISYSGSTYYNPSLKSRVTISVDTSKNQFSLKLSSVTAADTAVYYCARYYGDNYVGAMDYWGQGTLVTVSSGGGGSGGGGSGGGGSGGGGSDIVMTQSPLSLPVTPGEPASISCRSSKSLLHSNGITYLYWYLQKPGQSPQLLIYQMSNLASGVPDRFSGSGSGTDFTLKISRVEAEDVGVYYCAQNLELPWTFGGGTKVEIKGGGGSQVQLQESGPGLVKPSQTLSLTCTVSGDSITSGYWNWIRQHPGKGLEYMGYISYSGSTYYNPSLKSRVTISVDTSKNQFSLKLSSVTAADTAVYYCARYYGDNYVGAMDYWGQGTLVTVSSGGGGSGGGGSGGGGSGGGGSDIVMTQSPLSLPVTPGEPASISCRSSKSLLHSNGITYLYWYLQKPGQSPQLLIYQMSNLASGVPDRFSGSGSGTDFTLKISRVEAEDVGVYYCAQNLELPWTFGGGTKVEIKGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 191 - protein, 995 aa, synthetic construct
QVQLQESGPGLVKPSQTLSLTCTVSGDSITSGYWNWIRQHPGKGLEYMGYISYSGSTYYNPSLKSRVTISVDTSKNQFSLKLSSVTAADTAVYYCARYYGDNYVGAMDYWGQGTLVTVSSGGGGSGGGGSGGGGSGGGGSDIVMTQSPLSLPVTPGEPASISCRSSKSLLHSNGITYLYWYLQKPGQSPQLLIYQMSNLASGVPDRFSGSGSGTDFTLKISRVEAEDVGVYYCAQNLELPWTFGGGTKVEIKGGGGSQVQLQESGPGLVKPSQTLSLTCTVSGDSITSGYWNWIRQHPGKGLEYMGYISYSGSTYYNPSLKSRVTISVDTSKNQFSLKLSSVTAADTAVYYCARYYGDNYVGAMDYWGQGTLVTVSSGGGGSGGGGSGGGGSGGGGSDIVMTQSPLSLPVTPGEPASISCRSSKSLLHSNGITYLYWYLQKPGQSPQLLIYQMSNLASGVPDRFSGSGSGTDFTLKISRVEAEDVGVYYCAQNLELPWTFGGGTKVEIKGGGGSQVQLQESGPGLVKPSQTLSLTCTVSGDSITSGYWNWIRQHPGKGLEYMGYISYSGSTYYNPSLKSRVTISVDTSKNQFSLKLSSVTAADTAVYYCARYYGDNYVGAMDYWGQGTLVTVSSGGGGSGGGGSGGGGSGGGGSDIVMTQSPLSLPVTPGEPASISCRSSKSLLHSNGITYLYWYLQKPGQSPQLLIYQMSNLASGVPDRFSGSGSGTDFTLKISRVEAEDVGVYYCAQNLELPWTFGGGTKVEIKGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 192 - protein, 995 aa, synthetic construct
QVQLQESGPGLVKPSQTLSLTCTVSGDSITSGYWNWIRQHPGKGLEYMGYISYSGSTYYNPSLKSRVTISVDTSKNQFSLKLSSVTAADTAVYYCARYYGDNYVGAMDYWGQGTLVTVSSGGGGSGGGGSGGGGSGGGGSDIVMTQSPLSLPVTPGEPASISCRSSKSLLHSNGITYLYWYLQKPGQSPQLLIYQMSNLASGVPDRFSGSGSGTDFTLKISRVEAEDVGVYYCAQNLELPWTFGGGTKVEIKGGGGSQVQLQESGPGLVKPSQTLSLTCTVSGDSITSGYWNWIRQHPGKGLEYMGYISYSGSTYYNPSLKSRVTISVDTSKNQFSLKLSSVTAADTAVYYCARYYGDNYVGAMDYWGQGTLVTVSSGGGGSGGGGSGGGGSGGGGSDIVMTQSPLSLPVTPGEPASISCRSSKSLLHSNGITYLYWYLQKPGQSPQLLIYQMSNLASGVPDRFSGSGSGTDFTLKISRVEAEDVGVYYCAQNLELPWTFGGGTKVEIKGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSQVQLQESGPGLVKPSQTLSLTCTVSGDSITSGYWNWIRQHPGKGLEYMGYISYSGSTYYNPSLKSRVTISVDTSKNQFSLKLSSVTAADTAVYYCARYYGDNYVGAMDYWGQGTLVTVSSGGGGSGGGGSGGGGSGGGGSDIVMTQSPLSLPVTPGEPASISCRSSKSLLHSNGITYLYWYLQKPGQSPQLLIYQMSNLASGVPDRFSGSGSGTDFTLKISRVEAEDVGVYYCAQNLELPWTFGGGTKVEIK

SEQ ID NO: 193 - protein, 995 aa, synthetic construct
QVQLQESGPGLVKPSQTLSLTCTVSGDSITSGYWNWIRQHPGKGLEYMGYISYSGSTYYNPSLKSRVTISVDTSKNQFSLKLSSVTAADTAVYYCARYYGDNYVGAMDYWGQGTLVTVSSGGGGSGGGGSGGGGSGGGGSDIVMTQSPLSLPVTPGEPASISCRSSKSLLHSNGITYLYWYLQKPGQSPQLLIYQMSNLASGVPDRFSGSGSGTDFTLKISRVEAEDVGVYYCAQNLELPWTFGGGTKVEIKGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSQVQLQESGPGLVKPSQTLSLTCTVSGDSITSGYWNWIRQHPGKGLEYMGYISYSGSTYYNPSLKSRVTISVDTSKNQFSLKLSSVTAADTAVYYCARYYGDNYVGAMDYWGQGTLVTVSSGGGGSGGGGSGGGGSGGGGSDIVMTQSPLSLPVTPGEPASISCRSSKSLLHSNGITYLYWYLQKPGQSPQLLIYQMSNLASGVPDRFSGSGSGTDFTLKISRVEAEDVGVYYCAQNLELPWTFGGGTKVEIKGGGGSQVQLQESGPGLVKPSQTLSLTCTVSGDSITSGYWNWIRQHPGKGLEYMGYISYSGSTYYNPSLKSRVTISVDTSKNQFSLKLSSVTAADTAVYYCARYYGDNYVGAMDYWGQGTLVTVSSGGGGSGGGGSGGGGSGGGGSDIVMTQSPLSLPVTPGEPASISCRSSKSLLHSNGITYLYWYLQKPGQSPQLLIYQMSNLASGVPDRFSGSGSGTDFTLKISRVEAEDVGVYYCAQNLELPWTFGGGTKVEIK

SEQ ID NO: 194 - protein, 16 aa, synthetic construct
  VARIANT 1: note = R or K
  VARIANT 11: note = G, K or R
  VARIANT 16: note = Y, D or A
XSSKSLLHSQXITYLX

SEQ ID NO: 195 - protein, 16 aa, synthetic construct
  VARIANT 1: note = R or K
  VARIANT 10: note = N, Q or D
  VARIANT 11: note = K or R
  VARIANT 16: note = Y, D or A
XSSKSLLHSXXITYLX

SEQ ID NO: 196 - protein, 17 aa, synthetic construct
  VARIANT 7: note = G or V
  VARIANT 10: note = K or N
  VARIANT 12: note = N or A
  VARIANT 13: note = E or Q
  VARIANT 16: note = K or Q
WIFPGEXSTXYXXKFXG

SEQ ID NO: 197 - protein, 17 aa, synthetic construct
  VARIANT 6: note = D or E
  VARIANT 10: note = K or N
  VARIANT 12: note = N or A
  VARIANT 13: note = E or Q
  VARIANT 16: note = K or Q
WIFPGXVSTXYXXKFXG

SEQ ID NO: 198 - protein, 19 aa, Mus sp.
MGWSCIILFLVATATGVHS

SEQ ID NO: 199 - protein, 20 aa, Mus sp.
METDTLLLWVLLLWVPGSTG

SEQ ID NO: 200 - protein, 4 aa, synthetic construct
GGGG

SEQ ID NO: 201 - protein, 6 aa, synthetic construct
HHHHHH

SEQ ID NO: 202 - protein, 109 aa, synthetic construct
EVQLQESGPSLVKPSQTLSLTCSVTGDSITSGYWNWIRKFPGNKLEYMGYISYSGSTYYNPSLKSRISITRDTSKNQYSLHLNSVTAEDTATFYCARYYGDNYVGAMDY

SEQ ID NO: 203 - protein, 109 aa, synthetic construct
DVQLQESGPSLVKPSQALSLTCSVTGDSITSGYWNWIRKFPGNKLEYLGYISYSGSTYYNPSLKSRISITRDTSKNQFYLQLNSMTTEDTATYFCVRYPYNGHNGYLDY

SEQ ID NO: 204 - protein, 102 aa, synthetic construct
DIVMTQAAFSNPVTLGISASISCRSSKSLLHSNGITYLYWYLQKPGQSPQLLIYQMSNLASGVPDRFSSSGSGTDFTLRISRVEAEDVGVYYCAQNLELPWT

SEQ ID NO: 205 - protein, 102 aa, synthetic construct
DVVMTQATFSNPVTLGTSASISCRSSKSLLHRNGITYLYWFLQKPGQSPQLLIYQMSNLASGVPDRFSSSGSGTDFTLRISRVEAEDVGVYYCAQNLELPWT

SEQ ID NO: 206 - protein, 111 aa, synthetic construct
QVQLQQSGAELVKPGASVKLSCKASGYTFTNYDINWVRQRPEQGLEWIGWIFPGDGSTKYNEKFKGKATLTTDKSSSTAYMQLSRLTSEDSAVFFCATELVKDYYAMDYWG

SEQ ID NO: 207 - protein, 107 aa, synthetic construct
QVRLQQSGPELVKPGASVRISCKASGHTFTSYYIYWVKQRPGQGLEWIGWIYFGNINLKYNEKFKGKATLTADKSSSTAYIQLSSLTSEDSAVYFCASEDGPGAYWG

SEQ ID NO: 208 - protein, 97 aa, synthetic construct
QVQLQQSGAELVKPGASVKLSCKASGYTFTNYNIHWVRQRPEQGLEWIGWIFPGDGSTKYNEKFKGKTTLTTDKSSSTAYMQLSRLTSEDSAVFFCA

SEQ ID NO: 209 - protein, 101 aa, synthetic construct
DIVMTQSQKFMSTSVGDRVSVTCKASQNVGTNVAWYQQKPGQSPKALIYSASYRYSGVPDRFTGSGSGTDFTLTISNVQSEDLAEYFCQQYNSYPYTFGGG

SEQ ID NO: 210 - protein, 101 aa, synthetic construct
DIVMTQSQKFMSTSVGDRVSVTCKASQNVGTNVAWYQQKPGQSPKALIYSASYRYSGVPDRFTGSGSGTDFTLTISNVQSEDLAEYFCQQYNSYPLTFGAG

SEQ ID NO: 211 - protein, 95 aa, synthetic construct
DIVMTQSQKFMSTSVGDRVSVTCKASQNVGTNVAWYQQKPGQSPKALIYSASYRYSGVPDRFTGSGSGTDFTLTISNVQSEDLAEYFCQQYNSYP

SEQ ID NO: 212 - protein, 111 aa, synthetic construct
QVQLVQSGAEVKKPGASVKVSCKASGYTFTNYDINWVRQAPGQGLEWIGWIFPGEGSTKYNEKFKGRVTMTRDTSISTAYMELSRLRSDDTAVYYCATELVKDYYAMDYWG

SEQ ID NO: 213 - protein, 97 aa, synthetic construct
QVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCA

SEQ ID NO: 214 - protein, 101 aa, synthetic construct
DIQMTQSPSSLSASVGDRVTITCKASQNVGTNVAWFQQKPGKAPKALIYSASYRYSGVPSRFSGSGSGTDFTLTISSLQPEDFATYYCQQYNSYPLTFGQG

SEQ ID NO: 215 - protein, 95 aa, synthetic construct
DIQMTQSPSSLSASVGDRVTITCRASQGISNYLAWFQQKPGKAPKSLIYAASSLQSGVPSRFSGSGSGTDFTLTISSLQPEDFATYYCQQYNSYP

(19) United States
(12) Patent Application Publication
(10) Pub. No.: US 2026/0146105 A1
(43) Pub. Date: May 28, 2026

(54) ANTIBODIES AGAINST FENTANYL AND ANALOGS AND METHODS OF USE THEREOF

(71) Applicant: Regents of the University of Minnesota, Minneapolis, MN (US)

(72) Inventors: Marco PRAVETONI, Minneapolis, MN (US); Carly BAEHR, Minneapolis, MN (US); Dustin HICKS, Minneapolis, MN (US)

(73) Assignee: Regents of the University of Minnesota, Minneapolis, MN (US)

(21) Appl. No.: 19/109,890

(22) PCT Filed: Sep. 11, 2023
(86) PCT No.: PCT/US2023/032401

§ 371 Date: Mar. 7, 2025

Related U.S. Application Data
(60) Provisional application No. 63/405,244, filed on Sep. 9, 2022.

(51) Int. Cl. C07K 16/44; A61K 39/00; A61P 43/00

(52) U.S. Cl. CPC C07K 16/44; A61P 43/00; A61K 2039/505; C07K 2317/565; C07K 2317/76; C07K 2317/92

(57) ABSTRACT

Certain embodiments provide an isolated antibody or fragment thereof having affinity for fentanyl or analog thereof, CA as well as methods of use thereof. Certain embodiments provide a method for treating fentanyl or analog related overdose, poisoning, or disorder.

### CROSS REFERENCE TO RELATED APPLICATION

This application is a 35 U.S.C. § 371 application of International Application No. PCT/US2023/032401, filed on 11 Sep. 2023, which claims priority to U.S. Provisional Application No. 63/405,244 filed on 9 Sep. 2022. The entire content of the application referenced above is hereby incorporated by reference herein.

### GOVERNMENT FUNDING

This invention was made with government support under DA051658, and DA057850 awarded by the National Institutes of Health. The government has certain rights in the invention.

### SEQUENCE LISTING

The instant application contains a Sequence Listing that has been submitted in XML format via Patent Center and is hereby incorporated by reference in its entirety. Said XML copy, created on Nov. 7, 2023, is named 09531_550W01_SL and is 219,104 Bytes in size.

### BACKGROUND OF THE INVENTION

Fatal overdoses involving opioids are at an all-time high in the United States, and accidental overdose deaths involving synthetic opioids have increased annually since 2013. In addition, fentanyl and its analogs have been involved in cases of poisoning, and deliberate exposures. Fentanyl, an increasingly common and one of the most potent synthetic opioids, is associated with a significant number of overdose deaths: between May 2020 and May 2021, 62,136 overdose deaths associated with synthetic opioids were reported, accounting for 84.8% of all opioid-related overdose deaths. Between 2022 and present, drug fatal overdoses have surpassed>100,000/yr in United States, and ˜75% of cases involved fentanyl or its analogs, presented alone or in combination with other drugs (e.g., fentanyl/methamphetamine, fentanyl/xylazine). In addition, there is the potential for deliberate use of synthetic opioids in chemical warfare or poisoning events, as shown by the Moscow Theater hostage siege by terrorists, which was resolved by the Russian military by means of pumping aerosolized carfentanil/remifentanil mixtures through the ventilation system, causing the death of over 150 civilians. The current clinically approved treatments for reversal of opioid-related toxicity are naloxone, a μ-opioid receptor (MOR) antagonist, and nalmefene, a longer-acting MOR antagonist. These medications may also be used in Mass Casualty Incidents (MCI) or deliberate exposures such as chemical attacks or poisonings involving carfentanil and other ultrapotent synthetic opioids. Naloxone is typically effective for combating opioid toxicity and overdose; however, for MOR agonists that exhibit higher potency and a serum half-life longer than that of naloxone (30-90 minutes), additional doses of naloxone may be required to reverse overdose and protect against renarcotization. Specifically, fentanyl has a serum half-life of −8 hrs; consequently, additional dosing of naloxone and extended monitoring for signs of recurring fentanyl toxicity can be needed for 2 or more hours. Other fentanyl analogs may also display unique PK/PD profiles and overdose toxicity, which are not effectively counteracted by existing medications. The drastic and sustained increase in overdose deaths related to synthetic opioids indicates that current methods of intervention are inadequate; therefore, the development of alternative or complementary treatment options is needed. Alternative or complementary strategies that could be deployed in post-exposure treatment in a variety of additional exposure scenarios, including chemical attacks, poisoning or assassination attempts involving ultrapotent synthetic opioids such as carfentanil are also needed.

### SUMMARY OF THE INVENTION

Certain embodiments of the invention provide an isolated antibody specific for fentanyl and its analog(s), or fragment thereof (e.g., HY6-F9 family), comprising one or more complementarity determining regions (CDRs) selected from the group consisting of:
(a) a heavy chain CDR1 having at least 75% sequence identity to an amino acid sequence of any one of GDSITSGYWN (SEQ ID NO:62) and GDSITSGYWS (SEQ ID NO:63);
(b) a heavy chain CDR2 having at least 75% sequence identity to an amino acid sequence of YISYSGSTYYNPSLKS (SEQ ID NO:64);
(c) a heavy chain CDR3 having at least 75% sequence identity to an amino acid sequence of any one of ARYYGDNYVGAMDY (SEQ ID NO:65), ARYYGDNYVGALDY (SEQ ID NO:161), ARYYGDNYVGAQDY (SEQ ID NO:162), ARYYGDNYVGAIDY (SEQ ID NO:163), and ARYYGDNYVGAADY (SEQ ID NO:164);
(d) a light chain CDR1 having at least 75% sequence identity to an amino acid sequence of any one of RSSKSLLHSNGITYLY (SEQ ID NO:67), RSSKSLLHSNGITYLD (SEQ ID NO:68), KSSKSLLHSNGITYLA (SEQ ID NO:69), RSSKSLLHSQGITYLY (SEQ ID NO:70), RSSKSLLHSNKITYLY (SEQ ID NO:71), RSSKSLLHSNRITYLY (SEQ ID NO:72), and RSSKSLLHSDGITYLY (SEQ ID NO:73);
(e) a light chain CDR2 having at least 75% sequence identity to an amino acid sequence of any one of QMSNLAS (SEQ ID NO:75), QMSNRAS (SEQ ID NO:76), and QMSNRES (SEQ ID NO:77); and
(f) a light chain CDR3 having at least 75% sequence identity to an amino acid sequence of AQNLELPWT (SEQ ID NO:78).

Certain embodiments of the invention provide an isolated antibody specific for fentanyl and its analog(s), or fragment thereof (e.g., HY11-7E1 family), comprising one or more complementarity determining regions (CDRs) selected from the group consisting of:
(a) a heavy chain CDR1 having at least 75% sequence identity to an amino acid sequence of any one of GYTFTNYDIN (SEQ ID NO:80) and GYTFTNYDMH (SEQ ID NO:81);
(b) a heavy chain CDR2 having at least 75% sequence identity to an amino acid sequence of any one of WIFPGDGSTKYNEKFKG (SEQ ID NO:83), WIFPGDGSTNYAQKFQG (SEQ ID NO:84), WIFPGEGSTKYNEKFKG (SEQ ID NO:85), and WIFPGDVSTKYNEKFKG (SEQ ID NO:86);
(c) a heavy chain CDR3 having at least 75% sequence identity to an amino acid sequence of ATELVKDYYAMDY (SEQ ID NO:87);
(d) a light chain CDR1 having at least 75% sequence identity to an amino acid sequence of any one of KASQNVGTNVA (SEQ ID NO:89) and RASQNVGTNLA (SEQ ID NO:90)
(e) a light chain CDR2 having at least 75% sequence identity to an amino acid sequence of any one of SASYRYS (SEQ ID NO:92), and SASYLQS (SEQ ID NO:93); and (f) a light chain CDR3 having at least 75% sequence identity to an amino acid sequence of any one of QQYNSYPYT (SEQ ID NO:95), QQYYNYPYT (SEQ ID NO:96), and QQYNSYPLT (SEQ ID NO:97).

Certain embodiments of the invention provide an isolated antibody specific for fentanyl and its analog(s), or fragment thereof (e.g., HY19-1H6 family), comprising one or more complementarity determining regions (CDRs) selected from the group consisting of:
(a) a heavy chain CDR1 having at least 75% sequence identity to an amino acid sequence of any one of GYTFTESTMY (SEQ ID NO:121) and GYTFTESTMH (SEQ ID NO:182);
(b) a heavy chain CDR2 having at least 75% sequence identity to an amino acid sequence of any one of HINPNNGGTSYNQKFRG (SEQ ID NO:122), RINPNNGGTNYAQKFQG (SEQ ID NO:184), and HINPNQGGTSYNQKFRG (SEQ ID NO:185);
(c) a heavy chain CDR3 having at least 75% sequence identity to an amino acid sequence of AMELFYFDY (SEQ ID NO:123);
(d) a light chain CDR1 having at least 75% sequence identity to an amino acid sequence of KASQNVGTNVA (SEQ ID NO:89);
(e) a light chain CDR2 having at least 75% sequence identity to an amino acid sequence of SASYRYS (SEQ ID NO:92); and
(f) a light chain CDR3 having at least 75% sequence identity to an amino acid sequence of any one of QQYNISPYT (SEQ ID NO:124), QQYQISPYT (SEQ ID NO:187), and QQYNSYPLT (SEQ ID NO:97).

Certain embodiments of the invention provide an isolated antibody specific for fentanyl and its analog(s), or fragment thereof, comprising one or more complementarity determining regions (CDRs) selected from the group consisting of:
(a) a heavy chain CDR1 having at least 75% sequence identity to an amino acid sequence of any one CDR1 sequence listed in Tables A1-A2, Tables B1-B2, Tables B3-B4, or Tables C1-C2;
(b) a heavy chain CDR2 having at least 75% sequence identity to an amino acid sequence of any one CDR2 sequence listed in Tables A1-A2, Tables B1-B2, Tables B3-B4, or Tables C1-C2;
(c) a heavy chain CDR3 having at least 75% sequence identity to an amino acid sequence of any one CDR3 sequence listed in Tables A1-A2, Tables B1-B2, Tables B3-B4, or Tables C1-C2;
(d) a light chain CDR1 having at least 75% sequence identity to an amino acid sequence of any one CDR1 sequence listed in Tables A1-A2, Tables B1-B2, Tables B3-B4, or Tables C1-C2;
(e) a light chain CDR2 having at least 75% sequence identity to an amino acid sequence of any one CDR2 sequence listed in Tables A1-A2, Tables B1-B2, Tables B3-B4, or Tables C1-C2; and
(f) a light chain CDR3 having at least 75% sequence identity to an amino acid sequence of any one CDR3 sequence listed in Tables A1-A2, Tables B1-B2, Tables B3-B4, or Tables C1-C2.

Certain embodiments of the invention provide an isolated anti-fentanyl antibody or fragment thereof, comprises a heavy chain variable region comprising an amino acid sequence that has at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to the amino acid sequence of any VH sequence listed in Table A1, Table B1, Table B3, or Table C1, and a light chain variable region comprising an amino acid sequence that has at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to the amino acid sequence of any VL sequence listed in Table A1, Table B1, Table B3, or Table C1.

Certain embodiments of the invention provide a composition comprising an isolated antibody, or fragment thereof, as described herein and a carrier.

Certain embodiments of the invention provide an isolated polynucleotide comprising a nucleotide sequence encoding the isolated antibody or fragment thereof as described herein.

Certain embodiments of the invention provide a method of inhibiting the activity of fentanyl or analog(s) in a mammal, comprising administering an isolated antibody or fragment thereof as described herein to the mammal.

Certain embodiments of the invention provide a method for treating fentanyl or analog(s) related overdose, poisoning, or disorder in a mammal, comprising administering an effective amount of an isolated antibody or fragment thereof as described herein to the mammal.

Certain embodiments of the invention provide an isolated antibody or fragment thereof as described herein for the prophylactic or therapeutic treatment of fentanyl or analog(s) related overdose, poisoning, or disorder.

Certain embodiments of the invention provide the use of an isolated antibody or fragment thereof as described herein to prepare a medicament for the treatment of fentanyl or analog related overdose, poisoning, or disorder in a mammal.

Certain embodiments of the invention provide the use of an isolated antibody or fragment thereof as described herein to prepare a medicament for the treatment of fentanyl or analog related to overdose, poisoning, or opioid use disorder in a mammal exposed to mixtures of opioids (e.g., heroin/fentanyl) or mixtures of opioids and non-opioids (e.g., fentanyl/methamphetamine or fentanyl/xylazine).

Certain embodiments of the invention provide a kit comprising an isolated antibody or fragment thereof, packaging material, and instructions for administering the antibody or a fragment thereof to a mammal to treat fentanyl or analog related overdose, poisoning, or disorder.

### BRIEF DESCRIPTION OF THE FIGURES

FIGS. 1A-1B. Process of anti-fentanyl mAb humanization. (FIG. 1A) Cartoon depiction of a humanized mAb with antibody regions denoted. (FIG. 1B). Stepwise murine antibody humanization scheme: (I) murine hybridoma derived mAb, (II) chimerization onto human IgG1/IgK backbone, (III) humanization of HC only, via CDR grafting, (IV) combination of binding confirmed humanized HC from (III) and humanized LC resulting in a fully humanized mAb.

FIGS. 2A-2D. In vivo efficacy against fentanyl of murine mAbs. Mice (n=4 per group, 2 males and 2 females) were passively immunized with anti-fentanyl mAb (40 mg/kg, s.c.), and 24 hours later were challenged with 0.25 mg/kg fentanyl. Fentanyl-induced effects on: FIG. 2A) antinociception measured by hot plate; FIG. 2B) heart rate and FIG. 2C) breath rate measured by pulse oximetry. FIG. 2D) One week after challenge, serum concentration of mAb measured by ELISA. Data are expressed as mean±SEM; *p<0.05; **p<0.01; ***p<0.001.

FIGS. 3A-3C. In vivo comparison of murine and chimeric anti-fentanyl mAbs. Mice (n=3 male mice per group) were passively immunized with anti-fentanyl mAb (40 mg/kg, s.c.), and 24 hours later were challenged with 0.1 mg/kg fentanyl. Concentration of fentanyl in FIG. 3A) brain and FIG. 3B) serum measured by LCMS. FIG. 3C) Serum concentration of mAb and chAb prior to fentanyl challenge measured by ELISA. Data are expressed as mean±SEM; *p<0.05; ***p<0.001, ****p<0.0001.

FIGS. 4A-4F. In vivo comparison of murine and chimeric HY6-F9 in rats. Rats (n=3-4 male rats per group) were passively immunized with anti-fentanyl mAb (40 mg/kg, i.p.), and 24 hours later were challenged with 0.1 mg/kg fentanyl. FIG. 4A) Fentanyl-induced antinociception as latency to respond on a hot plate; FIG. 4B) oxygen saturation, FIG. 4C) heart rate, and FIG. 4D) breath rate measured by pulse oximetry. Concentrations of fentanyl in FIG. 4E) brain and FIG. 4F) serum measured by LCMS. Data are expressed as mean±SEM; *p<0.05; **p<0.01; ***p<0.001.

FIGS. 5A-5B. DSF Fab Tₘ comparison of chimeric and humanized mAb. HY6-F9 mAbs (FIG. 5A) and HY11-7E1 mAbs (FIG. 5B) at 1 mg/mL in PBS, pH 7.4 were combined with Protein Thermal Shift™ assay reagents and subjected to a continuous 0.3% (0.45° C./min) temperature ramp from 25 to 95° C. The Tₘ of each mAb fragment is determined by the temperature measurement at the derivative peak (dPeak). The initial, broad dPeak in each sample corresponds to the CH2domain, while the second dPeak in each sample corresponds to the Fab domain. All humanized HY6-F9 and HY11-7E1 mAbs show an increased temperature shift in Fab Tₘ upon humanization, indicating increased thermal stability compared to the murine chimeric counterpart.

FIGS. 6A-6G. Efficacy of humanized mAb in rats. Rats (n=4 male rats per group) were passively immunized with saline, HY6-F9_Ch as positive control, HY6-F9_Hu (NQ), or HY11-7E1_Hu (DE) (40 mg/kg), and 24 hours later were challenged with cumulative fentanyl doses up to 0.3 mg/kg. FIG. 6A) Fentanyl-induced antinociception as latency to respond on a hot plate; FIG. 6B) oxygen saturation, and FIG. 6C) heart rate measured by pulse oximetry. FIG. 6D) Concentration of mAb 1 hour prior to challenge measured by ELISA; and concentrations of fentanyl in FIG. 6E) serum and FIG. 6F) brain, and FIG. 6G) concentration of norfentanyl in serum 15 min after final fentanyl dose measured by LCMS. Data are expressed as mean±SEM; *p<0.05; **p<0.01; ***p<0.001; ****p<0.0001.

FIG. 7. Fab Derivative DSF Peak Isolation.

FIG. 8. Fab Tₘ comparison of anti-fentanyl mAbs to 137 FDA approved therapeutic mAbs and mAbs in 2nd or 3rd phase clinical trials from Jain T, et al., PNAS (2017) 114:944-9.

FIG. 9. Relative affinity of mAb for fentanyl and other target and off-target compounds as determined by competitive ELISA. *indicates no detectable binding; detection limit denoted as the highest concentration of competitor used.

FIGS. 10A-10C. Structure of HY6-F9.6 Fab in complex with fentanyl. FIG. 10A) Top down and cutaway side view surface representation of the HY6-F9.6 binding pocket, shown with fentanyl within the box. FIG. 10B) Right, the binding pocket is shown in depth. Dark bonds correspond to hydrogen bonds, light grey to van der Waals interactions, and black bonds (within oval) to intra-Fab hydrogen or salt bridge bonds. FIG. 1C) CSA plot of the fentanyl ligand and HY6-F9.6 residues, with a sequence alignment of HY6-F9.6 and P1C3H9 shown below. Residues involved in hydrogen bonding are marked with a “H”. Dots indicate conserved residues, dashes indicate gaps in the aligned sequence, and the numbering and CDRs are true to HY6-F9.6. FIG. 10C discloses SEQ ID NOS: 202-205, respectively, in order of appearance.

FIGS. 11A-11D. Effects of an anti-fentanyl mAb to reverse fentanyl-induced apnea. Hanford mini-pigs (n=3/group) were anesthetized with isoflurane to a light plane of anesthesia and received an infusion of fentanyl (10 μg/kg/min) until production of 2 min of apnea. At this point, saline 0.1 ml/kg, naloxone 10 μg/kg, or mAb (HY6-F9.6) 40 mg/kg was administered intravenously, and the fentanyl infusion was discontinued. Respiratory rate and tidal volume were determined every minute for 30 minutes following administration of test article. Latency to return to spontaneous breathing was determined. FIG. 11A shows fentanyl induced respiratory rate reduction was reversed by post-exposure administration of anti-fentanyl mAb. FIG. 11B shows fentanyl induced tidal volume reduction was reversed by post-exposure administration of anti-fentanyl mAb. FIG. 11C shows fentanyl induced end-tidal carbon dioxide (EtCO₂) reduction was reversed by post-exposure administration of anti-fentanyl mAb. FIG. 11D shows fentanyl induced minute volume reduction was reversed by post-exposure administration of anti-fentanyl mAb. All FIG. 11 panels show that mAb's effect was equivalent (not statistically different) to naloxone's effect in reversing pharmacological effects of fentanyl.

FIGS. 12A-12C. Fentanyl in plasma after mAb HY6-F9.6 administration in pigs. Blood samples were collected prior to fentanyl administration (baseline), after apnea, and at 1-30 min after reversal. Fentanyl concentration was quantitated by LC-MS. FIG. 12A shows antibody group as compared to the saline group. FIG. 12B shows antibody group as compared to the saline group or the naloxone group. FIG. 12C shows experimental set up and timeline.

FIG. 13. Potency ELISA study of stability samples.

FIG. 14. EC50 values from potency ELISA study of stability samples.

FIG. 15. Potency ELISA study of forced stress samples.

FIG. 16. EC50 values from potency ELISA study of forced stress samples.

FIG. 17. Anti-fentanyl mAb blocks mu-opioid receptor (MOR) signaling in vitro.

Cells stably expressing human MOR (Gai6-CHO-hMOR) were incubated with 30-300 nM fentanyl with or without a 2-fold excess of anti-fentanyl mAb (HY6-F9.19), and MOR activation-induced Ca²⁺ signal was quantitated with Calcium 5 dye. Data are mean±SEM from 3 replicates, expressed as signal relative to fentanyl alone signal.

FIG. 18. Anti-fentanyl mAb blocks mu-opioid receptor (MOR) signaling in vitro.

Cells stably expressing human MOR (Gai6-CHO-hMOR) were incubated with 30 nM fentanyl with or without a 3-fold excess of anti-fentanyl mAb (HY6-F9.19, HY11-7E1.17, or HY19-1H6.7), and MOR activation-induced Ca²⁺ signal was quantitated with Calcium 5 dye. Data are mean±SEM from 3 replicates, expressed as signal relative to fentanyl alone signal.

FIG. 19. Reversal of fentanyl with mAb 20 mg/kg in non-human primate (NHP). Data shown are representative of n=4 subjects.

FIGS. 20A-20D. Proof of scalability of mAb at a CRO: anti-fentanyl mAb was produced from CHO stable pool and tested for in vitro binding equivalency to in-house mAb.

FIG. 20A, titer; FIG. 20B, binding to fentanyl; FIG. 20C, binding to fentanyl analogs; FIG. 20D, binding to naloxone and naltrexone.

FIG. 21. Proof of scalability: in vivo efficacy in mice was assessed after challenge with 0.3 mg/mL fentanyl. Concentration of fentanyl in brain and serum was quantitated by LCMS.

FIG. 22. Reversal of fentanyl-induced respiratory depression in non-human primate (NHP). Fentanyl was administered to induce respiratory depression (V_{E}<70%), followed by treatment using saline, or NLX (32 μg/Kg), or mAb (HY6-F9.19)(20 mg/kg). Minute Ventilation (V_{E})=Respiratory Rate x Tidal Volume. Rhesus macaque was equipped with helmet connected to a pneumotachometer.

FIG. 23. SDS-PAGE data of HY19-1H clones (2 μg loaded).

FIGS. 24A-24D. Structures of HY11-7E1.1 and HY11-6B2.1 in complex with fentanyl. (FIG. 24A) Structure of HY11-7E1.1 in complex with fentanyl. (FIG. 24B) Structure of HY11-6B2.1 in complex with fentanyl. (FIG. 24C) Overlay of binding pockets of HY11-7E1.1 and HY11-6B2.1 containing fentanyl. (FIG. 24D) 6B2.1 vs 7E1.1 contact surface area by VH residue or VL residue. FIG. 24D discloses SEQ ID NOS: 206-211, respectively, in order of appearance.

FIGS. 25A-25D. Structure of HY11-7E1.17 apo and in complex with carfentanil. (FIG. 25A) Structure of HY11-7E1.17 apo. (FIG. 25B) Structure of HY11-7E1.17 in complex with carfentanil. (FIG. 25C) Overlay of HY11-7E1.17 binding pocket apo form and in complex with carfentanil. (FIG. 25D) HY11-7E1.1 and HY11-7E1.17 contact surface area by VH residue or VL residue. FIG. 25D discloses SEQ ID NOS: 212, 206, 213-214, 209 and 215, respectively, in order of appearance.

FIGS. 26A-26I. Efficacy of anti-fentanyl mAb against fentanyl, acetylfentanyl, and carfentanil. Mice (Balb/c, n=3/group) were passively immunized with mAb 40 mg/kg, s.c., then 24 hours later challenged with (FIGS. 26A-26C) fentanyl 0.1 mg/kg, (FIGS. 26D-26F) acetylfentanyl 1.0 mg/kg, or (FIGS. 26G-26I) carfentanil 0.02 mg/kg, s.c. Heart rate and oxygen saturation were measured by oximetry and concentration of drug in serum and brain were measured by LC-MS.

FIGS. 27A-27D. Efficacy of anti-fentanyl mAb against fentanyl and carfentanil. Mice (Balb/c, n=5/group) were passively immunized with mAb (HY11-7E1.3, HY17-2A2.1, HY17-4A5.1, HY18-5B1.1, or HY19-1H6.1) 40 mg/kg, s.c., then challenged with a mixture of fentanyl 0.05 mg/kg and carfentanil 0.005 mg/kg, s.c. The concentrations of fentanyl in serum (FIG. 27A) and brain (FIG. 27B) 30 minutes after administration were measured by LC-MS. The concentrations of carfentanil in serum (FIG. 27C) and brain (FIG. 27D) 30 minutes after administration were measured by LC-MS.

FIG. 28. DSF Fab ΔTₘ with and without carfentanil. HY11-7E1.17, HY17-2A2.1, HY17-4A5.1, HY17-4A5.8, HY18-5B1.1, HY19-1H6.1, and HY19-3A2.1 mAbs at 0.147 mg/mL in PBS, pH 7.4 were combined with Protein Thermal Shift™ assay reagents and subjected to a continuous 0.3% (0.45° C./min) temperature ramp from 25 to 95° C. The samples were tested with and without 10 μM carfentanil. The Tₘ of each mAb fragment is determined by the temperature measurement at the derivative peak (dPeak). All mAbs show varying increases in Tₘ upon incubation with carfentanil. Tₘ increase is indicative of a binding interaction.

FIGS. 29A-29D. DLS analysis of HY1 1-7E1.17 and HY6-F9.19 mAb in various buffer conditions. mAbs were buffer exchanged into listed buffers and hydrodynamic radius (Rh) and % polydispersity was determined using a Wyatt DynaPro III DLS instrument. Polydispersity below 20% and Rh between 4-6 nm is desirable for mAb. (FIG. 29A) HY6-F9.19 mAb % polydispersity. The bars in graph from left to right correspond to the labels from top to bottom (FIG. 29B) HY6-F9.19 mAb hydrodynamic radius (Rh). The bars in graph from left to right correspond to the labels from top to bottom. (FIG. 29C) HY11-7E1.17 mAb % polydispersity (PD). The bars in graph from left to right correspond to the labels from top to bottom (except the sixth bar from the left corresponds to the first label of 100 mM Histidine pH5.5+150 mM NaCl on the top). (FIG. 29D) HY11-7E1.17 mAb hydrodynamic radius (Rh). The bars in graph from left to right correspond to the labels from top to bottom.

FIG. 30. Constructs of standard mAb (HY11-7E1.17), bivalent scFv-Fc fusion (HY11-7E1.21), and tetravalent scFv-Fc fusion (HY11-7E1.22).

FIG. 31. Fentanyl induced antinociception was inhibited by 40 mg/kg standard mAb (HY11-7E1.17), 28.5 mg/kg (Molar equivalent of 40 mg/kg standard mAb) bivalent scFv-Fc fusion (HY11-7E1.21), or 21.6 mg/kg (Molar equivalent of 40 mg/kg standard mAb) tetravalent scFv-Fc fusion (HY11-7E1.22), see left graph. Serum mAb concentrations were tested (see middle graph). Fentanyl can be sequestered in serum by standard mAb (HY11-7E1.17), bivalent scFv-Fc fusion (HY11-7E1.21), or tetravalent scFv-Fc fusion (HY11-7E1.22), see right graph.

### DETAILED DESCRIPTION

Fentanyl or analogs thereof are small molecule synthetic opioid drug compounds that could activate opioid receptors and elicit analgesic and rewarding effects. Nonetheless, overdosing of such synthetic opioid compounds could, for example, depress respiration and induce apnea among other toxic effects, often resulting in death absent timely, effective overdose reversal intervention.

### Anti-fentanyl Antibodies or Fragments Thereof

Accordingly, certain embodiments of the invention provide antibodies and antigen-binding portions of antibodies that specifically bind to fentanyl (i.e., an anti-fentanyl antibody, or fragment thereof), or its analogs (e.g., acetylfentanyl, carfentanil). In certain embodiments, an anti-fentanyl antibody or fragment thereof as described herein is capable of cross-reacting with and binding one or more fentanyl analogs. In certain embodiments, anti-fentanyl antibodies or fragments thereof that bind fentanyl, or its analogs, are capable of counteracting or reversing the toxic or lethal effects of synthetic opioid drug accidental and deliberate overdosing or poisoning.

In some embodiments, an anti-fentanyl antibody, or fragment thereof, comprises: (1) one or more complementarity determining region (CDR) sequences; (2) a heavy chain variable region (VH) sequence; and/or (3) a light chain variable region (VL) sequence, as described herein (e.g., as described in Tables A1-A2, Tables B1-B2, Tables B3-B4, and Tables C1-C2 below).

Fentanyl and most representative fentanyl analogs are described herein and known in the art (e.g., see Patil Armenian, et al., Neuropharmacology. 2018 May 15;134(Pt A):121-132, which is incorporated herein by reference). New fentanyl analogs are introduced in the streets and are often not fully characterized as to chemistry or pharmacology. In some embodiments, the anti-fentanyl antibody, or fragment thereof, described herein may bind one or more fentanyl analogs (e.g., acetylfentanyl, carfentanil, or new or emerging fentanyl analog). For example, in certain embodiments, an anti-fentanyl antibody, or fragment thereof, described herein has affinity for both fentanyl and acetylfentanyl. In certain embodiments, an anti-fentanyl antibody, or fragment thereof, described herein has affinity for both fentanyl and carfentanil. In certain embodiments, an anti-fentanyl antibody, or fragment thereof, described herein has affinity for acetylfentanyl and/or carfentanil. In some embodiments, the anti-fentanyl antibody, or fragment thereof, bind two or more fentanyl analogs. In certain embodiments, an anti-fentanyl antibody, or fragment thereof, described herein has affinity for fentanyl, acetylfentanyl, and carfentanil.

In some embodiments, the anti-fentanyl antibody, or fragment thereof, described herein may also bind one or more fentanyl metabolites (e.g., norfentanyl). In some embodiments, the anti-fentanyl antibody, or fragment thereof, described herein may also bind one or more fentanyl analogs within a mixture of substances (e.g., fentanyl and cocaine, or fentanyl, heroin, and methamphetamine).

Fentanyl or analog thereof, as small molecule synthetic opioid drug compounds, can cross the blood brain barrier and enter the central nervous system. However, once fentanyl or analogs thereof are bound by an antibody or fragment thereof described herein, the pharmacokinetic and/or pharmacodynamic profile of small molecule synthetic opioid drug (fentanyl or analogs) could be altered by the large molecule biologics drug described herein. In certain embodiments, an antibody or fragment thereof described herein is capable of sequestering bound fentanyl or analog thereof in the blood (e.g., whole blood or serum sample). In certain embodiments, an antibody or fragment thereof described herein is capable of reducing the distribution of fentanyl or analog thereof into the brain. In certain embodiments, an antibody or fragment thereof described herein is capable of reducing the concentration of fentanyl or analog thereof in the brain. In certain embodiments, an antibody or fragment thereof described herein is capable of reducing the ratio of drug concentrations in brain over blood. Additionally, the presence of antibodies could reduce the concentration of unbound fentanyl or analog in organs such as lung or heart (Crouse, et al., ACS Pharmacol Transl Sci. 2022 Apr. 20; 5(5):331-343, which is incorporated herein by reference). In certain embodiments, an antibody or fragment thereof described herein is capable of reducing the concentration of free, unbound fentanyl or analog in blood and/or clinically relevant organs such as lung, heart, or brain.

In certain embodiments, an antibody or fragment thereof described herein is capable of reducing or reversing activity of fentanyl or analogs, such as fentanyl or analog-induced pharmacological or physiological effect(s). For example, in certain embodiments, an antibody or fragment thereof described herein is capable of reducing or reversing fentanyl-induced antinociception or analgesic effect. In certain embodiments, an antibody or fragment thereof described herein is capable of reducing, preventing, or reversing fentanyl-induced respiratory depression. In certain embodiments, an antibody or fragment thereof described herein is capable of reducing, preventing, or reversing fentanyl-induced apnea. In certain embodiments, an antibody or fragment thereof described herein is capable of increasing the depressed breath rate, tidal volume, minute volume, and/or EtCO₂ of a mammal that was under fentanyl or analog influence. In certain embodiments, an antibody or fragment thereof described herein is capable of increasing the lowered oxygen saturation of a mammal that was under fentanyl or analog influence (e.g., as shown in rats and non-human primates studies described herein). In certain embodiments, an antibody or fragment thereof described herein is capable of reversing the drug-induced apnea in a mammal that was under fentanyl or analog influence (e.g., as shown in pigs studies described herein). In certain embodiments, an antibody or fragment thereof described herein is capable of reducing, preventing, or reversing fentanyl-induced bradycardia. In certain embodiments, an antibody or fragment thereof described herein is capable of increasing the slow heart rate of a mammal under fentanyl or analog influence. In certain embodiments, an antibody or fragment thereof described herein is capable of reducing the risk of death from synthetic opioid overdose (fentanyl or analogs' related overdose) or poisoning.

In certain embodiments, an antibody or fragment thereof described herein may be given before a subject is exposed to fentanyl or analog to prevent or mitigate the effects of fentanyl or analog exposure. Alternatively, an antibody or fragment thereof described herein may be given after a subject is exposed to fentanyl or analog to reverse or mnitigate the effects of the exposure.

In certain embodiments, administration of an antibody or fragment thereof described herein may be given pre-exposure in a method of passive immunization against fentanyl or analog. In certain embodiments, the antibody or fragment thereof may be administered pre-exposure e.g. at least 6 hours, 12 hours, 24 hours, 2 days, 3 days, 4 days, 5 days, 6 days, 1 week, 2 weeks, 3 weeks, or 4 weeks before an exposure to fentanyl or analog. In certain embodiments., the anti-fentanyl antibody or fragment thereof may be administered post-exposure (after suspected or known exposure) e.g., about 1 minute, 5 minutes, 10 minutes, 20 minutes, 30 minutes, 1 hour, 2 hours, 3 hours, 6 hours, 12 hours, 24 hours, 2 days, 3 days, 4 days, 5 days, 6 days, 1 week, 2 weeks, 3 weeks, or 4 weeks after exposure to reverse or mitigate the acute and/or long-term effects of the exposure. In certain embodiments, the antibody or fragment thereof may be administered post-exposure in an ambulance, clinic, or hospital, after administration of Naloxone or along with administration of Naloxone, or after administration of Nalmefene or along with administration of Nalmefene.

### HY6-F9 Family and Clones

In certain embodiments, an isolated anti-fentanyl antibody or fragment thereof, comprises one or more CDRs selected from the group consisting of:
(a) a heavy chain CDR1 having at least 75% (e.g., 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%) sequence identity to an amino acid sequence of any one of GDSITSGYWN (SEQ ID NO:62) and GDSITSGYWS (SEQ ID NO:63);
(b) a heavy chain CDR2 having at least 75% (e.g., 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%) sequence identity to an amino acid sequence of YISYSGSTYYNPSLKS (SEQ ID NO:64);
(c) a heavy chain CDR3 having at least 75% (e.g., 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%) sequence identity to an amino acid sequence of any one of ARYYGDNYVGAMDY (SEQ ID NO:65), ARYYGDNYVGALDY (SEQ ID NO:161), ARYYGDNYVGAQDY (SEQ ID NO:162), ARYYGDNYVGAIDY (SEQ ID NO:163), and ARYYGDNYVGAADY (SEQ ID NO:164);
(d) a light chain CDR1 having at least 75% (e.g., 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%) sequence identity to an amino acid sequence of any one of RSSKSLLHSNGITYLY (SEQ ID NO:67), RSSKSLLHSNGITYLD (SEQ ID NO:68), KSSKSLLHSNGITYLA (SEQ ID NO:69), RSSKSLLHSQGITYLY (SEQ ID NO:70), RSSKSLLHSNKITYLY (SEQ ID NO:71), RSSKSLLHSNRITYLY (SEQ ID NO:72), and RSSKSLLHSDGITYLY (SEQ ID NO:73);
(e) a light chain CDR2 having at least 75% (e.g., 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%) sequence identity to an amino acid sequence of any one of QMSNLAS (SEQ ID NO:75), QMSNRAS (SEQ ID NO:76), and QMSNRES (SEQ ID NO:77); and
(f) a light chain CDR3 having at least 75% (e.g., 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%) sequence identity to an amino acid sequence of AQNLELPWT (SEQ ID NO:78).

For example, in certain embodiments, an isolated anti-fentanyl antibody, or fragment thereof, described herein comprises
(c) a heavy chain CDR3 having at least 80% (e.g., 86%, 87%, 88%, 89%, 90%, 91%, 92%, 93%, 94%, 95%, 96%, 97%, 98%, 99% or 100%) sequence identity to an amino acid sequence of any one of ARYYGDNYVGAMDY (SEQ ID NO:65), ARYYGDNYVGALDY (SEQ ID NO:161), ARYYGDNYVGAQDY (SEQ ID NO:162), ARYYGDNYVGAIDY (SEQ ID NO:163), and ARYYGDNYVGAADY (SEQ ID NO:164).

In certain embodiments, an isolated anti-fentanyl antibody, or fragment thereof, described herein comprises
(c) a heavy chain CDR3 having at least 80% (e.g., 86%, 87%, 88%, 89%, 90%, 91%, 92%, 93%, 94%, 95%, 96%, 97%, 98%, 99% or 100%) sequence identity to an amino acid sequence of ARYYGDNYVGAMDY (SEQ ID NO:65).

In certain embodiments, the isolated anti-fentanyl antibody or fragment thereof, comprising one or more CDRs selected from the group consisting of:
(a) a heavy chain CDR1 having at least 80% sequence identity to an amino acid sequence of any one of SEQ ID NOs:62-63;
(b) a heavy chain CDR2 having at least 80% sequence identity to an amino acid sequence of SEQ ID NO:64;
(c) a heavy chain CDR3 having at least 80% sequence identity to an amino acid sequence of SEQ ID NOs:65 and 161-164;
(d) a light chain CDR1 having at least 80% sequence identity to an amino acid sequence of any one of SEQ ID NOs:67-73;
(e) a light chain CDR2 having at least 80% sequence identity to an amino acid sequence of any one of SEQ ID NOs:75-77; and
(f) a light chain CDR3 having at least 80% sequence identity to an amino acid sequence of SEQ ID NO:78.

In certain embodiments, the isolated anti-fentanyl antibody or fragment thereof, comprising one or more CDRs selected from the group consisting of:
(a) a heavy chain CDR1 having at least 85% sequence identity to an amino acid sequence of any one of SEQ ID NOs:62-63;
(b) a heavy chain CDR2 having at least 85% sequence identity to an amino acid sequence of SEQ ID NO:64;
(c) a heavy chain CDR3 having at least 85% sequence identity to an amino acid sequence of SEQ ID NOs:65 and 161-164;
(d) a light chain CDR1 having at least 85% sequence identity to an amino acid sequence of any one of SEQI NOs:67-73;
(e) a light chain CDR2 having at least 85% sequence identity to an amino acid sequence of any one of SEQ ID NOs:75-77; and
(f) a light chain CDR3 having at least 85% sequence identity to an amino acid sequence of SEQ ID NO:78.

In certain embodiments, the isolated anti-fentanyl antibody or fragment thereof, comprising one or more CDRs selected from the group consisting of:
(a) a heavy chain CDR1 having at least 90% sequence identity to an amino acid sequence of any one of SEQ ID NOs:62-63;
(b) a heavy chain CDR2 having at least 90% sequence identity to an amino acid sequence of SEQ ID NO:64;
(c) a heavy chain CDR3 having at least 90% sequence identity to an amino acid sequence of SEQ ID NOs:65 and 161-164;
(d) a light chain CDR1 having at least 90% sequence identity to an amino acid sequence of any one of SEQ ID NOs:67-73;
(e) a light chain CDR2 having at least 90% sequence identity to an amino acid sequence of any one of SEQ ID NOs:75-77; and
(f) a light chain CDR3 having at least 90% sequence identity to an amino acid sequence of SEQ ID NO:78.

In certain embodiments, the isolated anti-fentanyl antibody or fragment thereof, comprising one or more CDRs selected from the group consisting of:
(a) a heavy chain CDR1 having at least 95% sequence identity to an amino acid sequence of any one of SEQ ID NOs:62-63;
(b) a heavy chain CDR2 having at least 95% sequence identity to an amino acid sequence of SEQ ID NO:64;
(c) a heavy chain CDR3 having at least 95% sequence identity to an amino acid sequence of SEQ ID NOs:65 and 161-164;
(d) a light chain CDR1 having at least 95% sequence identity to an amino acid sequence of any one of SEQ ID NOs:67-73;
(e) a light chain CDR2 having at least 95% sequence identity to an amino acid sequence of any one of SEQ ID NOs:75-77; and
(f) a light chain CDR3 having at least 95% sequence identity to an amino acid sequence of SEQ ID NO:78.

In some embodiments, an anti-fentanyl antibody, or fragment thereof, comprises two, three, four, five or six CDRs as described above (e.g., each CDR is selected from one of (a)-(f)).

For example, in certain embodiments, the anti-fentanyl antibody, or fragment thereof, as described herein comprises:
(a) a heavy chain CDR1 comprising the amino acid sequence of any one of SEQ ID NOs:62-63;
(b) a heavy chain CDR2 comprising the amino acid sequence of SEQ ID NO:64; (c) a heavy chain CDR3 comprising the amino acid sequence of any one of SEQ ID NOs:65 and 161-164;
(d) a light chain CDR1 comprising the amino acid sequence of any one of SEQ ID NOs:67-73;
(e) a light chain CDR2 comprising the amino acid sequence of any one of SEQ ID NOs:75-77; and
(f) a light chain CDR3 comprising the amino acid sequence of SEQ ID NO:78.

In certain embodiments, certain amino acid residue(s) in one or more CDR region(s) of the anti-fentanyl antibody or fragment thereof described herein may be prone to post-translational modification (PTM) such as asparagine deamidation, aspartate isomerization, or methionine oxidation. Such PTM could optionally be identified and modified to reduce the risk of PTM-induced mAb heterogeneity and immunogenicity. For example, an asparagine deamidation motif (N34/G35, IMGT numbering) in HY6-F9_Hu VL was identified and modified to mitigate PTM risks, N34Q, G35K, and G35R mutations were introduced into the VL of HY6-F9_Hu (see Example 1). The mitigated antibody or fragment thereof may maintain binding affinity for target that is comparable to the unmitigated counterpart antibody. The mitigated antibody or fragment thereof may maintain stability and physical chemical (e.g., structure, or melting temperature) characteristics that are comparable to the unmitigated counterpart antibody.

Thus, in certain embodiments, the anti-fentanyl antibody, or fragment thereof, comprising
(d) a light chain CDR1 comprising the amino acid sequence of X₁SSKSLLHSX₂X₃ITYLX₄ (SEQ ID NO:66), wherein X₁ is R or K, X₂ is N, Q or D, X₃ is G, K or R, X₄ is Y, D or A.

For example, in certain embodiments, the anti-fentanyl antibody, or fragment thereof, as described herein comprises:
(a) a heavy chain CDR1 comprising the amino acid sequence of any one of SEQ ID NOs:62-63;
(b) a heavy chain CDR2 comprising the amino acid sequence of SEQ ID NO:64;
(c) a heavy chain CDR3 comprising the amino acid sequence of any one of SEQ ID NOs:65 and 161-164;
(d) a light chain CDR1 comprising the amino acid sequence of X₁SSKSLLHSX₂X₃ITYLX₄ (SEQ ID NO:66), wherein X₁ is R or K, X₂ is N, Q or D, X₃ is G, K or R, X₄ is Y, D or A;
(e) a light chain CDR2 comprising the amino acid sequence of any one of SEQ ID NOs:75-77; and
(f) a light chain CDR3 comprising the amino acid sequence of SEQ ID NO:78.

In certain embodiments, the anti-fentanyl antibody, or fragment thereof, comprising
(d) a light chain CDR1 comprising the amino acid sequence of any one of X₁SSKSLLHSX₂X₃ITYLX₄ (SEQ ID NO:194), wherein X₂ is Q.

In certain embodiments, the anti-fentanyl antibody, or fragment thereof, comprising
(d) a light chain CDR1 comprising the amino acid sequence of X₁SSKSLLHSX₂X₃ITYLX₄ (SEQ ID NO:195), wherein X₃ is K or R.

In certain embodiments, the anti-fentanyl antibody, or fragment thereof, comprising
(d) a light chain CDR1 comprising the amino acid sequence of any one of RSSKSLLHSQGITYLY (SEQ ID NO:70), RSSKSLLHSNKITYLY (SEQ ID NO:71), and RSSKSLLHSNRITYLY(SEQ ID NO:72).

In certain embodiments, the anti-fentanyl antibody, or fragment thereof, comprising
(d) a light chain CDR1 comprising the amino acid sequence of RSSKSLLHSQGITYLY (SEQ ID NO:70).

In certain embodiments, the anti-fentanyl antibody, or fragment thereof, comprising
(c) a heavy chain CDR3 comprising the amino acid sequence of ARYYGDNYVGAOiDY (SEQ ID NO:165), wherein O₁ is M, L, Q, I, or A.

For example, potential methionine oxidation risk may be reduced wherein O₁ is L, Q, I, or A, in certain embodiments, the anti-fentanyl antibody, or fragment thereof, comprising
(c) a heavy chain CDR3 comprising the amino acid sequence of any one of ARYYGDNYVGALDY (SEQ ID NO:161), ARYYGDNYVGAQDY (SEQ ID NO:162), ARYYGDNYVGAIDY (SEQ ID NO:163), and ARYYGDNYVGAADY (SEQ ID NO:164).

In certain embodiments, an anti-fentanyl antibody, or a fragment thereof, comprises one or more light chain CDR sequence, and/or one or more heavy chain CDR sequence, derived from any of the following antibodies described herein: HY6-F9.6, HY6-F9.8, HY6-F9.9, HY6-F9.10, HY6-F9.14, HY6-F9.15, HY6-F9.16, HY6-F9.17, HY6-F9.19, HY6-F9.20, HY6-F9.21, HY6-F9.27, HY6-F9.32, HY6-F9.33, HY6-F9.34, and HY6-F9.35. The amino acid sequences of the VH CDRs and VL CDRs of these anti-fentanyl antibody clones are set forth in Tables A1-A2.

In certain embodiments, an anti-fentanyl antibody, or a fragment thereof, comprises a VL that has at least about 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to a VL amino acid sequence as in any of the embodiments provided herein, and/or a VH that has at least about 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to a VH amino acid sequence as in any of the embodiments provided herein (e.g., Tables A1-A2).

In certain embodiments, an anti-fentanyl antibody described herein, or fragment thereof, comprises a heavy chain variable region comprising an amino acid sequence that has at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to any one of SEQ ID NOs: 1, 3-5, and 166-169. For example, in certain embodiments, an anti-fentanyl antibody described herein, or fragment thereof, comprises a heavy chain variable region comprising an amino acid sequence that has at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to any one of:

```
  (SEQ ID NO: 3)
QVQLQESGPGLVKPSQTLSLTCTVSGDSITSGYWSWIRQHPGKGLEWIGY
ISYSGSTYYNPSLKSRVTISVDTSKNQFSLKLSSVTAADTAVYYCARYYG
DNYVGAMDYWGQGTLVTVSS;
  (SEQ ID NO: 4)
QVQLQESGPGLVKPSQTLSLTCTVSGDSITSGYWNWIRQHPGKGLEYMGY
ISYSGSTYYNPSLKSRVTISVDTSKNQFSLKLSSVTAADTAVYYCARYYG
DNYVGAMDYWGQGTLVTVSS;
and
  (SEQ ID NO: 5)
QVQLQESGPGLVKPSQTLSLTCTVSGDSITSGYWNWIRQHPGKGLEYMGY
ISYSGSTYYNPSLKSRVTISVDTSKNQFSLKLSSVTAADTATFYCARYYG
DNYVGAMDYWGQGTLVTVSS;
  (SEQ ID NO: 166)
QVQLQESGPGLVKPSQTLSLTCTVSGDSITSGYWNWIRQHPGKGLEYMGY
ISYSGSTYYNPSLKSRVTISVDTSKNQFSLKLSSVTAADTAVYYCARYYG
DNYVGALDYWGQGTLVTVSS;
  (SEQ ID NO: 167)
QVQLQESGPGLVKPSQTLSLTCTVSGDSITSGYWNWIRQHPGKGLEYMGY
ISYSGSTYYNPSLKSRVTISVDTSKNQFSLKLSSVTAADTAVYYCARYYG
DNYVGAQDYWGQGTLVTVS;
  (SEQ ID NO: 168)
QVQLQESGPGLVKPSQTLSLTCTVSGDSITSGYWNWIRQHPGKGLEYMGY
ISYSGSTYYNPSLKSRVTISVDTSKNQFSLKLSSVTAADTAVYYCARYYG
DNYVGAIDYWGQGTLVTVSS;
and
  (SEQ ID NO: 169)
QVQLQESGPGLVKPSQTLSLTCTVSGDSITSGYWNWIRQHPGKGLEYMGY
ISYSGSTYYNPSLKSRVTISVDTSKNQFSLKLSSVTAADTAVYYCARYYG
DNYVGAADYWGQGTLVTVSS.
```

In certain embodiments, an anti-fentanyl antibody described herein, or fragment thereof, comprises a heavy chain variable region comprising an amino acid sequence that has at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to SEQ ID NO:4.

In certain embodiments, an anti-fentanyl antibody described herein, or fragment thereof, comprises a light chain variable region comprising an amino acid sequence that has at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to any one of SEQ ID NOs: 2, and 6-13. For example, in certain embodiments, an anti-fentanyl antibody described herein, or fragment thereof, comprises a light chain variable region comprising an amino acid sequence that has at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to any one of:

```
  (SEQ ID NO: 6)
DIVMTQSPLSLPVTPGEPASISCRSSKSLLHSNGITYLDWYLQKPGQSPQ
LLIYQMSNRASGVPDRFSGSGSGTDFTLKISRVEAEDVGVYYCAQNLELP
WTFGGGTKVEIK;
  (SEQ ID NO: 7)
DIVMTQSPLSLPVTPGEPASISCRSSKSLLHSNGITYLYWYLQKPGQSPQ
LLIYQMSNLASGVPDRFSGSGSGTDFTLKISRVEAEDVGVYYCAQNLELP
WTFGGGTKVEIK;
  (SEQ ID NO: 8)
DIVLTQSPSSLAVSLGERATINCKSSKSLLHSNGITYLAWYQQKPGQPPK
LLIYQMSNRESGVPDRFSGSGSGTDFTLTISSLQAEDVAVYYCAQNLELP
WTFGGGTKVEIK;
  (SEQ ID NO: 9)
DIVLTQSPSSLAVSLGERATINCRSSKSLLHSNGITYLYWYQQKPGQPPK
LLIYQMSNLASGVPDRFSGSGSGTDFTLTISSLQAEDVAVYYCAQNLELP
WTFGGGTKVEIK;
  (SEQ ID NO: 10)
DIVMTQSPLSLPVTPGEPASISCRSSKSLLHSQGITYLYWYLQKPGQSPQ
LLIYQMSNLASGVPDRFSGSGSGTDFTLKISRVEAEDVGVYYCAQNLELP
WTFGGGTKVEIK;
  (SEQ ID NO: 11)
DIVMTQSPLSLPVTPGEPASISCRSSKSLLHSNKITYLYWYLQKPGQSPQ
LLIYQMSNLASGVPDRFSGSGSGTDFTLKISRVEAEDVGVYYCAQNLELP
WTFGGGTKVEIK;
  (SEQ ID NO: 12)
DIVMTQSPLSLPVTPGEPASISCRSSKSLLHSNRITYLYWYLQKPGQSPQ
LLIYQMSNLASGVPDRFSGSGSGTDFTLKISRVEAEDVGVYYCAQNLELP
WTFGGGTKVEIK;
and
  (SEQ ID NO: 13)
DIVMTQSPLSLPVTPGEPASISCRSSKSLLHSDGITYLYWYLQKPGQSPQ
LLIYQMSNLASGVPDRFSGSGSGTDFTLKISRVEAEDVGVYYCAQNLELP
WTFGGGTKVEIK.
```

In certain embodiments, an anti-fentanyl antibody, or fragment thereof, comprises a light chain variable region consisting of an amino acid sequence of any one of SEQ ID NOs: 2, 6, 7, 8, 9, 10, 11, 12, or 13 and further comprises a heavy chain variable region consisting of an amino acid sequence of any one of SEQ ID NOs: 1, 3, 4, 5, 166, 167, 168, or 169.

TABLE A1
HY6-F9 family and clones

| SEQ ID | | | | |
|---|---|---|---|---|
| NO | Sequence | Clone | Chain | Species |
| N/A | Variable region sequence (CDRs bolded) | HY6-F9 | N/A | N/A |
| 1 | EVQLQESGPSLVKPSQTLSLTCSVTGDSITSGYWNWIRKFPGNK | HY6-F9.6 | VH | Mouse |
| | LEYMGYISYSGSTYYNPSLKSRISITRDTSKNQYSLHLNSVTAEDT | | | |
| | ATFYCARYYGDNYVGAMDYWGQGTSVTVSS | | | |
| 2 | DIVMTQAAFSNPVTLGISASISCRSSKSLLHSNGITYLYWYLQKP | HY6-F9.6 | VL | Mouse |
| | GQSPQLLIYQMSNLASGVPDRFSSSGSGTDFTLRISRVEAEDVG | | | |
| | VYYCAQNLELPWTFGGGTKLEIK | | | |
| 3 | QVQLQESGPGLVKPSQTLSLTCTVSGDSITSGYWSWIRQHPGK | HY6-F9.8 | VH | Humanized |
| | GLEWIGYISYSGSTYYNPSLKSRVTISVDTSKNQFSLKLSSVTAA | | | |
| | DTAVYYCARYYGDNYVGAMDYWGQGTLVTVSS | | | |
| 2 | DIVMTQAAFSNPVTLGISASISCRSSKSLLHSNGITYLYWYLQKP | HY6-F9.8 | VL | Mouse |
| | GQSPQLLIYQMSNLASGVPDRFSSSGSGTDFTLRISRVEAEDVG | | | |
| | VYYCAQNLELPWTFGGGTKLEIK | | | |
| 4 | QVQLQESGPGLVKPSQTLSLTCTVSGDSITSGYWNWIRQHPGK | HY6-F9.9 | VH | Humanized |
| | GLEYMGYISYSGSTYYNPSLKSRVTISVDTSKNQFSLKLSSVTAA | | | |
| | DTAVYYCARYYGDNYVGAMDYWGQGTLVTVSS | | | |
| 2 | DIVMTQAAFSNPVTLGISASISCRSSKSLLHSNGITYLYWYLQKP | HY6-F9.9 | VL | Mouse |
| | GQSPQLLIYQMSNLASGVPDRFSSSGSGTDFTLRISRVEAEDVG | | | |
| | VYYCAQNLELPWTFGGGTKLEIK | | | |
| 5 | QVQLQESGPGLVKPSQTLSLTCTVSGDSITSGYWNWIRQHPGK | HY6-F9.10 | VH | Humanized |
| | GLEYMGYISYSGSTYYNPSLKSRVTISVDTSKNQFSLKLSSVTAA | | | |
| | DTATFYCARYYGDNYVGAMDYWGQGTLVTVSS | | | |
| 2 | DIVMTQAAFSNPVTLGISASISCRSSKSLLHSNGITYLYWYLQKP | HY6-F9.10 | VL | Mouse |
| | GQSPQLLIYQMSNLASGVPDRESSSGSGTDFTLRISRVEAEDVG | | | |
| | VYYCAQNLELPWTFGGGTKLEIK | | | |
| 4 | QVQLQESGPGLVKPSQTLSLTCTVSGDSITSGYWNWIRQHPGK | HY6-F9.14 | VH | Humanized |
| | GLEYMGYISYSGSTYYNPSLKSRVTISVDTSKNQFSLKLSSVTAA | | | |
| | DTAVYYCARYYGDNYVGAMDYWGQGTLVTVSS | | | |
| 6 | DIVMTQSPLSLPVTPGEPASISCRSSKSLLHSNGITYLDWYLQKP | HY6-F9.14 | VL | Humanized |
| | GQSPQLLIYQMSNRASGVPDRFSGSGSGTDFTLKISRVEAEDV | | | |
| | GVYYCAQNLELPWTFGGGTKVEIK | | | |
| 4 | QVQLQESGPGLVKPSQTLSLTCTVSGDSITSGYWNWIRQHPGK | HY6-F9.15 | VH | Humanized |
| | GLEYMGYISYSGSTYYNPSLKSRVTISVDTSKNQFSLKLSSVTAA | | | |
| | DTAVYYCARYYGDNYVGAMDYWGQGTLVTVSS | | | |
| 7 | DIVMTQSPLSLPVTPGEPASISCRSSKSLLHSNGITYLYWYLQKP | HY6-F9.15 | VL | Humanized |
| | GQSPQLLIYQMSNLASGVPDRFSGSGSGTDFTLKISRVEAEDVG | | | |
| | VYYCAQNLELPWTFGGGTKVEIK | | | |
| 4 | QVQLQESGPGLVKPSQTLSLTCTVSGDSITSGYWNWIRQHPGK | HY6-F9.16 | VH | Humanized |
| | GLEYMGYISYSGSTYYNPSLKSRVTISVDTSKNQFSLKLSSVTAA | | | |
| | DTAVYYCARYYGDNYVGAMDYWGQGTLVTVSS | | | |
| 8 | DIVLTQSPSSLAVSLGERATINCKSSKSLLHSNGITYLAWYQQKP | HY6-F9.16 | VL | Humanized |
| | GQPPKLLIYQMSNRESGVPDRFSGSGSGTDFTLTISSLQAEDVA | | | |
| | VYYCAQNLELPWTFGGGTKVEIK | | | |
| 4 | QVQLQESGPGLVKPSQTLSLTCTVSGDSITSGYWNWIRQHPGK | HY6-F9.17 | VH | Humanized |
| | GLEYMGYISYSGSTYYNPSLKSRVTISVDTSKNQFSLKLSSVTAA | | | |
| | DTAVYYCARYYGDNYVGAMDYWGQGTLVTVSS | | | |
| 9 | DIVLTQSPSSLAVSLGERATINCRSSKSLLHSNGITYLYWYQQKP | HY6-F9.17 | VL | Humanized |
| | GQPPKLLIYQMSNLASGVPDRFSGSGSGTDFTLTISSLQAEDVA | | | |
| | VYYCAQNLELPWTFGGGTKVEIK | | | |
| 4 | QVQLQESGPGLVKPSQTLSLTCTVSGDSITSGYWNWIRQHPGK | HY6-F9.19 | VH | Humanized |
| | GLEYMGYISYSGSTYYNPSLKSRVTISVDTSKNQFSLKLSSVTAA | | | |
| | DTAVYYCARYYGDNYVGAMDYWGQGTLVTVSS | | | |
| 10 | DIVMTQSPLSLPVTPGEPASISCRSSKSLLHSQGITYLYWYLQKP | HY6-F9.19 | VL | Humanized |
| | GQSPQLLIYQMSNLASGVPDRFSGSGSGTDFTLKISRVEAEDVG | | | |
| | VYYCAQNLELPWTFGGGTKVEIK | | | |
| 4 | QVQLQESGPGLVKPSQTLSLTCTVSGDSITSGYWNWIRQHPGK | HY6-F9.20 | VH | Humanized |
| | GLEYMGYISYSGSTYYNPSLKSRVTISVDTSKNQFSLKLSSVTAA | | | |
| | DTAVYYCARYYGDNYVGAMDYWGQGTLVTVSS | | | |
| 11 | DIVMTQSPLSLPVTPGEPASISCRSSKSLLHSNKITYLYWYLQKP | HY6-F9.20 | VL | Humanized |
| | GQSPQLLIYQMSNLASGVPDRFSGSGSGTDFTLKISRVEAEDVG | | | |
| | VYYCAQNLELPWTFGGGTKVEIK | | | |
| 4 | QVQLQESGPGLVKPSQTLSLTCTVSGDSITSGYWNWIRQHPGK | HY6-F9.21 | VH | Humanized |
| | GLEYMGYISYSGSTYYNPSLKSRVTISVDTSKNQFSLKLSSVTAA | | | |
| | DTAVYYCARYYGDNYVGAMDYWGQGTLVTVSS | | | |
| 12 | DIVMTQSPLSLPVTPGEPASISCRSSKSLLHSNRITYLYWYLQKP | HY6-F9.21 | VL | Humanized |
| | GQSPQLLIYQMSNLASGVPDRFSGSGSGTDFTLKISRVEAEDVG | | | |
| | VYYCAQNLELPWTFGGGTKVEIK | | | |
| 4 | QVQLQESGPGLVKPSQTLSLTCTVSGDSITSGYWNWIRQHPGK | HY6-F9.27 | VH | Humanized |
| | GLEYMGYISYSGSTYYNPSLKSRVTISVDTSKNQFSLKLSSVTAA | | | |
| | DTAVYYCARYYGDNYVGAMDYWGQGTLVTVSS | | | |
| 13 | DIVMTQSPLSLPVTPGEPASISCRSSKSLLHSDGITYLYWYLQKP | HY6-F9.27 | VL | Humanized |
| | GQSPQLLIYQMSNLASGVPDRFSGSGSGTDFTLKISRVEAEDVG | | | |
| | VYYCAQNLELPWTFGGGTKVEIK | | | |
| 166 | QVQLQESGPGLVKPSQTLSLTCTVSGDSITSGYWNWIRQHPGK | HY6-F9.32 | VH | Humanized |
| | GLEYMGYISYSGSTYYNPSLKSRVTISVDTSKNQFSLKLSSVTAA | | | |
| | DTAVYYCARYYGDNYVGALDYWGQGTLVTVSS | | | |
| 10 | DIVMTQSPLSLPVTPGEPASISCRSSKSLLHSQGITYLYWYLQKP | HY6-F9.32 | VL | Humanized |
| | GQSPQLLIYQMSNLASGVPDRFSGSGSGTDFTLKISRVEAEDVG | | | |
| | VYYCAQNLELPWTFGGGTKVEIK | | | |
| 167 | QVQLQESGPGLVKPSQTLSLTCTVSGDSITSGYWNWIRQHPGK | HY6-F9.33 | VH | Humanized |
| | GLEYMGYISYSGSTYYNPSLKSRVTISVDTSKNQFSLKLSSVTAA | | | |
| | DTAVYYCARYYGDNYVGAQDYWGQGTLVTVS | | | |
| 10 | DIVMTQSPLSLPVTPGEPASISCRSSKSLLHSQGITYLYWYLQKP | HY6-F9.33 | VL | Humanized |
| | GQSPQLLIYQMSNLASGVPDRFSGSGSGTDFTLKISRVEAEDVG | | | |
| | VYYCAQNLELPWTFGGGTKVEIK | | | |
| 168 | QVQLQESGPGLVKPSQTLSLTCTVSGDSITSGYWNWIRQHPGK | HY6-F9.34 | VH | Humanized |
| | GLEYMGYISYSGSTYYNPSLKSRVTISVDTSKNQFSLKLSSVTAA | | | |
| | DTAVYYCARYYGDNYVGAIDYWGQGTLVTVSS | | | |
| 10 | DIVMTQSPLSLPVTPGEPASISCRSSKSLLHSQGITYLYWYLQKP | HY6-F9.34 | VL | Humanized |
| | GQSPQLLIYQMSNLASGVPDRFSGSGSGTDFTLKISRVEAEDVG | | | |
| | VYYCAQNLELPWTFGGGTKVEIK | | | |
| 169 | QVQLQESGPGLVKPSQTLSLTCTVSGDSITSGYWNWIRQHPGK | HY6-F9.35 | VH | Humanized |
| | GLEYMGYISYSGSTYYNPSLKSRVTISVDTSKNQFSLKLSSVTAA | | | |
| | DTAVYYCARYYGDNYVGAADYWGQGTLVTVSS | | | |
| 10 | DIVMTQSPLSLPVTPGEPASISCRSSKSLLHSQGITYLYWYLQKP | HY6-F9.35 | VL | Humanized |
| | GQSPQLLIYQMSNLASGVPDRFSGSGSGTDFTLKISRVEAEDVG | | | |
| | VYYCAQNLELPWTFGGGTKVEIK | | | |

TABLE A2
Sequences of CDRs in HY6-F9 family and clones

| SEQ ID | | | | |
|---|---|---|---|---|
| NO | CDR Sequence | Clone | Chain | CDR |
| 61 | GDSITSGYWZ₁, wherein | HY6-F9 clones | VH | CDR1 |
| | Z₁ is N or S | | | |
| 62 | GDSITSGYWN | HY6-F9.6 | VH | CDR1 |
| | | HY6-F9.9 | | |
| | | HY6-F9.10 | | |
| | | HY6-F9.14 | | |
| | | HY6-F9.15 | | |
| | | HY6-F9.16 | | |
| | | HY6-F9.17 | | |
| | | HY6-F9.19 | | |
| | | HY6-F9.20 | | |
| | | HY6-F9.21 | | |
| | | HY6-F9.27 | | |
| | | HY6-F9.32 | | |
| | | HY6-F9.33 | | |
| | | HY6-F9.34 | | |
| | | HY6-F9.35 | | |
| 63 | GDSITSGYWS | HY6-F9.8 | VH | CDR1 |
| 64 | YISYSGSTYYNPSLKS | HY6-F9 clones | VH | CDR2 |
| 165 | ARYYGDNYVGAO₁DY, | HY6-F9 clones | VH | CDR3 |
| | wherein O₁ is M, L, Q, I, or A | | | |
| 65 | ARYYGDNYVGAMDY | HY6-F9.6 | VH | CDR3 |
| | | HY6-F9.8 | | |
| | | HY6-F9.9 | | |
| | | HY6-F9.10 | | |
| | | HY6-F9.14 | | |
| | | HY6-F9.15 | | |
| | | HY6-F9.16 | | |
| | | HY6-F9.17 | | |
| | | HY6-F9.19 | | |
| | | HY6-F9.20 | | |
| | | HY6-F9.21 | | |
| | | HY6-F9.27 | | |
| 161 | ARYYGDNYVGALDY | HY6-F9.32 | VH | CDR3 |
| 162 | ARYYGDNYVGAQDY | HY6-F9.33 | VH | CDR3 |
| 163 | ARYYGDNYVGAIDY | HY6-F9.34 | VH | CDR3 |
| 164 | ARYYGDNYVGAADY | HY6-F9.35 | VH | CDR3 |
| 66 | X₁SSKSLLHSX₂X₃ITYLX₄, wherein | HY6-F9 clones | VL | CDR1 |
| | X₁ is R or K, | | | |
| | X₂ is N, Q or D, | | | |
| | X₃ is G, K or R, | | | |
| | X₄ is Y, D or A | | | |
| 67 | RSSKSLLHSNGITYLY | HY6-F9.6 | VL | CDR1 |
| | | HY6-F9.8 | | |
| | | HY6-F9.9 | | |
| | | HY6-F9.10 | | |
| | | HY6-F9.15 | | |
| | | HY6-F9.17 | | |
| 68 | RSSKSLLHSNGITYLD | HY6-F9.14 | VL | CDR1 |
| 69 | KSSKSLLHSNGITYLA | HY6-F9.16 | VL | CDR1 |
| 70 | RSSKSLLHSQGITYLY | HY6-F9.19 | VL | CDR1 |
| | | HY6-F9.32 | | |
| | | HY6-F9.33 | | |
| | | HY6-F9.34 | | |
| | | HY6-F9.35 | | |
| 71 | RSSKSLLHSNKITYLY | HY6-F9.20 | VL | CDR1 |
| 72 | RSSKSLLHSNRITYLY | HY6-F9.21 | VL | CDR1 |
| 73 | RSSKSLLHSDGITYLY | HY6-F9.27 | VL | CDR1 |
| 74 | QMSNB₁B₂S, wherein | HY6-F9 clones | VL | CDR2 |
| | B₁ is L or R, | | | |
| | B₂ is A or E | | | |
| 75 | QMSNLAS | HY6-F9.6 | VL | CDR2 |
| | | HY6-F9.8 | | |
| | | HY6-F9.9 | | |
| | | HY6-F9.10 | | |
| | | HY6-F9.15 | | |
| | | HY6-F9.17 | | |
| | | HY6-F9.19 | | |
| | | HY6-F9.20 | | |
| | | HY6-F9.21 | | |
| | | HY6-F9.27 | | |
| | | HY6-F9.32 | | |
| | | HY6-F9.33 | | |
| | | HY6-F9.34 | | |
| | | HY6-F9.35 | | |
| 76 | QMSNRAS | HY6-F9.14 | VL | CDR2 |
| 77 | QMSNRES | HY6-F9.16 | VL | CDR2 |
| 78 | AQNLELPWT | HY6-F9 clones | VL | CDR3 |

### H1Y6-F9 Clones

In certain embodiments, an anti-fentanyl antibody, or a fragment thereof, comprises three light chain CDR sequences (VL. CDR1, CDR2 and CDR3), and/or three heavy chain CDR sequences (VH CDR1, CDR2 and CDR3), as described in any of the following antibody clones described herein: HY6-F9.6, HY6-F9.8, HY6-F9.9, HY6-F9.10, HY6-F79.14, HY6-F79.15, HY6-F9.16, HY6-F9.17, HY6-F79.19, HY6-F9.20, HY6-F9.21, HY6-F9.27, HY6-F9.32, HY6-F9.33, HY6-F9.34, and HY6-F9.35, as set forth in Table A1 (CDRs bolded) and Table A2.

For example, in certain embodiments, the isolated anti-fentanyl antibody, or fragment thereof, comprises three VH CDRs from clone HY6-F9.20, which are:
(a) a heavy chain CDR1 comprising the amino acid sequence of SEQ ID NO:62;
(b) a heavy chain CDR2 comprising the amino acid sequence of SEQ ID NO:64, and
(c) a heavy chain CDR3 comprising the amino acid sequence of SEQ ID NO:65.

For example, in certain embodiments, the isolated anti-fentanyl antibody, or fragment thereof, comprises three VL CDRs from clone HY6-F9.20, which are:
(a) a light chain CDR1 comprising the amino acid sequence of SEQ ID NO:71,
(b) a light chain CDR2 comprising the amino acid sequence of SEQ ID NO:75, and
(c) a light chain CDR3 comprising the amino acid sequence of SEQ ID NO:78.

For example, in certain embodiments, the isolated anti-fentanyl antibody, or fragment thereof, comprises three VH CDRs and three VL CDRs from clone HY6-F9.20, which are:
(a) a heavy chain CDR1 comprising the amino acid sequence of SEQ ID NO:62;
(b) a heavy chain CDR2 comprising the amino acid sequence of SEQ ID NO:64;
(c) a heavy chain CDR3 comprising the amino acid sequence of SEQ ID NO:65;
(d) a light chain CDR1 comprising the amino acid sequence of SEQ ID NO:71;
(e) a light chain CDR2 comprising the amino acid sequence of SEQ ID NO:75; and
(f) a light chain CDR3 comprising the amino acid sequence of SEQ ID NO:78.

In certain embodiments, an anti-fentanyl antibody, or a fragment thereof, comprises a light chain sequence comprising an amino acid sequence that has at least about 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to a VL amino acid sequence of any of the following antibody clones described herein: HY6-F9.6, HY6-F9.8, HY6-F9.9, HY6-F9.10, HY6-F9.14, HY6-F9.15, HY6-F9.16, HY6-F9.17, HY6-F9.19, HY6-F9.20, HY6-F9.21, HY6-F9.27, HY6-F9.32, HY6-F9.33, HY6-F9.34, and HY6-F9.35, and/or a heavy chain sequence comprising an amino acid sequence that has at least about 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to a VH amino acid sequence of any of the following antibody clones described herein: HY6-F9.6, HY6-F9.8, HY6-F9.9, HY6-F9.10, HY6-F9.14, HY6-F9.15, HY6-F9.16, HY6-F9.17, HY6-F9.19, HY6-F9.20, HY6-F9.21, and HY6-F9.27, HY6-F9.32, HY6-F9.33, HY6-F9.34, and HY6-F9.35. The amino acid sequences of the light chain variable domain (VL) and heavy chain variable domain (VH) of these anti-fentanyl antibody clones are set forth in Table A1.

For example, two representative clones of HY6-F9 family (HY6-F9.6 and HY6-F9.19) are illustrated below as non-limiting embodiments.

### Clone HY6-F9.6

In some embodiments, an anti-fentanyl antibody, or fragment thereof, comprises a light chain CDR1 comprising the amino acid sequence of SEQ ID NO:70, a light chain CDR2 comprising the amino acid sequence of SEQ ID NO:75, and a light chain CDR3 comprising the amino acid sequence of SEQ ID NO:78. In some embodiments, an anti-fentanyl antibody, or fragment thereof, comprises a heavy chain CDR1 comprising the amino acid sequence of SEQ ID NO:62, a heavy chain CDR2 comprising the amino acid sequence of SEQ ID NO:64, and a heavy chain CDR3 comprising the amino acid sequence of SEQ ID NO:65. In some embodiments, an anti-fentanyl antibody, or fragment thereof, comprises a light chain CDR1-3 and a heavy chain CDR1-3 comprising the amino acid sequences of SEQ ID NOs: 67, 75, 78, 62, 64 and 65, respectively. In some embodiments, an anti-fentanyl antibody, or fragment thereof, comprises a light chain CDR1-3 and a heavy chain CDR1-3 consisting of the amino acid sequences of SEQ ID NOs:67, 75, 78, 62, 64 and 65, respectively.

In some embodiments, an anti-fentanyl antibody, or fragment thereof, comprises a light chain variable region comprising an amino acid sequence that has at least about 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to SEQ ID NO:2. In some embodiments, an anti-fentanyl antibody, or fragment thereof, comprises a light chain variable region comprising the amino acid sequence of SEQ ID NO:2. In some embodiments, an anti-fentanyl antibody, or fragment thereof, comprises a light chain variable region consisting of the amino acid sequence of SEQ ID NO:2.

In some embodiments, an anti-fentanyl antibody, or fragment thereof, comprises a heavy chain variable region comprising an amino acid sequence that has at least about 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to SEQ ID NO: 1. In some embodiments, an anti-fentanyl antibody, or fragment thereof, comprises a heavy chain variable region comprising the amino acid sequence of SEQ ID NO:1. In some embodiments, an anti-fentanyl antibody, or fragment thereof, comprises a heavy chain variable region consisting of the amino acid sequence of SEQ ID NO:1.

In some embodiments, an anti-fentanyl antibody, or fragment thereof, comprises a light chain variable region comprising an amino acid sequence that has at least about 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to SEQ ID NO:2 and further comprises a heavy chain variable region comprising an amino acid sequence that has at least about 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to SEQ ID NO:1. In some embodiments, an anti-fentanyl antibody, or fragment thereof, comprises a light chain variable region comprising the amino acid sequence of SEQ ID NO:2 and further comprises a heavy chain variable region comprising the amino acid sequence of SEQ ID NO: 1. In some embodiments, an anti-fentanyl antibody, or fragment thereof, comprises a light chain variable region consisting of the amino acid sequence of SEQ ID NO:2 and further comprises a heavy chain variable region consisting of the amino acid sequence of SEQ ID NO:1.

### Clone HY6-F9.19

In some embodiments, an anti-fentanyl antibody, or fragment thereof, comprises a light chain CDR1 comprising the amino acid sequence of SEQ ID NO:70, a light chain CDR2 comprising the amino acid sequence of SEQ ID NO:75, and a light chain CDR3 comprising the amino acid sequence of SEQ ID NO:78. In some embodiments, an anti-fentanyl antibody, or fragment thereof, comprises a heavy chain CDR1 comprising the amino acid sequence of SEQ ID NO:62, a heavy chain CDR2 comprising the amino acid sequence of SEQ ID NO:64, and a heavy chain CDR3 comprising the amino acid sequence of SEQ ID NO:65. In some embodiments, an anti-fentanyl antibody, or fragment thereof, comprises a light chain CDR1-3 and a heavy chain CDR1-3 comprising the amino acid sequences of SEQ ID NOs: 70, 75, 78, 62, 64 and 65, respectively. In some embodiments, an anti-fentanyl antibody, or fragment thereof, comprises a light chain CDR1-3 and a heavy chain CDR1-3 consisting of the amino acid sequences of SEQ ID NOs:70, 75, 78, 62, 64 and 65, respectively.

In some embodiments, an anti-fentanyl antibody, or fragment thereof, comprises a light chain variable region comprising an amino acid sequence that has at least about 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to SEQ ID NO:10. In some embodiments, an anti-fentanyl antibody, or fragment thereof, comprises a light chain variable region comprising the amino acid sequence of SEQ ID NO: 10. In some embodiments, an anti-fentanyl antibody, or fragment thereof, comprises a light chain variable region consisting of the amino acid sequence of SEQ ID NO:10.

In some embodiments, an anti-fentanyl antibody, or fragment thereof, comprises a heavy chain variable region comprising an amino acid sequence that has at least about 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to SEQ ID NO:4. In some embodiments, an anti-fentanyl antibody, or fragment thereof, comprises a heavy chain variable region comprising the amino acid sequence of SEQ ID NO:4. In some embodiments, an anti-fentanyl antibody, or fragment thereof, comprises a heavy chain variable region consisting of the amino acid sequence of SEQ ID NO:4.

In some embodiments, an anti-fentanyl antibody, or fragment thereof, comprises a light chain variable region comprising an amino acid sequence that has at least about 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to SEQ ID NO:10 and further comprises a heavy chain variable region comprising an amino acid sequence that has at least about 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to SEQ ID NO:4. In some embodiments, an anti-fentanyl antibody, or fragment thereof, comprises a light chain variable region comprising the amino acid sequence of SEQ ID NO:10 and further comprises a heavy chain variable region comprising the amino acid sequence of SEQ ID NO:4. In some embodiments, an anti-fentanyl antibody, or fragment thereof, comprises a light chain variable region consisting of the amino acid sequence of SEQ ID NO:10 and further comprises a heavy chain variable region consisting of the amino acid sequence of SEQ ID NO:4.

### HY11-7E1 Family and Clones

In certain embodiments, an isolated anti-fentanyl antibody or fragment thereof, comprises one or more CDRs selected from the group consisting of:
(a) a heavy chain CDR1 having at least 75% (e.g., 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%) sequence identity to an amino acid sequence of any one of GYTFTNYDIN (SEQ ID NO:80) and GYTFTNYDMH (SEQ ID NO:81);
(b) a heavy chain CDR2 having at least 75% (e.g., 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%) sequence identity to an amino acid sequence of any one of WIFPGDGSTKYNEKFKG (SEQ ID NO:83), WIFPGDGSTNYAQKFQG (SEQ ID NO:84), WIFPGEGSTKYNEKFKG (SEQ ID NO:85), and WIFPGDVSTKYNEKFKG (SEQ ID NO:86);
(c) a heavy chain CDR3 having at least 75% (e.g., 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%) sequence identity to an amino acid sequence of ATELVKDYYAMDY (SEQ ID NO:87);
(d) a light chain CDR1 having at least 75% (e.g., 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%) sequence identity to an amino acid sequence of any one of KASQNVGTNVA (SEQ ID NO:89) and RASQNVGTNLA (SEQ ID NO:90)
(e) a light chain CDR2 having at least 75% (e.g., 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%) sequence identity to an amino acid sequence of any one of SASYRYS (SEQ ID NO:92), and SASYLQS (SEQ ID NO:93); and
(f) a light chain CDR3 having at least 75% (e.g., 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%) sequence identity to an amino acid sequence of any one of QQYNSYPYT (SEQ ID NO:95), QQYYNYPYT (SEQ ID NO:96), and QQYNSYPLT (SEQ ID NO:97).

In certain embodiments, an isolated anti-fentanyl antibody or fragment thereof, comprises one or more CDRs selected from the group consisting of:
(a) a heavy chain CDR1 having at least 80% sequence identity to an amino acid sequence of any one of SEQ ID NOs:80-81;
(b) a heavy chain CDR2 having at least 80% sequence identity to an amino acid sequence of any one of SEQ ID NOs:83-86;
(c) a heavy chain CDR3 having at least 80% sequence identity to an amino acid sequence of SEQ ID NO:87;
(d) a light chain CDR1 having at least 80% sequence identity to an amino acid sequence of any one of SEQ ID NOs:89-90;
(e) a light chain CDR2 having at least 80% sequence identity to an amino acid sequence of any one of SEQ ID NOs:92-93; and
(f) a light chain CDR3 having at least 80% sequence identity to an amino acid sequence of any one of SEQ ID NOs:95-97.

For example, in certain embodiments, the isolated anti-fentanyl antibody, or fragment thereof, comprises a heavy chain CDR1 having at least 80% sequence identity to the amino acid sequence of GYTFTNYDIN (SEQ ID NO:80).

In certain embodiments, the anti-fentanyl antibody, or fragment thereof, comprises a heavy chain CDR2 having at least 80% sequence identity to the amino acid sequence of WIFPGEGSTKYNEKFKG (SEQ ID NO:85).

In certain embodiments, the isolated anti-fentanyl antibody, or fragment thereof, comprises a heavy chain CDR3 having at least 80% sequence identity to the amino acid sequence of ATELVKDYYAMDY (SEQ ID NO:87).

In certain embodiments, the isolated anti-fentanyl antibody, or fragment thereof, comprises a light chain CDR1 having at least 80% sequence identity to the amino acid sequence of KASQNVGTNVA (SEQ ID NO:89).

In certain embodiments, the isolated anti-fentanyl antibody, or fragment thereof, comprises a light chain CDR2 having at least 80% sequence identity to the amino acid sequence of SASYRYS (SEQ ID NO:92).

In certain embodiments, the isolated anti-fentanyl antibody, or fragment thereof, comprises a light chain CDR3 having at least 80% sequence identity to an amino acid sequence of any one of QQYNSYPYT (SEQ ID NO:95), and QQYNSYPLT (SEQ ID NO:97).

In certain embodiments, an isolated anti-fentanyl antibody or fragment thereof, comprises one or more CDRs selected from the group consisting of:
(a) a heavy chain CDR1 having at least 85% sequence identity to an amino acid sequence of any one of SEQ ID NOs:80-81;
(b) a heavy chain CDR2 having at least 85% sequence identity to an amino acid sequence of any one of SEQ ID NOs:83-86;
(c) a heavy chain CDR3 having at least 85% sequence identity to an amino acid sequence of SEQ ID NO:87;
(d) a light chain CDR1 having at least 85% sequence identity to an amino acid sequence of any one of SEQ ID NOs:89-90;
(e) a light chain CDR2 having at least 85% sequence identity to an amino acid sequence of any one of SEQ ID NOs:92-93; and
(f) a light chain CDR3 having at least 85% sequence identity to an amino acid sequence of any one of SEQ ID NOs:95-97.

In certain embodiments, an isolated anti-fentanyl antibody or fragment thereof, comprises one or more CDRs selected from the group consisting of:
(a) a heavy chain CDR1 having at least 90% sequence identity to an amino acid sequence of any one of SEQ ID NOs:80-81;
(b) a heavy chain CDR2 having at least 90% sequence identity to an amino acid sequence of any one of SEQ ID NOs:83-86;
(c) a heavy chain CDR3 having at least 90% sequence identity to an amino acid sequence of SEQ ID NO:87;
(d) a light chain CDR1 having at least 90% sequence identity to an amino acid sequence of any one of SEQ ID NOs:89-90;
(e) a light chain CDR2 having at least 90% sequence identity to an amino acid sequence of any one of SEQ ID NOs:92-93; and
(f) a light chain CDR3 having at least 90% sequence identity to an amino acid sequence of any one of SEQ ID NOs:95-97.

In certain embodiments, an isolated anti-fentanyl antibody or fragment thereof, comprises one or more CDRs selected from the group consisting of:
(a) a heavy chain CDR1 having at least 95% sequence identity to an amino acid sequence of any one of SEQ ID NOs:80-81;
(b) a heavy chain CDR2 having at least 95% sequence identity to an amino acid sequence of any one of SEQ ID NOs:83-86;
(c) a heavy chain CDR3 having at least 95% sequence identity to an amino acid sequence of SEQ ID NO:87;
(d) a light chain CDR1 having at least 95% sequence identity to an amino acid sequence of any one of SEQ ID NOs:89-90;
(e) a light chain CDR2 having at least 95% sequence identity to an amino acid sequence of any one of SEQ ID NOs:92-93; and
(f) a light chain CDR3 having at least 95% sequence identity to an amino acid sequence of any one of SEQ ID NOs:95-97.

In some embodiments, an anti-fentanyl antibody, or fragment thereof, comprises two, three, four, five or six CDRs as described above (e.g., each CDR is selected from one of (a)-(f)).

For example, in certain embodiments, the anti-fentanyl antibody, or fragment thereof, as described herein comprises:
(a) a heavy chain CDR1 comprising the amino acid sequence of any one of SEQ ID NOs:80-81;
(b) a heavy chain CDR2 comprising the amino acid sequence of any one of SEQ ID NOs:83-86;
(c) a heavy chain CDR3 comprising the amino acid sequence of SEQ ID NO:87; (d) a light chain CDR1 comprising the amino acid sequence of any one of SEQ ID NOs:89-90;
(e) a light chain CDR2 comprising the amino acid sequence of any one of SEQ ID NOs:92-93; and
(f) a light chain CDR3 comprising the amino acid sequence of any one of SEQ ID NOs:95-97.

In certain embodiments, certain amino acid residue(s) in one or more CDR region(s) of the anti-fentanyl antibody or fragment thereof described herein may be prone to post-translational modification (PTM) such as asparagine deamidation or aspartate isomerization.

Such PTM could optionally be identified and modified to reduce the risk of PTM-induced mAb heterogeneity and immunogenicity. For example, an aspartate isomerization motif (D62/G63, IMGT numbering) in HY11-7E1_Hu VH was identified and modified to mitigate PTM risks, D62E and G63V mutations were introduced into the VL of HY11-7E1_Hu (see Example 1). The mitigated antibody or fragment thereof may maintain binding affinity for target that is comparable to the unmitigated counterpart antibody. The mitigated antibody or fragment thereof may maintain stability and physical chemical (e.g., structure, or melting temperature) characteristics that are comparable to the unmitigated counterpart antibody.

Thus, in certain embodiments, the anti-fentanyl antibody, or fragment thereof, comprising
(b) a heavy chain CDR2 comprising the amino acid sequence of WIFPGJ₁J₂STJ₃YJ₄J₅KFJ₆G (SEQ ID NO:82), wherein J₁ is D or E, J₂ is G or V, J₃ is K or N, J₄ is N or A, J₅ is E or Q, and J₆ is K or Q.

For example, in certain embodiments, the anti-fentanyl antibody, or fragment thereof, as described herein comprises:
(a) a heavy chain CDR1 comprising the amino acid sequence of any one of SEQ ID NOs:80-81;
(b) a heavy chain CDR2 comprising the amino acid sequence of WIFPGJ₁J₂STJ₃YJ₄J₅KFJ₆G (SEQ ID NO:82), wherein J₁ is D or E, J₂ is G or V, J₃ is K or N, J₄ is N or A, J₅ is E or Q, and J₆ is K or Q;
(c) a heavy chain CDR3 comprising the amino acid sequence of SEQ ID NO:87;
(d) a light chain CDR1 comprising the amino acid sequence of any one of SEQ ID NOs:89-90;
(e) a light chain CDR2 comprising the amino acid sequence of any one of SEQ ID NOs:92-93; and
(f) a light chain CDR3 comprising the amino acid sequence of any one of SEQ ID NOs:95-97.

In certain embodiments, the anti-fentanyl antibody, or fragment thereof, comprising
(b) a heavy chain CDR2 comprising the amino acid sequence of WIFPGJ₁J₂STJ₃YJ₄J₅KFJ₆G (SEQ ID NO:196), wherein J₁ is E.

In certain embodiments, the anti-fentanyl antibody, or fragment thereof, comprising (b) a heavy chain CDR2 comprising the amino acid sequence of WIFPGJ₁J₂STJ₃YJ₄J₅KFJ₆G (SEQ ID NO:197), wherein J₂ is V.

In certain embodiments, the anti-fentanyl antibody, or fragment thereof, comprises a heavy chain CDR2 comprising amino acid sequence of WIFPGEGSTKYNEKFKG (SEQ ID NO:85).

In certain embodiments, an anti-fentanyl antibody, or a fragment thereof, comprises one or more light chain CDR sequence, and/or one or more heavy chain CDR sequence, derived from any of the following antibodies described herein: HY11-7E1.1, HY11-7E1.2, HY11-7E1.3, HY11-7E1.4, HY11-7E1.5, HY11-7E1.6, HY11-7E1.10, HY11-7E1.11, HY11-7E1.12, HY11-7E1.13, HY11-7E1.14, HY11-7E1.15, HY11-7E1.17, HY11-7E1.18, and HY11-7E1.25. The amino acid sequences of the VL CDRs and VH CDRs of these anti-fentanyl antibody clones are set forth in Tables B1-B2.

In certain embodiments, an anti-fentanyl antibody, or a fragment thereof, comprises a VL that has at least about 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to a VL amino acid sequence as in any of the embodiments provided herein, and/or a VH that has at least about 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to a VH amino acid sequence as in any of the embodiments provided herein (e.g., Tables B1-B2).

In certain embodiments, an anti-fentanyl antibody described herein, or fragment thereof, comprises a heavy chain variable region comprising an amino acid sequence that has at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to any one of SEQ ID NOs: 14, 18, 19, 20, 25, and 26. For example, in certain embodiments, an anti-fentanyl antibody described herein, or fragment thereof, comprises a heavy chain variable region comprising an amino acid sequence that has at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to any one of:

(SEQ ID NO: 18)
QVQLVQSGAEVKKPGASVKVSCKASGYTFTNYDMHWVRQAPGQGLEWMG
WIFPGDGSTNYAQKFQGRVTMTRDTSISTAYMELSRLRSDDTAVYYCAT
ELVKDYYAMDYWGQGTLVTVSS;
(SEQ ID NO: 19)
QVQLVQSGAEVKKPGASVKVSCKASGYTFTNYDINWVRQAPGQGLEWIG
WIFPGDGSTKYNEKFKGRVTMTRDTSISTAYMELSRLRSDDTAVYYCAT
ELVKDYYAMDYWGQGTLVTVSS;
(SEQ ID NO: 20)
QVQLVQSGAEVKKPGASVKVSCKASGYTFTNYDINWVRQAPGQGLEWIG
WIFPGDGSTKYNEKFKGRVTMTRDTSISTAYMELSRLRSDDTAVFFCAT
ELVKDYYAMDYWGQGTLVTVSS;
(SEQ ID NO: 25)
QVQLVQSGAEVKKPGASVKVSCKASGYTFTNYDINWVRQAPGQGLEWIG
WIFPGEGSTKYNEKFKGRVTMTRDTSISTAYMELSRLRSDDTAVYYCAT
ELVKDYYAMDYWGQGTLVTVSS;
and
(SEQ ID NO: 26)
QVQLVQSGAEVKKPGASVKVSCKASGYTFTNYDINWVRQAPGQGLEWIG
WIFPGDVSTKYNEKFKGRVTMTRDTSISTAYMELSRLRSDDTAVYYCAT
ELVKDYYAMDYWGQGTLVTVSS.

In certain embodiments, an anti-fentanyl antibody described herein, or fragment thereof, comprises a heavy chain variable region comprising an amino acid sequence that has at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to any one of SEQ ID NOs:18, or 25.

In certain embodiments, an anti-fentanyl antibody described herein, or fragment thereof, comprises a light chain variable region comprising an amino acid sequence that has at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to any one of SEQ ID NOs: 15, 16, 17, 21, 22, 23, 24 and 170. For example, in certain embodiments, an anti-fentanyl antibody described herein, or fragment thereof, comprises a light chain variable region comprising an amino acid sequence that has at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to any one of:

(SEQ ID NO: 21)
DIQMTQSPSSLSASVGDRVTITCRASQNVGTNLAWFQQKPGKAPKSLIY
SASYLQSGVPSRFSGSGSGTDFTLTISSLQPEDFATYYCQQYNSYPYTF
GQGTKLEIK;
(SEQ ID NO: 22)
DIQMTQSPSSLSASVGDRVTITCKASQNVGTNVAWFQQKPGKAPKALIY
SASYRYSGVPSRFSGSGSGTDFTLTISSLQPEDFATYYCQQYNSYPYTF
GQGTKLEIK;
(SEQ ID NO: 23)
DIQMTQSPSSLSASVGDRVTITCRASQNVGTNLAWFQQKPGKAPKSLIY
SASYLQSGVPSRFSGSGSGTDFTLTISSLQPEDFATYYCQQYNSYPLTF
GQGTKLEIK;
(SEQ ID NO: 24)
DIQMTQSPSSLSASVGDRVTITCKASQNVGTNVAWFQQKPGKAPKALIY
SASYRYSGVPSRFSGSGSGTDFTLTISSLQPEDFATYYCQQYNSYPLTF
GQGTKLEIK;
and
(SEQ ID NO: 170)
DIQMTQSPSSLSASVGDRVTITCKASQNVGTNVAWYQQKPGKAPKALIY
SASYRYSGVPSRFSGSGSGTDFTLTISSLQPEDFATYYCQQYNSYPLTF
GQGTKLEIK.

In certain embodiments, an anti-fentanyl antibody, or fragment thereof, comprises a light chain variable region consisting of an amino acid sequence of any one of SEQ ID NOs: 15, 16, 17, 21, 22, 23, 24, or 170 and further comprises a heavy chain variable region consisting of an amino acid sequence of any one of SEQ ID NOs: 14, 18, 19, 20, 25, or 26.

TABLE B1
HY11-7E1 family and clones

| SEQ ID | | | | |
|---|---|---|---|---|
| NO | Sequence | Clone | Chain | Species |
| N/A | Variable region sequence (CDRs bolded) | HY11-7E1 | N/A | N/A |
| 14 | QVQLQQSGAELVKPGASVKLSCKASGYTFTNYDINWVR | HY11-7E1.1 | VH | Mouse |
| | QRPEQGLEWIGWIFPGDGSTKYNEKFKGKATLTTDKSSS | | | |
| | TAYMQLSRLTSEDSAVFFCATELVKDYYAMDYWGQGT | | | |
| | SVTVSS | | | |
| 15 | DIVMTQSQKFMSTSVGDRVSVTCKASQNVGTNVAWY | HY11-7E1.1 | VL | Mouse |
| | QQKPGQSPKALIYSASYRYSGVPDRFTGSGSGTDFTLTIS | | | |
| | NVQSEDLAEYFCQQYNSYPYTFGGGTKLEIK | | | |
| 14 | QVQLQQSGAELVKPGASVKLSCKASGYTFTNYDINWVR | HY11-7E1.2 | VH | Mouse |
| | QRPEQGLEWIGWIFPGDGSTKYNEKFKGKATLTTDKSSS | | | |
| | TAYMQLSRLTSEDSAVFFCATELVKDYYAMDYWGQGT | | | |
| | SVTVSS | | | |
| 16 | DIVMTQSQKFMSTSVGDRVSVTCKASQNVGTNVAWY | HY11-7E1.2 | VL | Mouse |
| | QQKPGQSPKTLIYSASYRYSGVPDRFTGSGSGTDFTLTIS | | | |
| | NVQSEDLAEYFCQQYYNYPYTFGGGTKLEIK | | | |
| 14 | QVQLQQSGAELVKPGASVKLSCKASGYTFTNYDINWVR | HY11-7E1.3 | VH | Mouse |
| | QRPEQGLEWIGWIFPGDGSTKYNEKFKGKATLTTDKSSS | | | |
| | TAYMQLSRLTSEDSAVFFCATELVKDYYAMDYWGQGT | | | |
| | SVTVSS | | | |
| 17 | DIVMTQSQKFMSTSVGDRVSVTCKASQNVGTNVAWY | HY11-7E1.3 | VL | Mouse |
| | QQKPGQSPKALIYSASYRYSGVPDRFTGSGSGTDFTLTIS | | | |
| | NVQSEDLAEYFCQQYNSYPLTFGAGTKLELK | | | |
| 18 | QVQLVQSGAEVKKPGASVKVSCKASGYTFTNYDMHWV | HY11-7E1.4 | VH | Humanized |
| | RQAPGQGLEWMGWIFPGDGSTNYAQKFQGRVTMTR | | | |
| | DTSISTAYMELSRLRSDDTAVYYCATELVKDYYAMDYW | | | |
| | GQGTLVTVSS | | | |
| 15 | DIVMTQSQKFMSTSVGDRVSVTCKASQNVGTNVAWY | HY11-7E1.4 | VL | Mouse |
| | QQKPGQSPKALIYSASYRYSGVPDRFTGSGSGTDFTLTIS | | | |
| | NVQSEDLAEYFCQQYNSYPYTFGGGTKLEIK | | | |
| 19 | QVQLVQSGAEVKKPGASVKVSCKASGYTFTNYDINWVR | HY11-7E1.5 | VH | Humanized |
| | QAPGQGLEWIGWIFPGDGSTKYNEKFKGRVTMTRDTSI | | | |
| | STAYMELSRLRSDDTAVYYCATELVKDYYAMDYWGQG | | | |
| | TLVTVSS | | | |
| 15 | DIVMTQSQKFMSTSVGDRVSVTCKASQNVGTNVAWY | HY11-7E1.5 | VL | Mouse |
| | QQKPGQSPKALIYSASYRYSGVPDRFTGSGSGTDFTLTIS | | | |
| | NVQSEDLAEYFCQQYNSYPYTFGGGTKLEIK | | | |
| 20 | QVQLVQSGAEVKKPGASVKVSCKASGYTFTNYDINWVR | HY11-7E1.6 | VH | Humanized |
| | QAPGQGLEWIGWIFPGDGSTKYNEKFKGRVTMTRDTSI | | | |
| | STAYMELSRLRSDDTAVFFCATELVKDYYAMDYWGQG | | | |
| | TLVTVSS | | | |
| 15 | DIVMTQSQKFMSTSVGDRVSVTCKASQNVGTNVAWY | HY11-7E1.6 | VL | Mouse |
| | QQKPGQSPKALIYSASYRYSGVPDRFTGSGSGTDFTLTIS | | | |
| | NVQSEDLAEYFCQQYNSYPYTFGGGTKLEIK | | | |
| 18 | QVQLVQSGAEVKKPGASVKVSCKASGYTFTNYDMHWV | HY11-7E1.10 | VH | Humanized |
| | RQAPGQGLEWMGWIFPGDGSTNYAQKFQGRVTMTR | | | |
| | DTSISTAYMELSRLRSDDTAVYYCATELVKDYYAMDYW | | | |
| | GQGTLVTVSS | | | |
| 21 | DIQMTQSPSSLSASVGDRVTITCRASQNVGTNLAWFQQ | HY11-7E1.10 | VL | Humanized |
| | KPGKAPKSLIYSASYLQSGVPSRFSGSGSGTDFTLTISSLQ | | | |
| | PEDFATYYCQQYNSYPYTFGQGTKLEIK | | | |
| 18 | QVQLVQSGAEVKKPGASVKVSCKASGYTFTNYDMHWV | HY11-7E1.11 | VH | Humanized |
| | RQAPGQGLEWMGWIFPGDGSTNYAQKFQGRVTMTR | | | |
| | DTSISTAYMELSRLRSDDTAVYYCATELVKDYYAMDYW | | | |
| | GQGTLVTVSS | | | |
| 22 | DIQMTQSPSSLSASVGDRVTITCKASQNVGTNVAWFQ | HY11-7E1.11 | VL | Humanized |
| | QKPGKAPKALIYSASYRYSGVPSRFSGSGSGTDFTLTISSL | | | |
| | QPEDFATYYCQQYNSYPYTFGQGTKLEIK | | | |
| 18 | QVQLVQSGAEVKKPGASVKVSCKASGYTFTNYDMHWV | HY11-7E1.12 | VH | Humanized |
| | RQAPGQGLEWMGWIFPGDGSTNYAQKFQGRVTMTR | | | |
| | DTSISTAYMELSRLRSDDTAVYYCATELVKDYYAMDYW | | | |
| | GQGTLVTVSS | | | |
| 23 | DIQMTQSPSSLSASVGDRVTITCRASQNVGTNLAWFQQ | HY11-7E1.12 | VL | Humanized |
| | KPGKAPKSLIYSASYLQSGVPSRFSGSGSGTDFTLTISSLQ | | | |
| | PEDFATYYCQQYNSYPLTFGQGTKLEIK | | | |
| 18 | QVQLVQSGAEVKKPGASVKVSCKASGYTFTNYDMHWV | HY11-7E1.13 | VH | Humanized |
| | RQAPGQGLEWMGWIFPGDGSTNYAQKFQGRVTMTR | | | |
| | DTSISTAYMELSRLRSDDTAVYYCATELVKDYYAMDYW | | | |
| | GQGTLVTVSS | | | |
| 24 | DIQMTQSPSSLSASVGDRVTITCKASQNVGTNVAWFQ | HY11-7E1.13 | VL | Humanized |
| | QKPGKAPKALIYSASYRYSGVPSRFSGSGSGTDFTLTISSL | | | |
| | QPEDFATYYCQQYNSYPLTFGQGTKLEIK | | | |
| 20 | QVQLVQSGAEVKKPGASVKVSCKASGYTFTNYDINWVR | HY11-7E1.14 | VH | Humanized |
| | QAPGQGLEWIGWIFPGDGSTKYNEKFKGRVTMTRDTSI | | | |
| | STAYMELSRLRSDDTAVFFCATELVKDYYAMDYWGQG | | | |
| | TLVTVSS | | | |
| 22 | DIQMTQSPSSLSASVGDRVTITCKASQNVGTNVAWFQ | HY11-7E1.14 | VL | Humanized |
| | QKPGKAPKALIYSASYRYSGVPSRFSGSGSGTDFTLTISSL | | | |
| | QPEDFATYYCQQYNSYPYTFGQGTKLEIK | | | |
| 20 | QVQLVQSGAEVKKPGASVKVSCKASGYTFTNYDINWVR | HY11-7E1.15 | VH | Humanized |
| | QAPGQGLEWIGWIFPGDGSTKYNEKFKGRVTMTRDTSI | | | |
| | STAYMELSRLRSDDTAVFFCATELVKDYYAMDYWGQG | | | |
| | TLVTVSS | | | |
| 24 | DIQMTQSPSSLSASVGDRVTITCKASQNVGTNVAWFQ | HY11-7E1.15 | VL | Humanized |
| | QKPGKAPKALIYSASYRYSGVPSRFSGSGSGTDFTLTISSL | | | |
| | QPEDFATYYCQQYNSYPLTFGQGTKLEIK | | | |
| 25 | QVQLVQSGAEVKKPGASVKVSCKASGYTFTNYDINWVR | HY11-7E1.17 | VH | Humanized |
| | QAPGQGLEWIGWIFPGEGSTKYNEKFKGRVTMTRDTSI | | | |
| | STAYMELSRLRSDDTAVYYCATELVKDYYAMDYWGQG | | | |
| | TLVTVSS | | | |
| 24 | DIQMTQSPSSLSASVGDRVTITCKASQNVGTNVAWFQ | HY11-7E1.17 | VL | Humanized |
| | QKPGKAPKALIYSASYRYSGVPSRFSGSGSGTDFTLTISSL | | | |
| | QPEDFATYYCQQYNSYPLTFGQGTKLEIK | | | |
| 26 | QVQLVQSGAEVKKPGASVKVSCKASGYTFTNYDINWVR | HY11-7E1.18 | VH | Humanized |
| | QAPGQGLEWIGWIFPGDVSTKYNEKFKGRVTMTRDTSI | | | |
| | STAYMELSRLRSDDTAVYYCATELVKDYYAMDYWGQG | | | |
| | TLVTVSS | | | |
| 24 | DIQMTQSPSSLSASVGDRVTITCKASQNVGTNVAWFQ | HY11-7E1.18 | VL | Humanized |
| | QKPGKAPKALIYSASYRYSGVPSRFSGSGSGTDFTLTISSL | | | |
| | QPEDFATYYCQQYNSYPLTFGQGTKLEIK | | | |
| 26 | QVQLVQSGAEVKKPGASVKVSCKASGYTFTNYDINWVR | HY11-7E1.25 | VH | Humanized |
| | QAPGQGLEWIGWIFPGDVSTKYNEKFKGRVTMTRDTSI | | | |
| | STAYMELSRLRSDDTAVYYCATELVKDYYAMDYWGQG | | | |
| | TLVTVSS | | | |
| 170 | DIQMTQSPSSLSASVGDRVTITCKASQNVGTNVAWYQ | HY11-7E1.25 | VL | Humanized |
| | QKPGKAPKALIYSASYRYSGVPSRFSGSGSGTDFTLTISSL | | | |
| | QPEDFATYYCQQYNSYPLTFGQGTKLEIK | | | |

TABLE B2
Sequences of CDRs in HY11-7E1 family and clones

| SEQ ID | | | | |
|---|---|---|---|---|
| NO | Sequence | Clone | Chain | CDR |
| 79 | GYTFTNYDO₁O₂ wherein | HY11-7E1 clones | VH | CDR1 |
| | O₁ is I or M, | | | |
| | O₂ is N or H | | | |
| 80 | GYTFTNYDIN | HY11-7E1.1 | VH | CDR1 |
| | | HY11-7E1.2 | | |
| | | HY11-7E1.3 | | |
| | | HY11-7E1.5 | | |
| | | HY11-7E1.6 | | |
| | | HY11-7E1.14 | | |
| | | HY11-7E1.15 | | |
| | | HY11-7E1.17 | | |
| | | HY11-7E1.18 | | |
| | | HY11-7E1.25 | | |
| 81 | GYTFTNYDMH | HY11-7E1.4 | VH | CDR1 |
| | | HY11-7E1.10 | | |
| | | HY11-7E1.11 | | |
| | | HY11-7E1.12 | | |
| | | HY11-7E1.13 | | |
| 82 | WIFPGJ₁J₂STJ₃YJ₄J₅KFJ₆G, wherein | HY11-7E1 clones | VH | CDR2 |
| | J₁ is D or E, | | | |
| | J₂ is G or V, | | | |
| | J₃ is K or N, | | | |
| | J₄ is N or A, | | | |
| | J₅ is E or Q, and | | | |
| | J₆ is K or Q | | | |
| 83 | WIFPGDGSTKYNEKFKG | HY11-7E1.1 | VH | CDR2 |
| | | HY11-7E1.2 | | |
| | | HY11-7E1.3 | | |
| | | HY11-7E1.5 | | |
| | | HY11-7E1.6 | | |
| | | HY11-7E1.14 | | |
| | | HY11-7E1.15 | | |
| 84 | WIFPGDGSTNYAQKFQG | HY11-7E1.4 | VH | CDR2 |
| | | HY11-7E1.10 | | |
| | | HY11-7E1.11 | | |
| | | HY11-7E1.12 | | |
| | | HY11-7E1.13 | | |
| 85 | WIFPGEGSTKYNEKFKG | HY11-7E1.17 | VH | CDR2 |
| 86 | WIFPGDVSTKYNEKFKG | HY11-7E1.18 | VH | CDR2 |
| | | HY11-7E1.25 | | |
| 87 | ATELVKDYYAMDY | HY11-7E1 clones | VH | CDR3 |
| 88 | O₃ASQNVGTNO₄A, wherein | HY11-7E1 clones | VL | CDR1 |
| | O₃ is K or R, and | | | |
| | O₄ is V or L | | | |
| 89 | KASQNVGTNVA | HY11-7E1.1 | VL | CDR1 |
| | | HY11-7E1.2 | | |
| | | HY11-7E1.3 | | |
| | | HY11-7E1.4 | | |
| | | HY11-7E1.5 | | |
| | | HY11-7E1.6 | | |
| | | HY11-7E1.11 | | |
| | | HY11-7E1.13 | | |
| | | HY11-7E1.14 | | |
| | | HY11-7E1.15 | | |
| | | HY11-7E1.17 | | |
| | | HY11-7E1.18 | | |
| | | HY11-7E1.25 | | |
| 90 | RASQNVGTNLA | HY11-7E1.10 | VL | CDR1 |
| | | HY11-7E1.12 | | |
| 91 | SASYB₃B₄S, wherein | HY11-7E1 clones | VL | CDR2 |
| | B₃ is R or L, and | | | |
| | B₄ is Y or Q | | | |
| 92 | SASYRYS | HY11-7E1.1 | VL | CDR2 |
| | | HY11-7E1.2 | | |
| | | HY11-7E1.3 | | |
| | | HY11-7E1.4 | | |
| | | HY11-7E1.5 | | |
| | | HY11-7E1.6 | | |
| | | HY11-7E1.11 | | |
| | | HY11-7E1.13 | | |
| | | HY11-7E1.14 | | |
| | | HY11-7E1.15 | | |
| | | HY11-7E1.17 | | |
| | | HY11-7E1.18 | | |
| | | HY11-7E1.25 | | |
| 93 | SASYLQS | HY11-7E1.10 | VL | CDR2 |
| | | HY11-7E1.12 | | |
| 94 | QQYZ₂Z₃YPZ₄T, wherein | HY11-7E1 clones | VL | CDR3 |
| | Z₂ is N or Y, and | | | |
| | Z₃ is S or N, | | | |
| | Z₄ is Y or L. | | | |
| 95 | QQYNSYPYT | HY11-7E1.1 | VL | CDR3 |
| | | HY11-7E1.4 | | |
| | | HY11-7E1.5 | | |
| | | HY11-7E1.6 | | |
| | | HY11-7E1.10 | | |
| | | HY11-7E1.11 | | |
| | | HY11-7E1.14 | | |
| 96 | QQYYNYPYT | HY11-7E1.2 | VL | CDR3 |
| 97 | QQYNSYPLT | HY11-7E1.3 | VL | CDR3 |
| | | HY11-7E1.12 | | |
| | | HY11-7E1.13 | | |
| | | HY11-7E1.15 | | |
| | | HY11-7E1.17 | | |
| | | HY11-7E1.18 | | |
| | | HY11-7E1.25 | | |

### HY11-7E1 Clones

In certain embodiments, an anti-fentanyl antibody, or a fragment thereof, comprises three light chain CDR sequences (VL. CDR1, CDR2 and CDR3), and/or three heavy chain CDR sequences (VH CDR1, CDR2 and CDR3), as described in any of the following antibody clones described herein: HY11-7E1.1, HY11-7E1.2, HY11-7E1.3, HY11-7E1.4, HY11-7E1.5, HY11-7E1.6, HY11-7E1.10, HY11-7E1.1 1, HY11-7E1.12, HY11-7E1.13, HY11-7E1.14, HY11-7E1.15, HY11-7E1.17, HY11-7E1.18 and HY11-7E1.25, as set forth in Table B1 (CDRs bolded) and Table B2.

For example, in certain embodiments, the isolated anti-fentanyl antibody, or fragment thereof, comprises three VH CDRs from clone HY11-7E1.17, which are:
(a) a heavy chain CDR1 comprising the amino acid sequence of SEQ ID NO:80;
(b) a heavy chain CDR2 comprising the amino acid sequence of SEQ ID NO: 85, and
(c) a heavy chain CDR3 comprising the amino acid sequence of SEQ ID NO:87.

For example, in certain embodiments, the isolated anti-fentanyl antibody, or fragment thereof, comprises three VI. CDRs from clone HY11-7E1.17, which are:
(a) a light chain CDR1 comprising the amino acid sequence of SEQ ID NO: 89,
(b) a light chain CDR2 comprising the amino acid sequence of SEQ ID NO:92, and
(c) a light chain CDR3 comprising the amino acid sequence of SEQ ID NO:97.

For example, in certain embodiments, the isolated anti-fentanyl antibody, or fragment thereof, comprises three VH CDRs and three VL CDRs from clone HY11-7E1.17, which are:
(a) a heavy chain CDR1 comprising the amino acid sequence of SEQ ID NO:80;
(b) a heavy chain CDR2 comprising the amino acid sequence of SEQ ID NO:85;
(c) a heavy chain CDR3 comprising the amino acid sequence of SEQ ID NO:87;
(d) a light chain CDR1 comprising the amino acid sequence of SEQ ID NO:89;
(e) a light chain CDR2 comprising the amino acid sequence of SEQ ID NO:92; and
(f) a light chain CDR3 comprising the amino acid sequence of SEQ ID NO:97.

In certain embodiments, an anti-fentanyl antibody, or a fragment thereof, comprises a light chain sequence comprising an amino acid sequence that has at least about 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to a VL amino acid sequence of any of the following antibody clones described herein: HY11-7E1.1, HY11-7E1.2, HY11-7E1.3, HY11-7E1.4, HY11-7E1.5, HY11-7E1.6, HY11-7E1.10, HY11-7E1.11, HY11-7E1.12, HY11-7E1.13, HY11-7E1.14, HY11-7E1.15, HY11-7E1.17, HY11-7E1.18, and HY11-7E1.25 and/or a heavy chain sequence comprising an amino acid sequence that has at least about 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to a VH amino acid sequence of any of the following antibody clones described herein: HY11-7E1.1, HY11-7E1.2, HY11-7E1.3, HY11-7E1.4, HY11-7E1.5, HY11-7E1.6, HY11-7E1.10, HY11-7E1.11, HY11-7E1.12, HY11-7E1.13, HY11-7E1.14, HY11-7E1.15, HY11-7E1.17, HY11-7E1.18, and HY11-7E1.25. The amino acid sequences of the light chain variable domain (VL) and heavy chain variable domain (VH) of these anti-fentanyl antibody clones are set forth in Table B1.

Clones of HY11-7E1 family bind fentanyl and at least two or more analogs (e.g., carfentanil and acetylfentanyl). For example, two representative clones of HY11-7E1 family (HY11-7E1.17 and HY11-7E1.18) are illustrated below as non-limiting embodiments.

### Clone HY11-7E1.17

In some embodiments, an anti-fentanyl antibody, or fragment thereof, comprises a light chain CDR1 comprising the amino acid sequence of SEQ ID NO:89, a light chain CDR2 comprising the amino acid sequence of SEQ ID NO:92, and a light chain CDR3 comprising the amino acid sequence of SEQ ID NO:97. In some embodiments, an anti-fentanyl antibody, or fragment thereof, comprises a heavy chain CDR1 comprising the amino acid sequence of SEQ ID NO:80, a heavy chain CDR2 comprising the amino acid sequence of SEQ ID NO:85, and a heavy chain CDR3 comprising the amino acid sequence of SEQ ID NO:87. In some embodiments, an anti-fentanyl antibody, or fragment thereof, comprises a light chain CDR1-3 and a heavy chain CDR1-3 comprising the amino acid sequences of SEQ ID NOs: 89, 92, 97, 80, 85 and 87, respectively. In some embodiments, an anti-fentanyl antibody, or fragment thereof, comprises a light chain CDR1-3 and a heavy chain CDR1-3 consisting of the amino acid sequences of SEQ ID NOs:89, 92, 97, 80, 85 and 87, respectively.

In some embodiments, an anti-fentanyl antibody, or fragment thereof, comprises a light chain variable region comprising an amino acid sequence that has at least about 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to SEQ ID NO:24. In some embodiments, an anti-fentanyl antibody, or fragment thereof, comprises a light chain variable region comprising the amino acid sequence of SEQ ID NO:24. In some embodiments, an anti-fentanyl antibody, or fragment thereof, comprises a light chain variable region consisting of the amino acid sequence of SEQ ID NO:24.

In some embodiments, an anti-fentanyl antibody, or fragment thereof, comprises a heavy chain variable region comprising an amino acid sequence that has at least about 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to SEQ ID NO:25. In some embodiments, an anti-fentanyl antibody, or fragment thereof, comprises a heavy chain variable region comprising the amino acid sequence of SEQ ID NO:25. In some embodiments, an anti-fentanyl antibody, or fragment thereof, comprises a heavy chain variable region consisting of the amino acid sequence of SEQ ID NO:25.

In some embodiments, an anti-fentanyl antibody, or fragment thereof, comprises a light chain variable region comprising an amino acid sequence that has at least about 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to SEQ ID NO:24 and further comprises a heavy chain variable region comprising an amino acid sequence that has at least about 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to SEQ ID NO:25. In some embodiments, an anti-fentanyl antibody, or fragment thereof, comprises a light chain variable region comprising the amino acid sequence of SEQ ID NO:24 and further comprises a heavy chain variable region comprising the amino acid sequence of SEQ ID NO:25. In some embodiments, an anti-fentanyl antibody, or fragment thereof, comprises a light chain variable region consisting of the amino acid sequence of SEQ ID NO:24 and further comprises a heavy chain variable region consisting of the amino acid sequence of SEQ ID NO:25.

### Clone HY11-7E1.18

In some embodiments, an anti-fentanyl antibody, or fragment thereof, comprises a light chain CDR1 comprising the amino acid sequence of SEQ ID NO:89, a light chain CDR2 comprising the amino acid sequence of SEQ ID NO:92, and a light chain CDR3 comprising the amino acid sequence of SEQ ID NO:97. In some embodiments, an anti-fentanyl antibody, or fragment thereof, comprises a heavy chain CDR1 comprising the amino acid sequence of SEQ ID NO:80, a heavy chain CDR2 comprising the amino acid sequence of SEQ ID NO:86, and a heavy chain CDR3 comprising the amino acid sequence of SEQ ID NO:87. In some embodiments, an anti-fentanyl antibody, or fragment thereof, comprises a light chain CDR1-3 and a heavy chain CDR1-3 comprising the amino acid sequences of SEQ ID NOs: 89, 92, 97, 80, 86 and 87, respectively. In some embodiments, an anti-fentanyl antibody, or fragment thereof, comprises a light chain CDR1-3 and a heavy chain CDR1-3 consisting of the amino acid sequences of SEQ ID NOs:89, 92, 97, 80, 86 and 87, respectively.

In some embodiments, an anti-fentanyl antibody, or fragment thereof, comprises a light chain variable region comprising an amino acid sequence that has at least about 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to SEQ ID NO:24. In some embodiments, an anti-fentanyl antibody, or fragment thereof, comprises a light chain variable region comprising the amino acid sequence of SEQ ID NO:24. In some embodiments, an anti-fentanyl antibody, or fragment thereof, comprises a light chain variable region consisting of the amino acid sequence of SEQ ID NO:24.

In some embodiments, an anti-fentanyl antibody, or fragment thereof, comprises a heavy chain variable region comprising an amino acid sequence that has at least about 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to SEQ ID NO:26. In some embodiments, an anti-fentanyl antibody, or fragment thereof, comprises a heavy chain variable region comprising the amino acid sequence of SEQ ID NO:26. In some embodiments, an anti-fentanyl antibody, or fragment thereof, comprises a heavy chain variable region consisting of the amino acid sequence of SEQ ID NO:26.

In some embodiments, an anti-fentanyl antibody, or fragment thereof, comprises a light chain variable region comprising an amino acid sequence that has at least about 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to SEQ ID NO:24 and further comprises a heavy chain variable region comprising an amino acid sequence that has at least about 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to SEQ ID NO:26. In some embodiments, an anti-fentanyl antibody, or fragment thereof, comprises a light chain variable region comprising the amino acid sequence of SEQ ID NO:24 and further comprises a heavy chain variable region comprising the amino acid sequence of SEQ ID NO:26. In some embodiments, an anti-fentanyl antibody, or fragment thereof, comprises a light chain variable region consisting of the amino acid sequence of SEQ ID NO:24 and further comprises a heavy chain variable region consisting of the amino acid sequence of SEQ ID NO:26.

### HY19-1H6 Family and Clones

In certain embodiments, an isolated anti-fentanyl antibody or fragment thereof, comprises one or more CDRs selected from the group consisting of:
(a) a heavy chain CDR1 having at least 75% (e.g., 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%) sequence identity to an amino acid sequence of any one of GYTFTESTMY (SEQ ID NO:121) and GYTFTESTMH (SEQ ID NO:182);
(b) a heavy chain CDR2 having at least 75% (e.g., 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%) sequence identity to an amino acid sequence of any one of HINPNNGGTSYNQKFRG (SEQ ID NO:122), RINPNNGGTNYAQKFQG (SEQ ID NO:184), and HINPNQGGTSYNQKFRG (SEQ ID NO:185);
(c) a heavy chain CDR3 having at least 75% (e.g., 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%) sequence identity to an amino acid sequence of AMELFYFDY (SEQ ID NO:123);
(d) a light chain CDR1 having at least 75% (e.g., 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%) sequence identity to an amino acid sequence of KASQNVGTNVA (SEQ ID NO:89);
(e) a light chain CDR2 having at least 75% (e.g., 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%) sequence identity to an amino acid sequence of SASYRYS (SEQ ID NO:92); and
(f) a light chain CDR3 having at least 75% (e.g., 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%) sequence identity to an amino acid sequence of any one of QQYNISPYT (SEQ ID NO:124), QQYQISPYT (SEQ ID NO:187), and QQYNSYPLT (SEQ ID NO:97).

For example, in certain embodiments, an isolated anti-fentanyl antibody, or fragment thereof, described herein comprises
(b) a heavy chain CDR2 having at least 80% (e.g., 86%, 87%, 88%, 89%, 90%, 91%, 92%, 93%, 94%, 95%, 96%, 97%, 98%, 99% or 100%) sequence identity to an amino acid sequence of any one of HINPNNGGTSYNQKFRG (SEQ ID NO:122), RINPNNGGTNYAQKFQG (SEQ ID NO:184), and HINPNQGGTSYNQKFRG (SEQ ID NO:185).

In certain embodiments, the isolated anti-fentanyl antibody or fragment thereof, comprising one or more CDRs selected from the group consisting of:
(a) a heavy chain CDR1 having at least 80% sequence identity to an amino acid sequence of any one of SEQ ID NOs:121 or 182;
(b) a heavy chain CDR2 having at least 80% sequence identity to an amino acid sequence of any one of SEQ ID NOs:122, 184, or 185;
(c) a heavy chain CDR3 having at least 80% sequence identity to an amino acid sequence of SEQ ID NO:123;
(d) a light chain CDR1 having at least 80% sequence identity to an amino acid sequence of SEQ ID NO:89;
(e) a light chain CDR2 having at least 80% sequence identity to an amino acid sequence of SEQ ID NO:92; and
(f) a light chain CDR3 having at least 80% sequence identity to an amino acid sequence of any one of SEQ ID NOs:97, 124, or 187.

In certain embodiments, the isolated anti-fentanyl antibody or fragment thereof, comprising one or more CDRs selected from the group consisting of:
(a) a heavy chain CDR1 having at least 85% sequence identity to an amino acid sequence of any one of SEQ ID NOs:121 or 182;
(b) a heavy chain CDR2 having at least 85% sequence identity to an amino acid sequence of any one of SEQ ID NOs:122, 184, or 185;
(c) a heavy chain CDR3 having at least 85% sequence identity to an amino acid sequence of SEQ ID NO:123;
(d) a light chain CDR1 having at least 85% sequence identity to an amino acid sequence of SEQ ID NO:89;
(e) a light chain CDR2 having at least 85% sequence identity to an amino acid sequence of SEQ ID NO:92; and
(f) a light chain CDR3 having at least 85% sequence identity to an amino acid sequence of any one of SEQ ID NOs:97, 124, or 187.

In certain embodiments, the isolated anti-fentanyl antibody or fragment thereof, comprising one or more CDRs selected from the group consisting of:
(a) a heavy chain CDR1 having at least 90% sequence identity to an amino acid sequence of any one of SEQ ID NOs:121 or 182;
(b) a heavy chain CDR2 having at least 90% sequence identity to an amino acid sequence of any one of SEQ ID NOs:122, 184, or 185;
(c) a heavy chain CDR3 having at least 90% sequence identity to an amino acid sequence of SEQ ID NO:123;
(d) a light chain CDR1 having at least 90% sequence identity to an amino acid sequence of SEQ ID NO:89;
(e) a light chain CDR2 having at least 90% sequence identity to an amino acid sequence of SEQ ID NO:92; and
(f) a light chain CDR3 having at least 90% sequence identity to an amino acid sequence of any one of SEQ ID NOs:97, 124, or 187.

In certain embodiments, the isolated anti-fentanyl antibody or fragment thereof, comprising one or more CDRs selected from the group consisting of:
(a) a heavy chain CDR1 having at least 95% sequence identity to an amino acid sequence of any one of SEQ ID NOs:121 or 182;
(b) a heavy chain CDR2 having at least 95% sequence identity to an amino acid sequence of any one of SEQ ID NOs:122, 184, or 185;
(c) a heavy chain CDR3 having at least 95% sequence identity to an amino acid sequence of SEQ ID NO:123;
(d) a light chain CDR1 having at least 95% sequence identity to an amino acid sequence of SEQ ID NO:89;
(e) a light chain CDR2 having at least 95% sequence identity to an amino acid sequence of SEQ ID NO:92; and
(f) a light chain CDR3 having at least 95% sequence identity to an amino acid sequence of any one of SEQ ID NOs:97, 124, or 187.

In some embodiments, an anti-fentanyl antibody, or fragment thereof, comprises two, three, four, five or six CDRs as described above (e.g., each CDR is selected from one of (a)-(f)).

For example, in certain embodiments, the anti-fentanyl antibody, or fragment thereof, as described herein comprises:
(a) a heavy chain CDR1 comprising the amino acid sequence of any one of SEQ ID NOs:121 or 182;
(b) a heavy chain CDR2 comprising the amino acid sequence of any one of SEQ ID NOs:122, 184, or 185;
(c) a heavy chain CDR3 comprising the amino acid sequence of SEQ ID NO:123;
(d) a light chain CDR1 comprising the amino acid sequence of SEQ ID NO:89;
(e) a light chain CDR2 comprising the amino acid sequence of SEQ ID NO:92; and
(f) a light chain CDR3 comprising the amino acid sequence of any one of SEQ ID NOs:97, 124, or 187.

In certain embodiments, the anti-fentanyl antibody, or fragment thereof, comprising
(b) a heavy chain CDR2 comprising the amino acid sequence of X₁INPNX₂GGTX₃Y X₄QKFX₅G (SEQ ID NO:183), wherein X₁ is H or R, X₂ is N or Q, X₃ is N, or S, X₄ is N or A, X₅ is R or Q.

For example, in certain embodiments, the anti-fentanyl antibody, or fragment thereof, as described herein comprises:
(a) a heavy chain CDR1 comprising the amino acid sequence of any one of SEQ ID NOs:121 or 182;
(b) a heavy chain CDR2 comprising the amino acid sequence of X₁INPNX₂GGTX₃Y X₄QKFX₅G (SEQ ID NO:183), wherein X₁ is H or R, X₂ is N or Q, X₃ is N, or S, X₄ is N or A, X₅ is R or Q;
(c) a heavy chain CDR3 comprising the amino acid sequence of SEQ ID NO:123;
(d) a light chain CDR1 comprising the amino acid sequence of SEQ ID NO:89;
(e) a light chain CDR2 comprising the amino acid sequence of SEQ ID NO:92; and
(f) a light chain CDR3 comprising the amino acid sequence of any one of SEQ ID NOs:97, 124, or 187.

In certain embodiments, the anti-fentanyl antibody, or fragment thereof, comprising
(f) a light chain CDR3 comprising the amino acid sequence of QQYZ₁Z₂Z₃PZ₄T (SEQ ID NO:186), wherein Z₁ is N or Q, Z₂ is I or S, Z₃ is S or Y, and Z₄ is Y or L.

For example, in certain embodiments, the anti-fentanyl antibody, or fragment thereof, as described herein comprises:
(a) a heavy chain CDR1 comprising the amino acid sequence of any one of SEQ ID NOs:121 or 182;
(b) a heavy chain CDR2 comprising the amino acid sequence of any one of SEQ ID NOs:122, 184, or 185;
(c) a heavy chain CDR3 comprising the amino acid sequence of SEQ ID NO:123;
(d) a light chain CDR1 comprising the amino acid sequence of SEQ ID NO:89;
(e) a light chain CDR2 comprising the amino acid sequence of SEQ ID NO:92; and
(f) a light chain CDR3 comprising the amino acid sequence of QQYZ₁Z₂Z₃PZ₄T (SEQ ID NO:186), wherein Z₁ is N or Q, Z₂ is I or S, Z₃ is S or Y, and Z₄ is Y or L.

In certain embodiments, an anti-fentanyl antibody, or a fragment thereof, comprises one or more light chain CDR sequence, and/or one or more heavy chain CDR sequence, derived from any of the following antibodies described herein: HY19-1H6.1, HY19-1H6.2, HY19-1H6.3, HY19-1H6.4, HY19-1H6.5, HY19-1H6.7, HY19-1H6.8, HY19-1H6.11, HY19-1H6.12, and HY19-1H6.15. The amino acid sequences of the VH CDRs and VL CDRs of these anti-fentanyl antibody clones are set forth in Tables B3-B4.

In certain embodiments, an anti-fentanyl antibody, or a fragment thereof, comprises a VL that has at least about 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to a VL amino acid sequence as in any of the embodiments provided herein, and/or a VH that has at least about 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to a VH amino acid sequence as in any of the embodiments provided herein (e.g., Tables B3-B4).

In certain embodiments, an anti-fentanyl antibody described herein, or fragment thereof, comprises a heavy chain variable region comprising an amino acid sequence that has at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to any one of SEQ ID NOs: 40, 176, 177, or 180.

For example, in certain embodiments, an anti-fentanyl antibody described herein, or fragment thereof, comprises a heavy chain variable region comprising an amino acid sequence that has at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to any one of:

```
  (SEQ ID NO: 176)
QVQLVQSGAEVKKPGASVKVSCKASGYTFTESTMHWVRQAPGQGLEWMGR
INPNNGGTNYAQKFQGRVTMTRDTSISTAYMELSRLRSDDTAVYYCAMEL
FYFDYWGQGTLVTVSS;
  (SEQ ID NO: 177)
QVQLVQSGAEVKKPGASVKVSCKASGYTFTESTMYWVRQAPGQGLEWIGH
INPNNGGTSYNQKFRGRVTMTRDTSISTAYMELSRLRSDDTAVYYCAMEL
FYFDYWGQGTLVTVSS;
and
  (SEQ ID NO: 180)
QVQLVQSGAEVKKPGASVKVSCKASGYTFTESTMYWVRQAPGQGLEWIGH
INPNQGGTSYNQKFRGRVTMTRDTSISTAYMELSRLRSDDTAVYYCAMEL
FYFDYWGQGTLVTVSS.
```

In certain embodiments, an anti-fentanyl antibody described herein, or fragment thereof, comprises a light chain variable region comprising an amino acid sequence that has at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to any one of SEQ ID NOs: 24, 41, 170, 178, and 179. For example, in certain embodiments, an anti-fentanyl antibody described herein, or fragment thereof, comprises a light chain variable region comprising an amino acid sequence that has at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to any one of:

(SEQ ID NO: 24)
DIQMTQSPSSLSASVGDRVTITCKASQNVGTNVAWFQQKPGKAPKALIYS
ASYRYSGVPSRFSGSGSGTDFTLTISSLQPEDFATYYCQQYNSYPLTFGQ
GTKLEIK;
(SEQ ID NO: 170)
DIQMTQSPSSLSASVGDRVTITCKASQNVGTNVAWYQQKPGKAPKALIYS
ASYRYSGVPSRFSGSGSGTDFTLTISSLQPEDFATYYCQQYNSYPLTFGQ
GTKLEIK;
(SEQ ID NO: 178)
DIQMTQSPSSLSASVGDRVTITCKASQNVGTNVAWFQQKPGKAPKALIYS
ASYRYSGVPSRFSGSGSGTDFTLTISSLQPEDFATYYCQQYNISPYTFGQ
GTKLEIK;
and
(SEQID NO: 179)
DIQMTQSPSSLSASVGDRVTITCKASQNVGTNVAWFQQKPGKAPKALIYS
ASYRYSGVPSRFSGSGSGTDFTLTISSLQPEDFATYYCQQYQISPYTFGQ
GTKLEIK.

In certain embodiments, an anti-fentanyl antibody, or fragment thereof, comprises a light chain variable region consisting of an amino acid sequence of any one of SEQ ID NOs: 24, 41, 170, 178, or 179 and further comprises a heavy chain variable region consisting of an amino acid sequence of any one of SEQ ID NOs: 40, 176, 177, or 180.

TABLE B3
HY19-1H6 clones

| SEQ | | | | |
|---|---|---|---|---|
| ID | | | | |
| NO | Sequence | Clone | Chain | Species |
| N/A | Variable region sequence (CDRs bolded) | HY19-1H6 | N/A | N/A |
| 40 | EVQLQQSGPELVKPGASVKISCKTSGYTFTESTMYWVKQ | HY19-1H6.1 | VH | Mouse |
| | SHGKSLEWIGHINPNNGGTSYNQKFRGKATLTVDKSSST | | | |
| | AYMELRSLTSEDSAVYYCAMELFYFDYWGQGTTLTVSS | | | |
| 41 | DIVMTQSQKFMSTSVGDRVSVTCKASQNVGTNVAWY | HY19-1H6.1 | VL | Mouse |
| | QQKPGQSPKALIYSASYRYSGVPDRFTGSGSGTDFTLTIS | | | |
| | NVQSEDLAEYFCQQYNISPYTFGGGTKLEIK | | | |
| 40 | EVQLQQSGPELVKPGASVKISCKTSGYTFTESTMYWVKQ | HY19-1H6.2 | VH | Mouse |
| | SHGKSLEWIGHINPNNGGTSYNQKFRGKATLTVDKSSST | | | |
| | AYMELRSLTSEDSAVYYCAMELFYFDYWGQGTTLTVSS | | | |
| 24 | DIQMTQSPSSLSASVGDRVTITCKASQNVGTNVAWFQ | HY19-1H6.2 | VL | Humanized |
| | QKPGKAPKALIYSASYRYSGVPSRFSGSGSGTDFTLTISSL | | | |
| | QPEDFATYYCQQYNSYPLTFGQGTKLEIK | | | |
| 176 | QVQLVQSGAEVKKPGASVKVSCKASGYTFTESTMHWV | HY19-1H6.3 | VH | Humanized |
| | RQAPGQGLEWMGRINPNNGGTNYAQKFQGRVTMTR | | | |
| | DTSISTAYMELSRLRSDDTAVYYCAMELFYFDYWGQGTL | | | |
| | VTVSS | | | |
| 41 | DIVMTQSQKFMSTSVGDRVSVTCKASQNVGTNVAWY | HY19-1H6.3 | VL | Mouse |
| | QQKPGQSPKALIYSASYRYSGVPDRFTGSGSGTDFTLTIS | | | |
| | NVQSEDLAEYFCQQYNISPYTFGGGTKLEIK | | | |
| 177 | QVQLVQSGAEVKKPGASVKVSCKASGYTFTESTMYWVR | HY19-1H6.4 | VH | Humanized |
| | QAPGQGLEWIGHINPNNGGTSYNQKFRGRVTMTRDTS | | | |
| | ISTAYMELSRLRSDDTAVYYCAMELFYFDYWGQGTLVTV | | | |
| | SS | | | |
| 41 | DIVMTQSQKFMSTSVGDRVSVTCKASQNVGTNVAWY | HY19-1H6.4 | VL | Mouse |
| | QQKPGQSPKALIYSASYRYSGVPDRFTGSGSGTDFTLTIS | | | |
| | NVQSEDLAEYFCQQYNISPYTFGGGTKLEIK | | | |
| 177 | QVQLVQSGAEVKKPGASVKVSCKASGYTFTESTMYWVR | HY19-1H6.5 | VH | Humanized |
| | QAPGOGLEWIGHINPNNGGTSYNQKFRGRVTMTRDTS | | | |
| | ISTAYMELSRLRSDDTAVYYCAMELFYFDYWGQGTLVTV | | | |
| | SS | | | |
| 24 | DIQMTQSPSSLSASVGDRVTITCKASQNVGTNVAWFQ | HY19-1H6.5 | VL | Humanized |
| | QKPGKAPKALIYSASYRYSGVPSRFSGSGSGTDFTLTISSL | | | |
| | QPEDFATYYCQQYNSYPLTFGQGTKLEIK | | | |
| 177 | QVQLVQSGAEVKKPGASVKVSCKASGYTFTESTMYWVR | HY19-1H6.7 | VH | Humanized |
| | QAPGQGLEWIGHINPNNGGTSYNQKFRGRVTMTRDTS | | | |
| | ISTAYMELSRLRSDDTAVYYCAMELFYFDYWGQGTLVTV | | | |
| | SS | | | |
| 178 | DIQMTQSPSSLSASVGDRVTITCKASQNVGTNVAWFQ | HY19-1H6.7 | VL | Humanized |
| | QKPGKAPKALIYSASYRYSGVPSRFSGSGSGTDFTLTISSL | | | |
| | QPEDFATYYCQQYNISPYTFGQGTKLEIK | | | |
| 177 | QVQLVQSGAEVKKPGASVKVSCKASGYTFTESTMYWVR | HY19-1H6.8 | VH | Humanized |
| | QAPGQGLEWIGHINPNNGGTSYNQKFRGRVTMTRDTS | | | |
| | ISTAYMELSRLRSDDTAVYYCAMELFYFDYWGQGTLVTV | | | |
| | SS | | | |
| 179 | DIQMTQSPSSLSASVGDRVTITCKASQNVGTNVAWFQ | HY19-1H6.8 | VL | Humanized |
| | QKPGKAPKALIYSASYRYSGVPSRFSGSGSGTDFTLTISSL | | | |
| | QPEDFATYYCQQYQISPYTFGQGTKLEIK | | | |
| 180 | QVQLVQSGAEVKKPGASVKVSCKASGYTFTESTMYWVR | HY19-1H6.11 | VH | Humanized |
| | QAPGQGLEWIGHINPNQGGTSYNQKFRGRVTMTRDTS | | | |
| | ISTAYMELSRLRSDDTAVYYCAMELFYFDYWGQGTLVTV | | | |
| | SS | | | |
| 178 | DIQMTQSPSSLSASVGDRVTITCKASQNVGTNVAWFQ | HY19-1H6.11 | VL | Humanized |
| | QKPGKAPKALIYSASYRYSGVPSRFSGSGSGTDFTLTISSL | | | |
| | QPEDFATYYCQQYNISPYTFGQGTKLEIK | | | |
| 180 | QVQLVQSGAEVKKPGASVKVSCKASGYTFTESTMYWVR | HY19-1H6.12 | VH | Humanized |
| | QAPGQGLEWIGHINPNQGGTSYNQKFRGRVTMTRDTS | | | |
| | ISTAYMELSRLRSDDTAVYYCAMELFYFDYWGQGTLVTV | | | |
| | SS | | | |
| 179 | DIQMTQSPSSLSASVGDRVTITCKASQNVGTNVAWFQ | HY19-1H6.12 | VL | Humanized |
| | QKPGKAPKALIYSASYRYSGVPSRFSGSGSGTDFTLTISSL | | | |
| | QPEDFATYYCQQYQISPYTFGQGTKLEIK | | | |
| 177 | QVQLVQSGAEVKKPGASVKVSCKASGYTFTESTMYWVR | HY19-1H6.15 | VH | Humanized |
| | QAPGQGLEWIGHINPNNGGTSYNQKFRGRVTMTRDTS | | | |
| | ISTAYMELSRLRSDDTAVYYCAMELFYFDYWGQGTLVTV | | | |
| | SS | | | |
| 170 | DIQMTQSPSSLSASVGDRVTITCKASQNVGTNVAWYQ | HY19-1H6.15 | VL | Humanized |
| | QKPGKAPKALIYSASYRYSGVPSRFSGSGSGTDFTLTISSL | | | |
| | QPEDFATYYCQQYNSYPLTFGQGTKLEIK | | | |

TABLE B4
Sequences of CDRs in HY19-1H6 clones

| SEQ | | | | |
|---|---|---|---|---|
| ID | | | | |
| NO | Sequence | Clone | Chain | CDR |
| 181 | GYTFTESTMO₁ | HY19-1H6 clones | VH | CDR1 |
| | wherein | | | |
| | O₁ is Y or H | | | |
| 121 | GYTFTESTMY | HY19-1H6.1 | VH | CDR1 |
| | | HY19-1H6.2 | | |
| | | HY19-1H6.4 | | |
| | | HY19-1H6.5 | | |
| | | HY19-1H6.7 | | |
| | | HY19-1H6.8 | | |
| | | HY19-1H6.11 | | |
| | | HY19-1H6.12 | | |
| | | HY19-1H6.15 | | |
| 182 | GYTFTESTMH | HY19-1H6.3 | VH | CDR1 |
| 183 | X₁INPNX₂GGTX₃Y | HY19-1H6 clones | VH | CDR2 |
| | X₄QKFX₅G | | | |
| | X₁ is H or R, | | | |
| | X₂ is N or Q, | | | |
| | X₃ is N or S, | | | |
| | X₄ is N or A, | | | |
| | X₅ is R or Q | | | |
| 122 | HINPNNGGTSYNQKFRG | HY19-1H6.1 | VH | CDR2 |
| | | HY19-1H6.2 | | |
| | | HY19-1H6.4 | | |
| | | HY19-1H6.5 | | |
| | | HY19-1H6.7 | | |
| | | HY19-1H6.8 | | |
| | | HY19-1H6.15 | | |
| 184 | RINPNNGGTNYAQKFQG | HY19-1H6.3 | VH | CDR2 |
| 185 | HINPNQGGTSYNQKFRG | HY19-1H6.11 | VH | CDR2 |
| | | HY19-1H6.12 | | |
| 123 | AMELFYFDY | HY19-1H6 clones | VH | CDR3 |
| 89 | KASQNVGTNVA | HY19-1H6 clones | VL | CDR1 |
| 92 | SASYRYS | HY19-1H6 clones | VL | CDR2 |
| 186 | QQYZ₁Z₂Z₃PZ₄T, | HY19-1H6 clones | VL | CDR3 |
| | wherein | | | |
| | Z₁ is N or Q, | | | |
| | Z₂ is I or S, | | | |
| | Z₃ is S or Y, | | | |
| | and | | | |
| | Z₄ is Y or L. | | | |
| 124 | QQYNISPYT | HY19-1H6.1 | VL | CDR3 |
| | | HY19-1H6.3 | | |
| | | HY19-1H6.4 | | |
| | | HY19-1H6.7 | | |
| | | HY19-1H6.11 | | |
| 97 | QQYNSYPLT | HY19-1H6.2 | VL | CDR3 |
| | | HY19-1H6.5 | | |
| | | HY19-1H6.15 | | |
| 187 | QQYQISPYT | HY19-1H6.8 | VL | CDR3 |
| | | HY19-1H6.12 | | |

### H1Y19-1H16 Clones

In certain embodiments, an anti-fentanyl antibody, or a fragment thereof, comprises three light chain CDR sequences (VL. CDR1, CDR2 and CDR3), and/or three heavy chain CDR sequences (VH CDR1, CDR2 and CDR3), as described in any of the following antibody clones described herein: HY19-11H6.1, HY19-11H6.2, HY19-11H6.3, HY19-11H6.4, HY19-11H6.5, HY19-11H6.7, HY19-11H6.8, HY19-11H6.11, HY19-11H6.12, and HY19-11H6.15, as set forth in Table B3 (CDRs bolded) and Table B4.

For example, in certain embodiments, the isolated anti-fentanyl antibody, or fragment thereof, comprises three VH CDRs from clone HY19-11H6.7, which are:
(a) a heavy chain CDR1 comprising the amino acid sequence of SEQ ID NO:121,
(b) a heavy chain CDR2 comprising the amino acid sequence of SEQ ID NO: 122, and
(c) a heavy chain CDR3 comprising the amino acid sequence of SEQ ID NO: 123.

For example, in certain embodiments, the isolated anti-fentanyl antibody, or fragment thereof, comprises three VL. CDRs from clone HY19-11H6.7, which are:
(a) a light chain CDR1 comprising the amino acid sequence of SEQ ID NO: 89;
(b) a light chain CDR2 comprising the amino acid sequence of SEQ ID NO:92, and
(c) a light chain CDR3 comprising the amino acid sequence of SEQ ID NO: 124.

For example, in certain embodiments, the isolated anti-fentanyl antibody, or fragment thereof, comprises three VH CDRs and three VL CDRs from clone HY19-1H6.7, which are:
(a) a heavy chain CDR1 comprising the amino acid sequence of SEQ ID NO:121;
(b) a heavy chain CDR2 comprising the amino acid sequence of SEQ ID NO:122;
(c) a heavy chain CDR3 comprising the amino acid sequence of SEQ ID NO:123;
(d) a light chain CDR1 comprising the amino acid sequence of SEQ ID NO:89;
(e) a light chain CDR2 comprising the amino acid sequence of SEQ ID NO:92; and
(f) a light chain CDR3 comprising the amino acid sequence of SEQ ID NO: 124.

In certain embodiments, an anti-fentanyl antibody, or a fragment thereof, comprises a light chain sequence comprising an amino acid sequence that has at least about 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to a VL amino acid sequence of any of the following antibody clones described herein: HY19-1H6.1, HY19-1H6.2, HY19-1H6.3, HY19-1H6.4, HY19-1H6.5, HY19-1H6.7, HY19-1H6.8, HY19-1H6.11, HY19-1H6.12, and HY19-1H6.15, and/or a heavy chain sequence comprising an amino acid sequence that has at least about 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to a VH amino acid sequence of any of the following antibody clones described herein: HY19-1H6.1, HY19-1H6.2, HY19-1H6.3, HY19-1H6.4, HY19-1H6.5, HY19-1H6.7, HY19-1H6.8, HY19-1H6.11, HY19-1H6.12, and HY19-1H6.15. The amino acid sequences of the light chain variable domain (VL) and heavy chain variable domain (VH) of these anti-fentanyl antibody clones are set forth in Table B3.

For example, two representative clones of HY19-1H6 family (HY19-1H6.12 and HY19-1H6.15) are illustrated below as non-limiting embodiments.

### Clone HY19-1H6.12

In some embodiments, an anti-fentanyl antibody, or fragment thereof, comprises a light chain CDR1 comprising the amino acid sequence of SEQ ID NO:89, a light chain CDR2 comprising the amino acid sequence of SEQ ID NO:92, and a light chain CDR3 comprising the amino acid sequence of SEQ ID NO: 187. In some embodiments, an anti-fentanyl antibody, or fragment thereof, comprises a heavy chain CDR1 comprising the amino acid sequence of SEQ ID NO:121, a heavy chain CDR2 comprising the amino acid sequence of SEQ ID NO:185, and a heavy chain CDR3 comprising the amino acid sequence of SEQ ID NO: 123. In some embodiments, an anti-fentanyl antibody, or fragment thereof, comprises a light chain CDR1-3 and a heavy chain CDR1-3 comprising the amino acid sequences of SEQ ID NOs: 89, 92, 187, 121, 185, and 123, respectively. In some embodiments, an anti-fentanyl antibody, or fragment thereof, comprises a light chain CDR1-3 and a heavy chain CDR1-3 consisting of the amino acid sequences of SEQ ID NOs:89, 92, 187, 121, 185, and 123, respectively.

In some embodiments, an anti-fentanyl antibody, or fragment thereof, comprises a light chain variable region comprising an amino acid sequence that has at least about 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to SEQ ID NO:179. In some embodiments, an anti-fentanyl antibody, or fragment thereof, comprises a light chain variable region comprising the amino acid sequence of SEQ ID NO: 179. In some embodiments, an anti-fentanyl antibody, or fragment thereof, comprises a light chain variable region consisting of the amino acid sequence of SEQ ID NO:179.

In some embodiments, an anti-fentanyl antibody, or fragment thereof, comprises a heavy chain variable region comprising an amino acid sequence that has at least about 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to SEQ ID NO:180. In some embodiments, an anti-fentanyl antibody, or fragment thereof, comprises a heavy chain variable region comprising the amino acid sequence of SEQ ID NO:180. In some embodiments, an anti-fentanyl antibody, or fragment thereof, comprises a heavy chain variable region consisting of the amino acid sequence of SEQ ID NO: 180.

In some embodiments, an anti-fentanyl antibody, or fragment thereof, comprises a light chain variable region comprising an amino acid sequence that has at least about 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to SEQ ID NO:179 and further comprises a heavy chain variable region comprising an amino acid sequence that has at least about 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to SEQ ID NO:180. In some embodiments, an anti-fentanyl antibody, or fragment thereof, comprises a light chain variable region comprising the amino acid sequence of SEQ ID NO:179 and further comprises a heavy chain variable region comprising the amino acid sequence of SEQ ID NO:180. In some embodiments, an anti-fentanyl antibody, or fragment thereof, comprises a light chain variable region consisting of the amino acid sequence of SEQ ID NO:179 and further comprises a heavy chain variable region consisting of the amino acid sequence of SEQ ID NO:180.

### Clone HY19-1H6.15

In some embodiments, an anti-fentanyl antibody, or fragment thereof, comprises a light chain CDR1 comprising the amino acid sequence of SEQ ID NO:89, a light chain CDR2 comprising the amino acid sequence of SEQ ID NO:92, and a light chain CDR3 comprising the amino acid sequence of SEQ ID NO:97. In some embodiments, an anti-fentanyl antibody, or fragment thereof, comprises a heavy chain CDR1 comprising the amino acid sequence of SEQ ID NO:121, a heavy chain CDR2 comprising the amino acid sequence of SEQ ID NO:122, and a heavy chain CDR3 comprising the amino acid sequence of SEQ ID NO: 123. In some embodiments, an anti-fentanyl antibody, or fragment thereof, comprises a light chain CDR1-3 and a heavy chain CDR1-3 comprising the amino acid sequences of SEQ ID NOs: 89, 92, 97, 121, 122 and 123, respectively. In some embodiments, an anti-fentanyl antibody, or fragment thereof, comprises a light chain CDR1-3 and a heavy chain CDR1-3 consisting of the amino acid sequences of SEQ ID NOs:89, 92, 97, 121, 122 and 123, respectively.

In some embodiments, an anti-fentanyl antibody, or fragment thereof, comprises a light chain variable region comprising an amino acid sequence that has at least about 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to SEQ ID NO:170. In some embodiments, an anti-fentanyl antibody, or fragment thereof, comprises a light chain variable region comprising the amino acid sequence of SEQ ID NO: 170. In some embodiments, an anti-fentanyl antibody, or fragment thereof, comprises a light chain variable region consisting of the amino acid sequence of SEQ ID NO:170.

In some embodiments, an anti-fentanyl antibody, or fragment thereof, comprises a heavy chain variable region comprising an amino acid sequence that has at least about 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to SEQ ID NO: 177. In some embodiments, an anti-fentanyl antibody, or fragment thereof, comprises a heavy chain variable region comprising the amino acid sequence of SEQ ID NO:177. In some embodiments, an anti-fentanyl antibody, or fragment thereof, comprises a heavy chain variable region consisting of the amino acid sequence of SEQ ID NO: 177.

In some embodiments, an anti-fentanyl antibody, or fragment thereof, comprises a light chain variable region comprising an amino acid sequence that has at least about 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to SEQ ID NO:170 and further comprises a heavy chain variable region comprising an amino acid sequence that has at least about 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to SEQ ID NO:177. In some embodiments, an anti-fentanyl antibody, or fragment thereof, comprises a light chain variable region comprising the amino acid sequence of SEQ ID NO:170 and further comprises a heavy chain variable region comprising the amino acid sequence of SEQ ID NO:177. In some embodiments, an anti-fentanyl antibody, or fragment thereof, comprises a light chain variable region consisting of the amino acid sequence of SEQ ID NO:170 and further comprises a heavy chain variable region consisting of the amino acid sequence of SEQ ID NO:177.

### Additional Anti-Fentanyl Families and Clones

In certain embodiments, an isolated anti-fentanyl antibody or fragment thereof, comprises one or more CDRs selected from the group consisting of:
(a) a heavy chain CDR1 having at least 75% (e.g., 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%) sequence identity to an amino acid sequence of any one of SEQ ID NOs:98, 110, 114, 118, 121, 125, 129, 133, 137, 141, 143, 146, 150, 153, 156, and 174;
(b) a heavy chain CDR2 having at least 75% (e.g., 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%) sequence identity to an amino acid sequence of any one of SEQ ID NOs:99, 101, 102, 103, 111, 115, 119, 122, 126, 130, 134, 138, 83, 144, 147, 151, 154, 157, and 175;
(c) a heavy chain CDR3 having at least 75% (e.g., 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%) sequence identity to an amino acid sequence of any one of SEQ ID NOs:104, 112, 116, 120, 123, 127, 131, 135, 139, 142, 145, 148, 152, 155, and 158;
(d) a light chain CDR1 having at least 75% (e.g., 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%) sequence identity to an amino acid sequence of any one of SEQ ID NOs: 89, 105, 106, 117, and 149;
(e) a light chain CDR2 having at least 75% (e.g., 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%) sequence identity to an amino acid sequence of any one of SEQ ID NOs:92, 107, 108 and 159; and
(f) a light chain CDR3 having at least 75% (e.g., 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%) sequence identity to an amino acid sequence of any one of SEQ ID NOs:95, 96, 97, 109, 113, 124, 128, 132, 136, 140, and 160.

In certain embodiments, an anti-fentanyl antibody, or a fragment thereof, comprises one or more light chain CDR sequence, and/or one or more heavy chain CDR sequence, derived from any of the following antibodies described herein: HY11-5C1.1, HY11-5C1.2, HY11-6B2.1, HY11-6B2.2, HY17-2A2.1, HY17-2A2.4, HY17-2A2.7, HY17-2A2.8, HY17-4A5.1, HY17-4A5.2, HY17-4A5.3, HY17-4A5.6, HY17-4A5.7, HY17-4A5.8, HY18-1B6.1, HY18-B1.1, HY18-5B1.2, HY18-5B1.4, HY18-5B1.5, HY18-5B1.6, HY18-5B1.9, HY18-5D1.1, HY18-5D1.2, HY19-2A10.1 (also referred to as HY19-1H6.1), HY19-3A2.1, HY19-3A2.2, HY11-2D4, HY11-2F5, HY11-2H1, HY18-2D12, HY18-3F1, and HY19-1G6. The amino acid sequences of the VL CDRs and VH CDRs of these anti-fentanyl antibody clones are set forth in Tables C1-C2.

In certain embodiments, an anti-fentanyl antibody described herein, or fragment thereof, comprises a heavy chain variable region comprising an amino acid sequence that has at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to any one of SEQ ID NOs: 27, 28, 29, 31, 34, 36, 38, 40, 42, 44, 46, 48, 50, 51, 53, 58, 59, 60, 171, 172, and 173.

In certain embodiments, an anti-fentanyl antibody described herein, or fragment thereof, comprises a light chain variable region comprising an amino acid sequence that has at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to any one of SEQ ID NOs:15, 16, 17, 30, 32, 33, 35, 37, 39, 41, 43, 45, 47, 49, 52, 54, 57, and 170.

In certain embodiments, an anti-fentanyl antibody, or a fragment thereof, comprises a VL that has at least about 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to a VL amino acid sequence as in any of the embodiments provided herein, and/or a VH that has at least about 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to a VH amino acid sequence as in any of the embodiments provided herein (e.g., Tables C1-C2).

TABLE C1
Additional anti-fentanyl families and clones

| SEQ | | | | |
|---|---|---|---|---|
| ID | | | | |
| NO | Sequence | Clone | Chain | Species |
| N/A | Variable region sequence (CDRs bolded) | HY11-5C1 | N/A | N/A |
| 27 | EVQLQQSGPELVKPGASVKMSCKASGYTFTSYVMHWV | HY11-5C1.1 | VH | Mouse |
| | KQKPGQGLEWIGYINPYNDGTKYNEKFKGKATLTSDKSS | | | |
| | STAYMELSSLTSEDSAIYYCASEGNYEDYWGQGTLVTVS | | | |
| | A | | | |
| 16 | DIVMTQSQKFMSTSVGDRVSVTCKASQNVGTNVAWY | HY11-5C1.1 | VL | Mouse |
| | QQKPGQSPKTLIYSASYRYSGVPDRFTGSGSGTDFTLTIS | | | |
| | NVQSEDLAEYFCQQYYNYPYTFGGGTKLEIK | | | |
| 27 | EVQLQQSGPELVKPGASVKMSCKASGYTFTSYVMHWV | HY11-5C1.2 | VH | Mouse |
| | KOKPGQGLEWIGYINPYNDGTKYNEKFKGKATLTSDKSS | | | |
| | STAYMELSSLTSEDSAIYYCASEGNYEDYWGQGTLVTVS | | | |
| | A | | | |
| 15 | DIVMTQSQKFMSTSVGDRVSVTCKASQNVGTNVAWY | HY11-5C1.2 | VL | Mouse |
| | QQKPGQSPKALIYSASYRYSGVPDRFTGSGSGTDFTLTIS | | | |
| | NVQSEDLAEYFCQQYNSYPYTFGGGTKLEIK | | | |
| N/A | Variable region sequence (CDRs bolded) | HY11-6B2 | N/A | N/A |
| 28 | QVRLQQSGPELVKPGASVRISCKASGHTFTSYYIYWVKQ | HY11-6B2.1 | VH | Mouse |
| | RPGQGLEWIGWIYFGNINLKYNEKFKGKATLTADKSSST | | | |
| | AYIQLSSLTSEDSAVYFCASEDGPGAYWGQGTLVTVSA | | | |
| 17 | DIVMTQSQKFMSTSVGDRVSVTCKASQNVGTNVAWY | HY11-6B2.1 | VL | Mouse |
| | QQKPGQSPKALIYSASYRYSGVPDRFTGSGSGTDFTLTIS | | | |
| | NVQSEDLAEYFCQQYNSYPLTFGAGTKLELK | | | |
| 28 | QVRLQQSGPELVKPGASVRISCKASGHTFTSYYIYWVKQ | HY11-6B2.2 | VH | Mouse |
| | RPGQGLEWIGWIYFGNINLKYNEKFKGKATLTADKSSST | | | |
| | AYIQLSSLTSEDSAVYFCASEDGPGAYWGQGTLVTVSA | | | |
| 15 | DIVMTQSQKFMSTSVGDRVSVTCKASQNVGTNVAWY | HY11-6B2.2 | VL | Mouse |
| | QQKPGQSPKALIYSASYRYSGVPDRFTGSGSGTDFTLTIS | | | |
| | NVQSEDLAEYFCQQYNSYPYTFGGGTKLEIK | | | |
| N/A | Variable region sequence (CDRs bolded) | HY17-2A2 | N/A | N/A |
| 29 | EVQLQQSGPELVKPGASMKISCKTSGYTFSENTIYWVKQ | HY17-2A2.1 | VH | Mouse |
| | RQGKSLEWIGGINPKNGGTTYNPEFKGKATLTVDKSSST | | | |
| | AYMDLRSLTSDDSAVYYCAIYDSDYFDYWGQGTTLTVSS | | | |
| 30 | DIVMTQSQKFMSTSVGDRVIVTCKASQNVGTNVAWYQ | HY17-2A2.1 | VL | Mouse |
| | QKPGQSPKALIYSASFRYGGVPDRFTGIGSGTEFTLTIRN | | | |
| | VQSEDLAVYFCQQYNSYPITFGGGTKLEIK | | | |
| 171 | QVQLVQSGAEVKKPGASVKVSCKASGYTFSENTIYWVR | HY17-2A2.4 | VH | humanized |
| | QAPGQGLEWIGGINPKNGGTTYNPEFKGRVTMTTDTS | | | |
| | TSTAYMELRSLRSDDTAVYYCAIYDSDYFDYWGQGTLVT | | | |
| | VSS | | | |
| 30 | DIVMTQSQKFMSTSVGDRVIVTCKASQNVGTNVAWYQ | HY17-2A2.4 | VL | Mouse |
| | QKPGQSPKALIYSASFRYGGVPDRFTGIGSGTEFTLTIRN | | | |
| | VQSEDLAVYFCQQYNSYPITFGGGTKLEIK | | | |
| 29 | EVQLQQSGPELVKPGASMKISCKTSGYTFSENTIYWVKQ | HY17-2A2.7 | VH | Mouse |
| | RQGKSLEWIGGINPKNGGTTYNPEFKGKATLTVDKSSST | | | |
| | AYMDLRSLTSDDSAVYYCAIYDSDYFDYWGQGTTLTVSS | | | |
| 170 | DIQMTQSPSSLSASVGDRVTITCKASQNVGTNVAWYQ | HY17-2A2.7 | VL | humanized |
| | QKPGKAPKALIYSASYRYSGVPSRFSGSGSGTDFTLTISSL | | | |
| | QPEDFATYYCQQYNSYPLTFGQGTKLEIK | | | |
| 171 | QVQLVQSGAEVKKPGASVKVSCKASGYTFSENTIYWVR | HY17-2A2.8 | VH | humanized |
| | QAPGQGLEWIGGINPKNGGTTYNPEFKGRVTMTTDTS | | | |
| | TSTAYMELRSLRSDDTAVYYCAIYDSDYFDYWGQGTLVT | | | |
| | VSS | | | |
| 170 | DIQMTQSPSSLSASVGDRVTITCKASQNVGTNVAWYQ | HY17-2A2.8 | VL | humanized |
| | QKPGKAPKALIYSASYRYSGVPSRFSGSGSGTDFTLTISSL | | | |
| | QPEDFATYYCQQYNSYPLTFGQGTKLEIK | | | |
| N/A | Variable region sequence (CDRs bolded) | HY17-4A5 | N/A | N/A |
| 31 | EVKLVESGGGLVLPGGSLRLSCATSGFTFTDYYMSWVRQ | HY17-4A5.1 | VH | Mouse |
| | PPGKALEWLGFIGNRPNGYTTEYNASVKGRFTISRDNSQ | | | |
| | SILYLQMNTLRAEDSATYYCARGSTMITWFAYWGQGTL | | | |
| | VTVSA | | | |
| 32 | DVLMTQSPLSLPVSLGDQASISCRSSQSIVHSNGDTYFD | HY17-4A5.1 | VL | Mouse |
| | WYLQKPGQSPKLLIYKVSNRFSGVPDRFSGSGSGTDFTL | | | |
| | KISRVEAEDLGVYYCFQGSLLPPAFGGGTKLEIK | | | |
| 31 | EVKLVESGGGLVLPGGSLRLSCATSGFTFTDYYMSWVRQ | HY17-4A5.2 | VH | Mouse |
| | PPGKALEWLGFIGNRPNGYTTEYNASVKGRFTISRDNSQ | | | |
| | SILYLQMNTLRAEDSATYYCARGSTMITWFAYWGQGTL | | | |
| | VTVSA | | | |
| 33 | DVLMTQSPKFMSTSVGDRVSVTCKASQNVGTNVAWY | HY17-4A5.2 | VL | Mouse |
| | ROKQGQSPKALIYSASYRYSGVPDRFTGSGSGTDFTLTIS | | | |
| | NVQSEDLADYFCQQYNSYPLTFGAGTKLEIK | | | |
| 31 | EVKLVESGGGLVLPGGSLRLSCATSGFTFTDYYMSWVRQ | HY17-4A5.3 | VH | Mouse |
| | PPGKALEWLGFIGNRPNGYTTEYNASVKGRFTISRDNSQ | | | |
| | SILYLQMNTLRAEDSATYYCARGSTMITWFAYWGQGTL | | | |
| | VTVSA | | | |
| 57 | DIVMTQSPLSLPVTPGEPASISCRSSQSIVHSNGDTYLD | HY17-4A5.3 | VL | Humanized |
| | WYLQKPGQSPQLLIYKVSNRASGVPDRFSGSGSGTDFTL | | | |
| | KISRVEAEDVGVYYCFQGSLLPPAFGGGTKVEIK | | | |
| 58 | QVQLVESGGGLVKPGGSLRLSCAASGFTFTDYYMSWIR | HY17-4A5.6 | VH | Humanized |
| | QAPGKGLEWVSYIGNRPNGYTTYYADSVKGRFTISRDN | | | |
| | AKNSLYLQMNSLRAEDTAVYYCARGSTMITWFAYWGQ | | | |
| | GTTVTVSS | | | |
| 57 | DIVMTQSPLSLPVTPGEPASISCRSSQSIVHSNGDTYLD | HY17-4A5.6 | VL | Humanized |
| | WYLQKPGQSPQLLIYKVSNRASGVPDRFSGSGSGTDFTL | | | |
| | KISRVEAEDVGVYYCFQGSLLPPAFGGGTKVEIK | | | |
| 59 | QVQLVESGGGLVKPGGSLRLSCAASGFTFTDYYMSWIR | HY17-4A5.7 | VH | Humanized |
| | QAPGKGLEWLGFIGNRPNGYTTEYNASVKGRFTISRDN | | | |
| | AKNSLYLQMNSLRAEDTAVYYCARGSTMITWFAYWGQ | | | |
| | GTTVTVSS | | | |
| 57 | DIVMTQSPLSLPVTPGEPASISCRSSQSIVHSNGDTYLD | HY17-4A5.7 | VL | Humanized |
| | WYLQKPGQSPQLLIYKVSNRASGVPDRFSGSGSGTDFTL | | | |
| | KISRVEAEDVGVYYCFQGSLLPPAFGGGTKVEIK | | | |
| 60 | QVQLVESGGGLVKPGGSLRLSCAASGFTFTDYYMSWIR | HY17-4A5.8 | VH | Humanized |
| | QAPGKGLEWLGFIGNRPNGYTTEYQASVKGRFTISRDN | | | |
| | AKNSLYLQMNSLRAEDTAVYYCARGSTMITWFAYWGQ | | | |
| | GTTVTVSS | | | |
| 57 | DIVMTQSPLSLPVTPGEPASISCRSSQSIVHSNGDTYLD | HY17-4A5.8 | VL | Humanized |
| | WYLQKPGQSPQLLIYKVSNRASGVPDRFSGSGSGTDFTL | | | |
| | KISRVEAEDVGVYYCFQGSLLPPAFGGGTKVEIK | | | |
| N/A | Variable region sequence (CDRs bolded) | HY18-1B6 | N/A | N/A |
| 34 | QVQLQQSAAELARPGASVKMSCEASGYTFTTYTMYWV | HY18-1B6.1 | VH | Mouse |
| | KQRPGQGLEWIGYINPGSGYTEYNQKFKDKTTLTADKSS | | | |
| | STAYMQLSSLTSEDSAVYYCATEEVRRGDYFDYWGQGT | | | |
| | TLTVSS | | | |
| 35 | DIVMTQSQKFMSTSVGNRVSVTCKASQNVGTNVAWY | HY18-1B6.1 | VL | Mouse |
| | QQKPGQSPKALIYSASYRYSGVPDRFTGSGSGTDFTLTIS | | | |
| | NVQSEDLADYFCQQYNSFPFTFGSGTKLEIK | | | |
| N/A | Variable region sequence (CDRs bolded) | HY18-5B1 | N/A | N/A |
| 36 | EVQLQQSGAELVKPGASVKLSCTASGFNIKDTYIYWVRQ | HY18-5B1.1 | VH | Mouse |
| | RPEQGLEWIGRIDPANGNTKYDPKVQGKATITADTSSN | | | |
| | TAYLQLSSLTFEDTAVYYCALEHGYYDVMDYWGQGTSV | | | |
| | TVSS | | | |
| 37 | DIVMTQSQKFMSTSVGDRVSVTCKASQNVDTNVAWY | HY18-5B1.1 | VL | Mouse |
| | QKKPGQSPKALIYSASYRYSGVPDRFTGSGSGTDFTLTIS | | | |
| | NVQSEDLAEYFCQQYNSYPYTFGGGTKLEIK | | | |
| 36 | EVQLQQSGAELVKPGASVKLSCTASGFNIKDTYIYWVRQ | HY18-5B1.2 | VH | Mouse |
| | RPEQGLEWIGRIDPANGNTKYDPKVQGKATITADTSSN | | | |
| | TAYLQLSSLTFEDTAVYYCALEHGYYDVMDYWGQGTSV | | | |
| | TVSS | | | |
| 24 | DIQMTQSPSSLSASVGDRVTITCKASQNVGTNVAWFQ | HY18-5B1.2 | VL | Humanized |
| | QKPGKAPKALIYSASYRYSGVPSRFSGSGSGTDFTLTISSL | | | |
| | QPEDFATYYCQQYNSYPLTFGQGTKLEIK | | | |
| 172 | QVQLVQSGAEVKKPGASVKVSCKASGFNIKDTYINWVR | HY18-5B1.4 | VH | Humanized |
| | QATGQGLEWMGWIDPANGNTGYAQKFQGRVTITRNT | | | |
| | SISTAYMELSSLRSEDTAVYYCALEHGYYDVMDYWGQG | | | |
| | TLVTVSS | | | |
| 37 | DIVMTQSQKFMSTSVGDRVSVTCKASQNVDTNVAWY | HY18-5B1.4 | VL | Mouse |
| | QKKPGQSPKALIYSASYRYSGVPDRFTGSGSGTDFTLTIS | | | |
| | NVQSEDLAEYFCQQYNSYPYTFGGGTKLEIK | | | |
| 173 | QVQLVQSGAEVKKPGASVKVSCKASGFNIKDTYIYWVR | HY18-5B1.5 | VH | Humanized |
| | QATGQGLEWIGRIDPANGNTKYDPKVQGRVTITRNTSI | | | |
| | STAYMELSSLRSEDTAVYYCALEHGYYDVMDYWGQGTL | | | |
| | VTVSS | | | |
| 37 | DIVMTQSQKFMSTSVGDRVSVTCKASQNVDTNVAWY | HY18-5B1.5 | VL | Mouse |
| | QKKPGQSPKALIYSASYRYSGVPDRFTGSGSGTDFTLTIS | | | |
| | NVQSEDLAEYFCQQYNSYPYTFGGGTKLEIK | | | |
| 36 | EVQLQQSGAELVKPGASVKLSCTASGFNIKDTYIYWVRQ | HY18-5B1.6 | VH | Mouse |
| | RPEQGLEWIGRIDPANGNTKYDPKVQGKATITADTSSN | | | |
| | TAYLQLSSLTFEDTAVYYCALEHGYYDVMDYWGQGTSV | | | |
| | TVSS | | | |
| 170 | DIQMTQSPSSLSASVGDRVTITCKASQNVGTNVAWYQ | HY18-5B1.6 | VL | Humanized |
| | QKPGKAPKALIYSASYRYSGVPSRFSGSGSGTDFTLTISSL | | | |
| | QPEDFATYYCQQYNSYPLTFGQGTKLEIK | | | |
| 172 | QVQLVQSGAEVKKPGASVKVSCKASGFNIKDTYINWVR | HY18-5B1.9 | VH | Humanized |
| | QATGQGLEWMGWIDPANGNTGYAQKFQGRVTITRNT | | | |
| | SISTAYMELSSLRSEDTAVYYCALEHGYYDVMDYWGQG | | | |
| | TLVTVSS | | | |
| 170 | DIQMTQSPSSLSASVGDRVTITCKASQNVGTNVAWYQ | HY18-5B1.9 | VL | Humanized |
| | QKPGKAPKALIYSASYRYSGVPSRFSGSGSGTDFTLTISSL | | | |
| | QPEDFATYYCQQYNSYPLTFGQGTKLEIK | | | |
| N/A | Variable region sequence (CDRs bolded) | HY18-5D1 | N/A | N/A |
| 38 | EIQLQQSGPELVKPGASVHISCKSSGYAFTSYNIYWVKQS | HY18-5D1.1 | VH | Mouse |
| | HGKSLEWIGHIDPDNGGIIYNQKFRGKATLTVDKSSSTA | | | |
| | YMHLNSLTSEDSAVYYCAREDGNYDYWGQGTTLTVSS | | | |
| 39 | DIVMTQSPKFMSTSVGDRVSVTCKASQNVGTNVAWYR | HY18-5D1.1 | VL | Mouse |
| | QKQGQSPKALIYSASYRYSGVPDRFTGSGSGTDFTLTISN | | | |
| | VQSEDLADYFCQQYNSYPLTFGAGTKLELK | | | |
| 38 | EIQLQQSGPELVKPGASVHISCKSSGYAFTSYNIYWVKQS | HY18-5D1.2 | VH | Mouse |
| | HGKSLEWIGHIDPDNGGIIYNQKFRGKATLTVDKSSSTA | | | |
| | YMHLNSLTSEDSAVYYCAREDGNYDYWGQGTTLTVSS | | | |
| 24 | DIQMTQSPSSLSASVGDRVTITCKASQNVGTNVAWFQ | HY18-5D1.2 | VL | Humanized |
| | QKPGKAPKALIYSASYRYSGVPSRFSGSGSGTDFTLTISSL | | | |
| | QPEDFATYYCQQYNSYPLTFGQGTKLEIK | | | |
| N/A | Variable region sequence (CDRs bolded) | HY19-2A10 | N/A | N/A |
| 40 | EVQLQQSGPELVKPGASVKISCKTSGYTFTESTMYWVKQ | HY19- | VH | Mouse |
| | SHGKSLEWIGHINPNNGGTSYNQKFRGKATLTVDKSSST | 2A10.1 | | |
| | AYMELRSLTSEDSAVYYCAMELFYFDYWGQGTTLTVSS | | | |
| 41 | DIVMTQSQKFMSTSVGDRVSVTCKASQNVGTNVAWY | HY19- | VL | Mouse |
| | QQKPGQSPKALIYSASYRYSGVPDRFTGSGSGTDFTLTIS | 2A10.1 | | |
| | NVQSEDLAEYFCQQYNISPYTFGGGTKLEIK | | | |
| N/A | Variable region sequence (CDRs bolded) | HY19-3A2 | N/A | N/A |
| 42 | QAYLQQSGAELVRSGASVKMSCKASGYTFTSFSMHWIK | HY19-3A2.1 | VH | Mouse |
| | QTPGQGLEWIGYIYPGNGYTNYNQKFKGKATLTADTSS | | | |
| | NTAYMQISSLTSEDSAVYFCGRDYGHTDYDYAMDYWG | | | |
| | QGTSVTVSS | | | |
| 43 | DIVMTQSQKFMSTSVGDRVSVSCKASQNVGTNVAWY | HY19-3A2.1 | VL | Mouse |
| | QQKPGRSPKALIYSASYRYSGVPDRFTGSGSGTDFTLTIS | | | |
| | NVQSEDLAEYFCQQYNTYPYTFGGGTKLEIK | | | |
| 42 | QAYLQQSGAELVRSGASVKMSCKASGYTFTSFSMHWIK | HY19-3A2.2 | VH | Mouse |
| | QTPGQGLEWIGYIYPGNGYTNYNQKFKGKATLTADTSS | | | |
| | NTAYMQISSLTSEDSAVYFCGRDYGHTDYDYAMDYWG | | | |
| | QGTSVTVSS | | | |
| 24 | DIQMTQSPSSLSASVGDRVTITCKASQNVGTNVAWFQ | HY19-3A2.2 | VL | Humanized |
| | QKPGKAPKALIYSASYRYSGVPSRFSGSGSGTDFTLTISSL | | | |
| | QPEDFATYYCQQYNSYPLTFGQGTKLEIK | | | |
| N/A | Variable region sequence (CDRs bolded) | HY11-2D4 | N/A | N/A |
| 44 | EVQLQQSGAELVRSGASVKLSCTASGFNIKDQFMHWV | HY11-2D4 | VH | Mouse |
| | NORPEQGLEWIGWIDPQNGNTEYAPKFQGKATMTAD | | | |
| | TSSNTAYLQLSSLTSEDTAVYYCNAEKYVNYETWFAYW | | | |
| | GQGTLVTVSA | | | |
| 45 | DIVMTQSQKFMSTSVGDRVSVTCKASQNVGTNVAWY | HY11-2D4 | VL | Mouse |
| | QQKPGQSPKALIYSASYRYSGVPDRFTGSGSGTDFTLTIS | | | |
| | NVQSEDLAEYFCQQYNTYPFTFGGGTKLEIK | | | |
| N/A | Variable region sequence (CDRs bolded) | HY11-2F5 | N/A | N/A |
| 46 | QVQLQQSGPELVRPGASVKMSCKASGYTFTNYWMYW | HY11-2F5 | VH | Mouse |
| | VKQRPGQGLEWIGMIDPSNSETRLNQKFKDKATLNVDK | | | |
| | SSNTAYMQLSSLTSEDSAVYYCASELGRYAYWGQGTLVT | | | |
| | VSA | | | |
| 47 | DIVMTQSQNFMSSSVGDRVSVTCKASQNVDTNVAWY | HY11-2F5 | VL | Mouse |
| | QQKPGQSPKALIYSASYRYSGVPDRFTGSGSGTDFTLTIS | | | |
| | NVQSEDLAEYFCQQYNSYPFTFGSGTKLEIK | | | |
| N/A | Variable region sequence (CDRs bolded) | HY11-2H1 | N/A | N/A |
| 48 | QVQLQQPGAELVRPGASVKLSCKASGYSFTSYWMYWV | HY11-2H1 | VH | Mouse |
| | KQRPGQGLEWIGMIHPSDSETRLNQKFKDKATLTVDKS | | | |
| | SSTAFMQLSSPTSEDSAVYYCASEIYYDYDNGWFAYWG | | | |
| | QGTLVTVSA | | | |
| 49 | DIVMTQSQKFMSTLVGDRVSVTCKASQNVDTNVAWY | HY11-2H1 | VL | Mouse |
| | QQKPGQSPKALIYSASYRYSGVPDRFTGSGSGTDFTLTIS | | | |
| | NVQSEDLAEYFCQQYNSHPYTFGGGTKLEIK | | | |
| N/A | Variable region sequence (CDRs bolded) | HY18-2D12 | N/A | N/A |
| 50 | QVQLQQSGAELVKPGASVKLSCKAFGYTFTNYDIHWVR | HY18-2D12 | VH | Mouse |
| | QRPEQGLEWIGWIFPGDGSTKYNEKFKGKATLTTDKSSS | | | |
| | TAYMQLSRLTSEDSAVYFCASELGREDYWGQGTTLTVSS | | | |
| 15 | DIVMTQSQKFMSTSVGDRVSVTCKASQNVGTNVAWY | HY18-2D12 | VL | Mouse |
| | QQKPGQSPKALIYSASYRYSGVPDRFTGSGSGTDFTLTIS | | | |
| | NVQSEDLAEYFCQQYNSYPYTFGGGTKLEIK | | | |
| N/A | Variable region sequence (CDRs bolded) | HY18-3F1 | N/A | N/A |
| 51 | QVQLQQSGPELVRPGASVKMSCKASGYTFTNYWIYWV | HY18-3F1 | VH | Mouse |
| | KQRPGQGLEWIGMIDPSNTEARLHQRFKDKATLNVDKS | | | |
| | SNTAYMQLSSLTSEDSAVYYCAIEYGNSDYWGQGTTLTV | | | |
| | SS | | | |
| 52 | DIVMTQSQKFMSTSVGDRVSVTCKASQNVGTNVAWY | HY18-3F1 | VL | Mouse |
| | QQKPGQSPKALIYSASYRYSGVPDRFTGSGSGTDFTLTIN | | | |
| | NVQSEDLAEYFCQQYNSYPYTFGGGTKLEIK | | | |
| N/A | Variable region sequence (CDRs bolded) | HY19-1G6 | N/A | N/A |
| 53 | EVHLQQSGAELVKPGASVKLSCTPSGFNIKDTYIHWVNQ | HY19-1G6 | VH | Mouse |
| | RPEQGLEWIGGIDPANVNPKYDPKFQAKATITADTSSN | | | |
| | TAYLQLSSLTSEDTAVYYCGIAYYRYDVWFAYWGQGTL | | | |
| | VTVSA | | | |
| 54 | DIVMTQSQKFMSTSVGDRVSVTCKASQNVNTNVVWY | HY19-1G6 | VL | Mouse |
| | QQKPGQSPKALIYSASYRYSGVPDRFTGSGSGTDFTLTIS | | | |
| | NVQSENLGKYFCQQYNSYPYTFGGGTKLEIK | | | |

TABLE C2
CDRs sequences of additional anti-fentanyl families and clones

| SEQ | | | | |
|---|---|---|---|---|
| ID | | | | |
| NO | Sequence | Clone | Chain | CDR |
| 98 | GFTFTDYYMS | HY17-4A5 clones | VH | CDR1 |
| 99 | T₁IGNRPNGYTT T₂YT₃T₄SVKG | HY17-4A5 clones | VH | CDR2 |
| | T₁ is F or Y, | | | |
| | T₂ is E or Y, | | | |
| | T₃ is N, A, or Q, and | | | |
| | T₄ is A or D | | | |
| 101 | FIGNRPNGYTTEYNASVKG | HY17-4A5.1 | VH | CDR2 |
| | | HY17-4A5.2 | | |
| | | HY17-4A5.3 | | |
| | | HY17-4A5.7 | | |
| 102 | YIGNRPNGYTTYYADSVKG | HY17-4A5.6 | VH | CDR2 |
| 103 | FIGNRPNGYTTEYQASVKG | HY17-4A5.8 | VH | CDR2 |
| 104 | ARGSTMITWFAY | HY17-4A5 clones | VH | CDR3 |
| 105 | RSSQSIVHSNGDTYFD | HY17-4A5.1 | VL | CDR1 |
| 89 | KASQNVGTNVA | HY17-4A5.2 | VL | CDR1 |
| 106 | RSSQSIVHSNGDTYLD | HY17-4A5.3 | VL | CDR1 |
| | | HY17-4A5.6 | | |
| | | HY17-4A5.7 | | |
| | | HY17-4A5.8 | | |
| 107 | KVSNRFS | HY17-4A5.1 | VL | CDR2 |
| 92 | SASYRYS | HY17-4A5.2 | VL | CDR2 |
| 108 | KVSNRAS | HY17-4A5.3 | VL | CDR2 |
| | | HY17-4A5.6 | | |
| | | HY17-4A5.7 | | |
| | | HY17-4A5.8 | | |
| 109 | FQGSLLPPA | HY17-4A5.1 | VL | CDR3 |
| | | HY17-4A5.3 | | |
| | | HY17-4A5.6 | | |
| | | HY17-4A5.7 | | |
| | | HY17-4A5.8 | | |
| 97 | QQYNSYPLT | HY17-4A5.2 | VL | CDR3 |
| 110 | GYTFTTYTMY | HY18-1B6.1 | VH | CDR1 |
| 111 | YINPGSGYTEYNQKFKD | HY18-1B6.1 | VH | CDR2 |
| 112 | ATEEVRRGDYFDY | HY18-1B6.1 | VH | CDR3 |
| 89 | KASQNVGTNVA | HY18-1B6.1 | VL | CDR1 |
| 92 | SASYRYS | HY18-1B6.1 | VL | CDR2 |
| 113 | QQYNSFPFT | HY18-1B6.1 | VL | CDR3 |
| 114 | GFNIKDTYIY | HY18-5B1.1 | VH | CDR1 |
| 115 | RIDPANGNTKYDPKVQG | HY18-5B1.1 | VH | CDR2 |
| 116 | ALEHGYYDVMDY | HY18-5B1.1 | VH | CDR3 |
| 117 | KASQNVDTNVA | HY18-5B1.1 | VL | CDR1 |
| 92 | SASYRYS | HY18-5B1.1 | VL | CDR2 |
| 95 | QQYNSYPYT | HY18-5B1.1 | VL | CDR3 |
| 114 | GFNIKDTYIY | HY18-5B1.2 | VH | CDR1 |
| 115 | RIDPANGNTKYDPKVQG | HY18-5B1.2 | VH | CDR2 |
| 116 | ALEHGYYDVMDY | HY18-5B1.2 | VH | CDR3 |
| 89 | KASQNVGTNVA | HY18-5B1.2 | VL | CDR1 |
| 92 | SASYRYS | HY18-5B1.2 | VL | CDR2 |
| 97 | QQYNSYPLT | HY18-5B1.2 | VL | CDR3 |
| 174 | GFNIKDTYIN | HY18-5B1.4 | VH | CDR1 |
| 175 | WIDPANGNTGYAQKFQG | HY18-5B1.4 | VH | CDR2 |
| 116 | ALEHGYYDVMDY | HY18-5B1.4 | VH | CDR3 |
| 117 | KASQNVDTNVA | HY18-5B1.4 | VL | CDR1 |
| 92 | SASYRYS | HY18-5B1.4 | VL | CDR2 |
| 95 | QQYNSYPYT | HY18-5B1.4 | VL | CDR3 |
| 114 | GFNIKDTYIY | HY18-5B1.5 | VH | CDR1 |
| 115 | RIDPANGNTKYDPKVQG | HY18-5B1.5 | VH | CDR2 |
| 116 | ALEHGYYDVMDY | HY18-5B1.5 | VH | CDR3 |
| 117 | KASQNVDTNVA | HY18-5B1.5 | VL | CDR1 |
| 92 | SASYRYS | HY18-5B1.5 | VL | CDR2 |
| 95 | QQYNSYPYT | HY18-5B1.5 | VL | CDR3 |
| 114 | GFNIKDTYIY | HY18-5B1.6 | VH | CDR1 |
| 115 | RIDPANGNTKYDPKVQG | HY18-5B1.6 | VH | CDR2 |
| 116 | ALEHGYYDVMDY | HY18-5B1.6 | VH | CDR3 |
| 89 | KASQNVGTNVA | HY18-5B1.6 | VL | CDR1 |
| 92 | SASYRYS | HY18-5B1.6 | VL | CDR2 |
| 97 | QQYNSYPLT | HY18-5B1.6 | VL | CDR3 |
| 174 | GFNIKDTYIN | HY18-5B1.9 | VH | CDR1 |
| 175 | WIDPANGNTGYAQKFQG | HY18-5B1.9 | VH | CDR2 |
| 116 | ALEHGYYDVMDY | HY18-5B1.9 | VH | CDR3 |
| 89 | KASQNVGTNVA | HY18-5B1.9 | VL | CDR1 |
| 92 | SASYRYS | HY18-5B1.9 | VL | CDR2 |
| 97 | QQYNSYPLT | HY18-5B1.9 | VL | CDR3 |
| 118 | GYAFTSYNIY | HY18-5D1.1 | VH | CDR1 |
| 119 | HIDPDNGGIIYNQKFRG | HY18-5D1.1 | VH | CDR2 |
| 120 | AREDGNYDY | HY18-5D1.1 | VH | CDR3 |
| 89 | KASQNVGTNVA | HY18-5D1.1 | VL | CDR1 |
| 92 | SASYRYS | HY18-5D1.1 | VL | CDR2 |
| 97 | QQYNSYPLT | HY18-5D1.1 | VL | CDR3 |
| 118 | GYAFTSYNIY | HY18-5D1.2 | VH | CDR1 |
| 119 | HIDPDNGGIIYNQKFRG | HY18-5D1.2 | VH | CDR2 |
| 120 | AREDGNYDY | HY18-5D1.2 | VH | CDR3 |
| 89 | KASQNVGTNVA | HY18-5D1.2 | VL | CDR1 |
| 92 | SASYRYS | HY18-5D1.2 | VL | CDR2 |
| 97 | QQYNSYPLT | HY18-5D1.2 | VL | CDR3 |
| 121 | GYTFTESTMY | HY19-2A10.1 | VH | CDR1 |
| 122 | HINPNNGGTSYNQKFRG | HY19-2A10.1 | VH | CDR2 |
| 123 | AMELFYFDY | HY19-2A10.1 | VH | CDR3 |
| 89 | KASQNVGTNVA | HY19-2A10.1 | VL | CDR1 |
| 92 | SASYRYS | HY19-2A10.1 | VL | CDR2 |
| 124 | QQYNISPYT | HY19-2A10.1 | VL | CDR3 |
| 125 | GYTFTSFSMH | HY19-3A2.1 | VH | CDR1 |
| 126 | YIYPGNGYTNYNQKFKG | HY19-3A2.1 | VH | CDR2 |
| 127 | GRDYGHTDYDYAMDY | HY19-3A2.1 | VH | CDR3 |
| 89 | KASQNVGTNVA | HY19-3A2.1 | VL | CDR1 |
| 92 | SASYRYS | HY19-3A2.1 | VL | CDR2 |
| 128 | QQYNTYPYT | HY19-3A2.1 | VL | CDR3 |
| 125 | GYTFTSFSMH | HY19-3A2.2 | VH | CDR1 |
| 126 | YIYPGNGYTNYNQKFKG | HY19-3A2.2 | VH | CDR2 |
| 127 | GRDYGHTDYDYAMDY | HY19-3A2.2 | VH | CDR3 |
| 89 | KASQNVGTNVA | HY19-3A2.2 | VL | CDR1 |
| 92 | SASYRYS | HY19-3A2.2 | VL | CDR2 |
| 97 | QQYNSYPLT | HY19-3A2.2 | VL | CDR3 |
| 129 | GFNIKDQFMH | HY11-2D4 | VH | CDR1 |
| 130 | WIDPQNGNTEYAPKFQG | HY11-2D4 | VH | CDR2 |
| 131 | NAEKYVNYETWFAY | HY11-2D4 | VH | CDR3 |
| 89 | KASQNVGTNVA | HY11-2D4 | VL | CDR1 |
| 92 | SASYRYS | HY11-2D4 | VL | CDR2 |
| 132 | QQYNTYPFT | HY11-2D4 | VL | CDR3 |
| 133 | GYTFTNYWMY | HY11-2F5 | VH | CDR1 |
| 134 | MIDPSNSETRLNQKFKD | HY11-2F5 | VH | CDR2 |
| 135 | ASELGRYAY | HY11-2F5 | VH | CDR3 |
| 117 | KASQNVDTNVA | HY11-2F5 | VL | CDR1 |
| 92 | SASYRYS | HY11-2F5 | VL | CDR2 |
| 136 | QQYNSYPFT | HY11-2F5 | VL | CDR3 |
| 137 | GYSFTSYWMY | HY11-2H1 | VH | CDR1 |
| 138 | MIHPSDSETRLNOKFKD | HY11-2H1 | VH | CDR2 |
| 139 | ASEIYYDYDNGWFAY | HY11-2H1 | VH | CDR3 |
| 117 | KASQNVDTNVA | HY11-2H1 | VL | CDR1 |
| 92 | SASYRYS | HY11-2H1 | VL | CDR2 |
| 140 | QQYNSHPYT | HY11-2H1 | VL | CDR3 |
| 141 | GYTFTNYDIH | HY18-2D12 | VH | CDR1 |
| 83 | WIFPGDGSTKYNEKFKG | HY18-2D12 | VH | CDR2 |
| 142 | ASELGREDY | HY18-2D12 | VH | CDR3 |
| 89 | KASQNVGTNVA | HY18-2D12 | VL | CDR1 |
| 92 | SASYRYS | HY18-2D12 | VL | CDR2 |
| 95 | QQYNSYPYT | HY18-2D12 | VL | CDR3 |
| 143 | GYTFTNYWIY | HY18-3F1 | VH | CDR1 |
| 144 | MIDPSNTEARLHQRFKD | HY18-3F1 | VH | CDR2 |
| 145 | AIEYGNSDY | HY18-3F1 | VH | CDR3 |
| 89 | KASQNVGTNVA | HY18-3F1 | VL | CDR1 |
| 92 | SASYRYS | HY18-3F1 | VL | CDR2 |
| 95 | QQYNSYPYT | HY18-3F1 | VL | CDR3 |
| 146 | GFNIKDTYIH | HY19-1G6 | VH | CDR1 |
| 147 | GIDPANVNPKYDPKFQA | HY19-1G6 | VH | CDR2 |
| 148 | GIAYYRYDVWFAY | HY19-1G6 | VH | CDR3 |
| 149 | KASQNVNTNVV | HY19-1G6 | VL | CDR1 |
| 92 | SASYRYS | HY19-1G6 | VL | CDR2 |
| 95 | QQYNSYPYT | HY19-1G6 | VL | CDR3 |
| 150 | GYTFTSYVMH | HY11-5C1.1 | VH | CDR1 |
| | | HY11-5C1.2 | | |
| 151 | YINPYNDGTKYNEKFKG | HY11-5C1.1 | VH | CDR2 |
| | | HY11-5C1.2 | | |
| 152 | ASEGNYEDY | HY11-5C1.1 | VH | CDR3 |
| | | HY11-5C1.2 | | |
| 89 | KASQNVGTNVA | HY11-5C1.1 | VL | CDR1 |
| | | HY11-5C1.2 | | |
| 92 | SASYRYS | HY11-5C1.1 | VL | CDR2 |
| | | HY11-5C1.2 | | |
| 96 | QQYYNYPYT | HY11-5C1.1 | VL | CDR3 |
| 95 | QQYNSYPYT | HY11-5C1.2 | VL | CDR3 |
| 153 | GHTFTSYYIY | HY11-6B2.1 | VH | CDR1 |
| | | HY11-6B2.2 | | |
| 154 | WIYFGNINLKYNEKFKG | HY11-6B2.1 | VH | CDR2 |
| | | HY11-6B2.2 | | |
| 155 | ASEDGPGAY | HY11-6B2.1 | VH | CDR3 |
| | | HY11-6B2.2 | | |
| 89 | KASQNVGTNVA | HY11-6B2.1 | VL | CDR1 |
| | | HY11-6B2.2 | | |
| 92 | SASYRYS | HY11-6B2.1 | VL | CDR2 |
| | | HY11-6B2.2 | | |
| 97 | QQYNSYPLT | HY11-6B2.1 | VL | CDR3 |
| 95 | QQYNSYPYT | HY11-6B2.2 | VL | CDR3 |
| 156 | GYTFSENTIY | HY17-2A2.1 | VH | CDR1 |
| 157 | GINPKNGGTTYNPEFKG | HY17-2A2.1 | VH | CDR2 |
| 158 | AIYDSDYFDY | HY17-2A2.1 | VH | CDR3 |
| 89 | KASQNVGTNVA | HY17-2A2.1 | VL | CDR1 |
| 159 | SASFRYG | HY17-2A2.1 | VL | CDR2 |
| 160 | QQYNSYPIT | HY17-2A2.1 | VL | CDR3 |
| 156 | GYTFSENTIY | HY17-2A2.4 | VH | CDR1 |
| 157 | GINPKNGGTTYNPEFKG | HY17-2A2.4 | VH | CDR2 |
| 158 | AIYDSDYFDY | HY17-2A2.4 | VH | CDR3 |
| 89 | KASQNVGTNVA | HY17-2A2.4 | VL | CDR1 |
| 159 | SASFRYG | HY17-2A2.4 | VL | CDR2 |
| 160 | QQYNSYPIT | HY17-2A2.4 | VL | CDR3 |
| 156 | GYTFSENTIY | HY17-2A2.7 | VH | CDR1 |
| 157 | GINPKNGGTTYNPEFKG | HY17-2A2.7 | VH | CDR2 |
| 158 | AIYDSDYFDY | HY17-2A2.7 | VH | CDR3 |
| 89 | KASQNVGTNVA | HY17-2A2.7 | VL | CDR1 |
| 92 | SASYRYS | HY17-2A2.7 | VL | CDR2 |
| 97 | QQYNSYPLT | HY17-2A2.7 | VL | CDR3 |
| 156 | GYTFSENTIY | HY17-2A2.8 | VH | CDR1 |
| 157 | GINPKNGGTTYNPEFKG | HY17-2A2.8 | VH | CDR2 |
| 158 | AIYDSDYFDY | HY17-2A2.8 | VH | CDR3 |
| 89 | KASQNVGTNVA | HY17-2A2.8 | VL | CDR1 |
| 92 | SASYRYS | HY17-2A2.8 | VL | CDR2 |
| 97 | QQYNSYPLT | HY17-2A2.8 | VL | CDR3 |

In certain embodiments, an anti-fentanyl antibody, or a fragment thereof, comprises three light chain CDR sequences (VL. CDR1, CDR2 and CDR3), and/or three heavy chain CDR sequences (VH CDR1, CDR2 and CDR3), as described in any of the following antibody clones described herein: HY11-5C1.1, HY11-5C2, HY11-6B2.1, HY11-6B2.2, HY17-2A2.1, HY17-2A2.4, HY17-2A2.7, HY17-2A2.8, HY17-4A5.1, HY17-4A5.2, HY17-4A5.3, HY17-4A5.6, HY17-4A5.7, HY17-4A5.8, HY18-11B6.1, HY18-51B1.1, HY18-51B1.2, HY18-51B1.4, HY18-S1f1.5, HY18-51B1.6, HY18-51B1.9, HY18-51D1.1, HY18-51D1.2, HY19-2A10.1 (also referred to as HY19-11H6.1), HY19-3A2.1, HY19-3A2.2, HY11-21D4, HY11-21F5, HY11-21H1, HY18-21D12, HY18-31F1, and HY19-1G6, as set forth in Table C1 (CDRs bolded) and Table C2.

In certain embodiments, an anti-fentanyl antibody, or a fragment thereof, comprises a light chain sequence comprising an amino acid sequence that has at least about 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to a VL amino acid sequence of any of the following antibody clones described herein: HY11-5C1.1, HY11-5C1.2, HY11-6B2.1, HY11-6B2.2, HY17-2A2.1, HY17-2A2.4, HY17-2A2.7, HY17-2A2.8, HY17-4A5.1, HY17-4A5.2, HY17-4A5.3, HY17-4A5.6, HY17-4A5.7, HY17-4A5.8, HY18-1B6.1, HY18-5B1.1, HY18-5B1.2, HY18-5B1.4, HY18-5B1.5, HY18-5B1.6, HY18-5B1.9, HY18-5D1.1, HY18-5D1.2, HY19-2A10.1 (also referred to as HY19-1H6.1), HY19-3A2.1, HY19-3A2.2, HY11-2D4, HY11-2F5, HY11-2H1, HY18-2D12, HY18-3F1, and HY19-1G6 and/or a heavy chain sequence comprising an amino acid sequence that has at least about 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to a VH amino acid sequence of any of the following antibody clones described herein: HY11-5C1.1, HY11-5C1.2, HY11-6B2.1, HY11-6B2.2, HY17-2A2.1, HY17-2A2.4, HY17-2A2.7, HY17-2A2.8, HY17-4A5.1, HY17-4A5.2, HY17-4A5.3, HY17-4A5.6, HY17-4A5.7, HY17-4A5.8, HY18-1B6.1, HY18-5B1.1, HY18-5B1.2, HY18-5B1.4, HY18-5B1.5, HY18-5B1.6, HY18-5B1.9, HY18-5D1.1, HY18-5D1.2, HY19-2A10.1 (also referred to as HY19-1H6.1), HY19-3A2.1, HY19-3A2.2, HY11-2D4, HY11-2F5, HY11-2H1, HY18-2D12, HY18-3F1, and HY19-1G6. The amino acid sequences of the light chain variable domain (VL) and heavy chain variable domain (VH) of these anti-fentanyl antibody clones are set forth in Table C1.

For example, two representative clones of HY17-4A5 family (HY17-4A5.6 and HY17-4A5.7), one representative clone of HY17-2A2 family, and one representative clone of HY18-5B1 family are illustrated below as non-limiting embodiments.

### Clone HY17-4A5.6

In some embodiments, an anti-fentanyl antibody, or fragment thereof, comprises a light chain CDR1 comprising the amino acid sequence of SEQ ID NO:106, a light chain CDR2 comprising the amino acid sequence of SEQ ID NO:108, and a light chain CDR3 comprising the amino acid sequence of SEQ ID NO: 109. In some embodiments, an anti-fentanyl antibody, or fragment thereof, comprises a heavy chain CDR1 comprising the amino acid sequence of SEQ ID NO:98, a heavy chain CDR2 comprising the amino acid sequence of SEQ ID NO: 101, and a heavy chain CDR3 comprising the amino acid sequence of SEQ ID NO:104. In some embodiments, an anti-fentanyl antibody, or fragment thereof, comprises a light chain CDR1-3 and a heavy chain CDR1-3 comprising the amino acid sequences of SEQ ID NOs: 106, 108, 109, 98, 101 and 104, respectively. In some embodiments, an anti-fentanyl antibody, or fragment thereof, comprises a light chain CDR1-3 and a heavy chain CDR1-3 consisting of the amino acid sequences of SEQ ID NOs:106, 108, 109, 98, 101 and 104, respectively.

In some embodiments, an anti-fentanyl antibody, or fragment thereof, comprises a light chain variable region comprising an amino acid sequence that has at least about 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to SEQ ID NO:57. In some embodiments, an anti-fentanyl antibody, or fragment thereof, comprises a light chain variable region comprising the amino acid sequence of SEQ ID NO:57. In some embodiments, an anti-fentanyl antibody, or fragment thereof, comprises a light chain variable region consisting of the amino acid sequence of SEQ ID NO:57.

In some embodiments, an anti-fentanyl antibody, or fragment thereof, comprises a heavy chain variable region comprising an amino acid sequence that has at least about 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to SEQ ID NO:58. In some embodiments, an anti-fentanyl antibody, or fragment thereof, comprises a heavy chain variable region comprising the amino acid sequence of SEQ ID NO:58. In some embodiments, an anti-fentanyl antibody, or fragment thereof, comprises a heavy chain variable region consisting of the amino acid sequence of SEQ ID NO:58.

In some embodiments, an anti-fentanyl antibody, or fragment thereof, comprises a light chain variable region comprising an amino acid sequence that has at least about 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to SEQ ID NO:57 and further comprises a heavy chain variable region comprising an amino acid sequence that has at least about 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to SEQ ID NO:58. In some embodiments, an anti-fentanyl antibody, or fragment thereof, comprises a light chain variable region comprising the amino acid sequence of SEQ ID NO:57 and further comprises a heavy chain variable region comprising the amino acid sequence of SEQ ID NO:58. In some embodiments, an anti-fentanyl antibody, or fragment thereof, comprises a light chain variable region consisting of the amino acid sequence of SEQ ID NO:57 and further comprises a heavy chain variable region consisting of the amino acid sequence of SEQ ID NO:58.

### Clone HY17-4A5.7

In some embodiments, an anti-fentanyl antibody, or fragment thereof, comprises a light chain CDR1 comprising the amino acid sequence of SEQ ID NO:106, a light chain CDR2 comprising the amino acid sequence of SEQ ID NO:108, and a light chain CDR3 comprising the amino acid sequence of SEQ ID NO: 109. In some embodiments, an anti-fentanyl antibody, or fragment thereof, comprises a heavy chain CDR1 comprising the amino acid sequence of SEQ ID NO:98, a heavy chain CDR2 comprising the amino acid sequence of SEQ ID NO:102, and a heavy chain CDR3 comprising the amino acid sequence of SEQ ID NO:104. In some embodiments, an anti-fentanyl antibody, or fragment thereof, comprises a light chain CDR1-3 and a heavy chain CDR1-3 comprising the amino acid sequences of SEQ ID NOs: 106, 108, 109, 98, 101 and 104, respectively. In some embodiments, an anti-fentanyl antibody, or fragment thereof, comprises a light chain CDR1-3 and a heavy chain CDR1-3 consisting of the amino acid sequences of SEQ ID NOs:106, 108, 109, 98, 102 and 104, respectively.

In some embodiments, an anti-fentanyl antibody, or fragment thereof, comprises a light chain variable region comprising an amino acid sequence that has at least about 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to SEQ ID NO:57. In some embodiments, an anti-fentanyl antibody, or fragment thereof, comprises a light chain variable region comprising the amino acid sequence of SEQ ID NO:57. In some embodiments, an anti-fentanyl antibody, or fragment thereof, comprises a light chain variable region consisting of the amino acid sequence of SEQ ID NO:57.

In some embodiments, an anti-fentanyl antibody, or fragment thereof, comprises a heavy chain variable region comprising an amino acid sequence that has at least about 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to SEQ ID NO:59. In some embodiments, an anti-fentanyl antibody, or fragment thereof, comprises a heavy chain variable region comprising the amino acid sequence of SEQ ID NO:59. In some embodiments, an anti-fentanyl antibody, or fragment thereof, comprises a heavy chain variable region consisting of the amino acid sequence of SEQ ID NO:59.

In some embodiments, an anti-fentanyl antibody, or fragment thereof, comprises a light chain variable region comprising an amino acid sequence that has at least about 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to SEQ ID NO:57 and further comprises a heavy chain variable region comprising an amino acid sequence that has at least about 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to SEQ ID NO:59. In some embodiments, an anti-fentanyl antibody, or fragment thereof, comprises a light chain variable region comprising the amino acid sequence of SEQ ID NO:57 and further comprises a heavy chain variable region comprising the amino acid sequence of SEQ ID NO:59. In some embodiments, an anti-fentanyl antibody, or fragment thereof, comprises a light chain variable region consisting of the amino acid sequence of SEQ ID NO:57 and further comprises a heavy chain variable region consisting of the amino acid sequence of SEQ ID NO:59.

### Clone HY17-2A2.8

In some embodiments, an anti-fentanyl antibody, or fragment thereof, comprises a light chain CDR1 comprising the amino acid sequence of SEQ ID NO:89, a light chain CDR2 comprising the amino acid sequence of SEQ ID NO:92, and a light chain CDR3 comprising the amino acid sequence of SEQ ID NO:97. In some embodiments, an anti-fentanyl antibody, or fragment thereof, comprises a heavy chain CDR1 comprising the amino acid sequence of SEQ ID NO:156, a heavy chain CDR2 comprising the amino acid sequence of SEQ ID NO:157, and a heavy chain CDR3 comprising the amino acid sequence of SEQ ID NO:158. In some embodiments, an anti-fentanyl antibody, or fragment thereof, comprises a light chain CDR1-3 and a heavy chain CDR1-3 comprising the amino acid sequences of SEQ ID NOs: 89, 92, 97, 156, 157 and 158, respectively. In some embodiments, an anti-fentanyl antibody, or fragment thereof, comprises a light chain CDR1-3 and a heavy chain CDR1-3 consisting of the amino acid sequences of SEQ ID NOs: 89, 92, 97, 156, 157 and 158, respectively.

In some embodiments, an anti-fentanyl antibody, or fragment thereof, comprises a light chain variable region comprising an amino acid sequence that has at least about 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to SEQ ID NO:170. In some embodiments, an anti-fentanyl antibody, or fragment thereof, comprises a light chain variable region comprising the amino acid sequence of SEQ ID NO: 170. In some embodiments, an anti-fentanyl antibody, or fragment thereof, comprises a light chain variable region consisting of the amino acid sequence of SEQ ID NO:170.

In some embodiments, an anti-fentanyl antibody, or fragment thereof, comprises a heavy chain variable region comprising an amino acid sequence that has at least about 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to SEQ ID NO:171. In some embodiments, an anti-fentanyl antibody, or fragment thereof, comprises a heavy chain variable region comprising the amino acid sequence of SEQ ID NO:171. In some embodiments, an anti-fentanyl antibody, or fragment thereof, comprises a heavy chain variable region consisting of the amino acid sequence of SEQ ID NO:171.

In some embodiments, an anti-fentanyl antibody, or fragment thereof, comprises a light chain variable region comprising an amino acid sequence that has at least about 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to SEQ ID NO:170 and further comprises a heavy chain variable region comprising an amino acid sequence that has at least about 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to SEQ ID NO:171. In some embodiments, an anti-fentanyl antibody, or fragment thereof, comprises a light chain variable region comprising the amino acid sequence of SEQ ID NO:170 and further comprises a heavy chain variable region comprising the amino acid sequence of SEQ ID NO:171. In some embodiments, an anti-fentanyl antibody, or fragment thereof, comprises a light chain variable region consisting of the amino acid sequence of SEQ ID NO:170 and further comprises a heavy chain variable region consisting of the amino acid sequence of SEQ ID NO:171.

### Clone HY18-5B1.9

In some embodiments, an anti-fentanyl antibody, or fragment thereof, comprises a light chain CDR1 comprising the amino acid sequence of SEQ ID NO:89, a light chain CDR2 comprising the amino acid sequence of SEQ ID NO:92, and a light chain CDR3 comprising the amino acid sequence of SEQ ID NO:97. In some embodiments, an anti-fentanyl antibody, or fragment thereof, comprises a heavy chain CDR1 comprising the amino acid sequence of SEQ ID NO:174, a heavy chain CDR2 comprising the amino acid sequence of SEQ ID NO:175, and a heavy chain CDR3 comprising the amino acid sequence of SEQ ID NO:116. In some embodiments, an anti-fentanyl antibody, or fragment thereof, comprises a light chain CDR1-3 and a heavy chain CDR1-3 comprising the amino acid sequences of SEQ ID NOs: 89, 92, 97, 174, 175 and 116, respectively. In some embodiments, an anti-fentanyl antibody, or fragment thereof, comprises a light chain CDR1-3 and a heavy chain CDR1-3 consisting of the amino acid sequences of SEQ ID NOs: 89, 92, 97, 174, 175 and 116, respectively.

In some embodiments, an anti-fentanyl antibody, or fragment thereof, comprises a light chain variable region comprising an amino acid sequence that has at least about 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to SEQ ID NO:170. In some embodiments, an anti-fentanyl antibody, or fragment thereof, comprises a light chain variable region comprising the amino acid sequence of SEQ ID NO: 170. In some embodiments, an anti-fentanyl antibody, or fragment thereof, comprises a light chain variable region consisting of the amino acid sequence of SEQ ID NO:170.

In some embodiments, an anti-fentanyl antibody, or fragment thereof, comprises a heavy chain variable region comprising an amino acid sequence that has at least about 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to SEQ ID NO: 172. In some embodiments, an anti-fentanyl antibody, or fragment thereof, comprises a heavy chain variable region comprising the amino acid sequence of SEQ ID NO:172. In some embodiments, an anti-fentanyl antibody, or fragment thereof, comprises a heavy chain variable region consisting of the amino acid sequence of SEQ ID NO: 172.

In some embodiments, an anti-fentanyl antibody, or fragment thereof, comprises a light chain variable region comprising an amino acid sequence that has at least about 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to SEQ ID NO:170 and further comprises a heavy chain variable region comprising an amino acid sequence that has at least about 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to SEQ ID NO:172. In some embodiments, an anti-fentanyl antibody, or fragment thereof, comprises a light chain variable region comprising the amino acid sequence of SEQ ID NO:170 and further comprises a heavy chain variable region comprising the amino acid sequence of SEQ ID NO:172. In some embodiments, an anti-fentanyl antibody, or fragment thereof, comprises a light chain variable region consisting of the amino acid sequence of SEQ ID NO:170 and further comprises a heavy chain variable region consisting of the amino acid sequence of SEQ ID NO:172.

Accordingly, certain embodiments of the invention provide an isolated anti-fentanyl antibody or fragment thereof, comprising one or more complementarity determining regions (CDRs) selected from the group consisting of:
(a) a heavy chain CDR1 having at least 75% (e.g., 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%) sequence identity to an amino acid sequence of any one VH CDR1 sequence listed in Tables A1-A2, Tables B1-B2, Tables B3-B4, or Tables C1-C2;
(b) a heavy chain CDR2 having at least 75% (e.g., 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%) sequence identity to an amino acid sequence of any one VH CDR2 sequence listed in Tables A1-A2, Tables B1-B2, Tables B3-B4, or Tables C1-C2;
(c) a heavy chain CDR3 having at least 75% (e.g., 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%) sequence identity to an amino acid sequence of any one VH CDR3 sequence listed in Tables A1-A2, Tables B1-B2, Tables B3-B4, or Tables C1-C2;
(d) a light chain CDR1 having at least 75% (e.g., 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%) sequence identity to an amino acid sequence of any one VL CDR1 sequence listed in Tables A1-A2, Tables B1-B2, Tables B3-B4, or Tables C1-C2;
(e) a light chain CDR2 having at least 75% (e.g., 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%) sequence identity to an amino acid sequence of any one VL CDR2 sequence listed in Tables A1-A2, Tables B1-B2, Tables B3-B4, or Tables C1-C2; and
(f) a light chain CDR3 having at least 75% (e.g., 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%) sequence identity to an amino acid sequence of any one VL CDR3 sequence listed in Tables A1-A2, Tables B1-B2, Tables B3-B4, or Tables C1-C2.

In certain embodiments, the isolated anti-fentanyl antibody or fragment thereof, comprises three heavy chain CDRs and three light chain CDRs that are all selected from one clone listed in Tables A1-A2, Tables B1-B2, Tables B3-B4, or Tables C1-C2.

In certain embodiments, the isolated anti-fentanyl antibody or fragment thereof, comprises a heavy chain variable region comprising an amino acid sequence that has at least 80% sequence identity to the amino acid sequence of any VH sequence listed in Table A1, Table B1, Table B3, or Table C1.

In certain embodiments, the isolated anti-fentanyl antibody or fragment thereof, comprises a light chain variable region comprising an amino acid sequence that has at least 80% sequence identity to the amino acid sequence of any VL sequence listed in Table A1, Table B1, Table B3, or Table C1.

In certain embodiments, the isolated anti-fentanyl antibody or fragment thereof, comprises a heavy chain variable region comprising an amino acid sequence that has at least 80% sequence identity to the amino acid sequence of any VH sequence listed in Table A1, Table B1, Table B3, or Table C1, and a light chain variable region comprising an amino acid sequence that has at least 80% sequence identity to the amino acid sequence of any VL sequence listed in Table A1, Table B1, Table B3, or Table C1.

In certain embodiments, the isolated anti-fentanyl antibody or fragment thereof, comprises a heavy chain variable region and a light chain variable region that are both selected from one clone listed in Tables A1-A2, Tables B1-B2, Tables B3-B4, or Tables C1-C2.

In certain embodiments, the isolated anti-fentanyl antibody or fragment thereof, comprises a mutation in the human framework region that may help to maintain or improve target (e.g., fentanyl and/or carfentanil) binding. In certain embodiments, the isolated anti-fentanyl antibody or fragment thereof, comprises Y at position 36 of light chain variable region according to Kabat numbering. For example, one mutation of F36Y (mutation denoted according to Kabat numbering) can be introduced to SEQ ID NO:24 to arrive at SEQ ID NO:170 (e.g., see clones HY19-1H6.15, HY17-2A2.7, HY17-2A2.8, HY11-7E1.25, HY18-5B1.6, and HY18-5B1.9).

In certain embodiments, the antibody, or fragment thereof, as described herein has a Fab domain melting temperature (Fab Tₘ) of at least about 80° C., 81° C., 82° C., 83° C., 84° C., 85° C., or 86° C. in the presence of fentanyl or analog (i.e., Fab domain bound with fentanyl or analog), for example, as measured by dynamic scanning fluorimetry (DSF).

In certain embodiments, the antibody, or fragment thereof, as described herein has a Fab domain melting temperature (Fab Tₘ) of at least about 72° C., 73° C., 74° C., 75° C., 76° C., 77° C., 78° C., 79° C., 80° C., or 81° C. in the absence of fentanyl or analog (i.e., Fab domain without bound fentanyl or analog), for example, as measured by dynamic scanning fluorimetry (DSF).

In certain embodiments, the antibody, or fragment thereof, as described herein has a Fab Tₘ difference (ΔTₘ) between in the presence and absence of fentanyl or analog that is at least about 4° C., 4.5° C., 5° C., 5.5° C., 6° C., 6.5° C., 7° C., 7.5° C., or 8° C.

In certain embodiments, the binding affinity (K_{D} value) of an antibody or fragment thereof described herein for fentanyl or analog thereof is less than 1000, 900, 800, 700, 600, 500, 400, 300, 200, 100, 90, 80, 70, 60, 50, 40, 30, 20, 10, 9, 8, 7, 6, 5, 4, 3, 2, 1, 0.9, 0.8, 0.7, 0.6, 0.5, 0.4, 0.3, 0.2, 0.1, 0.09, 0.08, 0.07, 0.06, 0.05, 0.04, 0.03, 0.02, or 0.01 nM. In certain embodiments, the binding affinity (K_{D} value) of an antibody or fragment thereof described herein for fentanyl or analog thereof is less than 10 nM, 1 nM, 0.1 nM, 0.01 nM, or 1 μM.

In certain embodiments, the binding affinity (K_{D} value) of an antibody or fragment thereof described herein for fentanyl or analog thereof is about 0.1 μM to 0.001 nM, 0.01 to 100 nM, 0.02 to 90 nM, 0.03 to 80 nM, 0.04 to 70 nM, 0.05 to 60 nM, 0.06 to 50 nM, 0.07 to 40 nM, 0.08 to 30 nM, 0.09 to 20, or 0.1 to 10 nM. In certain embodiments, the binding affinity value K_{D} of an antibody or fragment thereof described herein for fentanyl or analog thereof is about 0.01 to 10 nM, 0.02 to 9 nM, 0.03 to 8 nM, 0.04 to 7 nM, 0.05 to 6 nM, 0.06 to 5 nM, 0.07 to 4 nM, 0.08 to 3 nM, 0.09 to 2 nM, or 0.1 to 1 nM. In certain embodiments, the binding affinity value K_{D} (i.e., K_{D}=K_{dissociation} (K_{d})/K_{association} (kₐ)) of an antibody or fragment thereof described herein for fentanyl or analog thereof is about 0.1 to 0.9 nM, 0.1 to 0.8 nM, 0.1 to 0.7 nM, 0.1 to 0.6 nM, 0.1 to 0.5 nM, 0.1 to 0.4 nM, 0.1 to 0.3 nM, or 0.1 to 0.2 nM.

In certain embodiments, an isolated anti-fentanyl antibody described herein, or fragment thereof, further comprises at least one heavy chain constant region and/or at least one light chain constant region. Thus, in certain embodiments, the light chain variable region is linked (e.g., through a linker or a direct bond, such as a peptide bond) to a light chain constant region (e.g., kappa or lambda). In certain embodiments, the heavy chain variable region is linked to at least one heavy chain constant region (e.g., 1, 2, or 3). In certain embodiments, the heavy and light chains are linked via one or more disulfide bonds.

In certain embodiments, the antibody or fragment thereof is a recombinant antibody or fragment thereof. In certain embodiments, the antibody or fragment thereof is a chimeric antibody or fragment thereof. In certain embodiments, the antibody or fragment thereof is humanized.

In certain embodiments, an antibody of the invention is a monoclonal antibody or a fragment thereof.

In certain embodiments, antibody, or fragment thereof, is a fragment. In certain embodiments, the fragment comprises an antigen-binding domain or a variable region. For example, in certain embodiments, the fragment is a fragment antigen-binding (Fab), F(ab′)₂, Fv, single-chain Fv (scFv), diabody (diabodies), or a multispecific or multivalent antibody prepared from an antibody fragment. In certain embodiments, the fragment is a Fab fragment (e.g., a Fab comprising a human antibody scaffold). In certain embodiments, the fragment is a scFv.

In certain embodiments, the antibody or fragment thereof (e.g., Fab or scFv) may be assembled on a carrier (e.g., antibody or fragment thereof decorated liposome or nanoparticle), or conjugated to a polymer (e.g., antibody or fragment thereof is conjugated to polyethylene glycol (PEG)), or delivered/expressed via a vector (e.g., AAV), or delivered via mRNA construct encoding the antibody or fragment thereof for in vivo expression.

In another embodiment, the antibody is a substantially full-length antibody, e.g., an IgG antibody (IgG1, IgG2, IgG3 or IgG4), or other antibody class (e.g., IgA or IgM) or isotype as defined herein. Engineered Fc domain is described herein and known in the art (e.g., Wilkinson, et al., PLoS One. 2021 Dec. 21; 16(12):e0260954, Booth, et al., MAbs. 2018 October; 10(7): 1098-1110, and Mackness, et al., MAbs. 2019 October; 11(7): 1276-1288, which are incorporated herein by reference). In certain embodiments, the antibody described herein comprises an engineered Fc domain. For example, in certain embodiments, the antibody described herein comprises an engineered Fc domain comprising a mutation that reduces FcγR binding and mitigates FcγR-mediated immune cell activation or inflammatory response as compared to the wildtype Fc domain. In certain embodiments, the antibody described herein comprises an engineered Fc domain comprising a mutation that modulates the antibody-FcRn interaction and extend the circulation half time of the antibody as compared to the wildtype Fc domain. In certain embodiments, the antibody as described herein is conjugated to a polymer (e.g., polyethylene glycol (PEG)) on the Fc domain.

In certain embodiments, the antibody is a multivalent antibody.

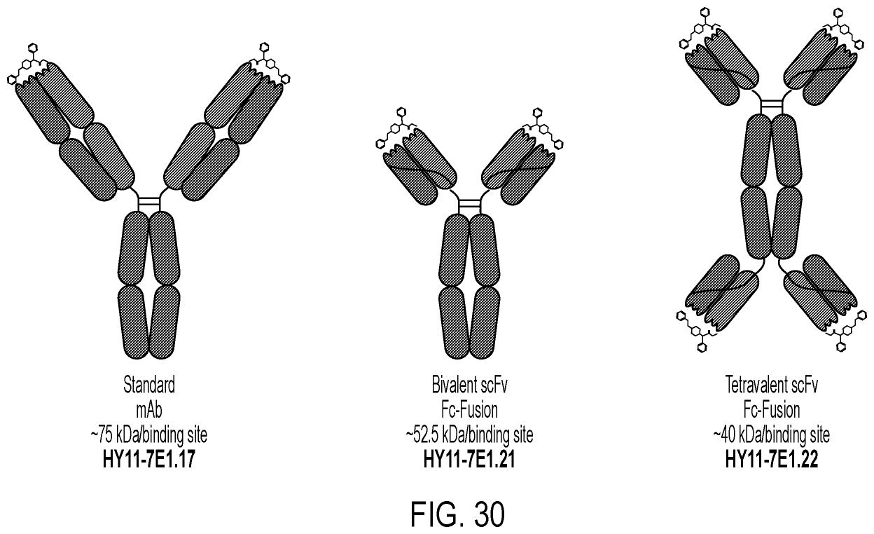

The term “antibody or fragment thereof” also encompasses antigen binding protein that comprises antigen binding domain (e.g., immunoglobulin variable region, scFv, or VHH). In certain embodiments, the antigen binding protein further comprises non-antigen binding domain (e.g., immunoglobulin constant region, Fc domain, or serum albumin (SA)), wherein antigen binding domain and non-antigen binding domain are linked (e.g., directly linked, or indirectly linked via a peptide or polypeptide linker). For instance, an anti-fentanyl binding protein described herein can be a full-length immunoglobulin (e.g., IgG1 mAb), or fusion proteins including but not limited to scFv-SA fusion, or scFv-Fc fusion as described herein (e.g., a bivalent or tetravalent or hexavalent scFv-Fc, also see FIG. 30). In certain embodiments, the non-antigen binding domain comprises immunoglobulin derived constant region. In certain embodiments, the non-antigen binding domain comprises Fc domain. In certain embodiments, the non-antigen binding domain does not comprise an immunoglobulin derived constant region or Fc. In certain embodiments, the non-antigen binding domain comprises serum albumin (SA) such as human serum albumin.

In certain embodiments, an isolated anti-fentanyl antibody, or fragment thereof, described herein does not comprise non-antigen binding domain (e.g., immunoglobulin constant region, Fc domain, or serum albumin (SA)). In certain embodiments, an isolated anti-fentanyl antibody, or fragment thereof, described herein comprises or consists of a scFv.

In certain embodiments, an isolated anti-fentanyl antibody, or fragment thereof, described herein comprises a non-antigen binding domain (e.g., immunoglobulin constant region, Fc domain, or serum albumin (SA)).

In certain embodiments, an isolated anti-fentanyl antibody, or fragment thereof, described herein does not comprise Fc domain.

In certain embodiments, an isolated anti-fentanyl antibody, or fragment thereof, described herein comprises Fc domain.

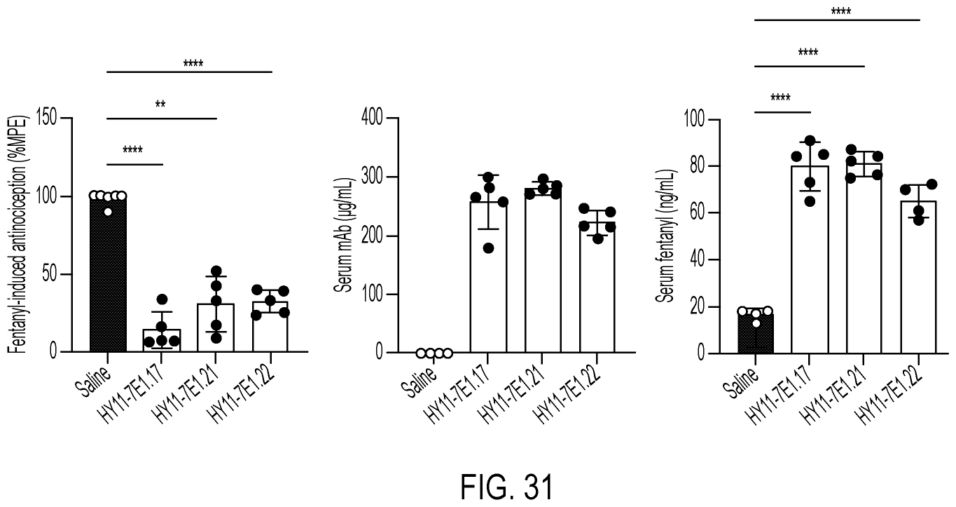

In certain embodiments, an isolated anti-fentanyl antibody, or fragment thereof, described herein is a scFv-Fc fusion protein that comprises Fc domain and one or more scFv (e.g., 2 or 4 or 6 scFv) linked to the Fc domain (e.g., linked to the N terminus of Fc domain, or linked to both the N terminus and C terminus of Fc domain, also see FIG. 30 and FIG. 31), e.g., to form bivalent, tetravalent, or hexavalent scFv-Fc fusion protein.

In certain embodiments, a scFv-Fc fusion polypeptide sequence comprises one or more scFv (e.g., 1, 2, or 3 scFv sequence) linked to the N terminus of the Fc domain sequence. In certain embodiments, a scFv-Fc fusion polypeptide sequence comprises two or more scFv (e.g., 2, or 3 scFv sequences) linked to the N terminus of the Fc domain sequence, wherein the two or more scFv are linked directly, or indirectly with linker sequence.

In certain embodiments, an isolated anti-fentanyl antibody, or fragment thereof, described herein comprises scFv-Fc fusion polypeptide sequence comprising an amino acid sequence that has at least about 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to any one of SEQ ID NOs:188, 190, or 191.

In certain embodiments, the scFv-Fc fusion polypeptide sequence further comprises one or more scFv (e.g., 1, 2, or 3 scFv) linked to the C terminus of the Fc domain sequence. In certain embodiments, the scFv-Fc fusion polypeptide sequence further comprises two or more scFv (e.g., 2 scFv) linked to the C terminus of the Fc domain sequence, wherein the two or more scFvs are linked directly, or indirectly with linker sequence.

In certain embodiments, an isolated anti-fentanyl antibody, or fragment thereof, described herein comprises scFv-Fc fusion polypeptide sequence comprising an amino acid sequence that has at least about 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to any one of SEQ ID NOs:189, 192, or 193.

TABLE E1

| SEQ ID | Amino acid sequence | |
|---|---|---|
| 188 | QVQLQESGPGLVKPSQTLSLTCTVSGDSITSGYWN | Bivalent scFv |
| | WIRQHPGKGLEYMGYISYSGSTYYNPSLKSRVTISV | construct HY11- |
| | DTSKNQFSLKLSSVTAADTAVYYCARYYGDNYVGA | 7E1.21 |
| | MDYWGQGTLVTVSSGGGGSGGGGSGGGGSGGGG | (scFv bolded, |
| | SDIVMTQSPLSLPVTPGEPASISCRSSKSLLHSNGITY | Linker underlined, |
| | LYWYLQKPGQSPQLLIYQMSNLASGVPDRFSGSGS | Fc domain |
| | GTDFTLKISRVEAEDVGVYYCAQNLELPWTFGGGT | italicized) |
| | KVEIKGSDKTHTCPPCPAPELLGGPSVFLFPPKPKDTL | |
| | MISRTPEVTCVVVDVSHEDPEVKFNWYVDGVEVHNAKTK | |
| | PREEQYNSTYRVVSVLTVLHODWLNGKEYKCKVSNKALP | |
| | APIEKTISKAKGOPREPQVYTLPPSRDELTKNQVSLTCLV | |
| | KGFYPSDIAVEWESNGQPENNYKTTPPVLDSDGSFFLYSK | |
| | LTVDKSRWQQGNVFSCSVMHEALHNHYTOKSLSLSPGK | |
| 189 | QVQLQESGPGLVKPSQTLSLTCTVSGDSITSGYWN | Tetravalent scFv |
| | WIRQHPGKGLEYMGYISYSGSTYYNPSLKSRVTISV | construct 1 |
| | DTSKNQFSLKLSSVTAADTAVYYCARYYGDNYVGA | HY11-7E1.22 |
| | MDYWGQGTLVTVSSGGGGSGGGGSGGGGSGGGG | (scFv bolded, |
| | SDIVMTQSPLSLPVTPGEPASISCRSSKSLLHSNGITY | Linker underlined, |
| | LYWYLQKPGQSPQLLIYQMSNLASGVPDRFSGSGS | Fc domain |
| | GTDFTLKISRVEAEDVGVYYCAQNLELPWTFGGGT | italicized) |
| | KVEIKGSDKTHTCPPCPAPELLGGPSVFLFPPKPKDTL | |
| | MISRTPEVTCVVVDVSHEDPEVKFNWYVDGVEVHNAKTK | |
| | PREEQYNSTYRVVSVLTVLHODWLNGKEYKCKVSNKALP | |
| | APIEKTISKAKGQPREPQVYTLPPSRDELTKNQVSLTCLV | |
| | KGFYPSDIAVEWESNGQPENNYKTTPPVLDSDGSFFLYSK | |
| | LTVDKSRWQQGNVFSCSVMHEALHNHYTQKSLSLSPGK | |
| | GGGGSQVQLQESGPGLVKPSQTLSLTCTVSGDSITS | |
| | GYWNWIRQHPGKGLEYMGYISYSGSTYYNPSLKS | |
| | RVTISVDTSKNQFSLKLSSVTAADTAVYYCARYYG | |
| | DNYVGAMDYWGQGTLVTVSSGGGGSGGGGSGGG | |
| | GSGGGGSDIVMTQSPLSLPVTPGEPASISCRSSKSLL | |
| | HSNGITYLYWYLQKPGQSPQLLIYQMSNLASGVPD | |
| | RFSGSGSGTDFTLKISRVEAEDVGVYYCAQNLELP | |
| | WTFGGGTKVEIK | |
| 190 | QVQLQESGPGLVKPSQTLSLTCTVSGDSITSGYWN | Tetravalent scFv |
| | WIRQHPGKGLEYMGYISYSGSTYYNPSLKSRVTISV | construct 2 |
| | DTSKNQFSLKLSSVTAADTAVYYCARYYGDNYVGA | (scFv bolded, |
| | MDYWGQGTLVTVSSGGGGSGGGGSGGGGSGGGG | Linker underlined, |
| | SDIVMTQSPLSLPVTPGEPASISCRSSKSLLHSNGITY | Fc domain |
| | LYWYLQKPGQSPQLLIYQMSNLASGVPDRFSGSGS | italicized) |
| | GTDFTLKISRVEAEDVGVYYCAQNLELPWTFGGGT | |
| | KVEIKGGGGSQVQLQESGPGLVKPSQTLSLTCTVS | |
| | GDSITSGYWNWIRQHPGKGLEYMGYISYSGSTYYN | |
| | PSLKSRVTISVDTSKNQFSLKLSSVTAADTAVYYCA | |
| | RYYGDNYVGAMDYWGQGTLVTVSSGGGGSGGGG | |
| | SGGGGSGGGGSDIVMTQSPLSLPVTPGEPASISCRS | |
| | SKSLLHSNGITYLYWYLQKPGQSPQLLIYQMSNLA | |
| | SGVPDRFSGSGSGTDFTLKISRVEAEDVGVYYCAQ | |
| | NLELPWTFGGGTKVEIKGSDKTHTCPPCPAPELLGG | |
| | PSVFLFPPKPKDTLMISRTPEVTCVVVDVSHEDPEVKFN | |
| | WYVDGVEVHNAKTKPREEQYNSTYRVVSVLTVLHQDWL | |
| | NGKEYKCKVSNKALPAPIEKTISKAKGOPREPQVYTLPPS | |
| | RDELTKNOVSLTCLVKGFYPSDIAVEWESNGOPENNYKT | |
| | TPPVLDSDGSFFLYSKLTVDKSRWQQGNVFSCSVMHEAL | |
| | HNHYTOKSLSLSPGK | |
| 191 | QVQLQESGPGLVKPSQTLSLTCTVSGDSITSGYWN | Hexavalent scFv |
| | WIRQHPGKGLEYMGYISYSGSTYYNPSLKSRVTISV | construct 1 |
| | DTSKNQFSLKLSSVTAADTAVYYCARYYGDNYVGA | (scFv bolded, |
| | MDYWGQGTLVTVSSGGGGSGGGGSGGGGSGGGG | Linker underlined, |
| | SDIVMTQSPLSLPVTPGEPASISCRSSKSLLHSNGITY | Fc domain |
| | LYWYLQKPGQSPQLLIYQMSNLASGVPDRFSGSGS | italicized) |
| | GTDFTLKISRVEAEDVGVYYCAQNLELPWTFGGGT | |
| | KVEIKGGGGSQVQLQESGPGLVKPSQTLSLTCTVS | |
| | GDSITSGYWNWIRQHPGKGLEYMGYISYSGSTYYN | |
| | PSLKSRVTISVDTSKNQFSLKLSSVTAADTAVYYCA | |
| | RYYGDNYVGAMDYWGQGTLVTVSSGGGGSGGGG | |
| | SGGGGSGGGGSDIVMTQSPLSLPVTPGEPASISCRS | |
| | SKSLLHSNGITYLYWYLQKPGQSPQLLIYQMSNLA | |
| | SGVPDRFSGSGSGTDFTLKISRVEAEDVGVYYCAQ | |
| | NLELPWTFGGGTKVEIKGGGGSQVQLQESGPGLV | |
| | KPSQTLSLTCTVSGDSITSGYWNWIRQHPGKGLEY | |
| | MGYISYSGSTYYNPSLKSRVTISVDTSKNQFSLKLSS | |
| | VTAADTAVYYCARYYGDNYVGAMDYWGQGTLVT | |
| | VSSGGGGSGGGGSGGGGSGGGGSDIVMTQSPLSLP | |
| | VTPGEPASISCRSSKSLLHSNGITYLYWYLQKPGQS | |
| | PQLLIYQMSNLASGVPDRFSGSGSGTDFTLKISRVE | |
| | AEDVGVYYCAQNLELPWTFGGGTKVEIKGSDKTHT | |
| | CPPCPAPELLGGPSVFLFPPKPKDTLMISRTPEVTCVVVD | |
| | VSHEDPEVKFNWYVDGVEVHNAKTKPREEQYNSTYRVVS | |
| | VLTVLHODWLNGKEYKCKVSNKALPAPIEKTISKAKGQP | |
| | REPQVYTLPPSRDELTKNOVSLTCLVKGFYPSDIAVEWES | |
| | NGQPENNYKTTPPVLDSDGSFFLYSKLTVDKSRWQQGNV | |
| | FSCSVMHEALHNHYTOKSLSLSPGK | |
| 192 | QVQLQESGPGLVKPSQTLSLTCTVSGDSITSGYWN | Hexavalent scFv |
| | WIRQHPGKGLEYMGYISYSGSTYYNPSLKSRVTISV | construct 2 |
| | DTSKNQFSLKLSSVTAADTAVYYCARYYGDNYVGA | (scFv bolded, |
| | MDYWGQGTLVTVSSGGGGSGGGGSGGGGSGGGG | Linker underlined, |
| | SDIVMTQSPLSLPVTPGEPASISCRSSKSLLHSNGITY | Fc domain |
| | LYWYLQKPGQSPQLLIYQMSNLASGVPDRFSGSGS | italicized) |
| | GTDFTLKISRVEAEDVGVYYCAQNLELPWTFGGGT | |
| | KVEIKGGGGSQVQLQESGPGLVKPSQTLSLTCTVS | |
| | GDSITSGYWNWIRQHPGKGLEYMGYISYSGSTYYN | |
| | PSLKSRVTISVDTSKNQFSLKLSSVTAADTAVYYCA | |
| | RYYGDNYVGAMDYWGQGTLVTVSSGGGGSGGGG | |
| | SGGGGSGGGGSDIVMTQSPLSLPVTPGEPASISCRS | |
| | SKSLLHSNGITYLYWYLQKPGQSPQLLIYQMSNLA | |
| | SGVPDRFSGSGSGTDFTLKISRVEAEDVGVYYCAQ | |
| | NLELPWTFGGGTKVEIKGSDKTHTCPPCPAPELLGG | |
| | PSVFLFPPKPKDTLMISRTPEVTCVVVDVSHEDPEVKEN | |
| | WYVDGVEVHNAKTKPREEQYNSTYRVVSVLTVLHQDWL | |
| | NGKEYKCKVSNKALPAPIEKTISKAKGOPREPQVYTLPPS | |
| | RDELTKNOVSLTCLVKGFYPSDIAVEWESNGOPENNYKT | |
| | TPPVLDSDGSFFLYSKLTVDKSRWQQGNVFSCSVMHEAL | |
| | HNHYTOKSLSLSPGKGGGGSQVQLQESGPGLVKPSQT | |
| | LSLTCTVSGDSITSGYWNWIRQHPGKGLEYMGYIS | |
| | YSGSTYYNPSLKSRVTISVDTSKNQFSLKLSSVTAAD | |
| | TAVYYCARYYGDNYVGAMDYWGQGTLVTVSSGG | |
| | GGSGGGGSGGGGSGGGGSDIVMTQSPLSLPVTPGE | |
| | PASISCRSSKSLLHSNGITYLYWYLQKPGQSPQLLIY | |
| | QMSNLASGVPDRFSGSGSGTDFTLKISRVEAEDVG | |
| | VYYCAQNLELPWTFGGGTKVEIK | |
| 193 | QVQLQESGPGLVKPSQTLSLTCTVSGDSITSGYWN | Hexavalent scFv |
| | WIRQHPGKGLEYMGYISYSGSTYYNPSLKSRVTISV | construct 3 |
| | DTSKNQFSLKLSSVTAADTAVYYCARYYGDNYVGA | (scFv bolded, |
| | MDYWGQGTLVTVSSGGGGSGGGGSGGGGSGGGG | Linker underlined, |
| | SDIVMTQSPLSLPVTPGEPASISCRSSKSLLHSNGITY | Fc domain |
| | LYWYLQKPGQSPQLLIYQMSNLASGVPDRFSGSGS | italicized) |
| | GTDFTLKISRVEAEDVGVYYCAQNLELPWTFGGGT | |
| | KVEIKGSDKTHTCPPCPAPELLGGPSVFLFPPKPKDTL | |
| | MISRTPEVTCVVVDVSHEDPEVKFNWYVDGVEVHNAKTK | |
| | PREEQYNSTYRVVSVLTVLHODWLNGKEYKCKVSNKALP | |
| | APIEKTISKAKGQPREPQVYTLPPSRDELTKNQVSLTCLV | |
| | KGFYPSDIAVEWESNGOPENNYKTTPPVLDSDGSFFLYSK | |
| | LTVDKSRWQQGNVFSCSVMHEALHNHYTQKSLSLSPGK | |
| | GGGGSQVQLQESGPGLVKPSQTLSLTCTVSGDSITS | |
| | GYWNWIRQHPGKGLEYMGYISYSGSTYYNPSLKS | |
| | RVTISVDTSKNQFSLKLSSVTAADTAVYYCARYYG | |
| | DNYVGAMDYWGQGTLVTVSSGGGGSGGGGSGGG | |
| | GSGGGGSDIVMTQSPLSLPVTPGEPASISCRSSKSLL | |
| | HSNGITYLYWYLQKPGQSPQLLIYQMSNLASGVPD | |
| | RFSGSGSGTDFTLKISRVEAEDVGVYYCAQNLELP | |
| | WTFGGGTKVEIKGGGGSQVQLQESGPGLVKPSQT | |
| | LSLTCTVSGDSITSGYWNWIRQHPGKGLEYMGYIS | |
| | YSGSTYYNPSLKSRVTISVDTSKNQFSLKLSSVTAAD | |
| | TAVYYCARYYGDNYVGAMDYWGQGTLVTVSSGG | |
| | GGSGGGGSGGGGSGGGGSDIVMTQSPLSLPVTPGE | |
| | PASISCRSSKSLLHSNGITYLYWYLQKPGQSPQLLIY | |
| | QMSNLASGVPDRFSGSGSGTDFTLKISRVEAEDVG | |
| | VYYCAQNLELPWTFGGGTKVEIK | |

In certain embodiments, an isolated anti-fentanyl antibody described herein, or fragment thereof, further comprises a detectable label.

Certain embodiments of the invention provide an antibody or fragment thereof as described herein.

Certain embodiments of the invention provide a method as described herein for making an antibody of the invention or fragment thereof.

Certain embodiments of the invention provide an antibody or fragment thereof isolated by a method as described herein.

Certain embodiments provide a composition comprising an anti-fentanyl antibody as described herein, or fragment thereof, and a carrier. In certain embodiments, the composition is a pharmaceutical composition comprising a pharmaceutically acceptable carrier.

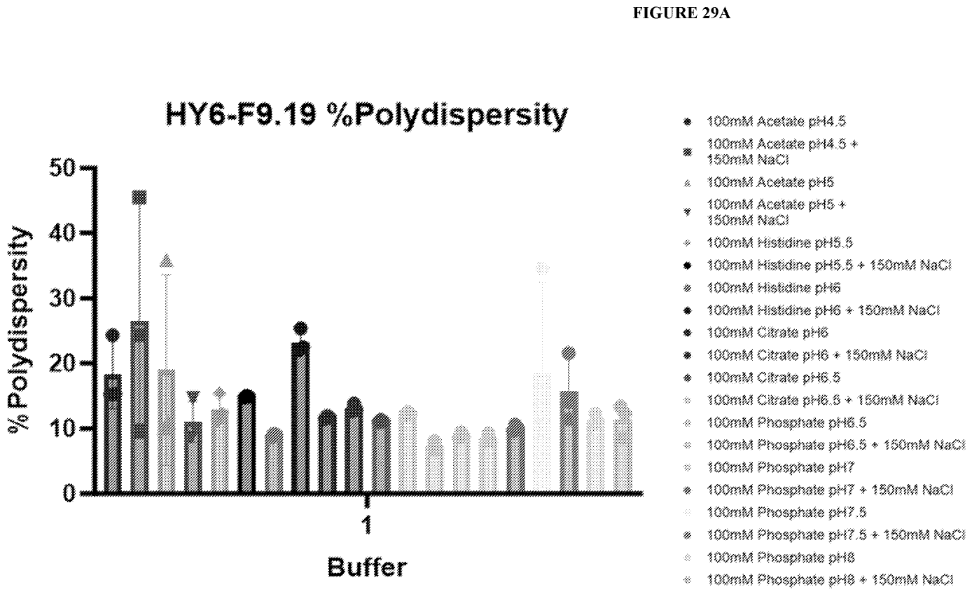

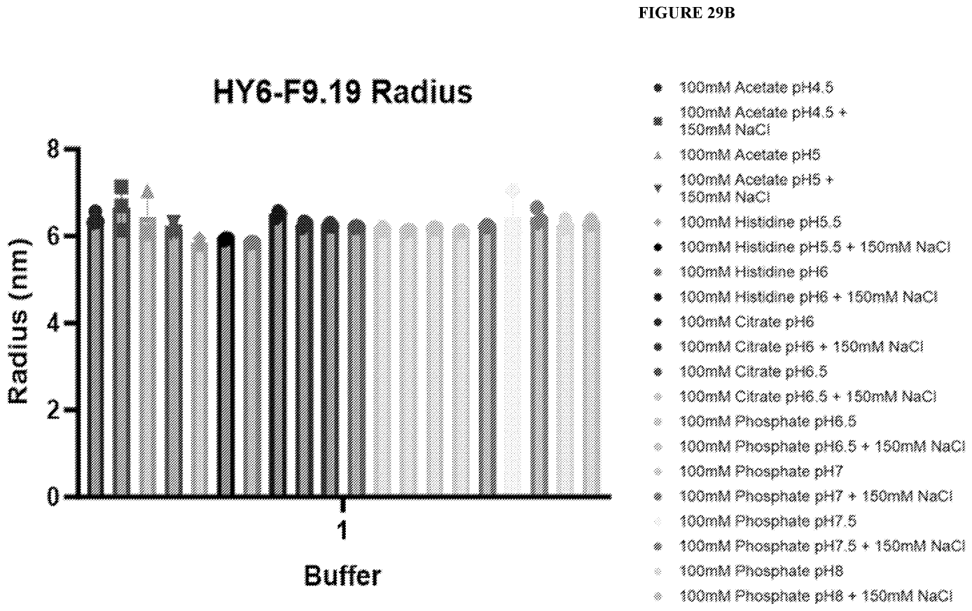

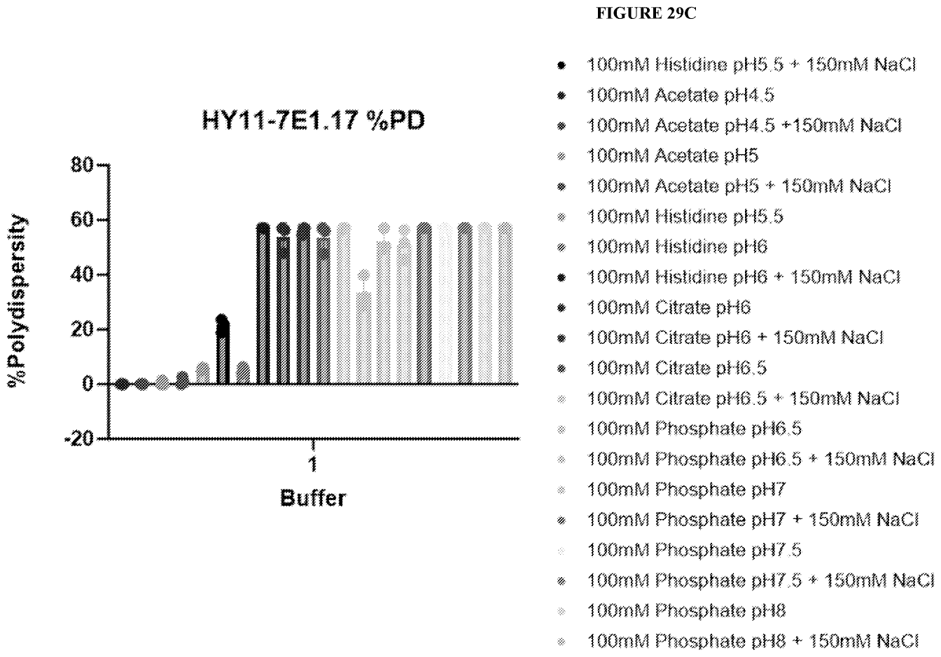

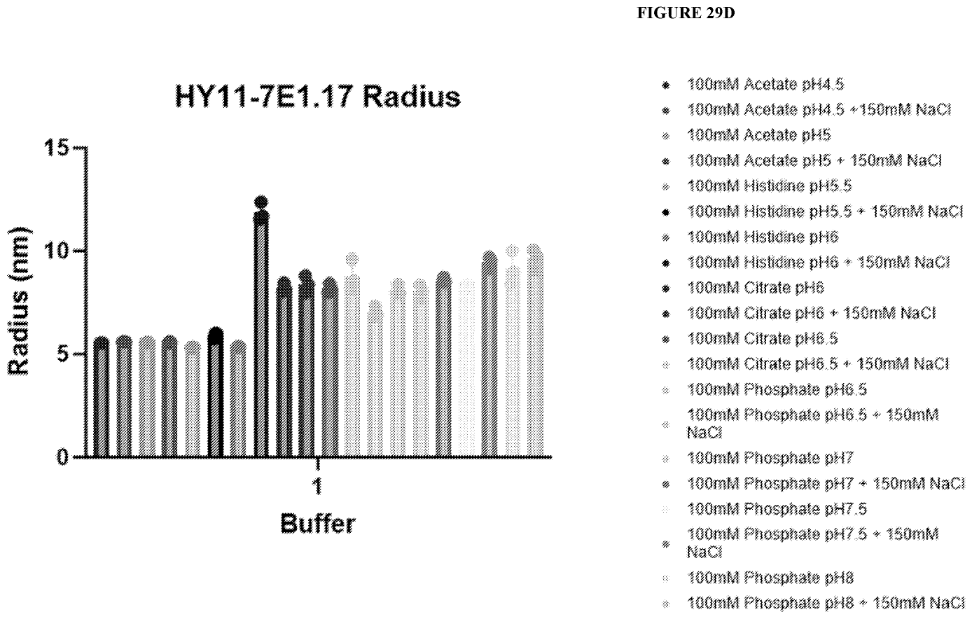

In certain embodiments, the antibody or fragment thereof is formulated in a buffer described herein (e.g., see FIG. 29). In certain embodiments, the antibody or fragment thereof is formulated in a buffer comprising acetate, histidine, citrate, or phosphate. In certain embodiments, the antibody or fragment thereof is formulated in buffer of pH 4.5 to 8 (e.g., pH4.5, pH5, pH5.5, pH6, pH6.5, pH7, pH7.5, or pH8). In certain embodiments, the antibody or fragment thereof is formulated in buffer of pH 4.5 to 5.5 (e.g., pH4.5, pH5, or pH5.5). In certain embodiments, the antibody or fragment thereof is formulated in buffer of pH 4.5 to 6, pH 4.5 to 7, pH 5 to 7, pH 5 to 6 or pH 6 to 7.5. In certain embodiments, the antibody or fragment thereof is formulated in buffer comprising NaCl (e.g., 150 mM). In certain embodiments, the antibody or fragment thereof is formulated in buffer that does not comprise NaCl. In certain embodiments, the hydrodynamic radius (Rh) of the antibody or fragment thereof in the buffer is at no greater than 10 nm, 9 nm, 8 nm, 7 nm, or 6 nm. In certain embodiments, the % polydispersity of the antibody or fragment thereof in the buffer is at no greater than 20%, 19%, 18%, 17%, 15%, 10%, or 5%.

In certain embodiments, the composition described herein may be given before a subject is exposed to fentanyl or analog to prevent or mitigate the effects of fentanyl or its analog(s) exposure. Alternatively, the composition described herein may be given after a subject is exposed to fentanyl or analog to reverse or mitigate the effects of the exposure.

In certain embodiments, administration of the composition described herein may be given pre-exposure in a method of passive immunization against fentanyl or analog(s). In certain embodiments., the composition may be administered pre-exposure., e.g., at least 6 hours, 12 hours, 24 hours, 2 days, 3 days, 4 days, 5 days, 6 days, 1 week, 2 weeks, 3 weeks, or 4 weeks before an anticipated exposure to fentanyl or analog. In certain embodiments, the composition may be administered post-exposure (after suspected or known exposure) e.g., about 1 minute, 5 minutes, 10 minutes, 20 minutes, 30 minutes, 1 hour, 2 hours, 3 hours, 6 hours, 12 hours, 24 hours, 2 days, 3 days, 4 days, 5 days, 6 days, 1 week, 2 weeks, 3 weeks, or 4 weeks to reverse or mitigate the effects of the exposure.

Certain embodiments provide a kit comprising an isolated anti-fentanyl antibody as described herein, or fragment thereof, packaging material, and instructions for administering the antibody, or a fragment thereof, to a mammal to treat fentanyl or analog related overdose, poisoning, disorder, or chemical incident or attack.

Certain embodiments provide a kit comprising an isolated anti-fentanyl antibody as described herein, or fragment thereof, packaging material, and instructions for administering the antibody, or a fragment thereof, to a mammal to treat fentanyl or analog related overdose or disorder. In certain embodiments, the kit further comprises at least one additional therapeutic agent. In certain embodiments, the at least one additional therapeutic agent is useful for treating fentanyl or analog related overdose or disorder.

As used herein, the term “antibody” includes a single-chain variable fragment (scFv), a single-domain antibody (e.g., “VHH”, “nanobody”), humanized, fully human or chimeric antibodies, single-chain antibodies, diabodies, antigen-binding fragments of antibodies that do not contain the Fc region (e.g., Fab fragments), fusion protein (e.g, scFv-Fc fusion, VHH-Fc fusion, scFv-SA fusion) that comprises antigen-binding regions that are fused to non-antigen binding carrier proteins such as an Fc region, serum albumin (SA) or other protein scaffold, and multispecific or multivalent antibodies/fusion proteins that comprise more than one unique antibody antigen-binding region of antibodies on a recombinant Fc region, SA, or other protein scaffold. In certain embodiments, the antibody is a humanized antibody. A “humanized” antibody contains the three CDRs (complementarity determining regions) and sometimes optionally a few carefully selected “framework” residues (the non-CDR portions of the variable regions) from each donor antibody variable region recombinantly linked onto the corresponding frameworks and constant regions of a human antibody sequence. A “fully human antibody” can be created in a hybridoma from mice genetically engineered to have only human-derived antibody genes or by selection from a phage-display library of human-derived antibody genes.

A scFv is a fusion protein of the variable region of the heavy (VH) and light chains (VL) of an immunoglobulin that is connected by means of a linker peptide. The linker is usually short, about 10-25 amino acids in length. If flexibility is important, the linker will contain a significant number of glycines. If solubility is important, serines or threonines will be utilized in the linker. The linker may link the amino-terminus of the VH to the carboxy-terminus of the VL, or the linker may link the carboxy-terminus of the VH to the amino-terminus of the VL. Divalent (also called bivalent) scFvs can be generated by linking two scFvs. For example, a divalent scFv can be made by generating a single peptide containing two VH and two VL regions.

Alternatively, two peptides, each containing a single VH and a single VL region can be dimerized (also called “diabodies”). Holliger et al., “Diabodies: small bivalent and bispecific antibody fragments,” PNAS, July 1993, 90:6444-6448. Bivalency allows antibodies to bind to multimeric antigens with high avidity, and bispecificity allows the cross-linking of two antigens.

As used herein, the term “monoclonal antibody” refers to an antibody obtained from a group of substantially homogeneous antibodies, that is, an antibody group wherein the antibodies constituting the group are homogeneous except for naturally occurring mutants that may exist in a small amount. Monoclonal antibodies are highly specific and interact with a single antigenic site. Furthermore, each monoclonal antibody targets a single antigenic determinant (epitope) on an antigen, as compared to common polyclonal antibody preparations that typically contain various antibodies against diverse antigenic determinants. In addition to their specificity, monoclonal antibodies are advantageous in that they are typically produced from hybridoma cultures not contaminated with other immunoglobulins.

The adjective “monoclonal” indicates a characteristic of antibodies obtained from a substantially homogeneous group of antibodies, and does not specify antibodies produced by a particular method. For example, a monoclonal antibody to be used in the present invention can be produced by, for example, hybridoma methods (Kohler and Milstein, Nature 256:495, 1975) or recombination methods (U.S. Pat. No. 4,816,567). The monoclonal antibodies used in the present invention can be also isolated from a phage antibody library (Clackson et al., Nature 352:624-628, 1991; Marks et al., J. Mol. Biol. 222:581-597, 1991). The monoclonal antibodies used in the present invention can be also isolated from synthetic opioid-specific animal B cells or human B cells by means of antigen-specific cell sorting paired with sequencing, and recombinantly generated by cloning, and expressing the B cell receptor or specific antibody binding regions (VH and VL). Alternatively, VH and VL could be generated directly from bulk B cells via 10× Genomics paired with deep sequencing or other state-of the-art strategy to isolate antigen-specific antibody binding regions. The monoclonal antibodies used in the present invention can be also derived from synthetic opioid-specific B cells isolated from humanized mouse or other humanized animal models by means of sorting, sequencing, cloning, and expressing the specific antibody binding regions. For example, the monoclonal antibodies used in the present invention can be isolated from antigen-specific B cell lymphocytes, e.g., by FACS paired with sequencing, cloning antibody binding regions into vector(s), transfection of the vector(s) to suitable cell line, and then expression/purification of the mAbs. The monoclonal antibodies of the present invention may comprise “chimeric” antibodies (immunoglobulins), wherein a part of a heavy (H) chain and/or light (L) chain is derived from a specific species or a specific antibody class or subclass, and the remaining portion of the chain is derived from another species, or another antibody class or subclass. Furthermore, mutant antibodies and antibody fragments thereof are also comprised in the present invention (U.S. Pat. No. 4,816,567; Morrison et al., Proc. Natl. Acad. Sci. USA 81:6851-6855, 1984).

As used herein, the term “mutant antibody” refers to an antibody comprising a variant amino acid sequence in which one or more amino acid residues have been altered. For example, the variable region of an antibody can be modified to improve its biological properties, such as antigen binding. Such modifications can be achieved by site-directed mutagenesis (see Kunkel, Proc. Natl. Acad. Sci. USA 82: 488 (1985)), PCR-based mutagenesis, cassette mutagenesis, and the like. Such mutants comprise an amino acid sequence which is at least 70% identical to the amino acid sequence of a CDR sequence or a heavy or light chain variable region of the antibody, more specifically at least 75%, even more specifically at least 80%, still more specifically at least 85%, yet more specifically at least 90%, and most specifically at least 95% identical. As used herein, the term “sequence identity” is defined as the percentage of residues identical to those in the antibody's original amino acid sequence, determined after the sequences are aligned and gaps are appropriately introduced to maximize the sequence identity as necessary.

Specifically, the identity of one nucleotide sequence or amino acid sequence to another can be determined using the algorithm BLAST, by Karlin and Altschul (Proc. Natl. Acad. Sci. USA, 90: 5873-5877, 1993). Programs such as BLASTN and BLASTX were developed based on this algorithm (Altschul et al., J. Mol. Biol. 215: 403-410, 1990). To analyze nucleotide sequences according to BLASTN based on BLAST, the parameters are set, for example, as score=100 and wordlength=12. On the other hand, parameters used for the analysis of amino acid sequences by BLASTX based on BLAST include, for example, score=50 and wordlength=3. Default parameters for each program are used when using the BLAST and Gapped BLAST programs. Specific techniques for such analyses are known in the art (see the website of the National Center for Biotechnology Information (NCBI), Basic Local Alignment Search Tool (BLAST); http://www.ncbi.nlm.nih.gov).

Polyclonal and monoclonal antibodies can be prepared by methods known to those skilled in the art.

In another embodiment, antibodies or antibody fragments can be isolated from an antibody phage library, produced by using the technique reported by McCafferty et al. (Nature 348:552-554 (1990)). Clackson et al. (Nature 352:624-628 (1991)) and Marks et al. (J. Mol. Biol. 222:581-597 (1991)) reported on the respective isolation of mouse and human antibodies from phage libraries. There are also reports that describe the production of high affinity (nM range) human antibodies based on chain shuffling (Marks et al., Bio/Technology 10:779-783 (1992)), and combinatorial infection and in vivo recombination, which are methods for constructing large-scale phage libraries (Waterhouse et al., Nucleic Acids Res. 21:2265-2266 (1993)). These technologies can also be used to isolate monoclonal antibodies, instead of using conventional hybridoma technology for monoclonal antibody production. In some embodiments, antibodies or antibody fragments can be isolated directly from B cells specific for the target antigens. In this case, synthetic opioid-specific B cells are isolated by FACS as either bulk or single cell preparation, and then sequences of variable regions are obtained by various strategies such as standard Sanger DNA sequencing, RNA sequencing, or 10× Genomics and then antibody binding regions are cloned/expressed using vectors and expression systems.

Antibodies to be used in the present invention can be purified by a method appropriately selected from known methods, such as the protein A-Sepharose method, hydroxyapatite chromatography, salting-out method with sulfate, ion exchange chromatography, and affinity chromatography, or by the combined use of the same.

The present invention may use recombinant antibodies, produced by gene engineering. The genes encoding the antibodies obtained by a method described above are isolated from the hybridomas or directly from antigen-specific B cell lymphocytes. The genes are inserted into an appropriate vector, and then introduced into a host (see, e.g., Carl, A. K. Borrebaeck, James, W.

Larrick, Therapeutic Monoclonal Antibodies, Published in the United Kingdom by Macmillan Publishers Ltd, 1990). The present invention provides the nucleic acids encoding the antibodies of the present invention, and vectors comprising these nucleic acids. Specifically, using a reverse transcriptase, cDNAs encoding the variable regions (V regions) of the antibodies are synthesized from the mRNAs of hybridomas or B cell lymphocytes. After obtaining the DNAs encoding the variable regions of antibodies of interest, they are ligated with DNAs encoding desired constant regions (C regions) of the antibodies, and the resulting DNA constructs are inserted into expression vectors. Alternatively, the DNAs encoding the variable regions of the antibodies may be inserted into expression vectors comprising the DNAs of the antibody C regions. These are inserted into expression vectors so that the genes are expressed under the regulation of an expression regulatory region, for example, an enhancer and promoter. Then, host cells are transformed with the expression vectors to express the antibodies. The present invention provides cells expressing antibodies of the present invention. The cells expressing antibodies of the present invention include cells and hybridomas transformed with a gene of such an antibody.

The antibodies of the present invention also include antibodies which comprise complementarity-determining regions (CDRs), or regions functionally equivalent to CDRs. The term “functionally equivalent” refers to comprising amino acid sequences similar to the amino acid sequences of CDRs of any of the monoclonal antibodies as described herein. The term “CDR” refers to a region in an antibody variable region (also called “V region”) and determines the specificity of antigen binding. The H chain and L chain each have three CDRs, designated from the N terminus as CDR1, CDR2, and CDR3. There are four regions flanking these CDRs: these regions are referred to as “framework,” and their amino acid sequences are highly conserved. The CDRs can be transplanted into other antibodies, and thus a recombinant antibody can be prepared by combining CDRs with the framework of a desired antibody. One or more amino acids of a CDR can be modified without losing the ability to bind to its antigen. For example, one or more amino acids in a CDR can be substituted, deleted, and/or added and yet maintain comparable binding affinity to the target.

In certain embodiments, an amino acid residue is mutated into one that allows the properties of the amino acid side-chain to be conserved. Examples of the properties of amino acid side chains comprise: hydrophobic amino acids (A, I, L, M, F, P, W, Y, V), hydrophilic amino acids (R, D, N, C, E, Q, G, H, K, S, T), and amino acids comprising the following side chains: aliphatic side-chains (G, A, V, L, I, P); hydroxyl group-containing side-chains (S, T, Y); sulfur atom-containing side-chains (C, M); carboxylic acid- and amide-containing side-chains (D, N, E, Q); base-containing side-chains (R, K, H); and aromatic-containing side-chains (H, F, Y, W). The letters within parenthesis indicate the one-letter amino acid codes. Amino acid substitutions within each group are called conservative substitutions. It is well known that a polypeptide comprising a modified amino acid sequence in which one or more amino acid residues is deleted, added, and/or substituted can retain the original biological activity (Mark D. F. et al., Proc. Natl. Acad. Sci. U.S.A. 81:5662-5666 (1984); Zoller M. J. and Smith M., Nucleic Acids Res. 10: 6487-6500 (1982); Wang A. et al., Science 224: 1431-1433; Dalbadie-McFarland G. et al., Proc. Natl. Acad. Sci. U.S.A. 79: 6409-6413 (1982)). The number of mutated amino acids is not limited, but in general, the number falls within 40% of amino acids of each CDR, and specifically within 35%, and still more specifically within 30% (e.g., within 25%). The identity of amino acid sequences can be determined as described herein.

In the present invention, recombinant antibodies artificially modified to reduce heterologous antigenicity in humans can be used. Examples include chimeric antibodies and humanized antibodies. These modified antibodies can be produced using known methods. A chimeric antibody includes an antibody comprising variable and constant regions of species that are different to each other, for example, an antibody comprising the antibody heavy chain and light chain variable regions of a nonhuman mammal such as a mouse, and the antibody heavy chain and light chain constant regions of a human. Such an antibody can be obtained by (1) ligating a DNA encoding a variable region of a mouse antibody to a DNA encoding a constant region of a human antibody; (2) incorporating this into an expression vector; and (3) introducing the vector into a host for production of the antibody.

A humanized antibody, which is also called a reshaped human antibody, may be obtained by substituting H or L chain complementarity determining region (CDR) of an antibody of a nonhuman mammal such as a mouse, for the CDR of a human antibody. Antibody humanization techniques are described herein (Example 1) and also known in the art. Conventional genetic recombination techniques for the preparation of such antibodies are known (see, for example, Jones et al., Nature 321: 522-525 (1986); Reichmann et al., Nature 332: 323-329 (1988); Presta Curr. Op. Struct. Biol. 2: 593-596 (1992)). Specifically, a DNA sequence designed to ligate CDRs of a mouse antibody with the framework regions (FRs) of a human antibody is synthesized by PCR, using several oligonucleotides constructed to comprise overlapping portions at their ends. A humanized antibody can be obtained by (1) ligating the resulting DNA to a DNA that encodes a human antibody constant region; (2) incorporating this into an expression vector; and (3) transfecting the vector into a host to produce the antibody (see, European Patent Application No. EP 239,400, and International Patent Application No. WO 96/02576). Human antibody FRs that are ligated via the CDR are selected where the CDR forms a favorable antigen-binding site. The humanized antibody may comprise additional amino acid residue(s) that are not included in the CDRs introduced into the recipient antibody. Such amino acid residues are usually introduced to more accurately optimize the antibody's ability to recognize and bind to an antigen. For example, as necessary, amino acids in the framework region of an antibody variable region may be substituted such that the CDR of a reshaped human antibody forms an appropriate antigen-binding site (Sato, K. et al., Cancer Res. (1993) 53, 851-856).

An antibody of the invention may also be a recombinant antibody (e.g., a humanized or chimeric antibody) or a fragment thereof. Accordingly, such an antibody of the invention or fragment thereof would not be a product of nature. Additionally, an antibody of the invention or a fragment thereof may comprise markedly different characteristics (e.g., structural, functional and/or other properties) as compared to naturally occurring antibody.

The isotypes of the antibodies of the present invention are not limited. The isotypes include, for example, IgG (IgG1, IgG2, IgG3, and IgG4), IgM, IgA (IgA1 and IgA2), IgD, and IgE. The antibodies of the present invention may also be antibody fragments comprising a portion responsible for antigen binding, or a modified fragment thereof. The term “antibody fragment” refers to a portion of a full-length antibody, and generally to a fragment comprising an antigen-binding domain or a variable region. Such antibody fragments include, for example, Fab, F(ab′)₂, Fv, single-chain Fv (scFv) which comprises a heavy chain Fv and a light chain Fv coupled together with an appropriate linker, diabody (diabodies), and multispecific antibodies prepared from antibody fragments. Previously, antibody fragments were produced by digesting natural antibodies with a protease; currently, methods for expressing them as recombinant antibodies using genetic engineering techniques are also known (see Morimoto et al., Journal of Biochemical and Biophysical Methods 24:107-117 (1992); Brennan et al., Science 229:81 (1985); Co, M. S. et al., J. Immunol., 1994, 152, 2968-2976; Better, M. & Horwitz, A. H., Methods in Enzymology, 1989, 178, 476-496, Academic Press, Inc.; Plueckthun, A. & Skerra, A., Methods in Enzymology, 1989, 178, 476-496, Academic Press, Inc.; Lamoyi, E., Methods in Enzymology, 1989, 121, 663-669; Bird, R. E. et al., TIBTECH, 1991, 9, 132-137).

An “Fv” fragment is the smallest antibody fragment, and contains a complete antigen recognition site and a binding site. This region is a dimer (VH-VL dimer) wherein the variable regions of each of the heavy chain and light chain are strongly connected by a noncovalent bond. The three CDRs of each of the variable regions interact with each other to form an antigen-binding site on the surface of the VH-VL dimer. In other words, a total of six CDRs from the heavy and light chains function together as an antibody's antigen-binding site. However, a variable region (or a half Fv, which contains only three antigen-specific CDRS) alone is also known to be able to recognize and bind to an antigen, although its affinity is lower than the affinity of the entire binding site. Thus, a specific antibody fragment of the present invention is an Fv fragment, but is not limited thereto. Such an antibody fragment may be a polypeptide which comprises an antibody fragment of heavy or light chain CDRs which are conserved, and which can recognize and bind its antigen.

A Fab fragment (also referred to as F(ab)) also contains a light chain constant region and heavy chain constant region (CH1). For example, papain digestion of an antibody produces the two kinds of fragments: an antigen-binding fragment, called a Fab fragment, containing the variable regions of a heavy chain and light chain, which serve as a single antigen-binding domain; and the remaining portion, which is called an “Fc” because it is readily crystallized. A Fab′ fragment is different from a Fab fragment in that a Fab′ fragment also has several residues derived from the carboxyl terminus of a heavy chain CH1 region, which contains one or more cysteine residues from the hinge region of an antibody. A Fab′ fragment is, however, structurally equivalent to Fab in that both are antigen-binding fragments which comprise the variable regions of a heavy chain and light chain, which serve as a single antigen-binding domain. Herein, an antigen-binding fragment comprising the variable regions of a heavy chain and light chain which serve as a single antigen-binding domain, and which is equivalent to that obtained by papain digestion, is referred to as a “Fab-like antibody,” even when it is not identical to an antibody fragment produced by protease digestion. Fab′-SH is Fab′ with one or more cysteine residues having free thiol groups in its constant region. A F(ab′) fragment is produced by cleaving the disulfide bond between the cysteine residues in the hinge region of F(ab′)₂. Other chemically crosslinked antibody fragments are also known to those skilled in the art. Pepsin digestion of an antibody yields two fragments; one is a F(ab′)₂ fragment which comprises two antigen-binding domains and can cross-react with antigens, and the other is the remaining fragment (referred to as pFc′). Herein, an antibody fragment equivalent to that obtained by pepsin digestion is referred to as a “F(ab′)₂-like antibody” when it comprises two antigen-binding domains and can cross-react with antigens. Such antibody fragments can also be produced, for example, by genetic engineering. Such antibody fragments can also be isolated, for example, from the antibody phage library described above. Alternatively, F(ab′)₂-SH fragments can be recovered directly from hosts, such as E. co/i, and then allowed to form F(ab′)₂ fragments by chemical crosslinking (Carter et al., Bio/Technology 10:163-167 (1992)). In an alternative method, F(ab′)₂ fragments can be isolated directly from a culture of recombinant hosts.

The term “diabody (Db)” refers to a bivalent antibody fragment constructed by gene fusion (for example, P. Holliger et al., Proc. Natl. Acad. Sci. USA 90: 6444-6448 (1993), EP 404,097, WO 93/11161). In general, a diabody is a dimer of two polypeptide chains. In the each of the polypeptide chains, a light chain variable region (VL) and a heavy chain variable region (V_{H}) in an identical chain are connected via a short linker, for example, a linker of about five residues, so that they cannot bind together. Because the linker between the two is too short, the V_{L} and V_{H} in the same polypeptide chain cannot form a single chain V region fragment, but instead form a dimer. Thus, a diabody has two antigen-binding domains. When the V_{L} and V_{H} regions against the two types of antigens (a and b) are combined to form V_{La}-V_{Hb} and V_{Lb}-V_{Ha} via a linker of about five residues, and then co-expressed, they are secreted as bispecific Dbs. The antibodies of the present invention may be such Dbs.

A single-chain antibody (also referred to as “scFv”) can be prepared by linking a heavy chain V region and a light chain V region of an antibody (for a review of scFv see Pluckthun “The Pharmacology of Monoclonal Antibodies” Vol. 113, eds. Rosenburg and Moore, Springer Verlag, N.Y., pp. 269-315 (1994)). Methods for preparing single-chain antibodies are known in the art (see, for example, U.S. Pat. Nos. 4,946,778; 5,260,203; 5,091,513; and 5,455,030). In such scFvs, the heavy chain V region and the light chain V region are linked together via a linker, such as a polypeptide linker (Huston, J. S. et al., Proc. Natl. Acad. Sci. U.S.A, 1988, 85, 5879-5883). The heavy chain V region and the light chain V region in a scFv may be derived from the same antibody, or from different antibodies. The peptide linker used to ligate the V regions may be any single-chain peptide consisting of 12 to 19 residues. A DNA encoding a scFv can be amplified by PCR using, as a template, either the entire DNA, or a partial DNA encoding a desired amino acid sequence, selected from a DNA encoding the heavy chain or the V region of the heavy chain of the above antibody, and a DNA encoding the light chain or the V region of the light chain of the above antibody; and using a primer pair that defines the two ends. Further amplification can be subsequently conducted using a combination of the DNA encoding the peptide linker portion, and the primer pair that defines both ends of the DNA to be ligated to the heavy and light chain respectively. After constructing DNAs encoding scFvs, conventional methods can be used to obtain expression vectors comprising these DNAs, and hosts transformed by these expression vectors. Furthermore, scFvs can be obtained according to conventional methods using the resulting hosts. These antibody fragments can be produced in hosts by obtaining genes that encode the antibody fragments and expressing these as outlined above. Antibodies bound to various types of molecules, such as polyethylene glycols (PEGs), may be used as modified antibodies. Methods for modifying antibodies are already established in the art. The term “antibody” in the present invention also encompasses the above-described antibodies.

The antibodies obtained can be purified to homogeneity. The antibodies can be isolated and purified by a method routinely used to isolate and purify proteins. The antibodies can be isolated and purified by the combined use of one or more methods appropriately selected from column chromatography, filtration, ultrafiltration, salting out, dialysis, preparative polyacrylamide gel electrophoresis, and isoelectro-focusing, for example (Strategies for Protein Purification and Characterization: A Laboratory Course Manual, Daniel R. Marshak et al. eds., Cold Spring Harbor Laboratory Press (1996); Antibodies: A Laboratory Manual. Ed Harlow and David Lane, Cold Spring Harbor Laboratory, 1988). Such methods are not limited to those listed above. Chromatographic methods include affinity chromatography, ion exchange chromatography, hydrophobic chromatography, gel filtration, reverse-phase chromatography, and adsorption chromatography. These chromatographic methods can be practiced using liquid phase chromatography, such as HPLC and FPLC. Columns to be used in affinity chromatography include protein A columns and protein G columns. For example, protein A columns include Hyper D, POROS, and Sepharose F. F. (Pharmacia). Antibodies can also be purified by utilizing antigen binding, using carriers on which antigens have been immobilized.

The antibodies of the present invention can be formulated according to standard methods (see, for example, Remington's Pharmaceutical Science, latest edition, Mark Publishing Company, Easton, U.S.A), and may comprise pharmaceutically acceptable carriers and/or additives. The present invention relates to compositions (including reagents and pharmaceuticals) comprising the antibodies of the invention, and pharmaceutically acceptable carriers and/or additives. When the composition is prepared as an aqueous solution for injection, it can comprise an isotonic solution comprising, for example, physiological saline, dextrose, and other adjuvants, including, for example, D-sorbitol, D-mannose, D-mannitol, and sodium chloride, which can also contain an appropriate solubilizing agent, for example, alcohol (for example, ethanol), polyalcohol (for example, propylene glycol and PEG), and non-ionic detergent (polysorbate 80 and HCO-50). In certain embodiments, antibody of the present invention or a fragment thereof is present in a liquid composition (e.g., saline or D5W). In certain embodiments, antibody of the present invention or a fragment thereof is present in a solid composition such as a lyophilized composition. In certain embodiments, the lyophilized composition comprises one or more excipients selected from the group consisting of a cryo-lyoprotectant (e.g., trehalose, sucrose) and a bulking agent (e.g., mannitol, glycine). Exemplary pharmaceutically acceptable carriers include surfactants (for example, PEG and Tween), excipients, antioxidants (for example, ascorbic acid), coloring agents, flavoring agents, preservatives, stabilizers, buffering agents (for example, phosphoric acid, citric acid, and other organic acids), chelating agents (for example, EDTA), suspending agents, isotonizing agents, binders, disintegrators, lubricants, fluidity promoters, and corrigents. However, the carriers that may be employed in the present invention are not limited to this list. In fact, other commonly used carriers can be appropriately employed: light anhydrous silicic acid, lactose, crystalline cellulose, mannitol, starch, carmelose calcium, carmelose sodium, hydroxypropylcellulose, hydroxypropylmethyl cellulose, polyvinylacetaldiethylaminoacetate, polyvinylpyrrolidone, gelatin, medium chain fatty acid triglyceride, polyoxyethylene hydrogenated castor oil 60, sucrose, carboxymethylcellulose, corn starch, inorganic salt, and so on. The composition may also comprise other low-molecular-weight polypeptides, proteins such as serum albumin, gelatin, and amino acids such as glycine, glutamine, asparagine, arginine, and lysine.

### Nucleic Acids, Expression Cassettes, Vectors and Cells

Certain embodiments of the invention provide an isolated nucleic acid encoding an antibody or fragment thereof as described herein. In certain embodiments, the nucleic acid further comprises a promoter. In certain embodiments, the isolated nucleic acid encoding an antibody or fragment thereof as described herein is DNA. In certain embodiments, the isolated nucleic acid encoding an antibody or fragment thereof as described herein is mRNA.

Certain embodiments of the invention provide an expression cassette comprising a nucleic acid as described herein and a promoter.

Certain embodiments of the invention provide a vector (e.g., a plasmid or phagemid) comprising a nucleic acid or an expression cassette as described herein. In certain embodiments, the vector is a viral vector, for example, an adeno-associated viral vector (AAV).

Certain embodiments provide a method of contacting/introducing an isolated nucleic acid described herein into a mammalian cell. Certain embodiments provide a method of administering an isolated nucleic acid described herein to a mammal in need of (e.g., administering a mRNA to a human for expression of the antibody or fragment thereof as described herein).

Certain embodiments provide a method of contacting/introducing a vector described herein into a mammalian cell. Certain embodiments provide a method of administering a vector described herein to a mammal in need of (e.g., administering a AAV vector to a human for expression of the antibody or fragment thereof as described herein).

Certain embodiments of the invention provide a cell (e.g., mammalian cell or bacterial cell) comprising a nucleic acid, expression cassette or vector as described herein.

Certain embodiments of the invention provide a phage particle comprising a vector as described herein.

The term “nucleic acid” refers to deoxyribonucleotides or ribonucleotides and polymers thereof in either single- or double-stranded form, composed of monomers (nucleotides) containing a sugar, phosphate and a base which is either a purine or pyrimidine. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucl. Acids Res. 19:508 (1991); Ohtsuka et al., JBC, 260:2605 (1985); Rossolini et al., Mol. Cell. Probes, 8:91 (1994). A “nucleic acid fragment” is a fraction of a given nucleic acid molecule.

Deoxyribonucleic acid (DNA) in the majority of organisms is the genetic material while ribonucleic acid (RNA) is involved in the transfer of information contained within DNA into proteins. The term “nucleotide sequence” refers to a polymer of DNA or RNA that can be single- or double-stranded, optionally containing synthetic, non-natural or altered nucleotide bases capable of incorporation into DNA or RNA polymers. The terms “nucleic acid,” “nucleic acid molecule,” “nucleic acid fragment,” “nucleic acid sequence or segment,” or “polynucleotide” may also be used interchangeably with gene, cDNA, DNA and RNA encoded by a gene.

By “portion” or “fragment,” as it relates to a nucleic acid molecule, sequence or segment of the invention, when it is linked to other sequences for expression, is meant a sequence having at least 80 nucleotides, more specifically at least 150 nucleotides, and still more specifically at least 400 nucleotides. If not employed for expressing, a “portion” or “fragment” means at least 9, specifically 12, more specifically 15, even more specifically at least 20, consecutive nucleotides, e.g., probes and primers (oligonucleotides), corresponding to the nucleotide sequence of the nucleic acid molecules of the invention.

The terms “protein,” “peptide” and “polypeptide” are used interchangeably herein.

The invention encompasses isolated or substantially purified nucleic acid or protein compositions. In the context of the present invention, an “isolated” or “purified” DNA molecule or an “isolated” or “purified” polypeptide is a DNA molecule or polypeptide that exists apart from its native environment and is therefore not a product of nature. An isolated DNA molecule or polypeptide may exist in a purified form or may exist in a non-native environment such as, for example, a transgenic host cell. For example, an “isolated” or “purified” nucleic acid molecule or protein, or biologically active portion thereof, is substantially free of other cellular material, or culture medium when produced by recombinant techniques, or substantially free of chemical precursors or other chemicals when chemically synthesized. In one embodiment, an “isolated” nucleic acid is free of sequences that naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecule can contain less than about 5 kb, 4 kb, 3 kb, 2 kb, 1 kb, 0.5 kb, or 0.1 kb of nucleotide sequences that naturally flank the nucleic acid molecule in genomic DNA of the cell from which the nucleic acid is derived. A protein that is substantially free of cellular material includes preparations of protein or polypeptide having less than about 30%, 20%, 10%, 5%, (by dry weight) of contaminating protein. When the protein of the invention, or biologically active portion thereof, is recombinantly produced, culture medium may represent less than about 30%, 20%, 10%, or 5% (by dry weight) of chemical precursors or non-protein-of-interest chemicals.

Fragments and variants of the disclosed nucleotide sequences and proteins or partial-length proteins encoded thereby are also encompassed by the present invention. “Naturally occurring” is used to describe an object that can be found in nature as distinct from being artificially produced. For example, a protein or nucleotide sequence present in an organism (including a virus), which can be isolated from a source in nature and which has not been intentionally modified by man in the laboratory, is naturally occurring.

A “variant” of a molecule is a sequence that is substantially similar to the sequence of the native molecule. For nucleotide sequences, variants include those sequences that, because of the degeneracy of the genetic code, encode the identical amino acid sequence of the native protein. Naturally occurring allelic variants such as these can be identified with the use of well-known molecular biology techniques, as, for example, with polymerase chain reaction (PCR) and hybridization techniques. Variant nucleotide sequences also include synthetically derived nucleotide sequences, such as those generated, for example, by using site-directed mutagenesis that encode the native protein, as well as those that encode a polypeptide having amino acid substitutions. Generally, nucleotide sequence variants of the invention will have at least 40, 50, 60, to 70%, e.g., 71%, 72%, 73%, 74%, 75%, 76%, 77%, 78%, to 79%, generally at least 80%, e.g., 81%-84%, at least 85%, e.g., 86%, 87%, 88%, 89%, 90%, 91%, 92%, 93%, 94%, 95%, 96%, 97%, to 98%, sequence identity to the native (endogenous) nucleotide sequence. “Conservatively modified variations” of a particular nucleic acid sequence refers to those nucleic acid sequences that encode identical or essentially identical amino acid sequences, or where the nucleic acid sequence does not encode an amino acid sequence, to essentially identical sequences. Because of the degeneracy of the genetic code, a large number of functionally identical nucleic acids encode any given polypeptide. For instance the codons CGT, CGC, CGA, CGG, AGA, and AGG all encode the amino acid arginine. Thus, at every position where an arginine is specified by a codon, the codon can be altered to any of the corresponding codons described without altering the encoded protein. Such nucleic acid variations are “silent variations” which are one species of “conservatively modified variations.” Every nucleic acid sequence described herein which encodes a polypeptide also describes every possible silent variation, except where otherwise noted. One of skill will recognize that each codon in a nucleic acid (except ATG, which is ordinarily the only codon for methionine) can be modified to yield a functionally identical molecule by standard techniques. Accordingly, each “silent variation” of a nucleic acid which encodes a polypeptide is implicit in each described sequence. “Recombinant DNA molecule” is a combination of DNA sequences that are joined together using recombinant DNA technology and procedures used to join together DNA sequences as described, for example, in Sambrook and Russell, Molecular Cloning: A Laboratory Manual, Cold Spring Harbor, NY: Cold Spring Harbor Laboratory Press (3^{rd} edition, 2001).

The terms “heterologous DNA sequence,” “exogenous DNA segment” or “heterologous nucleic acid,” each refer to a sequence that originates from a source foreign to the particular host cell or, if from the same source, is modified from its original form. Thus, a heterologous gene in a host cell includes a gene that is endogenous to the particular host cell but has been modified. The terms also include non-naturally occurring multiple copies of a naturally occurring DNA sequence. Thus, the terms refer to a DNA segment that is foreign or heterologous to the cell, or homologous to the cell but in a position within the host cell nucleic acid in which the element is not ordinarily found. Exogenous DNA segments are expressed to yield exogenous polypeptides.

A “homologous” DNA sequence is a DNA sequence that is naturally associated with a host cell into which it is introduced.

“Wild-type” refers to the normal gene, or organism found in nature without any known mutation.

“Genome” refers to the complete genetic material of an organism.

A “vector” is defined to include, inter alia, any plasmid, cosmid, phage or binary vector in double or single stranded linear or circular form which may or may not be self transmissible or mobilizable, and which can transform prokaryotic or eukaryotic host either by integration into the cellular genome or exist extrachromosomally (e.g., autonomous replicating plasmid with an origin of replication).

“Cloning vectors” typically contain one or a small number of restriction endonuclease recognition sites at which foreign DNA sequences can be inserted in a determinable fashion without loss of essential biological function of the vector, as well as a marker gene that is suitable for use in the identification and selection of cells transformed with the cloning vector. Marker genes typically include genes that provide tetracycline resistance, hygromycin resistance or ampicillin resistance.

“Expression cassette” as used herein means a DNA sequence capable of directing expression of a particular nucleotide sequence in an appropriate host cell, comprising a promoter operably linked to the nucleotide sequence of interest which is operably linked to termination signals. It also typically comprises sequences required for proper translation of the nucleotide sequence. The coding region usually codes for a protein of interest but may also code for a functional RNA of interest, for example antisense RNA or a nontranslated RNA, in the sense or antisense direction. The expression cassette comprising the nucleotide sequence of interest may be chimeric, meaning that at least one of its components is heterologous with respect to at least one of its other components. The expression cassette may also be one that is naturally occurring but has been obtained in a recombinant form useful for heterologous expression. The expression of the nucleotide sequence in the expression cassette may be under the control of a constitutive promoter or of an inducible promoter that initiates transcription only when the host cell is exposed to some particular external stimulus. In the case of a multicellular organism, the promoter can also be specific to a particular tissue or organ or stage of development.

Such expression cassettes will comprise the transcriptional initiation region of the invention linked to a nucleotide sequence of interest. Such an expression cassette is provided with a plurality of restriction sites for insertion of the gene of interest to be under the transcriptional regulation of the regulatory regions. The expression cassette may additionally contain selectable marker genes.

The term “RNA transcript” refers to the product resulting from RNA polymerase catalyzed transcription of a DNA sequence. When the RNA transcript is a perfect complementary copy of the DNA sequence, it is referred to as the primary transcript or it may be a RNA sequence derived from posttranscriptional processing of the primary transcript and is referred to as the mature RNA. “Messenger RNA” (mRNA) refers to the RNA that is without introns and that can be translated into protein by the cell. “cDNA” refers to a single- or a double-stranded DNA that is complementary to and derived from mRNA.

“Regulatory sequences” and “suitable regulatory sequences” each refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences include enhancers, promoters, translation leader sequences, introns, and polyadenylation signal sequences. They include natural and synthetic sequences as well as sequences that may be a combination of synthetic and natural sequences. As is noted above, the term “suitable regulatory sequences” is not limited to promoters. However, some suitable regulatory sequences useful in the present invention will include, but are not limited to constitutive promoters, tissue-specific promoters, development-specific promoters, inducible promoters and viral promoters.

“5′ non-coding sequence” refers to a nucleotide sequence located 5′ (upstream) to the coding sequence. It is present in the fully processed mRNA upstream of the initiation codon and may affect processing of the primary transcript to mRNA, mRNA stability or translation efficiency (Turner et al., Mol. Biotech., 3:225 (1995).

“3′ non-coding sequence” refers to nucleotide sequences located 3′ (downstream) to a coding sequence and include polyadenylation signal sequences and other sequences encoding regulatory signals capable of affecting mRNA processing or gene expression. The polyadenylation signal is usually characterized by affecting the addition of polyadenylic acid tracts to the 3′ end of the mRNA precursor.

The term “translation leader sequence” refers to that DNA sequence portion of a gene between the promoter and coding sequence that is transcribed into RNA and is present in the fully processed mRNA upstream (5′) of the translation start codon. The translation leader sequence may affect processing of the primary transcript to mRNA, mRNA stability or translation efficiency.

The term “mature” protein refers to a post-translationally processed polypeptide without its signal peptide. “Precursor” protein refers to the primary product of translation of an mRNA. “Signal peptide” refers to the amino terminal extension of a polypeptide, which is translated in conjunction with the polypeptide forming a precursor peptide and which is required for its entrance into the secretory pathway. The term “signal sequence” refers to a nucleotide sequence that encodes the signal peptide.

“Promoter” refers to a nucleotide sequence, usually upstream (5′) to its coding sequence, which controls the expression of the coding sequence by providing the recognition for RNA polymerase and other factors required for proper transcription. “Promoter” includes a minimal promoter that is a short DNA sequence comprised of a TATA-box and other sequences that serve to specify the site of transcription initiation, to which regulatory elements are added for control of expression. “Promoter” also refers to a nucleotide sequence that includes a minimal promoter plus regulatory elements that is capable of controlling the expression of a coding sequence or functional RNA. This type of promoter sequence consists of proximal and more distal upstream elements, the latter elements often referred to as enhancers. Accordingly, an “enhancer” is a DNA sequence that can stimulate promoter activity and may be an innate element of the promoter or a heterologous element inserted to enhance the level or tissue specificity of a promoter. Promoters may be derived in their entirety from a native gene, or be composed of different elements derived from different promoters found in nature, or even be comprised of synthetic DNA segments. A promoter may also contain DNA sequences that are involved in the binding of protein factors that control the effectiveness of transcription initiation in response to physiological or developmental conditions.

The “initiation site” is the position surrounding the first nucleotide that is part of the transcribed sequence, which is also defined as position +1. With respect to this site all other sequences of the gene and its controlling regions are numbered. Downstream sequences (i.e. further protein encoding sequences in the 3′ direction) are denominated positive, while upstream sequences (mostly of the controlling regions in the 5′ direction) are denominated negative.

Promoter elements, particularly a TATA element, that are inactive or that have greatly reduced promoter activity in the absence of upstream activation are referred to as “minimal or core promoters.” In the presence of a suitable transcription factor, the minimal promoter functions to permit transcription. A “minimal or core promoter” thus consists only of all basal elements needed for transcription initiation, e.g., a TATA box and/or an initiator.

“Constitutive expression” refers to expression using a constitutive or regulated promoter. “Conditional” and “regulated expression” refer to expression controlled by a regulated promoter.

“Operably-linked” may refer to the association of nucleic acid sequences on single nucleic acid fragment so that the function of one is affected by the other. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably-linked to regulatory sequences in sense or antisense orientation.

“Expression” refers to the transcription and/or translation in a cell of an endogenous gene, transgene, as well as the transcription and stable accumulation of sense (mRNA) or functional RNA. In the case of antisense constructs, expression may refer to the transcription of the antisense DNA only. Expression may also refer to the production of protein.

“Transcription stop fragment” refers to nucleotide sequences that contain one or more regulatory signals, such as polyadenylation signal sequences, capable of terminating transcription. Examples of transcription stop fragments are known to the art.

“Translation stop fragment” refers to nucleotide sequences that contain one or more regulatory signals, such as one or more termination codons in all three frames, capable of terminating translation. Insertion of a translation stop fragment adjacent to or near the initiation codon at the 5′ end of the coding sequence will result in no translation or improper translation. Excision of the translation stop fragment by site-specific recombination will leave a site-specific sequence in the coding sequence that does not interfere with proper translation using the initiation codon.

The terms “cis-acting sequence” and “cis-acting element” refer to DNA or RNA sequences whose functions require them to be on the same molecule.

The terms “trans-acting sequence” and “trans-acting element” refer to DNA or RNA sequences whose function does not require them to be on the same molecule.

The following terms are used to describe the sequence relationships between two or more sequences (e.g., nucleic acids, polynucleotides or polypeptides): (a) “reference sequence,” (b) “comparison window,” (c) “sequence identity,” (d) “percentage of sequence identity,” and (e) “substantial identity.”
(a) As used herein, “reference sequence” is a defined sequence used as a basis for sequence comparison. A reference sequence may be a subset or the entirety of a specified sequence; for example, as a segment of a full length cDNA, gene sequence or peptide sequence, or the complete cDNA, gene sequence or peptide sequence.
(b) As used herein, “comparison window” makes reference to a contiguous and specified segment of a sequence, wherein the sequence in the comparison window may comprise additions or deletions (i.e., gaps) compared to the reference sequence (which does not comprise additions or deletions) for optimal alignment of the two sequences. Generally, the comparison window is at least 20 contiguous nucleotides in length, and optionally can be 30, 40, 50, 100, or longer. Those of skill in the art understand that to avoid a high similarity to a reference sequence due to inclusion of gaps in the sequence a gap penalty is typically introduced and is subtracted from the number of matches.

Methods of alignment of sequences for comparison are well known in the art. Thus, the determination of percent identity between any two sequences can be accomplished using a mathematical algorithm. Non-limiting examples of such mathematical algorithms are the algorithm of Myers and Miller, CABIOS, 4:11 (1988); the local homology algorithm of Smith et al., Adv. Appl. Math., 2:482 (1981); the homology alignment algorithm of Needleman and Wunsch, J M B, 48:443 (1970); the search-for-similarity-method of Pearson and Lipman, Proc. Natl. Acad. Sci. USA, 85:2444 (1988); the algorithm of Karlin and Altschul, Proc. Natl. Acad. Sci. USA, 87:2264 (1990), modified as in Karlin and Altschul, Proc. Natl. Acad. Sci. USA, 90:5873 (1993).

Computer implementations of these mathematical algorithms can be utilized for comparison of sequences to determine sequence identity. Such implementations include, but are not limited to: CLUSTAL in the PC/Gene program (available from Intelligenetics, Mountain View, California); the ALIGN program (Version 2.0) and GAP, BESTFIT, BLAST, FASTA, and TFASTA in the Wisconsin Genetics Software Package, Version 8 (available from Genetics Computer Group (GCG), 575 Science Drive, Madison, Wisconsin, USA). Alignments using these programs can be performed using the default parameters. The CLUSTAL program is well described by Higgins et al., Gene, 73:237 (1988); Higgins et al., CABIOS, 5:151 (1989); Corpet et al., Nucl. Acids Res., 16:10881 (1988); Huang et al., CABIOS, 8:155 (1992); and Pearson et al., Meth. Mol. Biol., 24:307 (1994). The ALIGN program is based on the algorithm of Myers and Miller, supra. The BLAST programs of Altschul et al., J M B, 215:403 (1990); Nucl. Acids Res., 25:3389 (1990), are based on the algorithm of Karlin and Altschul supra.

Software for performing BLAST analyses is publicly available through the National Center for Biotechnology Information (available on the world wide web at ncbi.nlm.nih.gov). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached.

In addition to calculating percent sequence identity, the BLAST algorithm also performs a statistical analysis of the similarity between two sequences. One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleic acid sequence to the reference nucleic acid sequence is less than about 0.1, more specifically less than about 0.01, and most specifically less than about 0.001.

To obtain gapped alignments for comparison purposes, Gapped BLAST (in BLAST 2.0) can be utilized as described in Altschul et al., Nucleic Acids Res. 25:3389 (1997). Alternatively, PSI-BLAST (in BLAST 2.0) can be used to perform an iterated search that detects distant relationships between molecules. See Altschul et al., supra. When utilizing BLAST, Gapped BLAST, PSI-BLAST, the default parameters of the respective programs (e.g., BLASTN for nucleotide sequences, BLASTX for proteins) can be used. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix. See the world wide web at ncbi.nlm.nih.gov. Alignment may also be performed manually by visual inspection.

For purposes of the present invention, comparison of sequences for determination of percent sequence identity to another sequence may be made using the BlastN program (version 1.4.7 or later) with its default parameters or any equivalent program. By “equivalent program” is intended any sequence comparison program that, for any two sequences in question, generates an alignment having identical nucleotide or amino acid residue matches and an identical percent sequence identity when compared to the corresponding alignment generated by the preferred program.
(c) As used herein, “sequence identity” or “identity” in the context of two nucleic acid or polypeptide sequences makes reference to a specified percentage of residues in the two sequences that are the same when aligned for maximum correspondence over a specified comparison window, as measured by sequence comparison algorithms or by visual inspection. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, California).
(d) As used herein, “percentage of sequence identity” means the value determined by comparing two optimally aligned sequences over a comparison window, wherein the portion of the sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.
(e)(i) The term “substantial identity” of sequences means that a polynucleotide comprises a sequence that has at least 70%, 71%, 72%, 73%, 74%, 75%, 76%, 77%, 78%, or 79%, at least 80%, 81%, 82%, 83%, 84%, 85%, 86%, 87%, 88%, or 89%, at least 90%, 91%, 92%, 93%, or 94%, and at least 95%, 96%, 97%, 98%, or 99% sequence identity, compared to a reference sequence using one of the alignment programs described using standard parameters. One of skill in the art will recognize that these values can be appropriately adjusted to determine corresponding identity of proteins encoded by two nucleotide sequences by taking into account codon degeneracy, amino acid similarity, reading frame positioning, and the like. Substantial identity of amino acid sequences for these purposes normally means sequence identity of at least 70%, at least 80%, 90%, at least 95%.

Another indication that nucleotide sequences are substantially identical is if two molecules hybridize to each other under stringent conditions (see below). Generally, stringent conditions are selected to be about 5° C. lower than the thermal melting point (Tₘ) for the specific sequence at a defined ionic strength and pH. However, stringent conditions encompass temperatures in the range of about 1° C. to about 20° C., depending upon the desired degree of stringency as otherwise qualified herein. Nucleic acids that do not hybridize to each other under stringent conditions are still substantially identical if the polypeptides they encode are substantially identical. This may occur, e.g., when a copy of a nucleic acid is created using the maximum codon degeneracy permitted by the genetic code. One indication that two nucleic acid sequences are substantially identical is when the polypeptide encoded by the first nucleic acid is immunologically cross reactive with the polypeptide encoded by the second nucleic acid.
(e)(ii) The term “substantial identity” in the context of a peptide indicates that a peptide comprises a sequence with at least 70%, 71%, 72%, 73%, 74%, 75%, 76%, 77%, 78%, or 79%, 80%, 81%, 82%, 83%, 84%, 85%, 86%, 87%, 88%, or 89%, at least 90%, 91%, 92%, 93%, or 94%, or 95%, 96%, 97%, 98% or 99%, sequence identity to the reference sequence over a specified comparison window. Optimal alignment is conducted using the homology alignment algorithm of Needleman and Wunsch, J. Mol. Biol. 48:443 (1970). An indication that two peptide sequences are substantially identical is that one peptide is immunologically reactive with antibodies raised against the second peptide. Thus, a peptide is substantially identical to a second peptide, for example, where the two peptides differ only by a conservative substitution.

For sequence comparison, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

As noted above, another indication that two nucleic acid sequences are substantially identical is that the two molecules hybridize to each other under stringent conditions. The phrase “hybridizing specifically to” refers to the binding, duplexing, or hybridizing of a molecule only to a particular nucleotide sequence under stringent conditions when that sequence is present in a complex mixture (e.g., total cellular) DNA or RNA. “Bind(s) substantially” refers to complementary hybridization between a probe nucleic acid and a target nucleic acid and embraces minor mismatches that can be accommodated by reducing the stringency of the hybridization media to achieve the desired detection of the target nucleic acid sequence. “Stringent hybridization conditions” and “stringent hybridization wash conditions” in the context of nucleic acid hybridization experiments such as Southern and Northern hybridizations are sequence dependent, and are different under different environmental parameters. Longer sequences hybridize specifically at higher temperatures. The thermal melting point (Tₘ) is the temperature (under defined ionic strength and pH) at which 50% of the target sequence hybridizes to a perfectly matched probe. Specificity is typically the function of post-hybridization washes, the critical factors being the ionic strength and temperature of the final wash solution.

By “variant” polypeptide is intended a polypeptide derived from the native protein by deletion (so-called truncation) or addition of one or more amino acids to the N-terminal and/or C-terminal end of the native protein; deletion or addition of one or more amino acids at one or more sites in the native protein; or substitution of one or more amino acids at one or more sites in the native protein. Such variants may result from, for example, genetic polymorphism or from human manipulation. Methods for such manipulations are generally known in the art.

Thus, the polypeptides of the invention may be altered in various ways including amino acid substitutions, deletions, truncations, and insertions. Methods for such manipulations are generally known in the art. For example, amino acid sequence variants of the polypeptides can be prepared by mutations in the DNA. Methods for mutagenesis and nucleotide sequence alterations are well known in the art. See, for example, Kunkel, Proc. Natl. Acad. Sci. USA, 82:488 (1985); Kunkel et al., Meth. Enzymol., 154:367 (1987); U.S. Pat. No. 4,873,192; Walker and Gaastra, Techniques in Mol. Biol. (MacMillan Publishing Co. (1983), and the references cited therein. Guidance as to appropriate amino acid substitutions that do not affect biological activity of the protein of interest may be found in the model of Dayhoff et al., Atlas of Protein Sequence and Structure (Natl. Biomed. Res. Found. 1978). Conservative substitutions, such as exchanging one amino acid with another having similar properties, are preferred.

Thus, the genes and nucleotide sequences of the invention include both the naturally occurring sequences as well as mutant forms. Likewise, the polypeptides of the invention encompass naturally occurring proteins as well as variations and modified forms thereof. Such variants will continue to possess the desired activity. The deletions, insertions, and substitutions of the polypeptide sequence encompassed herein are not expected to produce radical changes in the characteristics of the polypeptide. However, when it is difficult to predict the exact effect of the substitution, deletion, or insertion in advance of doing so, one skilled in the art will appreciate that the effect will be evaluated by routine screening assays.

Individual substitutions deletions or additions that alter, add or delete a single amino acid or a small percentage of amino acids (typically less than 5%, more typically less than 1%) in an encoded sequence are “conservatively modified variations,” where the alterations result in the substitution of an amino acid with a chemically similar amino acid. Conservative substitution tables providing functionally similar amino acids are well known in the art. The following five groups each contain amino acids that are conservative substitutions for one another: Aliphatic: Glycine (G), Alanine (A), Valine (V), Leucine (L), Isoleucine (I); Aromatic: Phenylalanine (F), Tyrosine (Y), Tryptophan (W); Sulfur-containing: Methionine (M), Cysteine (C); Basic: Arginine (R), Lysine (K), Histidine (H); Acidic: Aspartic acid (D), Glutamic acid (E), Asparagine (N), Glutamine (Q). In addition, individual substitutions, deletions or additions which alter, add or delete a single amino acid or a small percentage of amino acids in an encoded sequence are also “conservatively modified variations.”

The term “transformation” refers to the transfer of a nucleic acid fragment into the genome of a host cell, resulting in genetically stable inheritance. Host cells containing the transformed nucleic acid fragments are referred to as “transgenic” cells, and organisms comprising transgenic cells are referred to as “transgenic organisms”.

“Transformed,” “transgenic,” and “recombinant” refer to a host cell or organism into which a heterologous nucleic acid molecule has been introduced. The nucleic acid molecule can be stably integrated into the genome generally known in the art and are disclosed in Sambrook and Russell, supra. See also Innis et al., PCR Protocols, Academic Press (1995); and Gelfand, PCR Strategies, Academic Press (1995); and Innis and Gelfand, PCR Methods Manual, Academic Press (1999). Known methods of PCR include, but are not limited to, methods using paired primers, nested primers, single specific primers, degenerate primers, gene-specific primers, vector-specific primers, partially mismatched primers, and the like. For example, “transformed,” “transformant,” and “transgenic” cells have been through the transformation process and contain a foreign gene integrated into their chromosome. The term “untransformed” refers to normal cells that have not been through the transformation process.

### Methods of Use

Certain embodiments provide a method of inhibiting the activity of fentanyl, or analog(s) thereof, in a mammal, comprising administering an anti-fentanyl antibody, or fragment thereof, as described herein to the mammal. In certain embodiments, an antibody of the invention or a fragment thereof inhibits the activity (e.g., analgesia, antinociception, respiratory depression, apnea, lowered oxygen saturation, and/or bradycardia, etc.) of fentanyl, or analog(s) thereof, by at least about 5%, at least about 10%, at least about 15%, at least about 20%, at least about 25%, at least about 30%, at least about 40%, at least about 50%, at least about 60%, at least about 70%, at least about 80%, at least about 90%, at least about 95%, at least about 99% or at least about 100% as compared to a control.

For example, in certain embodiments, fentanyl or analog-induced pharmacological or physiological effect(s) are reduced or reversed by at least about 5%, 10%, 15%, 20%, 25%, 30%, 35%, 40%, 50%, 60%, 70%, 80%, 90%, 95%, or 100% as compared to a control. In certain embodiments, fentanyl-induced antinociception or analgesic effect is reduced by at least about 5%, 10%, 15%, 20%, 25%, 30%, 35%, 40%, 50%, 60%, 70%, 80%, 90%, 95%, or 100% as compared to a control. In certain embodiments, fentanyl-induced respiratory depression is prevented or reduced by at least about 5%, 10%, 15%, 20%, 25%, 30%, 35%, 40%, 50%, 60%, 70%, 80%, 90%, 95%, or 100% as compared to a control. In certain embodiments, fentanyl-induced apnea is prevented or reduced or reversed by at least about 5%, 10%, 15%, 20%, 25%, 30%, 35%, 40%, 50%, 60%, 70%, 80%, 90%, 95%, or 100% as compared to a control. In certain embodiments, the depressed breath rate, tidal volume, minute volume, and/or EtCO₂ of a mammal under fentanyl influence is increased by at least about 5%, 10%, 15%, 20%, 25%, 30%, 35%, 40%, 50%, 60%, 70%, 80%, 90%, 95%, or 100% as compared to a control. In certain embodiments, the lowered oxygen saturation of a mammal under fentanyl influence is increased by at least about 5%, 10%, 15%, 20%, 25%, 30%, 35%, 40%, 50%, 60%, 70%, 80%, 90%, 95%, or 100% as compared to a control. In certain embodiments, fentanyl-induced bradycardia is prevented or reduced by at least about 5%, 10%, 15%, 20%, 25%, 30%, 35%, 40%, 50%, 60%, 70%, 80%, 90%, 95%, or 100% as compared to a control. In certain embodiments, the slowed heart rate of a mammal under fentanyl influence is increased by at least about 5%, 10%, 15%, 20%, 25%, 30%, 35%, 40%, 50%, 60%, 70%, 80%, 90%, 95%, or 100% as compared to a control.

In certain embodiments, fentanyl or analog thereof bound to an antibody or fragment thereof described herein is sequestered in blood (e.g., whole blood or serum sample). In certain embodiments, the distribution of fentanyl or analog into the brain is reduced or reversed by at least about 5%, 10%, 15%, 20%, 25%, 30%, 35%, 40%, 50%, 60%, 70%, 80%, 90%, 95%, or 100% as compared to a control. In certain embodiments, the concentration of fentanyl or analog in the brain is reduced or reversed by at least about 5%, 10%, 15%, 20%, 25%, 30%, 35%, 40%, 50%, 60%, 70%, 80%, 90%, 95%, or 100% as compared to a control. In certain embodiments, the ratio of fentanyl or analog drug concentrations in brain over blood is reduced by at least about 5%, 10%, 15%, 20%, 25%, 30%, 35%, 40%, 50%, 60%, 70%, 80%, 90%, 95%, or 100% (e.g., reduced by 200%, 300%, or 400%) as compared to a control. Accordingly, the severity of opioid use disorder, or the risk of death due to synthetic opioid overdose (fentanyl or analogs overdose) is reduced.

Certain embodiments also provide a method for treating fentanyl or analog related overdose or disorder in a mammal, comprising administering an effective amount of an isolated anti-fentanyl antibody, or fragment thereof, as described herein to the mammal. In certain embodiments, the fentanyl or analog related overdose is fentanyl overdose. In certain embodiments, the fentanyl or analog related overdose is carfentanil overdose. In certain embodiments, the fentanyl or analog related overdose is acetylfentanyl overdose. In certain embodiments, the fentanyl or analog related disorder is opioid use disorder. In certain embodiments, the fentanyl or analog related overdose is reversed.

In certain embodiments, the method further comprises administering at least one additional therapeutic agent to the mammal. In certain embodiments, the at least one additional therapeutic agent is useful for overdose reversal or treating opioid use disorder. In certain embodiments, the at least one additional therapeutic agent is a μ-opioid receptor (MOR) antagonist (e.g., naloxone, or naltrexone). In certain embodiments, the at least one additional therapeutic agent is a μ-opioid receptor (MOR) agonist (e.g., methadone) or partial agonist (e.g., buprenorphine). In certain embodiments, the at least one additional therapeutic agent is a medical countermeasure for chemical attacks (e.g., nerve agents). In certain embodiments, the at least one additional therapeutic agent is a medication used in reversal of anesthesia. In certain embodiments, the at least one additional therapeutic agent is a medication used in critical care such as anesthetics for intra-op care. In certain embodiments, the at least one additional therapeutic agent is a medication used in standard care such as anti-inflammatory agents (e.g., acetaminophen).

Certain embodiments provide an isolated anti-fentanyl antibody, or fragment thereof, as described herein for the prophylactic or therapeutic treatment of fentanyl or analog related overdose or disorder. For example, in certain embodiments, the prophylactic treatment of fentanyl or analog related overdose, poisoning, or disorder comprises administering the antibody or fragment thereof to a mammal prior to exposure to fentanyl or analog (pre-exposure). In certain embodiments, the therapeutic treatment of fentanyl or analog related overdose, poisoning, or disorder comprises administering the antibody or fragment thereof to a mammal after exposure to fentanyl or analog (post-exposure), for example, as an overdose reversal treatment.

Certain embodiments provide the use of an isolated anti-fentanyl antibody, or fragment thereof, as described herein to prepare a medicament for the treatment of fentanyl or analog related overdose or disorder in a mammal.

Certain embodiments provide the use of an isolated anti-fentanyl antibody, or fragment thereof, as described herein to prepare a medicament for the treatment of fentanyl or analog related overdose or disorder in a mammal.

Certain embodiments provide an isolated anti-fentanyl antibody, or fragment thereof, as described herein for use in medical therapy.

Certain embodiments of the invention provide a method of preventing or inhibiting renarcotization in a mammal, comprising administering an antibody or fragment thereof described herein to the mammal.

In certain embodiments, the mammal is a rodent (e.g., mouse or rat).

In certain embodiments, the mammal is a canine (e.g., police or military dogs). In certain embodiments, the mammal is a mammal (e.g., human) in need of (e.g., overdose reversal). In certain embodiments, the mammal is a human, for example, exposed to or under influence of fentanyl or analog. In certain embodiments, the mammal is a human having opioid use disorder. In certain embodiments, the mammal is a human having substance use disorder. In certain embodiments, the mammal is a human having opioid-based pain management.

### Administration

For in vivo use, an antibody of the invention, or fragment thereof, is generally incorporated into a pharmaceutical composition prior to administration. Within such compositions, one or more antibodies of the invention may be present as active ingredient(s) (i.e., are present at levels sufficient to provide a statistically significant effect on the symptoms of a relevant disease, as measured using a representative assay). A pharmaceutical composition comprises one or more such antibodies in combination with any pharmaceutically acceptable carrier(s) known to those skilled in the art to be suitable for the particular mode of administration. In addition, other pharmaceutically active ingredients (including other therapeutic agents) may, but need not, be present within the composition.

The term “therapeutically effective amount,” in reference to treating a disease state/condition, refers to an amount of an antibody or fragment thereof either alone or as contained in a pharmaceutical composition that is capable of having any detectable, positive effect on any symptom, aspect, or characteristics of a disease state/condition when administered as a single dose or in multiple doses. Such effect need not be absolute to be beneficial.

The terms “treat” and “treatment” refer to both therapeutic treatment and prophylactic or preventative measures, wherein the object is to prevent or decrease an undesired physiological change or disorder. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean reducing risk of death, reducing the severity of the disorder, or prolonging survival as compared to expected survival if not receiving treatment. Those in need of treatment include those already with the condition or disorder as well as those prone to have the condition or disorder or those in which the condition or disorder is to be prevented.

In certain embodiments, the present antibodies (i.e., antibody of the present invention or a fragment thereof) may be systemically administered, e.g., intravenously, in combination with a pharmaceutically acceptable vehicle. In certain embodiments, the antibody or fragment thereof may be administered intravenously, subcutaneously, intradermally, intramuscularly, intranasally, or intraosseously by infusion, injection, or other delivery method (e.g., nasal spray, nebulizer). In certain embodiments, the administration of antibody may be paired with intrathecal or epidural delivery of pain management drug. In certain embodiments, antibody of the present invention or a fragment thereof is present in a liquid composition (e.g., saline or D5W). In certain embodiments, antibody of the present invention or a fragment thereof is present in a solid composition. In certain embodiments, antibody of the present invention or a fragment thereof is present in a lyophilized composition. In certain embodiments, the lyophilized composition further comprises one or more excipients selected from the group consisting of a cryo-lyoprotectant (e.g., trehalose, sucrose) and a bulking agent (e.g., mannitol, glycine).

In certain embodiments, the present antibodies (i.e., antibody of the present invention or a fragment thereof) may be administered, e.g., intravenously, to a mammal in need of, for example, about once daily, once every three days, once per week, once every two weeks, once every three weeks, once per month, once every five weeks, or once every six weeks.

The antibody may be administered intravenously, or intraperitoneally by infusion or injection. Additionally, the antibody may be administered by local injection, such as by intrathecal injection, epidural injection or peri-neural injection using a scope. Solutions of the antibody may be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the antibody that are adapted for the extemporaneous preparation of sterile injectable or infusible solutions or dispersions. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The prevention of the action of microorganisms can be brought about by various antibacterial and antifungal agents, for example, parabens, chlorobutanol, phenol, sorbic acid, thimerosal, and the like. In many cases, it will be useful to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the antibody in the required amount in the appropriate solvent with various of the other ingredients enumerated above, as required, followed by filter sterilization. In the case of sterile powders for the preparation of sterile injectable solutions, the methods of preparation are vacuum drying and the freeze drying techniques, which yield a powder of the antibody plus any additional desired ingredient present in the previously sterile-filtered solutions.

In certain embodiments, the present antibodies (i.e., antibody of the present invention or a fragment thereof) may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the antibody may be combined with one or more excipients and used in the form of ingestible tablets, buccal tablets, troches, capsules, elixirs, suspensions, syrups, wafers, and the like. Such compositions and preparations should contain at least 0.1% of an antibody of the present invention. The percentage of the compositions and preparations may, of course, be varied and may conveniently be between about 2 to about 60% of the weight of a given unit dosage form. The amount of antibody in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the antibody, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the antibody may be incorporated into sustained-release preparations and devices.

For topical administration, the present antibodies may be applied in pure form, i.e., when they are liquids. However, it will generally be desirable to administer them to the skin as compositions or formulations, in combination with a dermatologically acceptable carrier, which may be a solid or a liquid.

In certain embodiments, the present antibody or fragment thereof may be delivered using a device (e.g., a device comprising one or two chambers, for example, containing the antibody or fragment, and/or liquid). In certain embodiments, the device is an injector device (e.g., a self-injector device comprising the antibody or fragment thereof). In certain embodiments, the device is a nasal drug delivery device (e.g., nasal spray device).

Useful solid carriers include finely divided solids such as talc, clay, microcrystalline cellulose, silica, alumina and the like. Useful liquid carriers include water, alcohols or glycols or water-alcohol/glycol blends, in which the present antibodies can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Examples of useful dermatological compositions are known to the art; for example, see Jacquet et al. (U.S. Pat. No. 4,608,392), Geria (U.S. Pat. No. 4,992,478), Smith et al. (U.S. Pat. No. 4,559,157) and Wortzman (U.S. Pat. No. 4,820,508).

Useful dosages of the antibodies of the present invention can be determined by comparing their in vitro activity, and in vivo activity in animal models. Methods for the extrapolation of effective dosages in mice, and other animals, to humans are known to the art; for example, see U.S. Pat. No. 4,938,949.

The amount of an antibody of the present invention required for use in treatment will vary with the route of administration, the nature of the condition being treated and the age and condition of the patient and will be ultimately at the discretion of the attendant physician, pharmacist, or clinician.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations.

Antibodies of the invention can also be administered in combination with other therapeutic agents and/or treatments, such as other agents or treatments that are useful for the treatment of overdose, opioid use disorder, or substance use disorder. Additionally, one or more antibodies of the invention, or fragments thereof, may be administered or co-administered (e.g., a combination of antibodies, or fragments thereof, may be administered). Accordingly, one embodiment the invention also provides a composition comprising an antibody of the invention, or a fragment thereof, at least one other therapeutic agent, and a pharmaceutically acceptable diluent or carrier. The invention also provides a kit comprising an antibody of the invention, or a fragment thereof, at least one other therapeutic agent, packaging material, and instructions for administering an antibody of the invention, or a fragment thereof, and the other therapeutic agent or agents to an animal to treat overdose, opioid use disorder, or substance use disorder (e.g., methamphetamine users accidentally exposed to fentanyl).

As used herein, the term “therapeutic agent” refers to any agent or material that has a beneficial effect on the mammalian recipient.

The invention will now be illustrated by the following non-limiting Examples.

### Example 1. Characterization of Humanized mAb to Counteract Fentanyl Toxicity to Support Advanced Development and Translation

With the incidence of opioid-related overdoses continuing to increase annually, innovative therapies to complement current treatments are needed. Murine monoclonal antibodies (mAb) specific for fentanyl and its analogs have demonstrated pre-clinical efficacy in preventing and reversing drug-induced toxicity in rodent models. However, mAb-based therapeutics may require further engineering as well as in vitro and in vivo characterization to support advancement to clinical development activities and first-in-human clinical trials. Here, novel murine anti-fentanyl mAbs were identified for further development based on affinity for fentanyl and its analogs, and efficacy in counteracting fentanyl effects in mice. Rounds of sequential grafting of murine CDR sequences onto human germline variable region sequences and mutations designed to eliminate predicted post-translational modifications resulted in two humanized mAbs that maintained sub-nanomolar affinity for fentanyl as measured by biolayer interferometry. These humanized mAbs were assessed by size exclusion chromatography to evaluate aggregation tendency, hydrophobic interaction chromatography for hydrophobicity, and dynamic scanning fluorimetry for thermostability. Humanized mAbs described herein were effective at preventing fentanyl-induced pharmacological effects up to 0.3 mg/kg in rats, and reduced fentanyl distribution to brain. These results collectively support that humanized anti-fentanyl mAbs warrant further clinical development for prevention of fentanyl toxicity.

### INTRODUCTION

Fatal overdoses involving opioids are at an all-time high in the United States,¹ and overdose deaths involving synthetic opioids have increased annually since 2013.² Fentanyl, an increasingly common and one of the most potent synthetic opioids,³⁻⁶ is associated with a significant number of overdose deaths: between May 2020 and May 2021, 62,136 overdose deaths associated with synthetic opioids were reported, accounting for 84.8% of all opioid-related overdose deaths.^{1,7} The current clinically approved treatment for reversal of opioid-related toxicity is naloxone, a μ-opioid receptor (MOR) antagonist.⁸ Naloxone is typically effective for combating opioid toxicity and overdose; however, for MOR agonists that exhibit a serum half-life longer than that of naloxone (30-90 minutes),^{9,10} additional doses of naloxone may be required to protect against renarcotization. Specifically, fentanyl has a serum half-life of −8 hrs;¹¹ consequently, additional dosing of naloxone and extended monitoring for signs of recurring fentanyl toxicity can be needed for 2 or more hours.^{12, 13} The drastic and sustained increase in overdose deaths related to synthetic opioids indicates that current methods of intervention are inadequate; therefore, the development of alternative or complementary treatment options is paramount.

Monoclonal antibodies (mAb) represent a promising alternative treatment modality for counteracting toxic doses of small-molecule drugs by sequestration of the drug molecules in serum, preventing their distribution to drug receptors in the brain. With a typical serum half-life of ˜21 days in humans, mAbs offer a ˜500-fold longer duration of efficacy compared to naloxone.^{14, 15} This long serum half-life may allow anti-opioid (α-opioid) mAb protection against renarcotization from fentanyl and/or other MOR agonists that persist after naloxone is metabolized, with the potential for extended protection against subsequent fentanyl exposure after mAb administration. Opioid overdose reversal treatment with a MOR antagonist like naloxone can have undesirable side effects, including precipitation of opioid withdrawal symptoms,^{10, 12} whereas α-opioid mAb treatment does not directly modulate MOR functioning. The target specificity of a mAb should prevent interference with endogenous opioid signaling, and with off-target MOR medications such as methadone, buprenorphine, or naltrexone, which could be administered concurrently. Recent data show that mAb is effective at reversing opioid toxicity in rodents.¹⁶ In addition to protection from and reversal of overdose, α-opioid mAb may provide utility in the prophylactic treatment of patients with opioid use disorder (OUD), as α-opioid vaccines have shown pre-clinical efficacy against various models of OUD.^{17, 18} Several mAbs targeting small molecule drugs are in various stages of clinical and pre-clinical study. Murine and chimeric anti-fentanyl (α-fentanyl) mAb have shown efficacy in rodent models.¹⁹⁻²¹ A partially humanized anti-cocaine mAb is in late-stage pre-clinical development.²² A chimeric anti-methamphetamine mAb (IXT-m200) that offers protection against methamphetamine toxicity has shown promising safety data in Phase I clinical trials,²³⁻²⁵ has completed one Phase II study and is preparing to initiate a second Phase II study (NCT05034874). Despite this success with murine and chimeric mAbs in pre-clinical and clinical settings, it has been well described that murine mAbs are not suitable as a therapeutic treatment in humans due to human anti-mouse antibody responses,²⁶ and chimeric monoclonal antibodies (chAb) with human constant regions and murine variable regions are also susceptible to immunogenicity.²⁷ Human mAb derived from humanized transgenic mouse or rat models are an industry-standard options; however, access to such models is often limited by the costly licensing fees associated with their use or by their proprietary ownership. Thus, humanization of murine-derived mAb remains a validated mAb engineering method for the development of therapeutic mAb candidates with a decreased immunogenicity risk compared to murine mAbs and chAbs,^{27, 28} and to date more than 50 humanized mAbs have been approved by the US Food and Drug Administration (FDA) or the European Medicines Agency.²⁹

This Example describes the isolation, humanization, and in vitro and in vivo characterization of novel α-fentanyl mAbs. Murine and chimeric mAbs prevented fentanyl-induced physiological effects and reduced fentanyl distribution to the brain in mice. Two mAbs were selected for humanization via a CDR grafting approach, evaluated to ensure that binding to fentanyl was maintained, and tested for biophysical properties consistent with mAb candidates suitable for clinical development and manufacturing.²⁰⁻²² These humanized mAbs showed high affinity for fentanyl, displayed favorable biophysical properties, and prevented physiological effects induced by fentanyl up to 0.3 mg/kg in rats.

### Results

### Overall α-Fentanyl mAb Discovery, Selection, and Humanization Strategy.

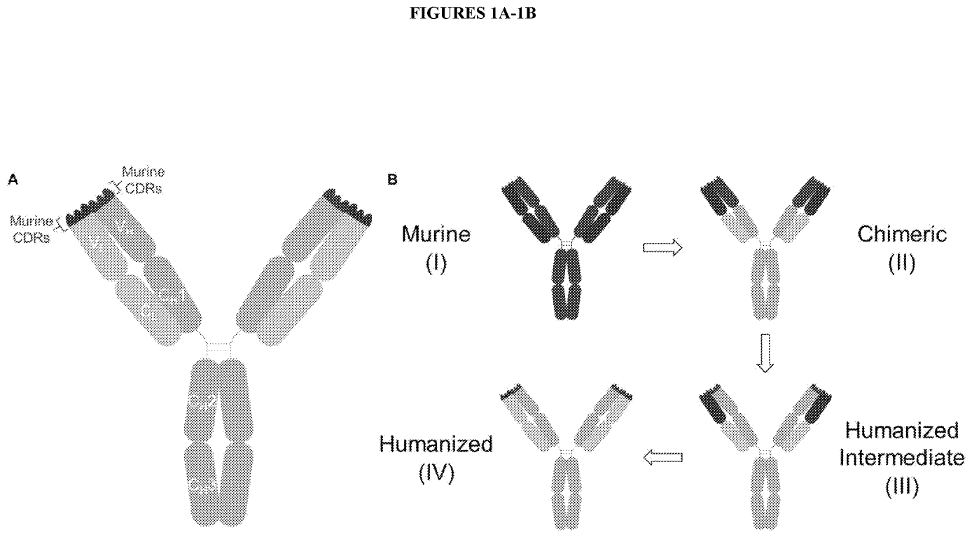

α-fentanyl murine mAbs were isolated from hybridomas generated from splenocytes of mice immunized with fentanyl vaccine F1-CRM.¹⁸ Hybridoma clones were screened for binding to fentanyl hapten by ELISA (data not shown), and positive clones were advanced for in vitro and in vivo characterization. Select murine mAbs (FIG. 1B-i) were sequenced and expressed as chAbs (FIG. 1B-ii). A stepwise humanization scheme was employed by first performing CDR grafting on heavy chains to produce a humanized intermediate, testing to ensure binding to fentanyl was maintained, then performing the grafting process on light chains (FIG. 1B). Humanized mAbs then underwent post-translational modification (PTM) mitigation, biophysical characterization, and efficacy testing in rats to select humanized mAb for further development.

### Characterization of Murine α-Fentanyl mAb.

Six murine mAbs that bound to fentanyl were identified: HY6-F9_Mu, HY11-2F5_Mu, HY11-4E6_Mu, HY11-5C1_Mu, HY11-6B2_Mu, and HY11-7E1_Mu. To determine affinity (K_{D}) and assess cross-reactivity of novel α-fentanyl murine mAbs to fentanyl analogs, binding of purified mAb to biotinylated haptens of fentanyl, acetylfentanyl, and carfentanil was measured by biolayer interferometry (BLI). All mAbs had measured K_{D} values of <0.1 nM to the fentanyl hapten (Table 1), while all HY11 mAbs also showed K_{D} values of <0.1 nM to the acetylfentanyl hapten, with HY6-F9_Mu yielding a K_{D} of 0.63 nM to the acetylfentanyl hapten. Only HY11-7E1_Mu showed measurable binding (K_{D}=1.18 nM) for the carfentanil hapten, whereas HY11-5C1_Mu and HY11-6B2_Mu showed low response to the biotinylated carfentanil hapten and a heterogenous binding profile, which indicates minimal or non-specific interaction of the mAb with the ligand-bound biosensor.

TABLE 1
Affinity of murine, chimeric and humanized mAb
determined by biolayer interferometry (BLI).

| | | Affinity (K_{D}, nM) for Ligand* | | |
|---|---|---|---|---|
| mAb ID | mAb Species | Fentanyl | Acetylfentanyl | Carfentanil |
| HY11-2F5 | Murine | <0.1 | <0.1 | 0.72 |
| HY11-4E6 | Murine | <0.1 | <0.1 | NDB |
| HY11-5C1 | Murine | <0.1 | <0.1 | NDB** |
| HY11-6B2 | Murine | <0.1 | <0.1 | NDB** |
| | Chimeric | <0.1 | <0.1 | NDB** |
| HY11-7E1 | Murine | <0.1 | <0.1 | 1.18 |
| | Chimeric | <0.1 | 0.28 | 0.51 |
| | Humanized | 0.26 | 0.80 | 2.26 |
| | Humanized | <0.1 | 0.88 | 1.31 |
| | VH D62E | | | |
| HY6-F9 | Murine | <0.1 | 0.63 | NDB |
| | Chimeric | <0.1 | 0.56 | NDB |
| | Humanized | 0.17 | 1.58 | NDB |
| | Humanized | <0.1 | 1.94 | NDB |
| | VL N34Q | | | |

NDB = No Detectable Binding;
*hapten;
**Binding detected by BLI, but binding curve profile is indicative of non-specific interaction

Certain antibodies were evaluated for fentanyl, acetylfentanyl, and carfentanil binding by BLI (Table 2).

TABLE 2
Extended BLI binding table of murine, chimeric, and humanized
mAb determined by biolayer interferometry (BLI)

| | | Fentanyl* | | | Acetylfentanyl* | | | Carfentanil* | | |
|---|---|---|---|---|---|---|---|---|---|---|
| mAb | mAb | K_{D} | K_{ON} | K_{OFF} | K_{D} | K_{ON} | K_{OFF} | K_{D} | K_{ON} | K_{OFF} |
| ID | Species | (nM) | (1/Ms) | (1/s) | (nM) | (1/Ms) | (1/s) | (nM) | (1/Ms) | (1/s) |
| HY11- | Murine | <0.1 | 2.15E+05 | <1.0E−07 | <0.1 | 2.03E+05 | <1.0E−07 | 0.72 | 5.71E+05 | 4.13E−04 |
| 2F5 | | | | | | | | | | |
| HY11- | Murine | <0.1 | 1.95E+05 | <1.0E−07 | <0.1 | 1.90E+05 | <1.0E−07 | NDB | NDB | NDB |
| 4E6 | | | | | | | | | | |
| HY11- | Murine | <0.1 | 3.07E+05 | <1.0E−07 | <0.1 | 2.31E+05 | 2.48E−06 | NDB | NDB | NDB |
| 5C1 | | | | | | | | | | |
| HY11- | Murine | <0.1 | 2.68E+05 | <1.0E−07 | <0.1 | 2.17E+05 | <1.0E−07 | NDB | NDB | NDB |
| 6B2 | Chimeric | <0.1 | 2.80E+05 | <1.0E−07 | <0.1 | 1.97E+05 | 7.41E−06 | NDB | NDB | NDB |
| HY11- | Murine | <0.1 | 2.74E+05 | <1.0E−07 | <0.1 | 2.32E+05 | <1.0E−07 | 1.18 | 1.47E+05 | 1.74E−04 |
| 7E1 | Chimeric | <0.1 | 1.83E+05 | <1.0E−07 | 0.28 | 1.84E+05 | 5.10E−05 | 0.51 | 1.12E+05 | 5.73E−05 |
| | Humanized | 0.24 | 2.44E+05 | 5.74E−05 | 0.80 | 2.98E+05 | 2.38E−04 | 2.48 | 3.89E+05 | 9.65E−04 |
| | Humanized_{—} | <0.1 | 1.85E+05 | 1.03E−05 | 0.88 | 2.11E+05 | 1.85E−04 | 1.31 | 2.81E+05 | 3.68E−04 |
| | VH D62E | | | | | | | | | |
| HY6- | Murine | <0.1 | 1.57E+05 | <1.0E−07 | 0.63 | 2.11E+05 | 1.32E−04 | NDB | NDB | NDB |
| F9 | Chimeric | <0.1 | 1.46E+05 | <1.0E−07 | 0.56 | 2.00E+05 | 1.11E−04 | NDB | NDB | NDB |
| | Humanized | 0.17 | 2.12E+05 | 3.51E−05 | 1.58 | 2.61E+05 | 4.11E−04 | NDB | NDB | NDB |
| | Humanized_{—} | <0.1 | 2.01E+05 | 1.93E−05 | 1.94 | 2.15E+05 | 4.17E−04 | NDB | NDB | NDB |
| | VL N34Q | | | | | | | | | |

NDB = No Detectable Binding,
*hapten

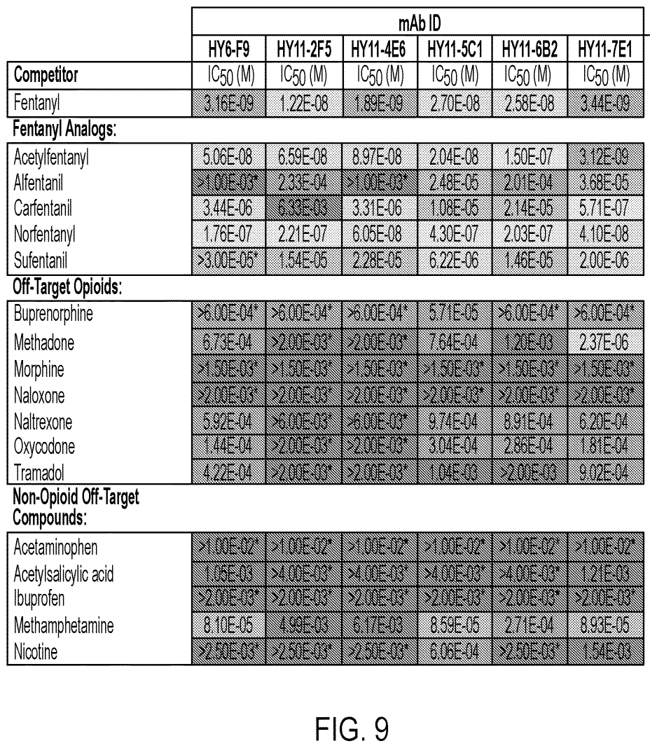

The relative affinity of mAbs for fentanyl compared to non-target compounds was evaluated by competitive ELISA. All mAbs showed nanomolar affinity for fentanyl, acetylfentanyl, and norfentanyl, a fentanyl metabolite (FIG. 9), and only HY11-7E1_Mu showed affinity for carfentanil <1 μM. By contrast, mAbs showed little to no binding to off-target opioid receptor ligands including buprenorphine, methadone, morphine, oxycodone, naloxone, naltrexone and tramadol, or to other non-opioid off-target drugs including methamphetamine, nicotine and common over-the-counter drugs acetaminophen, aspirin, or ibuprofen. The high specificity of mAb for fentanyl and its derivatives supports that these mAb would not interfere with commonly encountered compounds if present in a clinical setting.

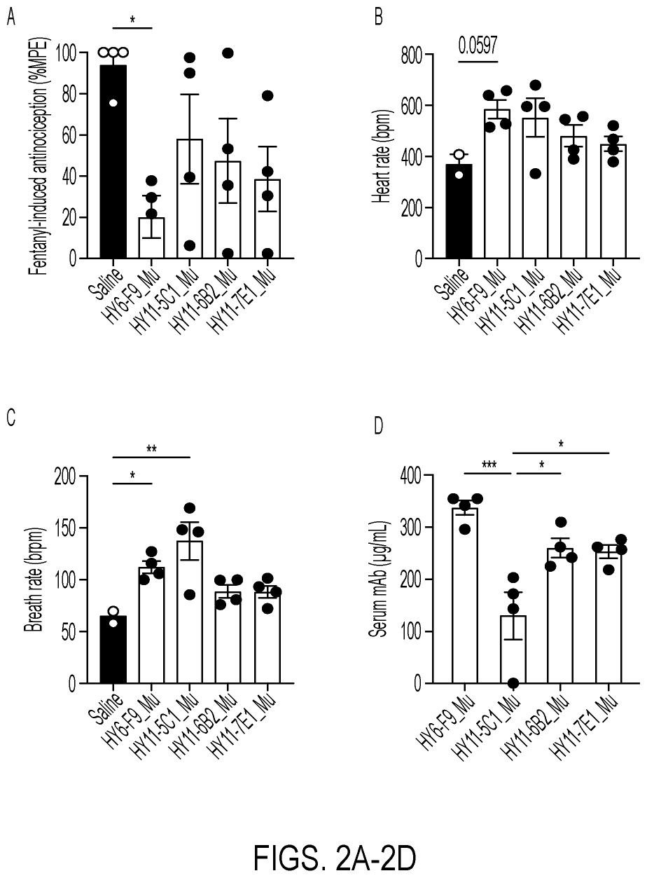

To determine whether isolated mAbs were effective at reducing fentanyl effects in vivo, mice were passively immunized with 40 mg/kg of HY6-F9_Mu, HY11-5C1_Mu, HY11-6B2_Mu, and HY11-7E1_Mu, and challenged with 0.25 mg/kg fentanyl s.c. (FIG. 2). HY1 1-2F5_Mu was not chosen due to sub-optimal levels of aggregation as measured by SEC-HPLC (data not shown) and HY11-4E6_Mu was not chosen due to high sequence similarity to HY6-F9_Mu. Treatment with mAb somewhat reduced fentanyl-induced antinociception, though only the effect of HY6-F9 was significant (FIG. 2A), and HY6-F9_Mu and HY11-5C1_Mu increased breath rate 30 minutes after fentanyl challenge relative to saline control FIG. 2C). One week after fentanyl challenge, concentration of mAb in serum was measured (FIG. 2D); the serum level of mAb in mice treated with HY11-5C1_Mu was significantly decreased compared to other mAbs, indicating this mAb may have lower serum stability than the other mAbs.

### Characterization and Efficacy of Chimeric α-Fentanyl mAb.

Heavy chain variable region (VH) and light chain variable region (VL) sequences from the murine mAbs HY6-F9_Mu, HY11-6B2_Mu, and HY11-7E1_Mu were cloned into expression vectors with human IgG1 and IgK constant region sequences to generate chAbs. Purified chimeric antibodies were assessed for binding to biotinylated fentanyl, acetylfentanyl, and carfentanil haptens by BLI (Table 1). All chAbs maintained K_{D} values below 0.1 nM for fentanyl hapten binding, matching their murine counterpart. The HY6-F9, HY11-5C1, and HY11-6B2 chimeric antibodies (HY6-F9_Ch, HY11-5C1_Ch, and HY11-6B2_Ch) bound to acetylfentanyl hapten with comparable binding affinities to their murine counterpart, while the affinity of chimeric HY11-7E1 (HY11-7E1_Ch) to acetylfentanyl hapten decreased (<0.1 nM to 0.51 nM). HY11-7E1_Ch maintained binding to the carfentanil hapten (0.57 nM).

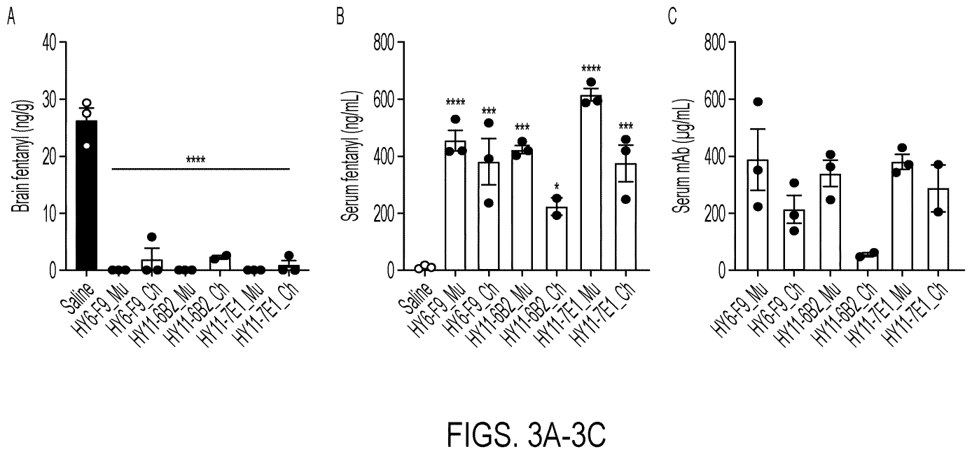

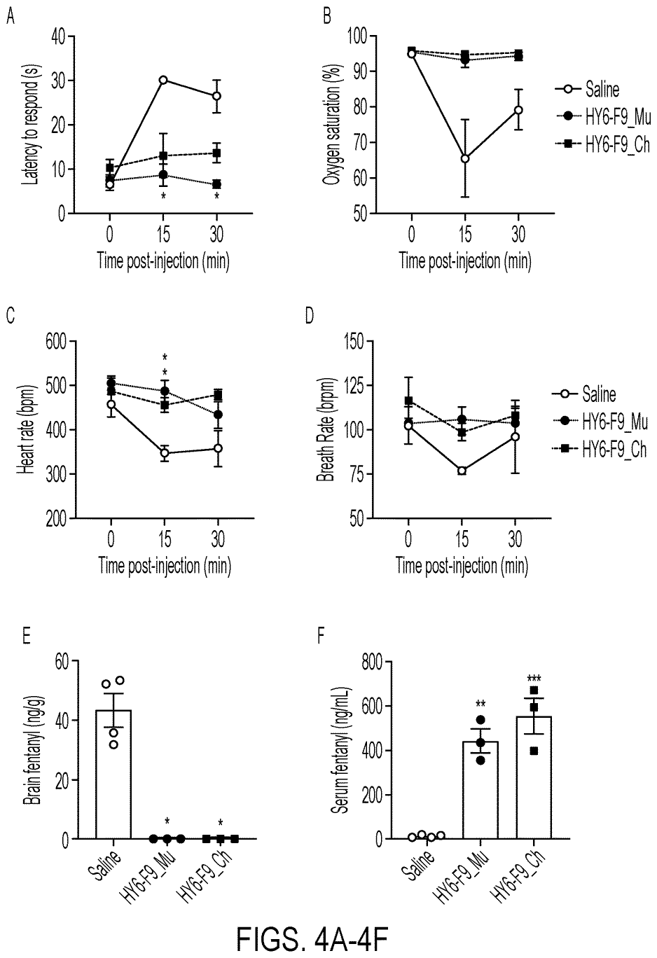

To evaluate whether in vivo efficacy was maintained in chAb, mice were passively immunized with 40 mg/kg of each murine mAb or chAb, and challenged with 0.1 mg/kg fentanyl (FIG. 3). Concentrations of fentanyl in brain and serum were measured 30 min after fentanyl administration. All mAbs significantly reduced brain fentanyl and increased fentanyl concentration in serum, though HY11-6B2_Ch was least effective at sequestering fentanyl in serum. The serum levels of chimeric mAb showed a slight reduction compared to murine mAb (FIG. 3C), which is likely due to loss of murine FcRn binding in mice rather than reduced distribution or stability of mAb, as binding to FcRn is critical for mAb half-life,^{30,31} and has been implicated in efficacy of α-opioid mAb.³² Because HY6-F9_Mu has previously shown efficacy in rats,^{16,19} the efficacy of HY6-F9_Ch was also compared to HY6-F9_Mu in rats (FIG. 4). Rats were passively immunized with 40 mg/kg of murine or chimeric mAb, and 24 hours later challenged with 0.1 mg/kg fentanyl. Both HY6-F9_Mu and HY6-F9_Ch prevented fentanyl-induced antinociception (FIG. 4A), respiratory depression and bradycardia (FIG. 4A-D), and reduced fentanyl distribution to brain (FIG. 4E-F). Considering these results, and the ability of HY11-7E1 to bind carfentanil, HY6-F9 and HY11-7E1 were selected for humanization and further development.

### Humanization of α-Fentanyl mAb.

To reduce potential immunogenicity in response to murine VH and VL regions present on the chAbs,^{27,33,34} complementarity-determining region (CDR) amino acids of HY6-F9_Ch and HY11-7E1_Ch were grafted onto the human germline VH and VL gene sequences with the highest homology to the murine germline sequences of each mAb. The CDR amino acids of human germline gene sequences were replaced with corresponding CDR amino acids from HY6-F9_Ch or HY11-7E1_Ch using two grafting schemes: CDR amino acid regions delineated by (i) IMGT definitions,³⁵ or (ii) combined IMGT, KABAT,³⁶ CHOTHIA,³⁷ AbM,³⁸ and paratome definitions.³⁹ Because IMGT definition encompasses fewer murine CDR amino acids relative to the combined definitions approach, humanized mAb grafted with the IMGT defined CDR amino acids resulted in the highest % homology for human sequence; therefore, this grafting scheme was denoted “high homology”. For the second approach, considering that the combined IMGT, KABAT, CHOTHIA, AbM, and paratome definitions encompassed a larger number of murine CDR amino acids relative to the IMGT only definition, this grafting scheme was denoted “low homology”. These two approaches were chosen to maximize the resulting homology to the human germline gene sequence while considering that the high homology approach may result in the replacement of peripheral CDR amino acid residues that contribute to fentanyl binding. To isolate any potential loss in binding to either the humanized VH or VL, humanization was conducted with a stepwise approach. First, high and low homology humanized VH were paired with chimeric VL to produce humanized intermediates (FIG. 1B). These humanized intermediates were evaluated for fentanyl and carfentanil binding by BLI. Then, the high and low homology VL were paired with the humanized VH that retained highest affinity for fentanyl hapten (Table 3) to produce “fully humanized” mAbs (FIG. 1). The fully humanized mAbs were evaluated for fentanyl and carfentanil binding by BLI (Table 3).

TABLE 3
Homology of mAbs to human germline and affinity to fentanyl and carfentanil.

| | | | | Homology to Human | | | | |
|---|---|---|---|---|---|---|---|---|
| mAb | mAb | VH | VL | Sequence | | | KD by BLI (nM) | |
| Series | Species | Description | Description | VH* | VL** | Overall*** | Fentanyl† | Carfentanil† |
| HY6-F9 | Murine | Murine | Murine | 73.7% | 80.0% | 67.8% | <0.1 | N/A |
| | Chimeric | Murine | Murine | 73.7% | 80.0% | 92.4% | <0.1 | N/A |
| | Intermediate | Low | Murine | 91.9% | 80.0% | 95.2% | <0.1 | N/A |
| | | Homology | | | | | | |
| | Intermediate | High | Murine | 95.0% | 80.0% | 95.7% | 1.31 | N/A |
| | | Homology | | | | | | |
| | Humanized | Low | Low | 91.9% | 89.0% | 96.7% | 0.17 | N/A |
| | | Homology | Homology | | | | | |
| | Humanized | Low | High | 91.9% | 91.0% | 97.0% | 9.24 | N/A |
| | | Homology | Homology | | | | | |
| HY11- | Murine | Murine | Murine | 68.0% | 66.3% | 65.5% | <0.1 | 1.18 |
| 7E1 | Chimeric | Murine | Murine | 68.0% | 66.3% | 87.4% | <0.1 | 0.51 |
| | Intermediate | Low | Murine | 85.6% | 66.3% | 91.0% | <0.1 | 2.556 |
| | | Homology | | | | | | |
| | Intermediate | High | Murine | 92.8% | 66.3% | 92.3% | <0.1 | 1.329 |
| | | Homology | | | | | | |
| | Humanized | High | Low | 92.8% | 86.3% | 95.7% | <0.1 | 4.461 |
| | | Homology | Homology | | | | | |
| | Humanized | High | High | 92.8% | 91.6% | 96.6% | 3.263 | NDB |
| | | Homology | Homology | | | | | |

*Amino acid homology to human IGHV V-Gene germline sequence chosen for CDR grafting¹.
**Amino acid homology to human IGKV V-Gene germline sequence chosen for CDR grafting¹.
***Amino acid homology to human IGHV and IGKV V-Gene germline sequence chosen for CDR grafting¹, with human IgG1 and IgK constant regions.
†Hapten.

The amino acid sequence % homology of the hybridoma-derived murine IgG1/IgK α-fentanyl mAb to a human IgG1/IgK mAb containing the corresponding germline gene VH/VL sequences used for humanization were 67.8% for HY6-F9_Mu and 65.5% for HY11-7E1_Mu (Table 3). For HY6-F9, the humanized mAb with low homology VH and VL displayed a higher affinity for fentanyl hapten by BLI compared to humanized mAb containing high homology VH and VL (Table 3). For HY11-7E1, both low and high homology humanized intermediate mAb showed similar binding to fentanyl and carfentanil haptens. However, when fully humanized, the humanized mAb containing the high homology VL showed a marked reduction in affinity for fentanyl hapten, and binding to carfentanil hapten was ablated (Table 3). Therefore, the mAbs chosen for further development were HY6-F9_Hu with the low homology VH and VL, and HY1 1-7E1_Hu with the high homology VH and low homology VL, resulting in 96.7% and 95.7% homology to human IgG1/IgK mAb containing the corresponding germline gene VH/VL, respectively.

### PTM Mitigation of Humanized α-Fentanyl mAb.

Amino acids residues in the combined KABAT+IMGT defined CDR regions of HY11-7E1_Hu and HY6-F9_Hu prone to post-translational modification (PTM) were identified and modified to reduce the risk of PTM-induced mAb heterogeneity and immunogenicity.⁴⁰ One potential PTM risk was identified in the CDRs of each mAb: An asparagine deamidation motif (N34/G35, IMGT numbering) in HY6-F9_Hu VL,^{41,42} and an aspartate isomerization motif (D62/G63, IMGT numbering) in HY11-7E1_Hu VH.⁴³ To mitigate PTM risks, N34Q, G35K, and G35R mutations were introduced into the VL of HY6-F9_Hu, and D62E and G63V mutations were introduced into the VH of HY11-7E1_Hu. Additionally, to assess whether deamidation of VL N34 in HY6-F9_Hu would affect binding to the fentanyl hapten, a N34D mutation was introduced to mimic deamidation. To determine whether the introduction of PTM mitigating or mimicking mutations at these residues would impact affinity for fentanyl, mAbs incorporating these mutations were produced and evaluated by BLI. The mitigated humanized HY6-F9 mAbs (HY6-F9_Hu (NQ), HY6-F9_Hu (GK), HY6-F9_Hu (GR)) and HY11-7E1 mAbs (HY11-7E1_Hu (DE), HY11-7E1_Hu (GV)) all maintained K_{D} values comparable to the unmitigated humanized counterpart (Table 5). The HY6-F9_Hu deamidation mimic (HY6-F9_Hu (ND)) resulted in a ˜10-fold decrease in K_{D} (Table 5), indicating that deamidation of this asparagine in HY6-F9_Hu may decrease efficacy against fentanyl in vivo. Post-translational modification (PTM) mitigation.

TABLE 5
Visualization of CDRs with PTM risks and KD result
from subsequent BLI analysis.

| | | K_{D} (nM) by Biolayer | |
|---|---|---|---|
| | | Interferometry | |
| | | Fentanyl | Carfentanil |
| mAb ID | LC CDR1 | Hapten | Hapten |
| HY6-F9_Hu | RSSKSLLHSNGITYLY | 0.17 | Not Tested |
| | (SEQ ID NO: 67) | | |
| HY6-F9_Hu (NQ) | RSSKSLLHSQGITYLY | <0.1 | Not Tested |
| | (SEQ ID NO: 70) | | |
| HY6-F9_Hu (GK) | RSSKSLLHSNKITYLY | <0.1* | Not Tested |
| | (SEQ ID NO: 71) | | |
| HY6-F9_Hu (GR) | RSSKSLLHSNRITYLY | <0.1* | Not Tested |
| | (SEQ ID NO: 72) | | |
| HY6-F9_Hu (ND) | RSSKSLLHSDGITYLY | 1.91 | Not Tested |
| | (SEQ ID NO: 73) | | |
| | | Fentanyl | Carfentanil |
| mAb ID | HC CDR2 | Hapten | Hapten |
| HY11-7E1_Hu | WIFPGDGSTKYNEKFKG | <0.1 | 2.36 |
| | (SEQ ID NO: 83) | | |
| HY11-7E1_Hu (DE) | WIFPGEGSTKYNEKFKG | <0.1 | 1.31 |
| | (SEQ ID NO: 85) | | |
| HY11-7E1_Hu (GV) | WIFPGDVSTKYNEKFKG | <0.1* | 5.15* |
| | (SEQ ID NO: 86) | | |

PTM Motif (underlined)
Mitigating AA
*K_{D} measured in BLI
titered cell culture
supernatant

Biophysical Characterization of humanized and PTM-mitigated α-Fentanyl mAb.

Biophysical characterization and developability assessment of candidate mAbs may predict potential developability liabilities prior to the initiation of costly stable cell line development (CLD) and upstream and downstream process development.^{40,44} Additionally, when considering the selection of a candidate for clinical development, biophysical characterization provides further criteria with which to rank the α-fentanyl mAb candidates against one another based on key characteristics for predicting manufacturing process development success.

Chimeric, humanized, and PTM mitigated humanized HY6-F9 and HY11-7E1 α-fentanyl mAbs were transiently expressed in CHO cells (ExpiCHO, ThermoFisher) to approximate the cell line to be used for future CLD and formulated in phosphate buffered saline (PBS), pH 7.4 at 1.0 mg/mL. Aggregation by size-exclusion high pressure liquid chromatography (SEC-HPLC),⁴⁵ hydrophobicity by hydrophobic interaction high pressure liquid chromatography (HIC-HPLC),^{46,47} and Fab domain melting temperature (Tₘ) by dynamic scanning fluorimetry (DSF)^{48,49} were assessed for all mAbs (Table 6). A commercially approved therapeutic mAb (Rituximab) was produced internally to provide a comparator for SEC-HPLC and HIC-HPLC analysis.

### Aggregation Analysis by SEC-HPLC.

Monoclonal antibodies prone to aggregation can lead to immunogenicity,⁵⁰ precipitation, and other deleterious properties for a potential therapeutic candidate.⁵¹ In a study of 152 human or humanized mAbs, analysis by SEC-HPLC showed that 72% of mAbs displayed >95% monomer, and 89% showed >90% monomer.⁴⁴ Another study of 21 FDA-approved therapeutic mAbs showed 20/21 with >97.5% monomer.⁵²

For the mAbs analyzed in this Example, all humanized versions of the HY6-F9 and HY11-7E1 mAbs displayed >99.5% monomer by SEC-HPLC (Table 4). HY6-F9_Ch and HY11-7E1_Ch displayed 98% and 98.5% monomer respectively, and the in-house Rituximab showed 99.2% monomer. When measured by ref.⁵², Rituximab showed 99.0% monomer by SEC-HPLC. Hydrophobicity Analysis byHIC-HPLC. Monoclonal antibodies with high surface hydrophobicity are prone to aggregation, non-specific binding and self-interaction, substandard concentratability, and high viscosity.⁴⁴ To assess hydrophobicity by HIC-HPLC, retention time of the antibody on the column under a high-to-low salt gradient was measured. Under conditions used in this experiment, mAbs with high hydrophobicity will display a retention time of >16.67 min, as mAbs that elute after that time point are subjected to a salt-free mobile phase in which there is no longer a salting-out effect.⁵³ In HIC-HPLC assays performed by another group, see ref. 54, on 32 mAbs representing FDA approved therapeutic mAbs and mAbs in early-to-late stage clinical trials, 27 mAbs eluted prior to the transition to a salt-free mobile phase. For the mAbs analyzed in this Example, all HY11-7E1 mAbs displayed similar retention times, between 13.0 and 13.1 min (Table 4). The retention times of the HY6-F9 mAbs were more varied, with HY6-F9_Hu mAb having the longest retention time (therefore highest hydrophobicity) at 16.17 min, and the VL N34Q PTM mitigated HY6-F9_Hu (HY6-F9_Hu (NQ)) eluting at 14.51 min. The HIC-HPLC results for the candidate α-fentanyl mAbs indicate that the HY6-F9 series of mAbs show higher retention times, and therefore slightly elevated levels of hydrophobicity, compared to the HY11-7E1 mAbs. Additionally, the HY11-7E1 series of mAbs all had lower hydrophobicity than the Rituximab control (Table 4).

TABLE 4
Biophysical characterization of chimeric and humanized mAb.

| | | | HIC-HPLC | |
|---|---|---|---|---|
| | SEC-HPLC | | Retention | DSF |
| | % mAb | % | Time | Fab Tₘ |
| mAb ID | Monomer | Aggregate | (min)* | (° C.) |
| HY6-F9_Ch | 98.0 | 2.0 | 14.88 | 77.0 |
| HY6-F9_Hu | >99.5 | <0.5 | 16.17 | 81.5 |
| HY6-F9_Hu (NQ) | >99.5 | <0.5 | 14.51 | 78.5 |
| HY6-F9_Hu (GR) | >99.5 | <0.5 | 15.73 | 78.7 |
| HY11-7E1_Ch | 98.5 | 1.5 | 13.01 | 72.5-76.5 |
| HY11-7E1_Hu | >99.5 | <0.5 | 13.11 | 78.0 |
| HY11-7E1_Hu (DE) | >99.5 | <0.5 | 13.06 | 80.5 |
| HY11-7E1_Hu (GV) | >99.5 | <0.5 | 13.07 | 77.6 |
| In-house | 99.2 | 0.8 | 13.97 | Not |
| Rituximab | | | | Tested |

*HIC-HPLC salt gradient ends at 16.67 min.

### Tₘ Analysis by DSF.

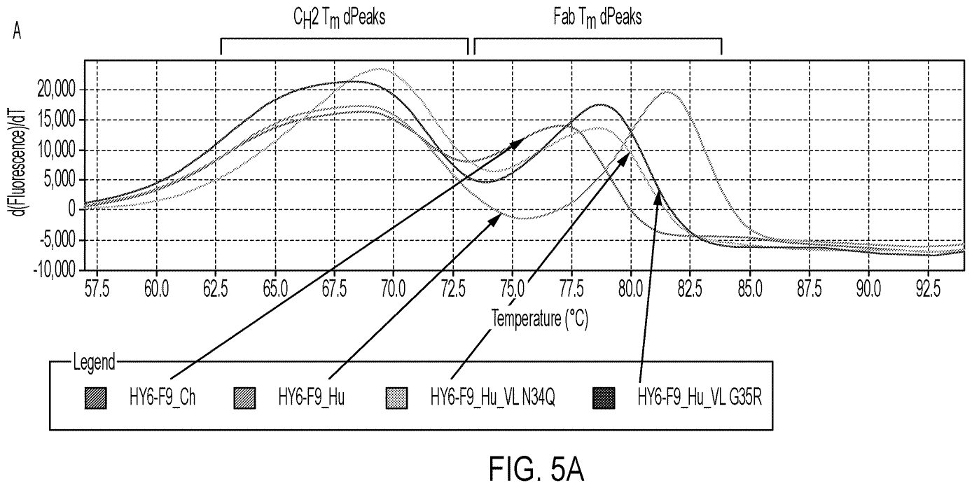

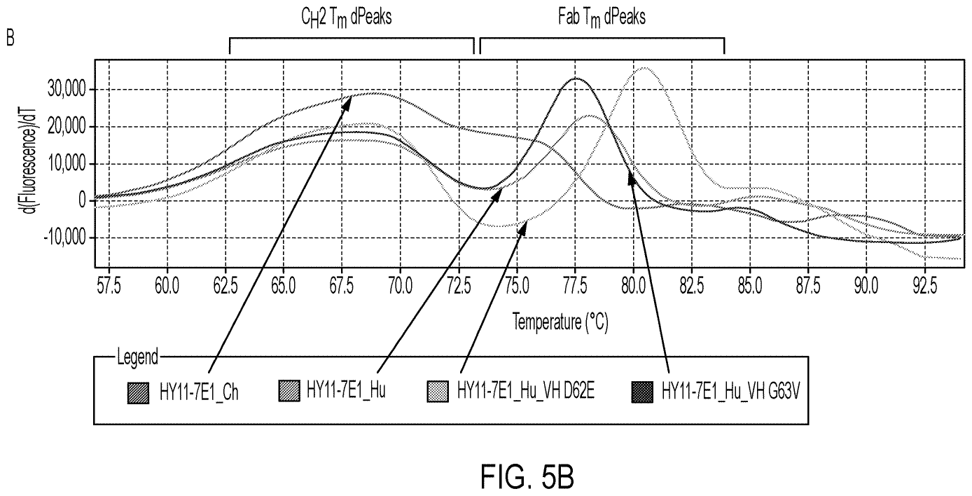

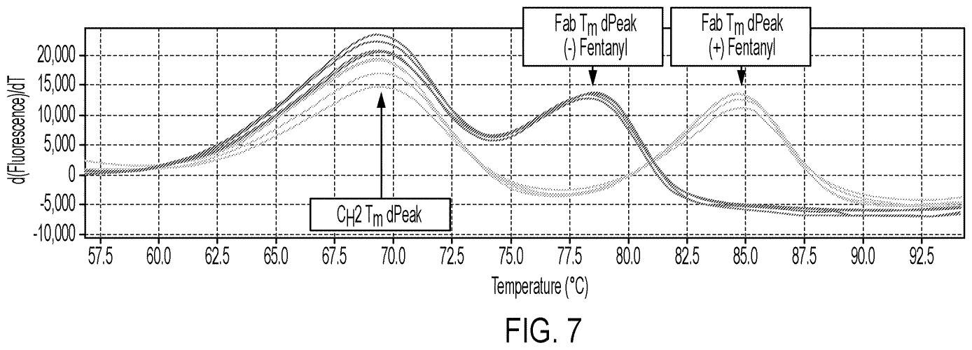

Monoclonal antibodies that contain Fab domains with a melting temperature (Tₘ) less than 65° C. may have conformational stability liabilities,^{44,55} and are susceptible to instability under stressed conditions.⁵⁶ These liabilities introduce complexities and greater expense in manufacturing, in addition to complications with respect to long-term storage of mAb drug product. To assess Fab domain Tₘ by DSF, the temperature of the derivative DSF peak corresponding to thermal unfolding of the Fab domain of each antibody was measured (FIG. 5). The Fab, CH2, and CH3 domains of IgG typically have distinct unfolding transitions,⁵⁸ and the Fab domain unfolding transition may overlap with the unfolding transition of the CH2 domain. As described by ref. 59, thermal stabilization of the Fab domain of an α-cocaine mAb occurred upon binding to its small molecule ligand, resulting in a Tₘ increase of the Fab domain in the presence of cocaine. Therefore, Tₘ was determined in the absence and presence of fentanyl (FIG. 7, Table 6) to differentiate Fab Tₘ from the Tₘ of the CH2 domain.

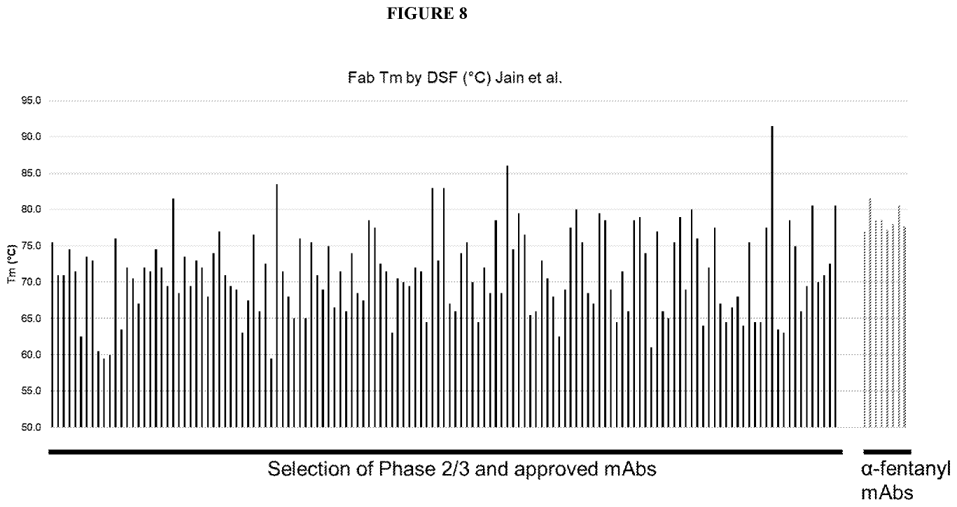

HY6-F9_Hu showed increased Fab thermostability compared to the HY6-F9_Ch, with a ΔTₘ of 4.5° C. (Table 6). The PTM mitigated HY6-F9 mAbs had lower thermostability than HY6-F9_Hu, but still resulted in increased Fab Tₘ compared to HY6-F9_Ch. The HY11-7E1 mAb with the highest Fab thermostability was HY11-7E1_Hu (DE). The Fab domain of HY11-7E1_Ch showed an unfolding transition that overlapped with the unfolding transition of the CH2 Tₘ; therefore, a discrete Tₘ value was not captured, and a range of 72.5-76.5° C. was estimated. As was the case for the HY6-F9 mAbs, the humanized HY11-7E1 mAbs all displayed improved thermostability compared to their murine chimeric counterpart. In Fab Tₘ determination assays on 137 FDA approved therapeutic mAbs and mAbs in 2^{nd} or 3^{rd} phase clinical trials, 117 mAbs tested displayed Fab Tₘ values >65° C. under DSF conditions similar to those used in this Example, with a mean of 71.3° C.;⁵⁷ all candidate α-fentanyl mAbs displayed Fab Tₘ values above the mean Fab Tₘ value (FIG. 8).

TABLE 6
Change in Fab Tₘ in presence of fentanyl

| | | Tₘ1* | Tₘ2* | ΔTₘ1 | ΔTₘ2 |
|---|---|---|---|---|---|
| mAb ID | Ligand | (° C.) | (° C.) | (° C.) | (° C.) |
| HY6-F9_Ch | (−) Fentanyl | 68.8 | 77.0 | −0.5 | 8.1 |
| | (+) Fentanyl | 68.3 | 85.1 | | |
| HY6-F9_Hu | (−) Fentanyl | 68.6 | 81.5 | −0.2 | 5.3 |
| | (+) Fentanyl | 68.5 | 86.7 | | |
| HY6-F9_Hu | (−) Fentanyl | 69.4 | 78.5 | 0.06.2 | |
| (NQ) | (+) Fentanyl | 69.4 | 84.7 | | |
| HY6-F9_Hu | (−) Fentanyl | 68.4 | 78.7 | 0.2 | 6.5 |
| (GR) | (+) Fentanyl | 68.6 | 85.2 | | |
| HY11-7E1_CH | (−) Fentanyl | 69.0 | 72.5-76.5 | −1.5 | 8.1-12.1 |
| | (+) Fentanyl | 67.5 | 84.6 | | |
| HY11-7E1_Hu | (−) Fentanyl | 68.1 | 78.0 | −0.4 | 5.4 |
| | (+) Fentanyl | 67.7 | 83.4 | | |
| HY11-7E1_Hu | (−) Fentanyl | 68.4 | 80.5 | 0.0 | 4.8 |
| (DE) | (+) Fentanyl | 68.4 | 85.3 | | |
| HY11-7E1_Hu | (−) Fentanyl | 68.1 | 77.6 | 0.2 | 5.0 |
| (GV) | (+) Fentanyl | 68.3 | 82.6 | | |

*Tₘ1 = C_{H}2 Tₘ and Tₘ2 = Fab Tₘ

### Efficacy of Humanized α-Fentanyl mAb.

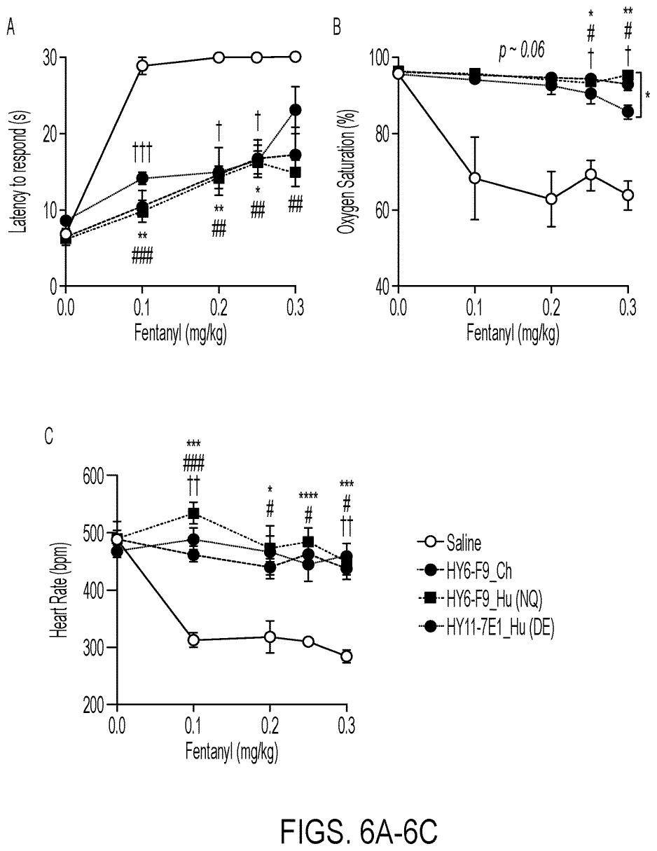

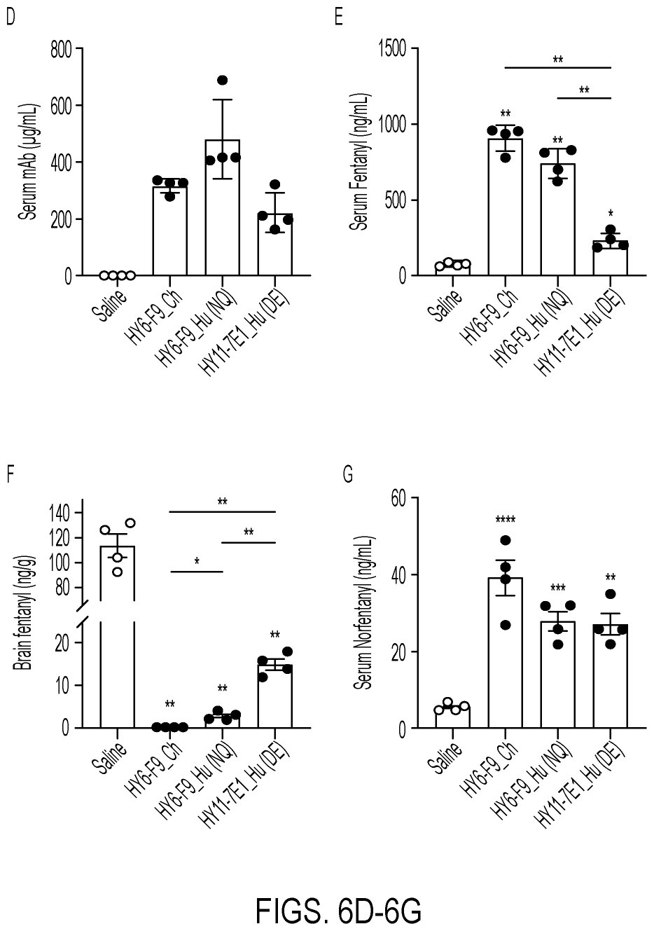

Two humanized α-fentanyl mAbs, HY6-F9_Hu (NQ) and HY11-7E1_Hu (DE), were selected for further evaluation of in vivo efficacy. HY6-F9_Hu (NQ) was chosen as the HY6-F9 candidate despite the higher Fab Tₘ of un-mitigated HY6-F9_Hu due to the finding that the asparagine deamidation mimicking HY6-F9_Hu (ND) mAb showed decreased binding to fentanyl, indicating potential loss of binding from deamidation of N34 (Table 5). HY6-F9_Hu (NQ) also displayed a decreased apparent hydrophobicity compared to HY6-F9_Hu (Table 4). HY11-7E1_Hu (DE) was chosen as the HY11-7E1 candidate due to its apparent higher affinity to fentanyl hapten compared to HY11-7E1_Hu (Table 1), its superior Fab Tₘ value relative to other HY11-7E1 mAbs (Table 6), and due to the presence of the aspartate isomerization mitigating VH D62E mutation. Rats were passively immunized with 40 mg/kg of HY6-F9_Ch (+control), HY6-F9_Hu (NQ), or HY11-7E1_Hu (DE). 24 hours later, rats were challenged with cumulative doses of fentanyl, 0.1-0.3 mg/kg, and monitored for fentanyl-induced antinociception, respiratory depression, and bradycardia (FIG. 6A-C). All mAbs significantly reduced fentanyl-induced antinociception up to 0.25 mg/kg fentanyl, and prevented reduction in oxygen saturation and heart rate compared to saline control. At the highest dose of 0.3 mg/kg fentanyl, rats treated with HY11-7E1_Hu (DE) showed a slight reduction in oxygen saturation to approximately 86%, and rats treated with HY6-F9_Hu (NQ) showed significantly higher protection at that dose (p=0.043). Finally, while all three mAbs sequestered fentanyl in serum and significantly reduced brain concentration of fentanyl (FIG. 6D-E), HY11-7E1_Hu (DE) was less effective than HY6-F9_Ch or HY6-F9_Hu (NQ) at preventing brain distribution.

### DISCUSSION

The present-day record high overdose death counts involving synthetic opioids are an attestation that current methods of prevention and therapeutic intervention against opioid-related overdose deaths are insufficient. Potentially exacerbating the overdose death rate is that naloxone, the current standard therapeutic intervention to reverse opioid toxicity in overdose scenarios, may be less effective at counteracting fentanyl compared to other opioids.^{60,61} An additional drawback to treatment of opioid toxicity with naloxone is the possibility of precipitated opioid withdrawal.^{10,12} As the mechanism of action of an α-opioid mAb is drug sequestration rather than receptor antagonism, precipitated withdrawal is not expected to occur with mAb treatment; though this has not yet been explored with opioid mAb, an anti-nicotine mAb was shown to not precipitate withdrawal in nicotine dependent rats.⁶² To address the increased prevalence of synthetic opioids such as fentanyl, innovative treatments that specifically counteract synthetic opioid toxicity are required. This Example sought to investigate the viability such a treatment through the discovery and development of a humanized mAb specific to fentanyl and its potent analogs.

Murine and chimeric α-fentanyl mAbs have been shown to be efficacious in rodent models;¹9-21 however, murine and chimeric mAbs may not be suitable for use in humans due to anti-drug antibody responses directed against murine antibody epitopes present in these mAbs.²⁶-28 Humanized α-fentanyl mAbs were developed in this Example to reduce potential immunogenicity while minimizing any subsequent decrease in affinity for fentanyl. In this Example, a stepwise humanization approach (FIG. 1) and CDR grafting onto human germline gene VH/VL backbones with either minimal or maximal human CDR replacement was employed. With this approach, humanized mAbs maintained <1.0 nM binding to fentanyl hapten, and <3.0 nM binding to carfentanil hapten (HY11-7E1 mAb series) by BLI. Additionally, any alteration or heterogeneity induced by PTMs in mAb CDRs, such as asparagine deamidation and aspartate isomerization, pose a risk of loss of antibody efficacy.^{63,64} Where such risks were present in mAb CDRs, PTM mitigating mutations were introduced. PTM mitigated humanized mAbs retained equivalent affinity to fentanyl and carfentanil by BLI (Table 1).

Successful therapeutic mAb development necessitates biophysical properties that make mAb well-suited for efficient manufacturing and long-term stability. The in vitro biophysical characterization assays performed on the mAbs described in this Example provide evidence that these mAbs are suitable for further clinical development as therapeutic candidates, as they displayed desired biophysical properties for aggregation and fragmentation, hydrophobicity, and Fab thermostability when analyzed by SEC-HPLC, HIC-HPLC, and DSF, respectively. These biophysical characteristics predict successful manufacturing process development and long-term storage stability. Combined, the results of these biophysical characterization assays indicate that the candidate α-fentanyl mAbs developed in this Example are aligned with currently approved mAb therapeutics, and that they lack liabilities with respect to aggregation and subsequent immunogenicity, non-specific/off-target binding, self-interaction, substandard concentrateability, high viscosity, poor conformational stability, and problematic long-term storage.

Finally, based on in vitro affinity and biophysical properties, two humanized mAbs containing PTM mitigating mutations were identified. When assessed for in vivo efficacy against fentanyl in rats, the mAbs protected against effects of fentanyl toxicity by preventing the distribution of fentanyl to the brain. Importantly, HY6-F9_Hu (NQ) was significantly more effective at sequestering fentanyl in serum compared to HY11-7E1_Hu (DE). Additionally, while HY6-F9_Hu (NQ) prevented respiratory effects of fentanyl (i.e. SaO₂>95%) up to 0.3 mg/kg, the protection afforded by HY11-7E1_Hu (DE) was overcome at the higher doses, with SaO₂ significantly lower in these rats than in rats treated with HY6-F9-derived mAb. Concentration of norfentanyl was increased in serum in all mAb-treated rats, suggesting that metabolism of fentanyl to norfentanyl is not ablated by mAb binding, though the overall effect of mAb on fentanyl metabolism and elimination remains to be fully explored.

There are potential limitations of mAb treatments against synthetic opioid toxicity. The US Drug Enforcement Agency considers a 2 mg dose of fentanyl to be lethal in humans (dea.gov/onepill). Due to the difference in molecular weight between mAb (˜150,000 g/mol) and fentanyl (336 g/mol), ˜450 mg of α-fentanyl mAb might be required to completely neutralize 2 mg of fentanyl. Furthermore, information regarding confirmed identification of fentanyl-induced toxicity and the fentanyl serum concentration of an overdosing patient may be unknown at the point of care. Considering these factors that favor high α-fentanyl mAb dosing, in addition to limitations with respect to contemporary therapeutic mAb formulations,⁷¹ counteracting fentanyl overdose with α-fentanyl mAb may require i.v. administration. Additional clinical applications of α-fentanyl mAb would be made available if it could be delivered via s.c. or i.m. routes of administration. Additionally, even as synthetic opioids like fentanyl are involved in a high proportion of opioid overdose deaths,³⁻⁷ instances of exposure to multiple opioids are frequent.^{76,77} In these circumstances, treatment with α-fentanyl mAb alone may not be effective; however, if naloxone and α-fentanyl mAb were administered concurrently, α-fentanyl mAb would limit fentanyl concentration in the brain while naloxone would block MOR receptor agonism of non-fentanyl opioids without competition from fentanyl. Furthermore, after naloxone is metabolized, high levels of α-fentanyl mAb should persist in serum, resulting in continued sequestration of fentanyl.

Additional mAb characterization and development activities are helpful to prepare an α-fentanyl mAb for the clinic. Regarding the HY6-F9_Hu (NQ) and HY11-7E1 (DE) α-fentanyl mAbs developed in this Example, in vitro and in vivo assays to measure immunogenicity,^{65,66} and forced oxidation, deamidation, and isomerization studies will be performed to assess their propensity to elicit an immunogenic response or produce deleterious PTMs in any remaining unmitigated CDR amino acid residues.⁶⁷⁻⁶⁹ Upon final selection, stable CLD will be initiated to support further mAb characterization and IND-enabling studies with the goal of establishing a manufacturing process and an in vivo efficacy and safety dataset that enables first-in-human clinical trials. To address the potential limitations of a mAb based therapeutic for counteracting fentanyl toxicity, studies will be initiated with next-generation α-fentanyl mAbs in mind. To enable s.c. or i.m. routes of administration, methods for obtaining high concentration mAb formulations, such as those described in refs.⁷²⁻⁷⁴, will be investigated. Furthermore, alternate mechanisms for decreasing α-fentanyl mAb doses may be attained by increasing the fentanyl binding capacity of mAb and/or reducing the molecular weight ratio of a recombinant α-fentanyl mAb to fentanyl. For example, a single-chain variable fragment specific to methamphetamine fused to Fc has been developed that displays efficacy against the psychostimulant effects of methamphetamine.⁷⁵ To address polydrug exposure, research into the delivery of cross-reactive and multi-specific α-opioid antibody formats, and/or multiple α-opioid mAbs that bind to structurally distinct opioids will be undertaken. Lastly, crystal structures of α-fentanyl mAb from this Example have been generated, (see Example 2) which can be used to support structure-guided design of humanized α-fentanyl mAbs to engineer cross-reactivity to fentanyl analogs, remove potentially immunogenic murine amino acids in CDRs, and introduce amino acid residues that improve protein stability and support high-concentration formulations.

Overall, the α-fentanyl mAbs HY6-F9_Hu (NQ) and HY11-7E1_Hu (DE) developed in this Example exhibit promising in vivo efficacy and demonstrate biophysical characteristics suitable for further clinical development. Although HY6-F9_Hu (NQ) showed superior fentanyl sequestration in rats (FIG. 6), HY11-7E1_Hu (DE) was also effective at preventing fentanyl toxicity in rats, and further optimization of mAb sequence and formulation may improve the sequestration properties of HY11-7E1_Hu (DE). These mAbs offer a promising new treatment for the prevention of overdose deaths involving synthetic opioids by providing an alternative to treatment with naloxone, or via dual administration of mAb and naloxone.¹⁶ The further clinical development of the α-fentanyl mAbs described herein provides a first-in-class opportunity to assess the clinical potential of a mAb-based therapeutic at preventing the loss of life caused by synthetic opioids.

### Materials and Methods

Animals. All experiments were approved by the University of Minnesota Animal Care and Use Committee prior to initiation and were conducted according to the Guide for the Care and Use of Laboratory Animals, 8th ed. Male and female Balb/c mice and male Sprague Dawley rats were 8-10 weeks on arrival, were housed in standard conditions with a 14/10 hr light/dark cycle, and provided with food and water ad libitum. Animals were acclimated to the housing environment for 1 week prior to initiation of experiments.

Hybridomas. HY6-F9_Mu was previously described in ref¹⁹. For HY11 mAbs, mice (n=2 male and 2 female) were immunized s.c. with 75 μg F1-CRM¹⁸ adsorbed on alum adjuvant (Alhydrogel-85, Invivogen, Catalog #vac-alu-250). Serum was collected via facial vein sampling on day 14 post-immunization. Due to work interruptions related to the COVID-19 pandemic, no additional boosts were performed, and splenocytes were collected and frozen in FBS+7% DMSO on day 18 after initial vaccination. Serum fentanyl-specific antibody level was determined by ELISA, and splenocytes from the mouse with the highest response were thawed, allowed to recover in ClonaCell HY Medium A (Stemcell Technologies Catalog #03801) for 30 min at 37° C. with 5% CO₂, and fused with Sp2/0 cells (ATCC CRL-1581) using ClonaCell HY kit according to manufacturer's protocol (Stemcell Technologies Catalog #03800). Two weeks after fusion, individual colonies were transferred to 96-well plates, cultured for 3 days in Medium E, and supernatant was screened for secretion of fentanyl-specific IgG by ELISA with fentanyl-BSA as described in ref. 19. Upon confirmation of secretion of fentanyl-specific IgG, sequencing of IgG antibody variable regions was performed as described in ref 78.

Generation of chimeric and humanized mAb expression vectors. Fentanyl-binding mAb VH and VL sequences were cloned into pcDNA3.4 mammalian expression vectors prepared by Genscript. A heavy chain (HC) pcDNA3.4 expression vector was modified to contain an ORF with a Kozak consensus sequence, murine IGHV signal peptide (MGWSCIILFLVATATGVHS (SEQ ID NO: 198)), and human IgG1 constant region (Accession #P01857). The light chain (LC) pcDNA3.4 expression vector was modified to contain an ORF with a Kozak consensus sequence, a murine IGKV signal peptide (METDTLLLWVLLLWVPGSTG (SEQ ID NO: 199)), and IgK constant region (Accession #P01834). Both pcDNA3.4 expression vectors were designed with cloning sites between the signal peptide and the constant region to facilitate an in-frame Gibson assembly cloning strategy with Gibson Assembly® Master Mix (New England Biolabs Catalog #E2611). Inserts for Gibson assembly of chAb expressing plasmid were prepared by variable region PCR amplification of the PCR product generated during the murine mAb VH/VL sequencing procedure. PCR primers were designed to introduce ˜30 base pairs of expression vector sequence homology onto the 5′ and 3′ ends of the amplified PCR product to facilitate Gibson assembly into the expression vector. Inserts for Gibson assembly of humanized antibodies were codon optimized and synthesized by Twist Bioscience. The synthesized gene fragments were designed to contain ˜30 base pairs of vector sequence homology on the 5′ and 3′ ends of the gene fragment to facilitate Gibson assembly into the expression vector.

Expression and purification of mAb. Hybridomas were cultured in 25-100 mL ClonaCell™-HY Medium E (Stemcell Technologies Catalog #03805) that had been depleted of bovine IgG via liquid chromatography on an AKTA pure (Cytiva) with a HiTrap Protein G HP column (Cytiva Product #29048581). Cell culture supernatant was harvested when cell viability fell below 20%, and IgG titer was determined by BLI on an Octet Red96e (Sartorious), as described below. mAb was purified via liquid chromatography on an AKTA pure with a HiTrap Protein G HP column (running buffer PBS, pH 7.4, elution buffer 0.1 M glycine, pH 2.5). Chimeric and humanized mAb was produced via transient expression in the Expi293 or ExpiCHO expression system (ThermoFisher Catalog #A14635 and A29133). Cells were cultured using manufacturer specified reagents and environmental conditions. Transfections were performed using a 1.5-2.5:1 ratio of LC vector:HC vector, with 1p g of total vector DNA/mL of culture volume. Cell culture supernatant was harvested 7-10 days following transfection. mAb was purified from filtered cell culture supernatant via liquid chromatography on an AKTA pure with a HiTrap MabSelect PrismA protein A column (Cytiva Product #17549851), running buffer PBS, pH 7.4, and elution buffer 0.2 M Na-Acetate, pH 3.5.

For both hybridoma and transiently produced mAb, eluted mAb was neutralized by dilution with ⅓^{rd} final volume 2.5 M Tris, pH 7.2, and buffer exchanged into PBS, pH 7.4. The purified mAb concentration was determined by absorbance at 280 nm on a Nanodrop.

Confirmatory analysis of purified mAb was performed by SDS-PAGE under reducing and non-reducing conditions.

In-house Rituximab was generated using the standard human IgG1 and IgK pcDNA3.4 expression vectors and the ExpiCHO expression and Protein A purification procedure described above. The VH and VL sequence of Rituximab was obtained from go.drugbank.com, Accession Number DB00073.

Determination of antibody affinity and selectivity by competitive ELISA. Relative affinity of mAb to various compounds was performed by competitive ELISA as described in ref¹⁹. Briefly, 96-well plates were coated with fentanyl hapten conjugated to BSA, 0.5 ng/well in carbonate buffer, and blocked with 1% gelatin. Free drug was serially diluted from 100 μM-300 μM and applied to the plates, and α-fentanyl mAb was diluted to 40-60 ng/mL in PBS pH 7.5 with 0.05% Tween-20 (PBS-T, Fisher Scientific Catalog #28348) and incubated with plates in the presence of competitor for 2 hr. Plates were washed, and mAb bound to plate coating was detected with HRP-conjugated goat anti-mouse secondary antibody (Jackson ImmunoResearch, Catalog #115-035-003) and visualized with OPD substrate (Sigma, Catalog #P9187). Estimated affinity was quantitated as IC₅₀, or concentration of competitor that reduced antibody binding to plates by 50%, and % relative affinity was expressed as (fentanyl IC₅₀)/(competitor IC₅₀)*100. Determination of antibody titer, affinity, and selectivity by BLI. Antibody titer determination of hybridoma supernatant was performed using an Octet Red96e. Protein G biosensors (Sartorius Part #18-5082) were pre-incubated in conditioned medium from Sp2/0 cells grown in ClonaCell™-HY Medium A depleted of bovine IgG for 60 sec. Next, biosensors were incubated in murine IgG containing hybridoma supernatant for 60 sec and the association between the Protein G biosensor and antibody in supernatant was measured. The standard curve used for titer determination was generated with known quantities of murine IgG1 in conditioned medium from Sp2/0 cells grown in ClonaCell™-HY Medium A depleted of bovine IgG. All calculations were performed with Octet analysis software (Sartorius).

Affinity and antigen selectivity measurements by BLI were performed with an Octet Red96e. Streptavidin-coated biosensors (Sartorius) were pre-incubated in PBS-T and loaded with 0.2 μg/mL of biotinylated fentanyl, acetylfentanyl, or carfentanil hapten for 60 sec. After a 60 sec baseline measurement in PBS-T, association of 1-100 nM mAb with hapten-biotin was measured for 3-5 min, followed by dissociation measurement in PBS-T for 5-10 min. The Octet analysis software (Sartorius) performed all measurements of on-rate (kₒₙ), off-rate (k_{off}), and K_{D} (k_{off}/kₒₙ) using the embedded 1:1 binding model.

Identification of human VH and VL germline gene for humanization by CDR grafting. To identify human VH and VL germline genes for humanization by CDR grafting, the murine VH and VL sequences from α-fentanyl mAbs were aligned with the human immunoglobulin germline gene sequence database available at www.IMGT.org using the DomainGapAlign webtool.^{79,80} for both the VH and VL chain, three human germline gene sequences with the highest homology and/or Smith-Waterman score were chosen for in silico characterization and developability assessment with TAP: Therapeutic Antibody Profiler to determine if any chosen germline gene sequences harbored developability risks.⁸¹ Based on homology/Smith-Waterman score from DomainGapAlign and in silico characterization results from TAP, a final human VH or VL germline gene sequence was chosen for humanization by CDR grafting.

Post-translational modification mitigation. Amino acid residues within the antibody CDRs susceptible to deamidation, glycosylation, and isomerization were identified with the abYsis webtool.⁸² Methionine and tryptophan oxidation were not considered for post-translational modification mitigation.

In VL CDR1 of HY6-F9_Hu, an asparagine residue at position 34 (IMGT numbering) is followed by a glycine residue, forming the highly susceptible asparagine deamidation motif, “NG” (Table 5). Three preemptive PTM mitigation mutations were introduced into VL CDR1 of the HY6-F9_Hu: N34Q, G35K, and G35R. Additionally, to determine if deamidation of N34 would affect binding to the fentanyl hapten, a N34D mutation was introduced to mimic deamidation of N34 42 In the VH CDR2 of HY11-7E1_Hu, an aspartic acid residue at position 62 (IMGT numbering) is followed by a glycine residue, forming the highly susceptible aspartate isomerization motif, “DG” (Table 5). Two preemptive PTM mitigation mutations were introduced into VH CDR2 of the HY11-7E1_Hu: D62E, and G63V. The G63V mutation was chosen based on the “N+1” strategy for aspartate isomerization mitigation described by ref⁸³ Point mutations to introduce an appropriate PTM mitigating amino acid residue into the mAb expression vector(s) were introduced via site-directed mutagenesis with a QuikChange Multi Site-Directed Mutagenesis Kit (Agilent Catalog #200514) and mutations were confirmed by Sanger sequencing.

Biophysical characterization mAb aggregation and fragmentation analysis by SEC-HPLC. The relative amount of aggregate, monomer, and fragment in a purified mAb sample was determined by SEC-HPLC on an Agilent 1200 series HPLC with a quaternary pump and multi-wave detector. Purified mAb (20 μg) was run on a Shodex KW-803 column preceded by an Shodex KW-G guard column. SEC-HPLC method details: mobile phase=PBS, pH 7.0, flow rate =0.75 mL/min, sample injection=20 μg, run time=30 min, operating temperature=24° C., detection=absorbance at 280 nm. Integration of the chromatogram was performed with ChemStation software (Agilent), using system standard settings for “new exponential”. Peaks eluting off the column before the main peak were assessed as aggregate. Peaks eluting off the column after the main peak were assessed as fragment. All SEC-HPLC runs were performed by the Biotechnology Resource Center of the BioTechnology Institute at the University of Minnesota. Biophysical characterization mAb hydrophobicity analysis by HIC-HPLC. The relative hydrophobicity of a purified mAb sample was determined by HIC-HPLC on an Agilent 1200 series HPLC with a quaternary pump and multi-wave detector. Purified mAb was run on a MAbPac HIC-10, 4.6×100 column (ThermoFisher Catalog #088480) preceded by a MAbPac HIC-10, 4.6×10 guard column (ThermoFisher Catalog #088482). HIC-HPLC method details: mobile phase A=100 mM sodium phosphate, 1.5 M ammonium sulfate, pH 7.0, mobile phase B =100 mM sodium phosphate, pH 7.0, flow rate=0.75 mL/min, operating temperature=25° C., detection=absorbance at 280 nm. The HPLC method proceeded as follows: (1) Equilibration; 100% mobile phase A, ˜3.0 to 0.0 min. (2) Sample injection; 25 μg of purified mAb diluted 1:1 with mobile phase A. (3) Gradient phase; 100% mobile phase A to 100% mobile phase B, 0 to 15.0 min. (4) Column flush; 100% mobile phase B, 15.0 to 18.0 min. (5) Shutdown, 100% mobile phase A, 18.0 to 19.0 min.. Integration of the chromatogram was performed with ChemStation, using system standard settings for “new exponential”. The manufacturer specified void volume of the MAbPac HIC-10, 4.6×100 column is ˜1.25 mL, or ˜1.67 minutes when operating at a flow rate of 0.75 mL/min. All HIC-HPLC runs were performed by the Biotechnology Resource Center of the BioTechnology Institute at the University of Minnesota.

Biophysical characterization Fab Tₘ determination by DSF. The melting temperature (Tₘ) of the Fab fragment of the purified mAb was determined by dynamic scanning fluorimetry (DSF) with a StepOnePlus™ Real-Time PCR System (Applied Biosystems Catalog #4376600). For the measurement, mAb at 1 mg/mL in PBS, pH 7.4 was combined with the assay reagents from the Protein Thermal Shift™ Dye Kit (Applied Biosystems Catalog #4461146) to a final volume of 20 μL and subjected to a continuous 0.3% temperature ramp from 25 to 95° C. Fluorescence measurements were recorded during the temperature ramp. Fab Tₘ determination was performed with Protein Thermal Shift™ Software v1.4 (Applied Biosystems Catalog #4466038). Four replicates of each mAb sample were run in this assay.

Efficacy of mAb against fentanyl in vivo. Animals were acclimated to the testing environment for 1 hr prior to drug challenge. For determination of mAb efficacy against fentanyl, mice were passively immunized s.c. with 40 mg/kg mAb. 24 hours post-immunization, mice were challenged with 0.1-0.25 mg/kg fentanyl s.c. as indicated in figure legends. Rats were passively immunized with 40 mg/kg mAb i.p. and challenged with 0.1-0.3 mg/kg fentanyl s.c. At 15-minute intervals post-challenge, animals were evaluated for drug-induced antinociception by hot plate set to 54° C. (Columbus Instruments) and for drug-induced respiratory depression and bradycardia with MouseOx Plus pulse oximeter (Starr Life Science). Following the final behavioral measurement, animals were euthanized by CO₂ inhalation, and brain and serum were collected for analysis of fentanyl and norfentanyl concentration by LCMS as described.^{18,19} Differences between groups in bradycardia, respiratory depression and antinociception were evaluated by 2-way ANOVA followed by Dunnett's or Tukey's multiple comparisons test. Analyses were conducted in Prism v9.2 (GraphPad).

Identification of Fab Tₘ by DSF analysis in the presence of fentanyl. DSF analysis was performed on anti-fentanyl mAbs with and without free fentanyl in the reaction mixture to identify the derivative DSF peak corresponding to the thermal unfolding of the Fab domain of the antibody. Test samples contained a ˜2.5-fold molar excess of fentanyl to purified mAb, or a ˜1.25-fold molar excess of fentanyl to available fentanyl binding sites. All mAbs displayed a shift in the Tₘ2 corresponding to the expected a Fab Tₘ increase (5-8° C.) in the presence of fentanyl, while the Tₘ1 (or CH2 Tₘ) was minimally affected by presence of fentanyl in the reaction mixture (FIG. 7, Table 6).

### Documents Cited in Example 1

1. Ahmad F B, Rossen L M, Sutton P. Provisional Drug Overdose Death Counts. 2021.
2. Centers for Disease Control and Prevention. Overdose Death Rates Involving Opioids, by Type, United States, 1999-2019 [Internet]. 2021 [cited 2021 Nov 12]; Available from: https://www.cdc.gov/drugoverdose/data/OD-deaths-2019.html
3. Matt Gladden R, Goldberger B A, Mattson C L, Kariisa M. Morbidity and Mortality Weekly Report Notes from the Field Opioid-Involved Overdose Deaths with Fentanyl or Fentanyl Analogs Detected-28 States and the District of Columbia. 2018; 69.
4. Shover C L, Falasinnu T O, Dwyer C L, Santos N B, Cunningham N J, Freedman R B, Vest NA, Humphreys K. Steep increases in fentanyl-related mortality west of the Mississippi River: Recent evidence from county and state surveillance. Drug and Alcohol Dependence 2020; 216.
5. Krotulski A J, Chapman B P, Marks S J, Ontiveros S T, Devin-Holcombe K, Fogarty M F, Trieu H, Logan B K, Merchant R C, Babu K M. Sentanyl: a comparison of blood fentanyl concentrations and naloxone dosing after non-fatal overdose. Clinical Toxicology 2021;
6. Armenian P, Vo K T, Barr-Walker J, Lynch K L. Fentanyl, fentanyl analogs and novel synthetic opioids: A comprehensive review. Neuropharmacology2018; 134:121-32.
7. Baumgartner J C, Radley D C. The Drug Overdose Toll in 2020 and Near-Term Actions for Addressing It. The Commonwealth Fund2021;
8. Martin W R. Drugs Five Years Later: Naloxone. Annals of Internal Medicine 1976; 85:765.
9. Berkowitz B A. The Relationship of Pharmacokinetics to Pharmacological Activity. Clinical Pharmacokinetics 1976; 1:219-30.
10. Rzasa Lynn R, Galinkin J L. Naloxone dosage for opioid reversal: current evidence and clinical implications. Therapeutic Advances in Drug Safety2018; 9:63-88.
11. Ahonen J, Olkkola K T, Hynynen M, Seppa Ela E T, Evalko H I, Remmerie B, Salmenpera M. Comparison of alfentanil, fentanyl and sufentanil for total intravenous anaesthesia with propofol in patients undergoing coronary artery bypass surgery2.
12. Clarke S F J, Dargan P I, Jones A L. Naloxone in opioid poisoning: walking the tightrope. Emergency Medicine Journal 2005; 22:612-6.
13. Moss R B, Carlo D J. Higher doses of naloxone are needed in the synthetic opioid era. Substance Abuse Treatment, Prevention, and Policy 2019; 14:6.
14. Morell A, Terry W D, Waldmann T A. Metabolic properties of IgG subclasses in man. The Journal of clinical investigation 1970; 49:673-80.
15. Mould D R, Sweeney K R. The pharmacokinetics and pharmacodynamics of monoclonal antibodies-mechanistic modeling applied to drug development. Current Opinion in Drug Discovery & Development 2007; 10:84-96.
16. Baehr C A, Wu M M, Pandit S G, Arias-Umana J, AuCoin D, Pravetoni M. Pharmacological profiling of anti-fentanyl monoclonal antibodies in combination with naloxone in pre- and post-exposure models of fentanyl toxicity. Journal of Pharmacology and Experimental Therapeutics 2022; JPET-AR-2021-001048.
17. Raleigh M D, King S J, Baruffaldi F, Saykao A, Hamid F A, Winston S, LeSage M G, Pentel P R, Pravetoni M. Pharmacological mechanisms underlying the efficacy of antibodies generated by a vaccine to treat oxycodone use disorder. Neuropharmacology 2021; 195:108653.
18. Robinson C, Gradinati V, Hamid F, Baehr C, Crouse B, Averick S, Kovaliov M, Harris D, Runyon S, Baruffaldi F, et al. Therapeutic and Prophylactic Vaccines to Counteract Fentanyl Use Disorders and Toxicity. Journal of Medicinal Chemistry 2020; 63:14647-67.
19. Baehr C, Kelcher A H, Khaimraj A, Reed D E, Pandit S G, AuCoin D, Averick S, Pravetoni M. Monoclonal antibodies counteract opioid-induced behavioral and toxic effects in mice and rats. Journal of Pharmacology and Experimental Therapeutics 2020; 375:469-77.
20. Ban B, Barrientos R C, Oertel T, Komla E, Whalen C, Sopko M, You Y, Banerjee P, Sulima A, Jacobson A E, et al. Novel chimeric monoclonal antibodies that block fentanyl effects and alter fentanyl biodistribution in mice. mAbs 2021; 13.
21. Smith L C, Bremer P T, Hwang C S, Zhou B, Ellis B, Hixon MS, Janda K_{D}. Monoclonal Antibodies for Combating Synthetic Opioid Intoxication. Journal of the American Chemical Society 2019; 141:10489-503.
22. Wetzel H N, Webster R P, Saeed F O, Kirley T L, Ball W J, Norman A B. Characterization of a recombinant humanized anti-cocaine monoclonal antibody produced from multiple clones for the selection of a master cell bank candidate. Biochemical and Biophysical Research Communications 2017; 487:690-4.
23. Gentry W B, Laurenzana E M, Williams D K, West J R, Berg R J, Terlea T, Owens S M. Safety and efficiency of an anti-(+)-methamphetamine monoclonal antibody in the protection against cardiovascular and central nervous system effects of (+)-methamphetamine in rats. International immunopharmacology 2006; 6:968-77.
24. Stevens M W, Tawney R L, West C M, Kight A D, Henry R L, Owens S M, Gentry W B. Preclinical characterization of an anti-methamphetamine monoclonal antibody for human use. 50 mAbs 6:547-55.
25. Stevens M W, Henry R L, Owens S M, Schutz R, Gentry W B. First human study of a chimeric anti-methamphetamine monoclonal antibody in healthy volunteers. mAbs 2014; 6:1649-56.
26. Kuus-Reichel K, Grauer L S, Karavodin L M, Knott C, Krusemeier, And M, Kay2 NE. MINIREVIEW Will Immunogenicity Limit the Use, Efficacy, and Future Development of Therapeutic Monoclonal Antibodies?[Internet]. 1994. Available from: http://cvi.asm.org/
27. Hwang W Y K, Foote J. Immunogenicity of engineered antibodies. Methods 2005; 36:3-10.
28. Harding F A, Stickler M M, Razo J, DuBridge R. The immunogenicity of humanized and fully human antibodies. mAbs 2010; 2:256-65.
29. Therapeutic monoclonal antibodies approved or in review in the EU or US. [Internet]. The Antibody Society2021 [cited 2021 Nov 18]; Available from: www.antibodysociety.org
30. Lencer W I, Blumberg R S. A passionate kiss, then run: exocytosis and recycling of IgG by FcRn. Trends in Cell Biology 2005; 15:5-9.
31. Petkova S B, Akilesh S, Sproule T J, Christianson G J, Al Khabbaz H, Brown A C, Presta L G, Meng Y G, Roopenian D C. Enhanced half-life of genetically engineered human IgG1 antibodies in a humanized FcRn mouse model: potential application in humorally mediated autoimmune disease. International Immunology 2006; 18:1759-69.
32. Huseby Kelcher A M, Baehr C A, Hamid F A, Hart G T, Pravetoni M. Contribution of Antibody-Mediated Effector Functions to the Mechanism of Efficacy of Vaccines for Opioid Use Disorders. Journal of immunology (Baltimore, Md: 1950) [Internet]2021 [cited 2022-Mar 6]; 207:860-7. Available from: https://pubmed.ncbi.nlm.nih.gov/34281999/
33. Safdari Y, Farajnia S, Asgharzadeh M, Khalili M. Antibody humanization methods—A review and update. Biotechnology and Genetic Engineering Reviews2013; 29:175-86.
34. Garcés S, Demengeot J. The Immunogenicity of Biologic Therapies. 2018. page 37-48.
35. Lefranc M P. IMGT Unique Numbering. In: The Immunologist-New York, NY: Springer New York; 1999. page 132-6.
36. Kabat E A, Te Wu T, Perry HM, Foeller C, Gottesman K S. Sequences of proteins of immunological interest. 5th ed. DIANE publishing; 1992.
37. Al-Lazikani B, Lesk A M, Chothia C. Standard conformations for the canonical structures of immunoglobulins. Journal of molecular biology 1997; 273.
38. Martin A C, Cheetham J C, Rees A R. Modeling antibody hypervariable loops: a combined algorithm. Proceedings of the National Academy of Sciences of the United States of America 1989; 86.
39. Kunik V, Ashkenazi S, Ofran Y. Paratome: An online tool for systematic identification of antigen-binding regions in antibodies based on sequence or structure. Nucleic Acids Research 2012; 40.
40. Xu Y, Wang D, Mason B, Rossomando T, Li N, Liu D, Cheung J K, Xu W, Raghava S, Katiyar A, et al. Structure, heterogeneity and developability assessment of therapeutic antibodies. mAbs2019; 11:239-64.
41. Robinson N E, Robinson A B. Deamidation of human proteins. Proceedings of the National Academy of Sciences 2001; 98.
42. Qiu H, Wei R, Jaworski J, Boudanova E, Hughes H, VanPatten S, Lund A, Day J, Zhou Y, McSherry T, et al. Engineering an anti-CD52 antibody for enhanced deamidation stability. mAbs 2019; 11.
43. Cacia J, Keck R, Presta L G, Frenz J. Isomerization of an Aspartic Acid Residue in the Complementarity-Determining Regions of a Recombinant Antibody to Human IgE: Identification and Effect on Binding Affinity. 1996.
44. Bailly M, Mieczkowski C, Juan V, Metwally E, Tomazela D, Baker J, Uchida M, 50 Kofman E, Raoufi F, Motlagh S, et al. Predicting Antibody Developability Profiles Through Early Stage Discovery Screening. mAbs 2020; 12.
45. Fekete S, Beck A, Veuthey J L, Guillarme D. Theory and practice of size exclusion chromatography for the analysis of protein aggregates. Journal of Pharmaceutical and Biomedical Analysis2014; 101:161-73.
46. Haverick M, Mengisen S, Shameem M, Ambrogelly A. Separation of mAbs molecular variants by analytical hydrophobic interaction chromatography HPLC: Overview and applications. mAbs2014; 6:852-8.
47. Geng X, Wang L. Liquid chromatography of recombinant proteins and protein drugs. Journal of Chromatography B: Analytical Technologies in the Biomedical and Life Sciences2008; 866:133-53.
48. Pantoliano M W, Petrella E C, Kwasnoski J D, Lobanov V S, Myslik J, Graf E, Carver T, Asel E, Springer B A, Lane P, et al. High-Density Miniaturized Thermal Shift Assays as a General Strategy for Drug Discovery. Journal of Biomolecular Screening 2001; 6.
49. Lavinder J J, Hari S B, Sullivan B J, Magliery T J. High-throughput thermal scanning: A general, rapid dye-binding thermal shift screen for protein engineering. Journal of the American Chemical Society 2009; 131:3794-5.
50. Rosenberg A S. Effects of protein aggregates: An immunologic perspective. The AAPS Journal 2006; 8.
51. Ma H, O'Figain C, O'Kennedy R. Antibody stability: A key to performance—Analysis, influences and improvement. Biochimie2020; 177:213-25.
52. Goyon A, D′Atri V, Colas 0, Fekete S, Beck A, Guillarme D. Characterization of 30 therapeutic antibodies and related products by size exclusion chromatography: Feasibility assessment for future mass spectrometry hyphenation. Journal of Chromatography B: Analytical Technologies in the Biomedical and Life Sciences 2017; 1065-1066:35-43.
53. Arakawa T, Timasheff S N. Preferential interactions of proteins with salts in concentrated solutions. Biochemistry 1982; 21.
54. Estep P, Caffry I, Yu Y, Sun T, Cao Y, Lynaugh H, Jain T, Vasquez M, Tessier P M, Xu Y. An alternative assay to hydrophobic interaction chromatography for high-throughput characterization of monoclonal antibodies. mAbs 2015; 7:553-61.
55. Wang W, Singh S, Zeng D L, King K, Nema S. Antibody structure, instability, and formulation. Journal of Pharmaceutical Sciences2007; 96:1-26.
56. Thiagarajan G, Semple A, James J K, Cheung J K, Shameem M. A comparison of biophysical characterization techniques in predicting monoclonal antibody stability. mAbs 2016; 8:1088-97.
57. Jain T, Sun T, Durand S, Hall A, Houston N R, Nett J H, Sharkey B, Bobrowicz B, Caffry I, Yu Y, et al. Biophysical properties of the clinical-stage antibody landscape. Proceedings of the National Academy of Sciences of the United States of America 2017; 114:944-9.
58. Garber E, Demarest S J. A broad range of Fab stabilities within a host of therapeutic IgGs. Biochemical and Biophysical Research Communications 2007; 355:751-7.
59. Kirley T L, Norman A B, Wetzel H N. A novel differential scanning fluorimetry analysis of a humanized anti-cocaine mAb and its ligand binding characteristics. Journal of Immunological Methods 2020; 476.
60. Hill R, Santhakumar R, Dewey W, Kelly E, Henderson G. Fentanyl depression of respiration: Comparison with heroin and morphine. British Journal of Pharmacology 2020; 177:254-65.
61. Pergolizzi J V., Dahan A, Ann LeQuang J, Raffa R B. Overdoses due to fentanyl and its analogues (F/FAs) push naloxone to the limit. Journal of Clinical Pharmacy and Therapeutics 2021; 46:1501-4.
62. Roiko S A, Harris A C, LeSage M G, Keyler D E, Pentel P R. Passive immunization with a nicotine-specific monoclonal antibody decreases brain nicotine levels but does not precipitate 50 withdrawal in nicotine-dependent rats. Pharmacology Biochemistry and Behavior 2009; 93:105-11.
63. Gervais D. Protein deamidation in biopharmaceutical manufacture: understanding, control and impact. Journal of Chemical Technology & Biotechnology 2016; 91:569-75.
64. Zhou K, Cao X, Bautista J, Chen Z, Hershey N, Ludwig R, Tao L, Zeng M, Das TK. Structure-Function Assessment and High-Throughput Quantification of Site-Specific Aspartate Isomerization in Monoclonal Antibody Using a Novel Analytical Tool Kit. Journal of Pharmaceutical Sciences 2020; 109:422-8.
65. Groell F, Jordan 0, Borchard G. In vitro models for immunogenicity prediction of therapeutic proteins. European Journal of Pharmaceutics and Biopharmaceutics 2018; 130:128-42.
66. van Meer P J K, Kooijman M, Brinks V, Gispen-de Wied C C, Silva-Lima B, Moors E H M, Schellekens H. Immunogenicity of mAbs in non-human primates during nonclinical safety assessment. mAbs 2013; 5:810-6.
67. Yang R, Jain T, Lynaugh H, Nobrega R P, Lu X, Boland T, Burnina I, Sun T, Caffry I, Brown M, et al. Rapid assessment of oxidation via middle-down LCMS correlates with methionine side-chain solvent-accessible surface area for 121 clinical stage monoclonal antibodies. mAbs 2017; 9:646-53.
68. Lu X, Nobrega R P, Lynaugh H, Jain T, Barlow K, Boland T, Sivasubramanian A, Visquez M, Xu Y. Deamidation and isomerization liability analysis of 131 clinical-stage antibodies. mAbs 2019; 11:45-57.
69. Nowak C, K. Cheung J, M. Dellatore S, Katiyar A, Bhat R, Sun J, Ponniah G, Neill A, Mason B, Beck A, et al. Forced degradation of recombinant monoclonal antibodies: A practical guide. mAbs 2017; 9:1217-30.
70. Goldberg D S, Bishop S M, Shah A U, Sathish H A. Formulation Development of Therapeutic Monoclonal Antibodies Using High-Throughput Fluorescence and Static Light Scattering Techniques: Role of Conformational and Colloidal Stability. Journal of Pharmaceutical Sciences 2011; 100:1306-15.
71. Wang S S, Yan Y, Ho K. US FDA-approved therapeutic antibodies with high-concentration formulation: summaries and perspectives. Antibody therapeutics [Internet]2021 [cited 2022 Mar 20]; 4:262-73. Available from: https://pubmed.ncbi.nlm.nih.gov/34909579/
72. Dear B J, Hung J J, Laber J R, Wilks L R, Sharma A, Truskett™, Johnston K P. Enhancing Stability and Reducing Viscosity of a Monoclonal Antibody With Cosolutes by Weakening Protein-Protein Interactions. Journal of Pharmaceutical Sciences 2019; 108:2517-26.
73. Shahfar H, Du Q, Parupudi A, Shan L, Esfandiary R, Roberts C J. Electrostatically Driven Protein-Protein Interactions: Quantitative Prediction of Second Osmotic Virial Coefficients to Aid Antibody Design. The Journal of Physical Chemistry Letters 2022; 13:1366-72.
74. Calero-Rubio C, Saluja A, Sahin E, Roberts C J. Predicting High-Concentration Interactions of Monoclonal Antibody Solutions: Comparison of Theoretical Approaches for Strongly Attractive Versus Repulsive Conditions. The Journal of Physical Chemistry B 2019; 123:5709-20.
75. Hay C E, Ewing L E, Hambuchen M D, Zintner S M, Small J C, Bolden C T, Fantegrossi WE, Margaritis P, Michael Owens S, Peterson EC. The Development and Characterization of an scFv-Fc Fusion-Based Gene Therapy to Reduce the Psychostimulant effect of Methamphetamine Abuse. The Journal of pharmacology and experimental therapeutics [Internet]2020 [cited 2022 Mar 17]; 374:16-23. Available from: https://pubmed.ncbi.nlm.nih.gov/32245884/
76. Hedegaard H, Bastian B A, Trinidad J P, Spencer M, Warner M. Drugs Most Frequently Involved in Drug Overdose Deaths: United States, 2011-2016. National vital statistics reports: 50 from the Centers for Disease Control and Prevention, National Center for Health Statistics, National Vital Statistics System 2018; 67:1-14.
77. Lockwood T-L E, Huynh P, Richard A, Sightes E, Bailey K, Ray B, Lieberman M. Community overdose surveillance: Comparing substances collected from the death scene investigation to toxicology results. Drug and Alcohol Dependence 2021; 224:108722.
78. Meyer L, L6pez T, Espinosa R, Arias C F, Vollmers C, DuBois R M. A simplified workflow for monoclonal antibody sequencing. PLoS ONE 2019; 14.
79. Ehrenmann F, Kaas Q, Lefranc M P. IMGT/3Dstructure-D B and IMGT/DomainGapAlign: a database and a tool for immunoglobulins or antibodies, T cell receptors, MHC, IgSF and MhcSF. Nucleic acids research 2010; 38.
80. Ehrenmann F, Lefranc M P. IMGT/DomainGapAlign: IMGT standardized analysis of amino acid sequences of variable, constant, and groove domains (IG, TR, MH, IgSF, MhSF). Cold Spring Harbor protocols 2011; 2011.
81. Raybould M I J, Marks C, Krawczyk K, Taddese B, Nowak J, Lewis A P, Bujotzek A, Shi J, Deane C M. Five computational developability guidelines for therapeutic antibody profiling. Proceedings of the National Academy of Sciences of the United States of America 2019; 116:4025-30.
82. Swindells M B, Porter C T, Couch M, Hurst J, Abhinandan K R, Nielsen J H, Macindoe G, Hetherington J, Martin A C R. abYsis: Integrated Antibody Sequence and Structure-Management, Analysis, and Prediction. Journal of molecular biology 2017; 429.
83. Patel C N, Bauer S P, Davies J, Durbin J D, Shiyanova T L, Zhang K, Tang J X. N+1 Engineering of an Aspartate Isomerization Hotspot in the Complementarity-Determining Region of a Monoclonal Antibody. Journal of Pharmaceutical Sciences 2016; 105:512-8.

### Example 2. Structural characterization of HY6-F9 in complex with fentanyl

Opioid-related fatal overdoses have reached epidemic proportions. Yet existing treatments for opioid use disorder (OUD) offer limited options for long-term preventive protection, and accelerating development of newer approaches is critical. Monoclonal antibodies (mAbs) are a promising emerging treatment strategy that target and sequester selected opioids in the bloodstream, reducing drug access through the blood-brain barrier, thus preventing or reversing opioid toxicity. A murine mAb which binds fentanyl with high specificity was identified (see clone HY6-F9.6 VH and VL, Seq ID Nos:1-2 in Table A1). To determine the binding mechanism of the mAb, X-ray crystallography was used to solve the structure when bound to the target, to 2.2 A resolution or higher. Structural analysis showed important antibody-target binding interactions, including a hydrogen bonding mode that is dependent on residue(s) in the mAb's heavy chain and a tertiary amine of the ligand. Characterizing drug-mAb complexes represents a significant step towards rational antibody engineering and future manufacturing activities to support clinical evaluation.

### INTRODUCTION

Over the past two decades, annual fatal overdoses linked to opioids in the U.S. have rapidly increased, from less than 10,000 in 2000 to 49,860 in 2019. In 2020 that number spiked to 69,090, an increase of 38.6% over 2019 (Ahmad et al., Provisional drug overdose death counts. National Center for Health Statistics. 2022). Data from 2021 indicates an even steeper increase due to the additional multifactorial impact of the COVID-19 pandemic (Ahmad et al., 2022), surpassing 100,000 deaths in the U.S. alone. Current pharmacotherapies and behavioral interventions against opioid use disorder (OUD) are suboptimal, and the COVID-19 pandemic has further complicated outreach and treatment (Alexander et al., 2020, doi.org/10.7326/M20-1141; Sharma et al., Current psychiatry reports 19, 35-35, 2017). More effective treatments are needed to help control the opioid epidemic, particularly ones which impose minimal burden on strained public health resources. To this end, immunotherapeutics such as anti-opioid vaccines and monoclonal antibodies (mAbs) would represent a significant advancement in therapeutics for OUD and overdose (Pravetoni and Comer, Neuropharmacology 158, 107662, 2019).

In 2020 the NIH reported that 2.7 million people in the U.S. are currently living with an OUD (SAMHSA, Key substance use and mental health indicators in the United States, 2021). Worldwide ˜40-80 million people use opioids, although the frequency of that use is not well defined (UNODC, World Drug Report. Sales No. E.21.XI.8 ed. United Nations, 2021). In addition to outreach programs such as Narcotics Anonymous and behavioral treatments, several pharmacotherapeutic interventions exist for OUD, including opioid receptor agonists (methadone), partial agonists (buprenorphine), antagonists (naloxone and naltrexone), and agonist/antagonist combinations (e.g., suboxone) (Noble and Marie, Front Psychiatry 9, 742, 2018). However, these treatments have significant limitations which have only been further complicated by the ongoing COVID-19 pandemic. Methadone regimens need daily dosing, requiring staff for observation and patient transport to the facility; staffing shortages have had negative impacts on these services (Joudrey et al., JAMA Network Open 4, e2118223-e2118223, 2021). Naloxone, while effective, often requires higher or multiple doses to treat overdoses from fentanyl, carfentanil, and their analogues (Moss and Carlo, Substance Abuse: Treatment, Prevention, and Policy 14, 1-6. 2019). For patients outside of treatment, access to and the use of illegal narcotics carries a risk of infection and health complications from bloodborne and airborne pathogens such as HIV, Hepatitis C, and SARS-CoV-2 (Harvey et al., J Addict Med 15, 461-467. 2021; Rodda et al., Journal of Urban Health 97, 808-813. 2020). While policy changes have the potential to abate some of the complications of existing treatments in light of the COVID-19 pandemic (Alexander et al., 2020; Joudrey et al., 2021; Sharma et al., Current psychiatry reports 19, 35-35. 2017), novel treatments for OUD were already considered a necessary innovation prior to 2019 (Stuart et al., Substance Abuse: Research and Treatment 12, 2018; Volkow and Collins, New England Journal of Medicine 377, 391-394, 2017).

Drug conjugate vaccines and monoclonal antibodies are promising treatments for OUD and may overcome some of the limitations of current pharmacotherapies. Conjugate vaccines consist of a drug-based hapten conjugated to an immunogenic carrier protein which elicits a drug-specific polyclonal antibody response against a targeted drug. Antibodies, elicited or ready-made, bind the target drug and prevent its distribution to the brain, reducing overall unbound drug concentrations. Unlike small molecule therapies, antibody-based therapies are effective on a weeks-(mAb) or months-(vaccine) long timescale (Smith et al., Journal of the American Chemical Society 141, 10489-10503, 2019; Tenney et al., 2019, doi.org/10.1101/580100). Additionally, antibodies do not directly affect signaling at opioid receptors and carry fewer side effects. Perhaps most importantly, mAbs against highly potent synthetic opioids have the potential to negate or rescue from otherwise lethal doses of these drugs, providing both a preventive and therapeutic intervention to directly reduce opioid overdose deaths (Baehr et al., 2022; Smith et al., 2019). Drug-specific mAbs have been shown to reduce behavioral and pharmacological effects of opioids such as self-administration, respiratory depression, bradycardia, antinociception, locomotor activity in mice, rats, and non-human primates (Baehr et al., J Pharmacol Exp Ther 381, 129-136, 2022; Smith et al., 2019; Tenney et al., 2019). Vaccines and/or mAb could be administered to specific patient populations depending upon the clinical scenario related to either treatment of OUD or rescue from overdose. Both approaches have been shown to not interfere with current pharmacotherapies (Baehr et al., J Pharmacol Exp Ther 381, 129-136, 2022; Ban et al., mAbs 13, 2021). The longevity of antibody-based treatments can reduce the treatment burden on healthcare facilities and the need for daily or weekly compliance by patients. It is also not possible to abuse, resell, or overdose on vaccines or mAbs, reducing regulatory burdens, risk of illegal diversion, and abuse liability associated with agonists such as methadone.

High-potency synthetic drugs such as fentanyl and carfentanil present favorable targets for mAb-based therapies, as drugs with larger effective doses can quickly saturate antibody titers. This limitation was a major obstacle to an effective nicotine vaccine (Pentel and LeSage, Adv Pharmacol 69, 553-580, 2014), as minimum effective titers could only bind the molar equivalent of one cigarette. However effective mAb-based therapies against drugs of abuse have seen recent successes. An anti-cocaine mAb, h2E2, is in late-stage preclinical development (Marckel et al., Drug Metab Dispos 47, 184-188, 2019), and an anti-methamphetamine mAb, IXT-m200, has advanced to clinical trials (NCT05034874), supporting the continued investigation of mAb targeting small molecule drugs. The OXY-KLH vaccine targeting oxycodone is currently in Phase Ia/Ib clinical trials (Baehr et al., 2020). An overview of completed and ongoing clinical trials is reviewed elsewhere (Bloom and Bushell, Vaccines (Basel) 10, 2022). The potency and diversity of emerging synthetic opioids highlights the need for more effective counteragents (Ahmad et al., Provisional drug overdose death counts. 2022), but also presents a more favorable molar ratio for sequestration by mAb compared to higher LD₅₀ drugs such as nicotine. Considering current treatment limitations, and the properties of these emerging synthetic drugs, mAb-based therapies show significant promise as additional treatments to help address the rising opioid epidemic.

Previously, the anti-drug mAb HY6-F9 was elicited through immunizing mice with a vaccine consisting of the target drug conjugated to the keyhole limpet hemocyanin subunit (drug-sKLH vaccine). In mice, passive immunization with the mAb showed serum sequestration of the target drug, reduced drug concentration in the brain, and reduced pharmacological effects (see Example 1). Competitive ELISA revealed mAb in vitro has strong binding affinity to the target drug fentanyl (Baehr et al., Journal of Pharmacology and Experimental Therapeutics 375, 469-477, 2020).

To date, no data regarding the specific binding mechanism of the fentanyl-mAb complex were available. Herein, we report the crystal structure of the above-mentioned mAb complexed with fentanyl. Kinetic affinity of mAb for hapten derived from the drug was assessed by biolayer interferometry (BLI) and the structures were compared to other known mAb and endogenous receptor that bind structurally similar ligand. By investigating these structures at near-atomic resolution, we were able to observe specific functional group coordination, and identify conserved features that contribute to mAb affinity for the drug target. Characterizing the mode of antibody-drug binding will support humanization, manufacturing, and qualification of mAb against fentanyl and its analogs being readied for clinical trials. By illuminating the mechanism of this molecular recognition, we hope to use rational design of more effective develop vaccines and mAbs against emerging synthetic opioids.

### Results

HY6-F9 was isolated from mice immunized with a conjugate vaccine consisting of a fentanyl-based hapten attached at the terminal carbon of the two-carbon chain and replacing the aromatic ring with a glutaric amide (Gly)₄ linker (SEQ ID NO: 200) to sKLH. The F-sKLH has been shown effective against fentanyl (Baehr et al., Journal of Pharmacology and Experimental Therapeutics 375, 469-477, 2020). BLI indicated HY6-F9 has an affinity of <100 μM for the biotinylated fentanyl hapten (Table 7). The structure of HY6-F9 Fab bound to fentanyl was solved to a resolution of 1.75 A, with nine Fabs in the unit cell (Table 8). This solution is notable as there are nine tNCS-related copies within the unit cell in the P31 space group.

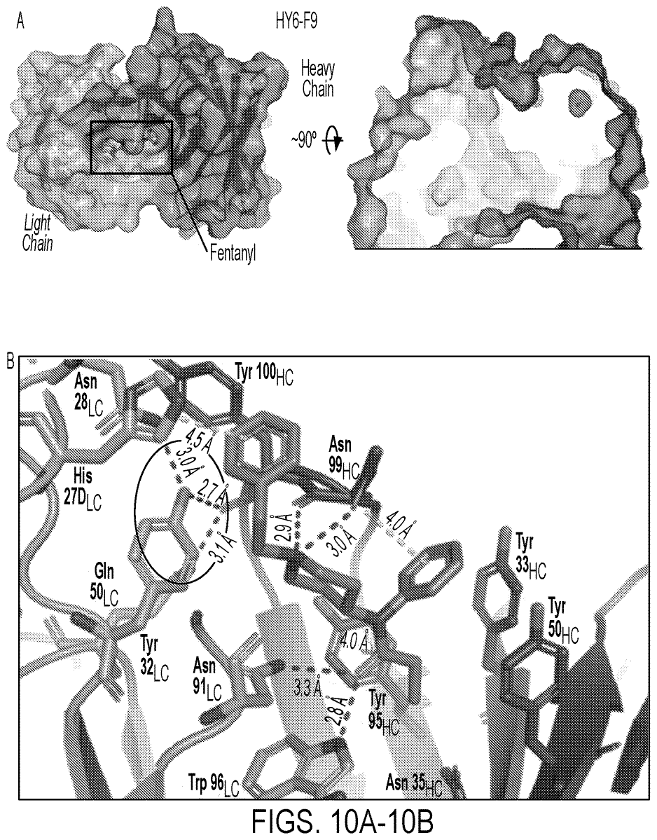

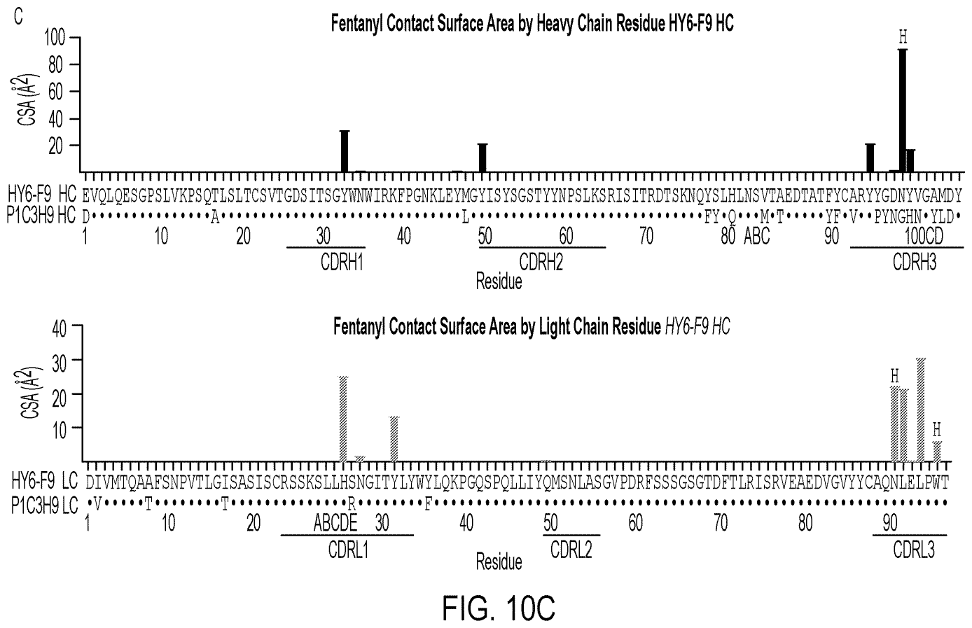

The binding site of fentanyl in HY6-F9 is difficult to characterize, as each chain runs along each “side” of the ligand, forming a long pocket with multiple points of contact (FIG. 10A), where the ligand is ˜85% buried. Fentanyl has a pKa of 8.99 and its protonation state is uncertain in this structure (Roy and Flynn, Pharmaceutical research 6, 147-151, 1989), although the residues Asn99_{HC}, Asn91_{LC}, and Trp96_{LC}, are in position to form multiple hydrogen bonds (FIG. 10B). At a physiological pH of ˜7.4, these bonds are likely formed. The CDRH3 folds over the ligand and forms a hydrogen bond between the protonated tertiary amine of the piperidine ring, and either the Asn99_{HC} side chain or backbone (FIG. 10B). In the event of the side chain contributing to this interaction, the aromatic ring of the N-phenyl group in fentanyl may also form a π-cation interaction with the partially charged sidechain. The clamp-like action of fentanyl on Asn99_{HC} is a key interaction, with fentanyl burying nearly 100 Å² of the residue, by far the largest single contact surface area (CSA) observed (FIG. 10C). This also may be the structural basis for the low K_{dis} of HY6-F9.

Opposite Asn99_{HC}, the Asn91_{LC} side chain hydrogen bonds the ketone group of fentanyl, and that same ketone can hydrogen bond with the Trp96_{LC} side chain, possibly in equilibrium with Asn91_{LC} (FIG. 10B), showing hydrogen bond redundancy of this mAb:ligand complex. The hydrogen bond between Trp96_{LC} or Asn91_{LC} and the fentanyl ketone forms a partial charge, allowing 7r-cation interaction of Tyr95_{HC} with the adjacent carbon. Additionally, a 7L-7L interaction appears to form between the phenethyl ring of fentanyl and His27D_{LC} (FIG. 10B). This phenethyl ring is not present in the fentanyl-sKLH hapten and thus this bond is likely not a result of affinity maturation.

HY6-F9 has two additional binding site features beyond distinct bonds. First, a small hydrophobic pocket is formed at the base of the ligand. This shallow pocket sequesters only the ethyl group and part of the N-phenyl ring, a large hydrophobic region of the ligand (FIG. 10A). As the phenethyl ring would be replaced by the fentanyl-sKLH vaccine linker, no hydrophobic pocket is observed for that group. Second, there is a hydrogen bond network between the CDRL1, CDRL2, and CRDH3 (FIG. 10B). These bonds are not involved in ligand binding; rather they pull the CDRH3 towards the LC and over the fentanyl. A recent preprint (Triller et al., 2022, doi.org/10.1101/2022.05.02.487070) identified other fentanyl binding mAbs. They note that apo-form crystallization was not successful, an observation we also made. They attribute this to significant CDR flexibility, suggesting an induced-fit mechanism, and support this with ITC experiments (Triller et al., 2022, doi.org/10.1101/2022.05.02.48707). With the large CSA of fentanyl to Asn99_{HC}, without being bound by theory, we believe that the tip of the CDRH3 loop may move into place following drug binding, an induced fit mechanism, with the observed hydrogen bond network assisting in positioning this loop.

Prior to our structure of HY6-F9, no fentanyl:mAb structures were deposited in the PDB, making HY6-F9 the first structure of a mAb bound to fentanyl to be reported in the PDB. Another study described two isolated fentanyl binding mAbs, and performed in silico modeling of those mAbs (Ban et al., 2021, mAbs 13. 10.1080/19420862.2021.1991552). One mAb (P1C3H9) was aligned to HY6-F9 (FIG. 10C). Within the HC, the hydrophobic pocket appears conserved, although significant differences are present within the CDRH3. However, one important residue for binding, Asn99_{HC}, is present in an adjacent location. Their model shows a similar Fab structure and CDR arrangement, but the authors report a differently structured binding site with no tertiary amine coordination.

TABLE 7
BLI Results of HY6-F9

| | | K_{D} | Kₒₙ | K_{dis} | Full |
|---|---|---|---|---|---|
| mAb | Ligand | (M) | (1/Ms) | (1/s) | R² |
| HY6-F9 | OXY- | NDB | NDB | NDB | NDB |
| | Biotin | | | | |
| HY6-F9 | MOR- | NDB | NDB | NDB | NDB |
| | Biotin | | | | |
| HY6-F9 | F- | (5.0 ± | (1.4 ± | (6.8 ± | 0.9999 |
| | Biotin | 1.2) × 10⁻¹¹ | 0.01) × 10⁵ | 1.6) × 10⁻⁶ | |

^{a}NDB = No detectable binding

TABLE 8
Data Collection and Refinement Statistics for Crystal Structures.

| | HY6-F9 |
|---|---|
| Data collection | |
| Space group | P3₁ |
| Cell dimensions | |
| a, b, c (Å) | 218.14, |
| | 218.14, 89.02 |
| α, β, γ (°) | 90, 90, 120 |
| Resolution (Å) | 50.00-1.75 |
| | (1.78-1.75) |
| R_{merge}^{a} | 0.155 (0.998) |
| <I/σ(I)> | 7.7 (1.3) |
| CC_{1/2} | 0.994 (0.989) |
| Completeness | 99.6 (98.9) |
| Redundancy | 5.1 (5.0) |
| Refinement | |
| Resolution (Å) | 43.3-1.75 |
| | (1.81-1.75) |
| No. unique reflections | 475987 (47293) |
| R_{work}^{b}/R_{free}^{c} | 19.7/22.6 |
| | (27.2/30.5) |
| No. atoms | 33436 |
| Protein | 30132 |
| Water | 2829 |
| Ligand | 475 |
| B-factors (Å²) | 23.94 |
| Protein | 23.20 |
| Water | 31.19 |
| Ligand | 28.12 |
| RMS bond length (Å) | 0.006 |
| RMS bond angle (°) | 0.90 |
| Ramachandran Plot Statistics ^{d} | |
| Residues | 3942 |
| Most Favored region | 97.24 |
| Allowed Region | 2.76 |
| Disallowed Region | 0.0 |
| Clashscore | 3.48 |
| PDB ID | 7U64 |

^{a}R_{merge} = [ΣₕΣᵢ|Iₕ − Iₕᵢ|/ΣₕΣᵢIₕᵢ] where Iₕ is the mean of Iₕᵢ observations of reflection h. Numbers in parenthesis represent highest resolution shell.
^{b}R_{factor} and ^{c}R_{free} = Σ∥F_{obs}| − |F_{calc}∥ / Σ|F_{obs}| × 100 for 95% of recorded data (R_{factor}) or 5% data (R_{free}).
^{c}MolProbity reference

### Discussion

Vaccine and mAb development are a complex process: vaccine-induced polyclonal antibodies and mAbs require high affinity and specificity for the drug target, functional groups require more specific targeting due to their relative scarcity on small ligands, and hapten linker placement must consider functional group availability and linker egress. However, despite robust work in the field of anti-opioid and anti-drug mAbs over the last 50 years, few structures of mAb against drugs of abuse, and even fewer opioid-mAb structures, have been deposited in the PDB. This limitation has significantly impacted the field's ability to rationally design new vaccines or modify existing mAbs. The structure of unique mAb bound to target synthetic opioid drug fentanyl show that such mAb may have a protonated tertiary amine binding motif, providing specificity, high affinity, and low off-rate for the target ligand.

Understanding ligand-protein binding poses unique challenges beyond those observed in engineering protein-protein interactions. Through examining synthetic opioid drug fentanyl that constitutes a drug class of significant interest and identifying binding principles and applying them to understand hapten:BCR interactions, we hope to develop potent mAbs to treat fentanyl or analogs related overdose and disorder.

### Methods

### BLI Analysis

Biolayer interferometry measurements of purified HY6-F9 mAb were performed on an Octet Red96e system (Sartorius). Streptavidin biosensors were loaded with biotinylated hapten of fentanyl (F1-biotin) at 0.1-0.2 ug/mL in PBS-T for 60 sec (Baehr et al., 2020). Following a 60 sec baseline measurement in PBS-T, association rate was measured with purified mAb, 5 nM-40 nM for 3-5 min, and then dissociation rate was measured in PBS-T for 5-10 min. For analysis at pH 5.8 and 10.0, following the 60 sec baseline measurement in PBS-T, hapten loaded biosensors were equilibrated in 0.1 M MES+0.05% Tween-20, pH 5.8 or 0.1 M carbonate-bicarbonate+0.05% Tween-20, pH 10.0 for 10 min prior to the association step. Dissociation constant was calculated as k_{off}/kₒₙ by Octet analysis software (Sartorius).

### Generation of HY6-F9 mAb Expression Vectors

Fentanyl-binding mAb VH and VL sequences were cloned into pcDNA3.4 mammalian expression vectors prepared by Genscript. For the heavy chain (HC), the pcDNA3.4 expression vector was modified to contain a CMV promoter driven open-reading frame (ORF) preceded by a Kozak consensus sequence and a murine IGHV signal peptide (MGWSCIILFLVATATGVHS (SEQ ID NO: 198)). The ORF terminates with a human IgG1 constant region (Accession #P01857). For the light chain (LC), a pcDNA3.4 expression vector was modified to contain a CMV promoter driven ORF preceded by a Kozak consensus sequence and a murine IGKV signal peptide (METDTLLLWVLLLWVPGSTG (SEQ ID NO: 199)). The ORF terminates with a human IgK constant region (Accession #P01834). Both pcDNA3.4 expression vectors were designed with cloning sites between the signal peptide and the constant region to facilitate an in-frame Gibson assembly cloning strategy for variable region insertion with Gibson Assembly® Master Mix (New England Biolabs Catalog #E2611). Antibody variable region gene fragments for Gibson assembly of anti-fetnanyl antibody expression vectors were codon optimized and synthesized by Twist Bioscience. The synthesized gene fragments were designed to contain ˜30 base pairs of vector sequence homology on the 5′ and 3′ ends of the gene fragment to facilitate Gibson assembly into the expression vector.

Generation of HY6-F9 VH his-Fab Expression Vector

The VH region of HY6-F9 was PCR amplified from pcDNA™3.4 plasmid DNA, and a pMN destination backbone was linearized using Platinum™ SuperFi II PCR Master Mix. The VH region was cloned into the linearized pMN backbone containing a generic human CH1 region with a C-term 6xHis-tag (SEQ ID NO: 201), and an N-term secretion tag, using the Infusion HD Cloning Plus kit (Takara Bio). The HY6-F9 chimeric His-Fab expression vector was transformed into NEB5 a E. coli cells (New England BioLabs) and DNA was isolated using a MidiPrep (Qiagen). Expression vector sequencing was performed by Genewiz (Genewiz Inc, Seattle, WA).

### Fab Preparation

HY6-F9 His-Fab was expressed in HEK293e cells as described. The supernatant was harvested through centrifugation at 4000×g for 20 mins. The supernatant was then sterile filtered using a 0.2 μm bottle-top filter, batch bound to 4 mL Ni-NTA resin for 1 hour at 23° C. with shaking at 120 rpm, and then HY6-F9 Fab was eluted with 5 mM Tris buffer containing 300 mM imidazole.

### HY6-F9 mAb for BLI Purification

mAb was purified from filtered cell culture supernatant via liquid chromatography on an AKTA pure (Cytiva) with a HiTrap MabSelect PrismA protein A column (Cytiva Product #17549851) (running buffer PBS, pH 7.4, elution buffer 0.1 M Na-Acetate, pH 3.5). Eluted mAb was neutralized by dilution with ⅓rd final volume 2.5 M Tris, pH 7.2, and buffer exchanged into PBS, pH 7.4. Purified mAb concentration was determined by absorbance at 280 nm on a Nanodrop (ThermoFisher). Confirmatory analysis of purified mAb was performed by SDS-PAGE under reducing and non-reducing conditions.

### SEC Purification and Concentration of Fabs for Crystallization

Following affinity column purification, Fabs were concentrated to 2 mL using 10 kDa Amicon® (Millipore Sigma) and sterile filtered (UltraFree-CL, Millipore Sigma), before injection onto a Superdex 200 16/600 size exclusion column (Cytivia) equilibrated with HEPES (5 mM HEPES, 150 mM NaCl, pH 7.5) buffer using an AKTApure (GE Biosciences) system. Fab peak fraction was pooled and concentrated to 20 mg/mL concentration for crystallization trials.

### Crystallization and Structure Determination

Fabs were incubated for >2 hours at 23° C. with 2-fold molar excess of their target ligand before being used for crystallization trials. Swissci® MRC 2 Well UVXPO plates were used to screen conditions from commercial 96-well screens using an NT8 drop setter (Formulatrix). Screens used include MCSG1-3 (Microlytic), WPS2 (Rigaku), Xtal High Throughput and Additive Screen (Hampton Research). Crystallizing conditions were optimized in EASYXTAL® 15-well crystallization trays (NextalBiotech). Final well condition that provided a diffracting crystal is 2.8 M Sodium Acetate. Crystals were flash cooled in their crystallizing condition buffered with 30% ethylene glycol, without cryo-protectant due to high salt content (HY6-F9). Data were collected at either Sector 19 of the Advanced Photon Source (Argonne National Labs), or Beamline 5.0.1 at the Advanced Light Source (Lawrence Berkeley National Lab). Beamline data were processed using XDS (Kabsch, Acta Crystallographica Section D Biological Crystallography 66, 125-132, 2010) or HKL-2000 (Otwinowski and Minor, Processing of X-ray diffraction data collected in oscillation mode. In (Elsevier), pp. 307-326, 1997), data were reduced as necessary using CCP4 (Winn et al., Acta Crystallographica Section D Biological Crystallography 67, 235-242, 2011), and the structures were phased and solved using the Phenix software suite (Liebschner et al., Acta Crystallographica Section D Structural Biology 75, 861-877, 2019), the Coot toolkit (Emsley et al., Acta Crystallographica Section D Biological Crystallography 66, 486-501, 2010), and ChimeraX (Pettersen et al., Protein Science 30, 70-82, 2021) with the ISOLDE plug-in (Croll, Acta Crystallographica Section D Structural Biology 74, 519-530, 2018). Structure visualization, comparisons, and molecular representations were created in PyMol (Schrodinger, The PyMOL Molecular Graphics System, Version 2.1.1, 2015). Protein interaction data were analyzed using GraphPad Prism (version 9.0.1 for Mac). All collection and refinement data are available in Table 8.

### Example 3. Characterization of Humanized HY19-1H6 mAbs

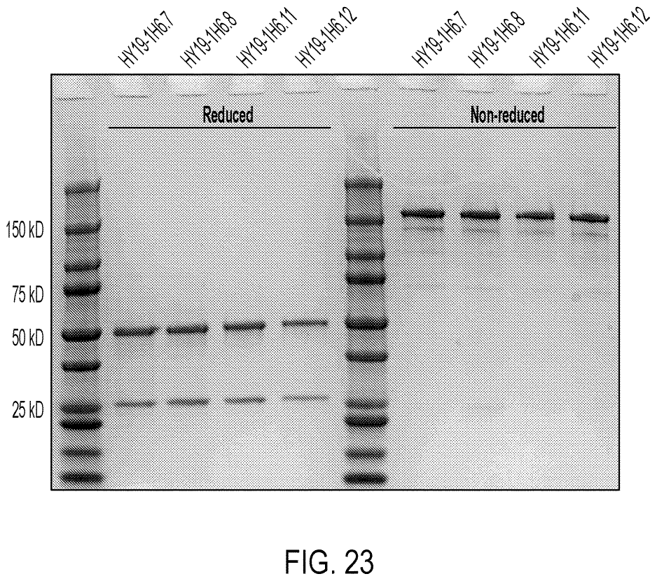

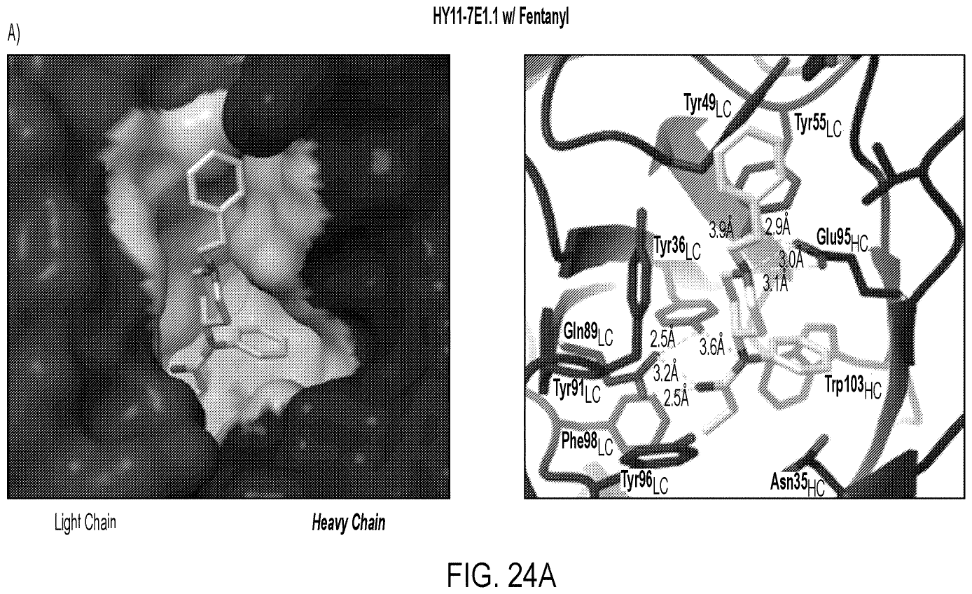

Four representative, fully humanized mAbs (Table 9) were tested to mitigate potential PTM risks. Fully humanized HY19-1H6 mAbs maintained binding to fentanyl hapten but lost binding to carfentanil hapten (Table 10), however, fully humanized HY19-1H6 mAbs displayed favorable fentanyl hapten binding properties relative to certain HY6-F9 and HY11-7E1 humanized mAbs (Table 11). SDS-PAGE data of these mAbs is shown in FIG. 23.

TABLE 9
Four representative, fully humanized mAbs.

| | | Potential CDR |
|---|---|---|
| | | PTM Risks |
| | | (deamidation, |
| | CDR | isomerization, |
| mAb ID | Mutation | glycosylation) |
| HY19-1H6.7 | None | VH N62 deamidation |
| | | VL N108 glycosylation |
| | | (NIS motif) |
| HY19-1H6.8 | VL N108Q | VH N62 deamidation |
| HY19-1H6.11 | VH N62Q | VL N108 glycosylation |
| | | (NIS motif) |
| HY19-1H6.12 | VH N62Q | None |
| | VL N108Q | |

TABLE 10
Fully humanized HY19-1H6 mAbs bind fentanyl hapten.
BLI Kinetics Result

| | | Fentanyl | | Carfentanil | |
|---|---|---|---|---|---|
| | mAb | K_{D} | K_{ON} | K_{D} | K_{ON} |
| mAb ID | Species | (nM) | (1/ms) | (nM) | (1/ms) |
| HY19-1H6 | Murine | <0.001* | 1.51E+05 | <0.001* | 2.26E+05 |
| HY19- | Chimeric | <0.001* | 1.41E+05 | <0.001* | 7.49E+04 |
| 1H6.1 | | | | | |
| HY19- | Hu_HC + | <0.001* | 3.33E+05 | <0.001* | 1.87E+05 |
| 1H6.4 | Mu_LC | | | | |
| HY19- | Humanized, | <0.001* | 2.38E+05 | No Detectable | |
| 1H6.7 | no PTM | | | Binding | |
| | mitigation | | | | |
| HY19- | Humanized, | <0.001* | 2.15E+05 | | |
| 1H6.8 | Partially | | | | |
| | PTM | | | | |
| | Mitigated | | | | |
| HY19- | Humanized, | <0.001* | 2.10E+05 | | |
| 1H6.11 | Partially | | | | |
| | PTM | | | | |
| | Mitigated | | | | |
| HY19- | Humanized, | <0.001* | 2.09E+05 | | |
| 1H6.12 | Fully PTM | | | | |
| | Mitigated | | | | |

*Undetectable off-rate by BLI

TABLE 11
Fully humanized HY19-1H6 mAbs displayed favorable
fentanyl binding property.
BLI Kinetics Result

| | Fentanyl | | Carfentanil | |
|---|---|---|---|---|
| Humanized mAb ID | K_{D} (nM) | K_{ON} (1/ms) | K_{D} (nM) | K_{ON} (1/ms) |
| HY6-F9.19 | 0.010 | 2.01E+05 | NDB | |
| HY11-7E1.17 | 0.060 | 1.85E+05 | 1.31 | 2.81E+05 |

| | | | |
|---|---|---|---|
| HY19-1H6.12 | <0.001* | 2.09E+05 | NDB |

*Undetectable off-rate by BLI

HY19-1H6.7 displayed decreased aggregation compared to high aggregation observed in PTM mitigated counterparts (Table 12). HY19-1H6.7/8 showed lower hydrophobicity relative to HY19-1H6.11/12 (Table 13). HY19-1H6.7/8 displayed higher Fab thermal stability than HY19-1116.11/12 (Table 14).

TABLE 12
SEC-HPLC Data.

| mAb ID | % Monomer | % Aggregate |
|---|---|---|
| HY19-1H6.7 | 97.6 | 2.4 |
| HY19-1H6.8 | 87.6 | 12.4 |
| HY19-1H6.11 | 93.1 | 6.9 |
| HY19-1H6.12 | 94.1 | 5.9 |

TABLE 13
HIC-HPLC Data.

| mAb ID | Main Peak RT (min) |
|---|---|
| HY19-1H6.7 | 12.939 |
| HY19-1H6.8 | 13.031 |
| HY19-1H6.11 | 13.557 |
| HY19-1H6.12 | 13.64 |

TABLE 14
Tm Data.

| mAb ID | Fab Tm (° C.) |
|---|---|
| HY19-1H6.7 | 76.4 |
| HY19-1H6.8 | 76.5 |
| HY19-1H6.11 | 75.6 |
| HY19-1H6.12 | 75.1 |

TABLE 15
Data Summary.

| | | HPLC | | | | |
|---|---|---|---|---|---|---|
| | | HIC | SEC | | | |
| | | Main Peak | % | % | Fab Tm | BLI, KD |
| mAb ID | CDR PTM | RT (min) | Monomer | Aggregate | (° C.) | (nM) |
| HY19-1H6.7 | N62 DeAm | 12.939 | 97.6 | 2.4 | 76.4 | <0.001 |
| | N108 GlyC | | | | | |
| HY19-1H6.8 | N62 DeAm | 13.031 | 87.6 | 12.4 | 76.5 | <0.001 |
| HY19-1H6.11 | N108 GlyC | 13.557 | 93.1 | 6.9 | 75.6 | <0.001 |
| HY19-1H6.12 | None | 13.64 | 94.1 | 5.9 | 75.1 | <0.001 |

### Example 4. Stability and Stress Testing of Certain α-Fentanyl mAB Clones

Biophysical Characterization. HY19-1H6.7 displays the lowest Fab melting temperature Tₘ among the three clones (Table 16) tested in this Example. HY19-1H6.7 and HY11-7E1 tested clones show better hydrophobicity profiles than HY6-F9 clone (Table 17).

TABLE 16
Tₘ data.

| | | Fab Tₘ | C_{H}2 |
|---|---|---|---|
| mAb ID | mAb Description | (° C.) | Tₘ |
| HY6-F9.19 | HY6-F9 Humanized Lead w/WT Fc | 78.2 | 69.5 |
| HY11-7E1.17 | HY11-7E1 Humanized Lead w/WT Fc | 80.4 | 68.3 |
| HY19-1H6.7 | HY19-1H6.7 Humanized Lead | 76.4 | 69.3 |

TABLE 17
HIC-HPLC data.

| | | Main Peak |
|---|---|---|
| mAb ID | mAb Description | RT (min)* |
| HY6-F9.19 | HY6-F9 Humanized Lead w/WT Fc | 14.3 |
| HY11-7E1.17 | HY11-7E1 Humanized Lead w/WT Fc | 11.9 |
| HY19-1H6.7 | HY19-1H6.7 Humanized Lead | 12.9 |

*HIC-HPLC salt gradient ends at 16.67 min.

Stability. Samples were concentrated to 10 mg/mL in PBS (pH 7.2-7.4). Samples were subjected to 3 times freeze/thaw (˜80° C./4° C.) cycles, or stored at 37° C. for 1 week, 2 weeks, or 3 weeks. Samples were then analyzed using SEC-HPLC to monitor aggregation, or ELISA for potency.

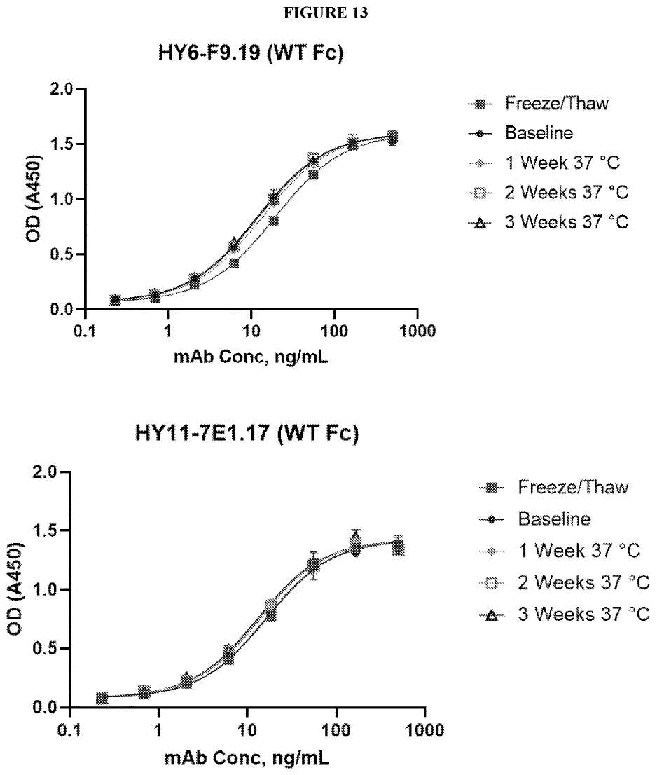

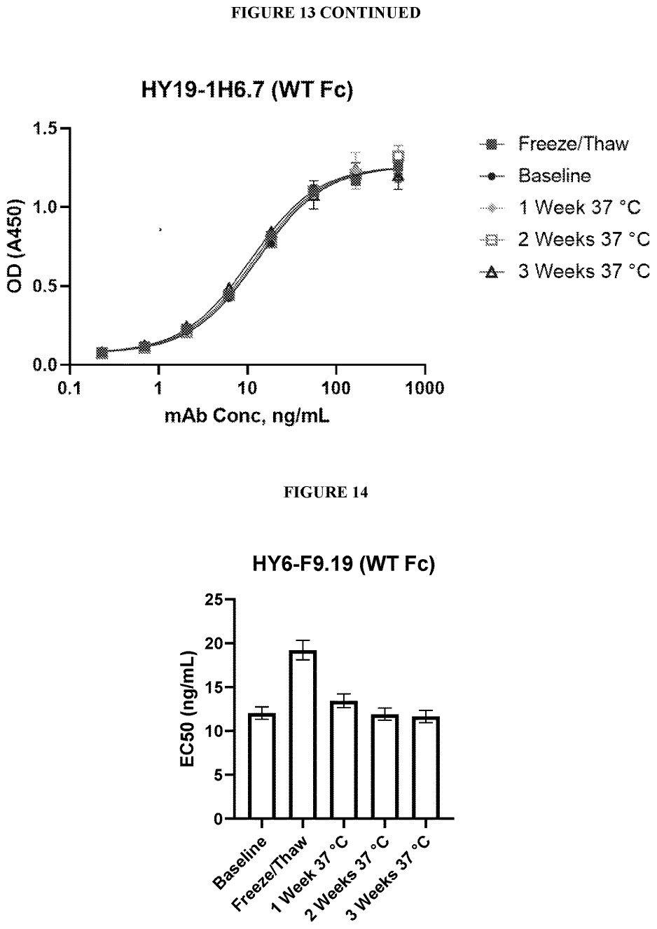

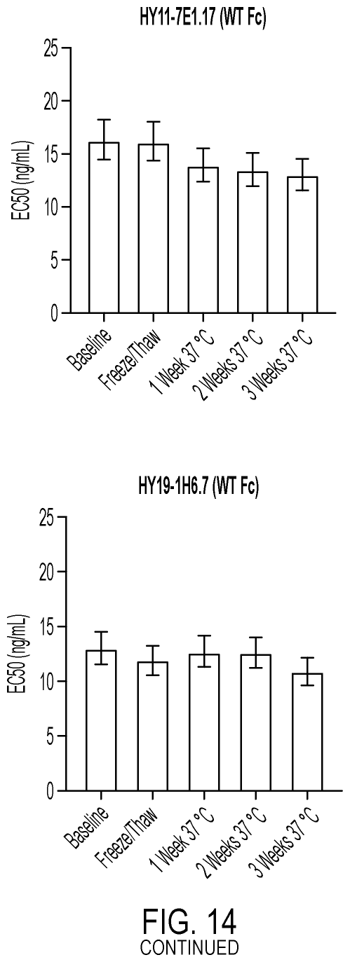

All samples displayed similar potency to reference at each 37° C. storage time point, although tested HY6-F9 clone appears to show decreased potency after freeze/thaw cycles in PBS (Table 18, also see FIG. 13 and FIG. 14).

In the SEC-HPLC assay, the tested HY9-F9 clone (HY6-F9.19) only showed minimal aggregation in the 3 weeks sample, demonstrating good aggregation profile for 3 weeks at 37° C. For the tested HY11-7E1 clone, baseline sample at 10 mg/mL concentration in this Example showed more aggregation (93.6% monomer) than in lower concentration sample of previous study (100% monomer) not shown herein. In this Example, the tested HY11-7E1 clone (HY11-7E1.17) displayed decreasing aggregation over time upon extended storage at 37° C. (Table 19). In contrast, the tested HY19-1H6 clone (HY19-1H6.7) in this example showed increasing aggregation over time upon extended storage at 37° C.

TABLE 18
Potency ELISA. Plates were coated with 0.05 μg/mL F1-BSA,
antibody samples were added at eight different concentrations
from 0.5 μg/mL highest concentration with 1:2 serial dilution
to lower concentrations. Secondary antibody of anti-human
IgG conjugated to HRP was used to develop ELISA signal.

| | | Relative Potency |
|---|---|---|
| mAb ID | Condition | to Reference |
| HY6-F9.19 | Reference | 100% |
| WT Fc | 3 × F/T | 63% |
| | 37 C., 1 wk | 90% |
| | 37 C., 2 wk | 101% |
| | 37 C., 3 wk | 103% |
| HY11-7E1.17 | Reference | 100% |
| WT Fc | 3 × F/T | 101% |
| | 37 C., 1 wk | 117% |
| | 37 C., 2 wk | 121% |
| | 37 C., 3 wk | 125% |
| HY19-1H6.7 | Reference | 100% |
| WT Fc | 3 × F/T | 109% |
| | 37 C., 1 wk | 102% |
| | 37 C., 2 wk | 103% |
| | 37 C., 3 wk | 119% |

TABLE 19
SEC-HPLC on stability samples. Baseline reference samples
and 37° C. for 1 week, or 2 weeks samples were diluted
to 1 mg/mL and tested using running buffer of PBS (pH 7.2).

| | | SEC-HPLC | | |
|---|---|---|---|---|
| | Stability | % | % | % |
| mAb ID | Condition | Monomer | Aggregate | Fragment |
| HY6-F9 WT | Baseline | 100.0 | 0.0 | 0.0 |
| | 37 C., 1 wk | 100.0 | 0.0 | 0.0 |
| | 37 C., 3 wk | 98.7 | 0.9 | 0.4 |
| HY11-7E1 WT | Baseline | 93.6 | 6.4 | 0.0 |
| | 37 C., 1 wk | 98.7 | 1.3 | 0.0 |
| | 37 C., 3 wk | 98.1 | 1.0 | 0.9 |
| HY19-1H6 WT | Baseline | 97.5 | 2.3 | 0.2 |
| | 37 C., 1 wk | 95.6 | 4.4 | 0.0 |
| | 37 C., 3 wk | 92.8 | 7.2 | 0.0 |

Forced Stress Studies (low pH hold and forced oxidation). During manufacturing, samples may be subjected to a low pH (˜3.5) hold to inactivate, e.g., viruses carried through during protein purification. Some mAbs may show instability or increased aggregation at low pH. In this study, all tested mAbs were subjected to pH3.5 for 90 minutes.

Oxidation of amino acids (e.g., Trp/Met) such as in the binding pocket can result in decreased or ablated target binding, shortened half-life, and immunogenicity. Oxidative stress in therapeutic mAbs may come from direct contact with oxygen, degradation of excipients in formulation, metal ion traces from production equipment or cell culture, and exposure to light. All tested mAbs herein were subjected to 0.10% H₂O₂ for 24 hours.

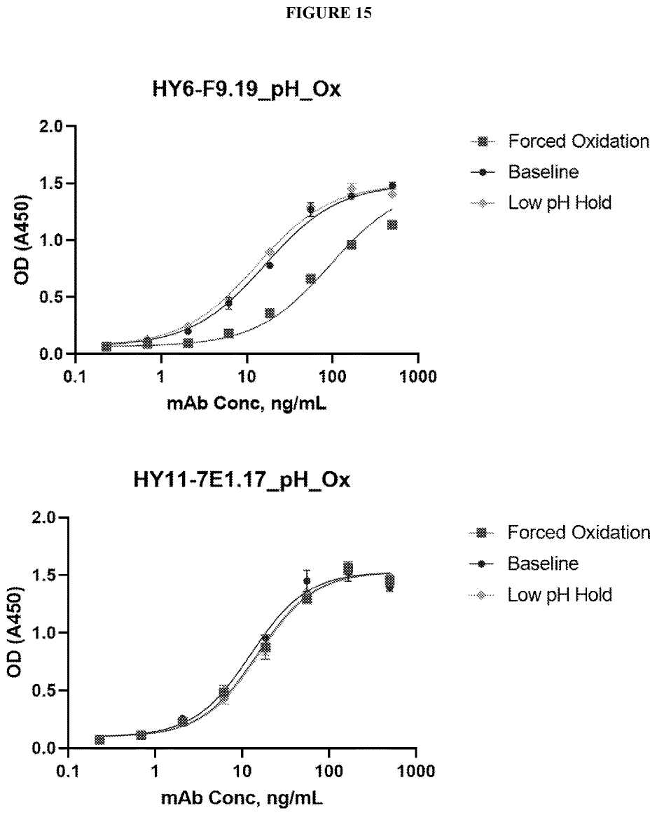

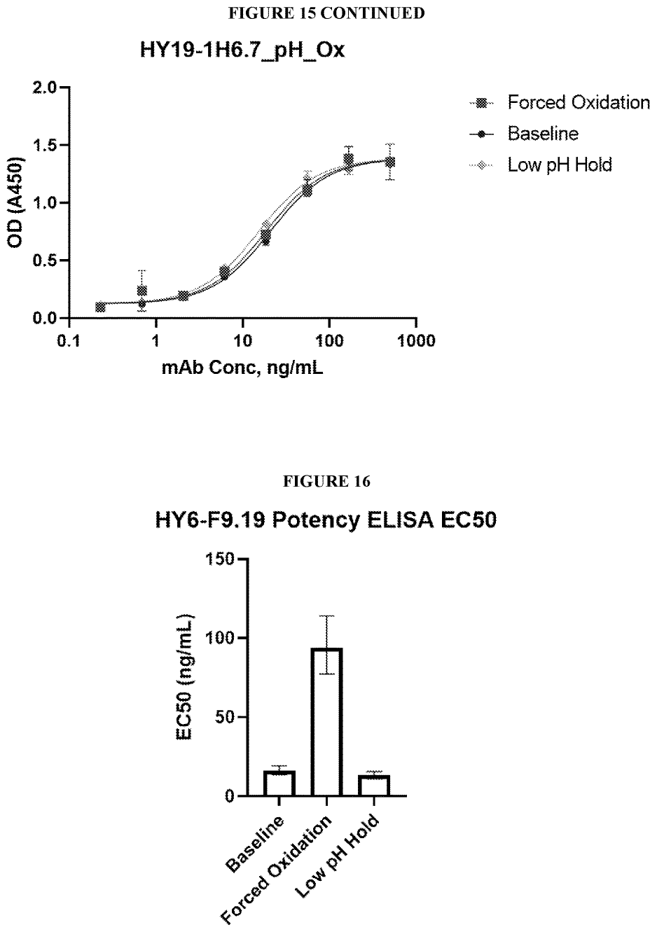

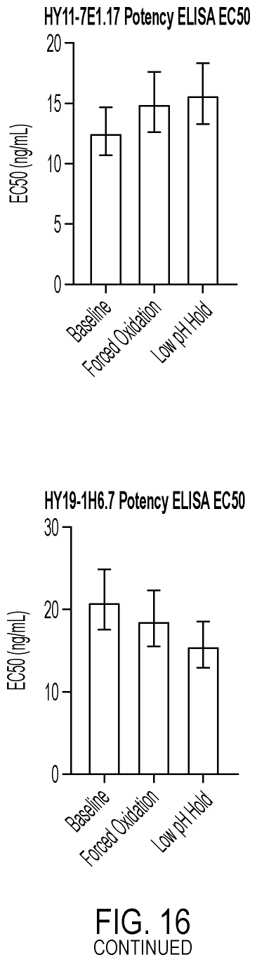

There was significant decrease in potency of HY6-1F9.19 after forced oxidation (Table 20, also see FIG. 15 and FIG. 16). All other tested mAbs show only modest differences in potency following forced oxidation and low pH hold.

Additional aggregation vs baseline was not observed in low pH hold samples (Table 21). Some fragmentation was observed in all samples, although this may be a buffer effect on the HPLC.

Overall, by SEC-HPLC and potency ELISA assay, all tested mAbs show acceptable stability during low pH hold stress studies. After exposure to forced oxidation condition, HY11-7E1.17 and HY19-1H6.7 did not display any decrease in potency, while HY6-F9.19 displayed a-fold decrease in potency.

TABLE 20
Potency ELISA on forced stress samples

| | | Relative |
|---|---|---|
| | | Potency to |
| mAb ID | Condition | Reference |
| HY6-F9.19 | Baseline | 100% |
| WT Fc | Forced Ox | 17% |
| | Low pH | 124% |
| HY11-7E1.17 | Baseline | 100% |
| WT Fc | Forced Ox | 84% |
| | Low pH | 80% |
| HY19-1H6.7 | Baseline | 100% |
| WT Fc | Forced Ox | 112% |
| | Low pH | 135% |

TABLE 21
SEC-HPLC on low pH hold samples

| | | SEC-HPLC | | |
|---|---|---|---|---|
| | Stability | % | % | % |
| mAb ID | Condition | Monomer | Aggregate | Fragment |
| HY6-F9.19 | Low | 98.6 | 0.0 | 1.4 |
| HY11-7E1.17 | pH Hold | 98.6 | 0.0 | 1.4 |
| HY19-1H6.7 | | 98.1 | 0.0 | 1.9 |

### Example 5. Antibody-Based Countermeasures Against Deliberate and Accidental Exposure to Fentanyl and Its Analogues

Highly specific, high affinity anti-fentanyl monoclonal antibodies (mAb) were developed for reversal of fentanyl overdose. Such mAbs may be used both alone and in combination with naloxone. Anti-fentanyl mAbs were isolated from mice using hybridoma technology, then sequences were humanized in vitro by CDR grafting on human mAb framework. To identify mAb candidates, in vitro affinity was assessed by biolayer interferometry and competitive ELISA, and in vivo efficacy was assessed in mice and rats including demonstration of reversal efficacy post-exposure. Proof of scalability and efficacy in large animal models includes Hanford mini-pigs and Rhesus macaque for in-depth assessment of respiratory parameters during fentanyl-induced respiratory depression and subsequent reversal with mAb.

### Anti-Fentanyl mAb Reverses Fentanyl-Induced Apnea in Pigs

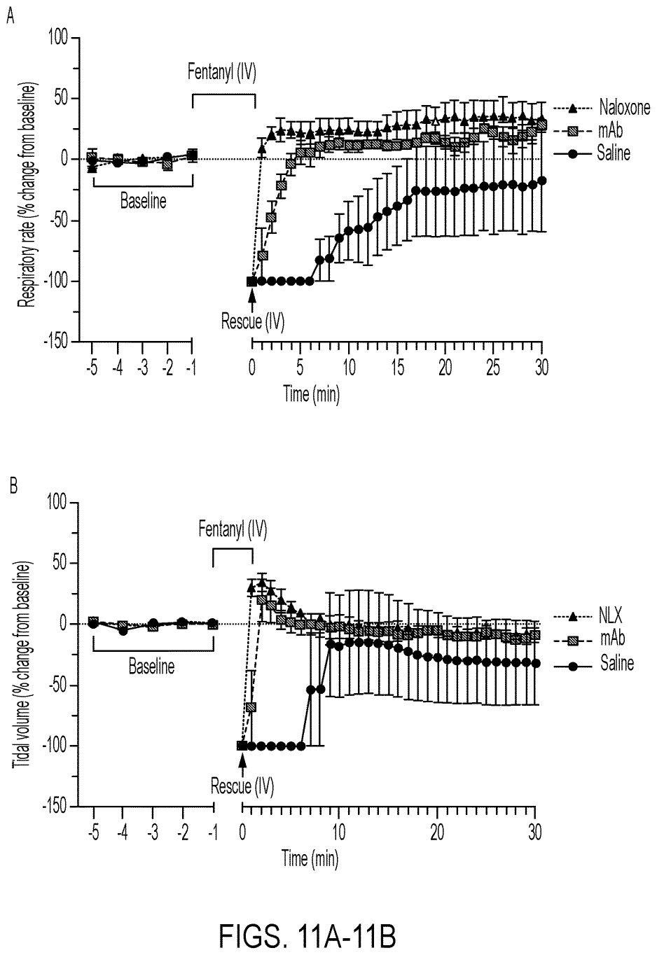

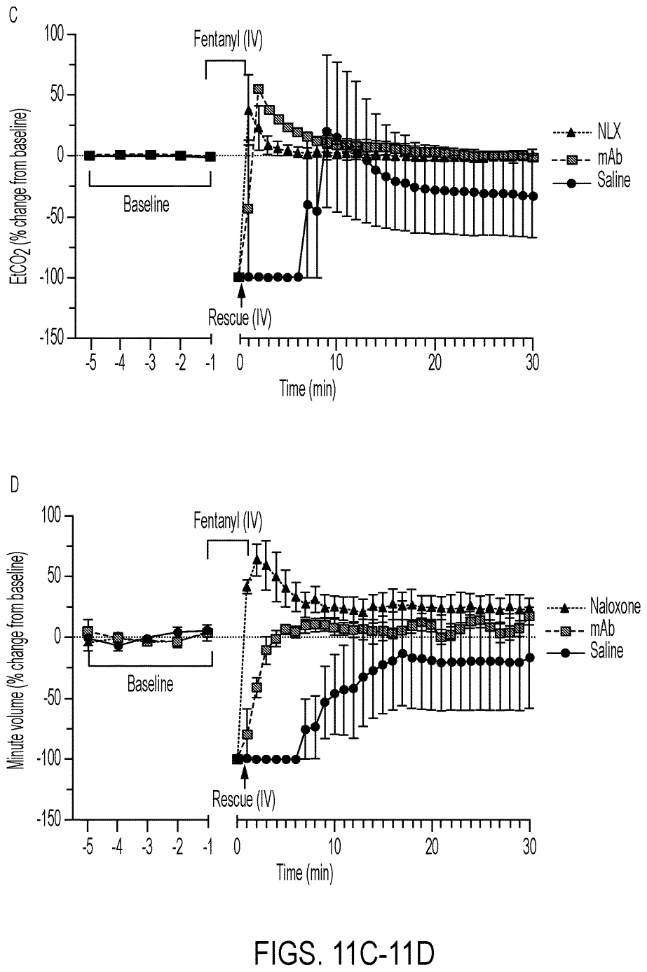

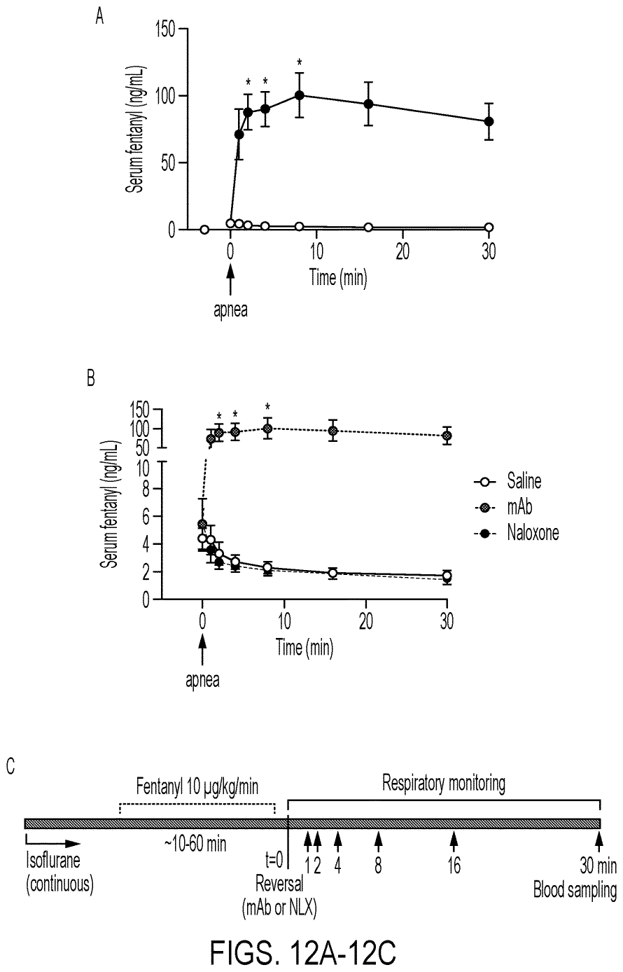

Hanford mini-pigs (n=3/group) were anesthetized with isoflurane. Fentanyl (10 μg/kg/min) was infused until apnea persisted for 2 min. Then, fentanyl infusion was stopped, and at t 0 apnea was reversed with saline control, anti-fentanyl mAb 40 mg/kg (HY6-F9.6), or naloxone 10 μg/kg. Respiratory parameters were recorded at 1-min intervals. It was shown that anti-Fentanyl mAb reverses apnea in pigs (FIGS. 11A-11D). Blood samples were collected prior to fentanyl administration (baseline), after apnea, and at 1-30 min after reversal. Fentanyl serum concentration was quantitated by LC-MS (FIGS. 12A-12C). The antibody group showed higher concentration of fentanyl that is sequestered in blood by the antibody.

### Mu-Opioid Receptor Activation In Vitro.

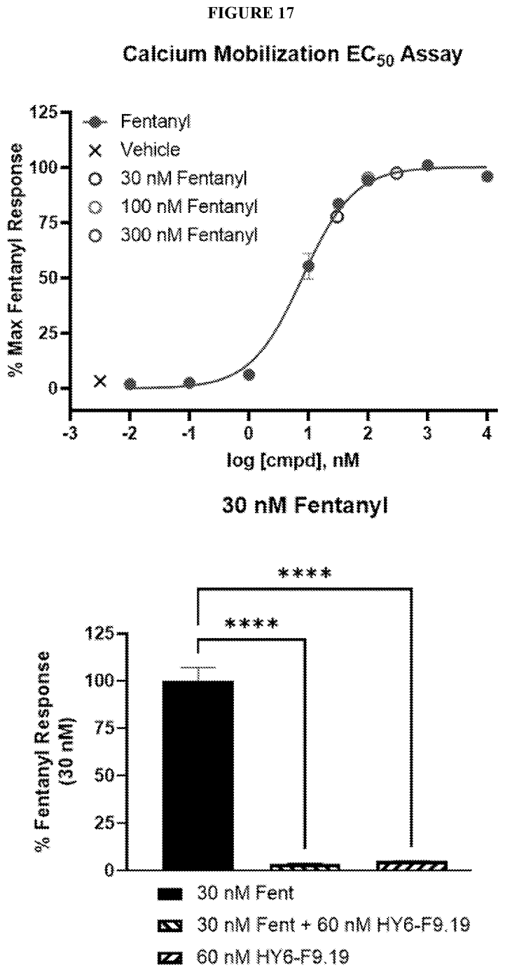

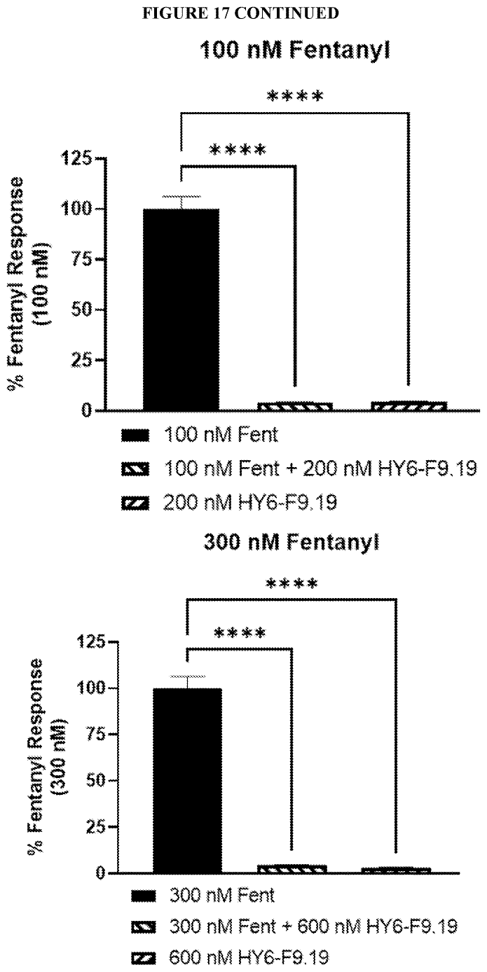

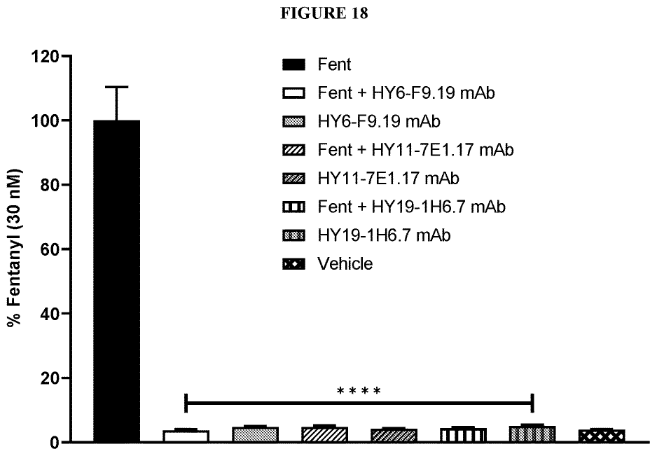

Cells stably expressing human MOR (Gα₁₆-CHO-hMOR) were incubated with 30-300 nM fentanyl with or without a 2-fold excess of anti-fentanyl mAb (HY6-F9.19), and MOR activation-induced Ca²⁺ signal was quantitated with Calcium 5 dye. It was shown that anti-fentanyl mAb blocks mu-opioid receptor (MOR) signaling in vitro (FIG. 17).

Cells stably expressing human MOR (Gα16-CHO-hMOR) were incubated with 30 nM fentanyl with or without a 3-fold excess of anti-fentanyl mAb (HY6-F9.19, HY11-7E1.17, and HY19-1H6.7), and MOR activation-induced Ca²⁺ signal was quantitated with Calcium 5 dye (FIG. 18).

### Anti-Fentanyl mAb Reverses Respiratory Depression in Non-Human Primates.

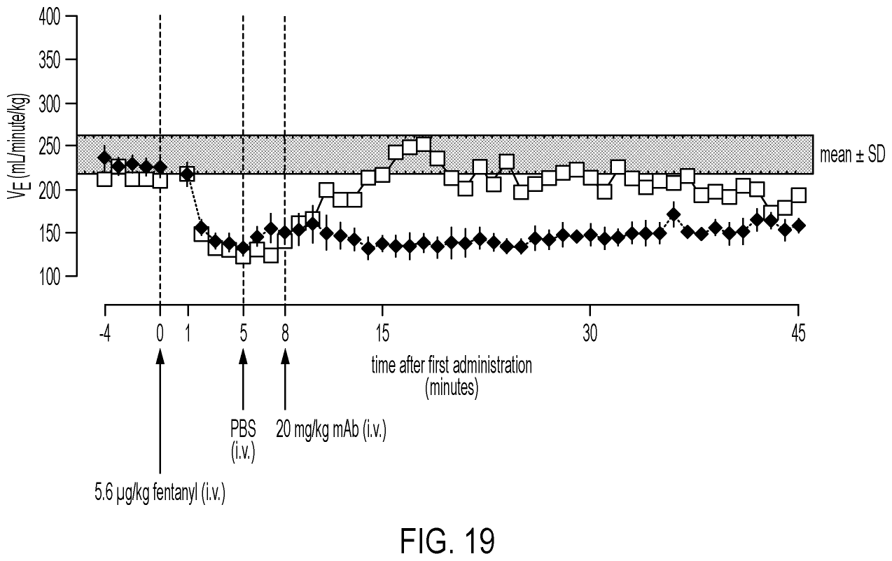

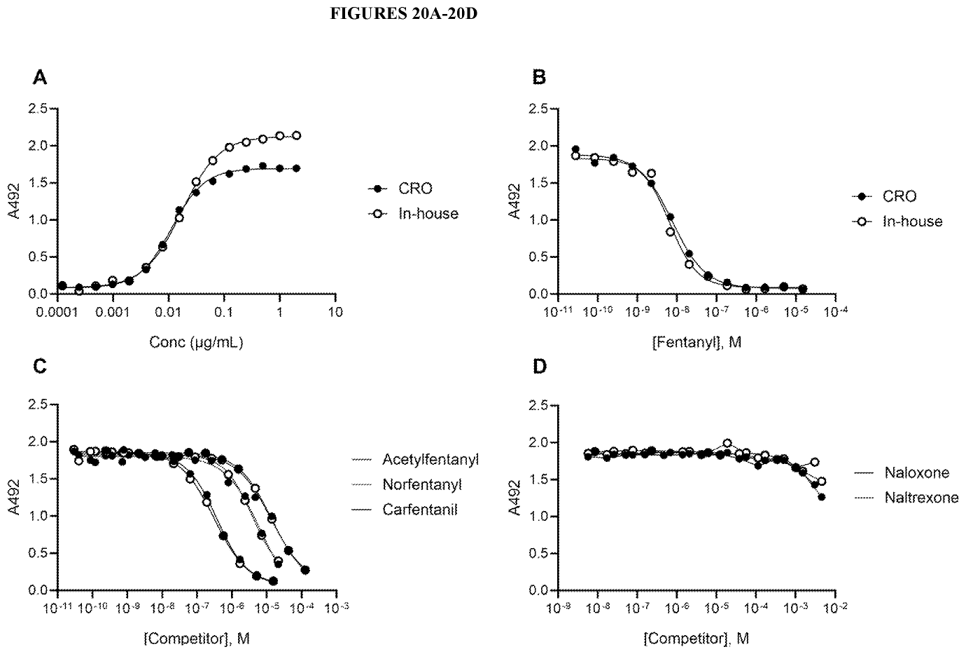

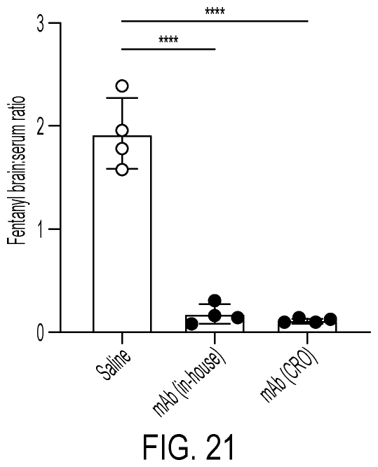

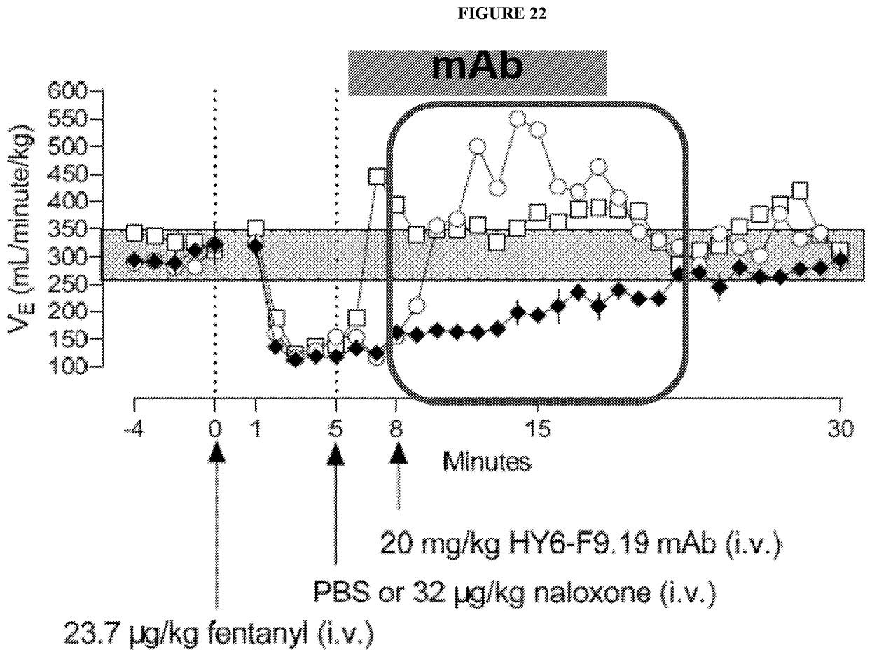

Fentanyl dose-response was first established in Rhesus macaques to determine the dose of fentanyl that induced stable respiratory depression. Reversal of fentanyl effects with naloxone was recorded prior to experiment initiation. Reversal of fentanyl with mAb 20 mg/kg was tested, and subsequent protection from additional fentanyl challenge was tested at 2-week intervals. It was shown that anti-Fentanyl mAb (HY6-F9.19) reversed respiratory depression in non-human primates (FIG. 19 and FIG. 22).

### References in Example 5

Hicks D, Baehr C et al. Advancing humanized monoclonal antibody for counteracting fentanyl toxicity towards clinical development. Hum Vaccin Immunother. 2022 Nov. 30; 18(6):2122507.

Rodarte J V et al. Structures of drug-specific monoclonal antibodies bound to opioids and nicotine reveal a common mode of binding. Structure. 2023 Jan. 5; 31(1):20-32.e5.

Baehr C A et al. Pharmacological Profiling of Antifentanyl Monoclonal Antibodies in Combination with Naloxone in Pre- and Postexposure Models of Fentanyl Toxicity. J Pharmacol Exp Ther. 2022 May; 381(2):129-136.

### Example 6. Binding Data of Certain Antibody Clones Described Herein

TABLE 22

| | BLI KD for | BLI KD for |
|---|---|---|
| | Fentanyl | Carfentanil |
| | Hapten | Hapten |
| mAb ID | (F1) (nM) | (F1) (nM) |
| HY11-7E1.25 | <0.001 | 0.001 |
| HY17-2A2.4 | 0.170 | <0.001 |
| HY17-2A2.7 | 1.062 | 0.002 |
| HY17-2A2.8 | 3.787 | 1.278 |
| HY18-5B1.2 | <0.001 | 7.523 |
| HY18-5B1.4 | 0.050 | 16.340 |
| HY18-5B1.6 | <0.001 | 0.870 |
| HY18-5B1.9 | 0.325 | 5.661 |
| HY18-5D1.2 | 0.134 | 6.139 |
| HY19-3A2.2 | 1.484 | 0.613 |
| HY19-1H6.1 | <0.001 | <0.001 |
| HY19-1H6.2 | <0.001 | 4.718 |
| HY19-1H6.3 | | 1.300 |
| HY19-1H6.4 | 0.030 | 0.590 |
| HY19-1H6.7 | <0.001 | No Binding |
| HY19-1H6.8 | <0.001 | No Binding |
| HY19-1H6.11 | <0.001 | No Binding |
| HY19-1H6.12 | <0.001 | No Binding |
| HY19-1H6.15 | <0.001 | 0.461 |

The isolated anti-fentanyl antibody or fragment thereof, may comprise a mutation in the non-CDR region that may help maintain or improve target (e.g., fentanyl and/or carfentanil) binding. As a non-limiting example, the isolated anti-fentanyl antibody or fragment thereof, may comprise Y at position 36 of light chain variable region according to Kabat numbering. For instance, one mutation of F36Y (mutation denoted according to Kabat numbering) can be introduced to SEQ ID NO:24 to arrive at SEQ ID NO:170 (e.g., see clones HY19-1H6.15, HY17-2A2.7, HY17-2A2.8, HY11-7E1.25, HY18-5B1.6 and HY18-5B1.9).

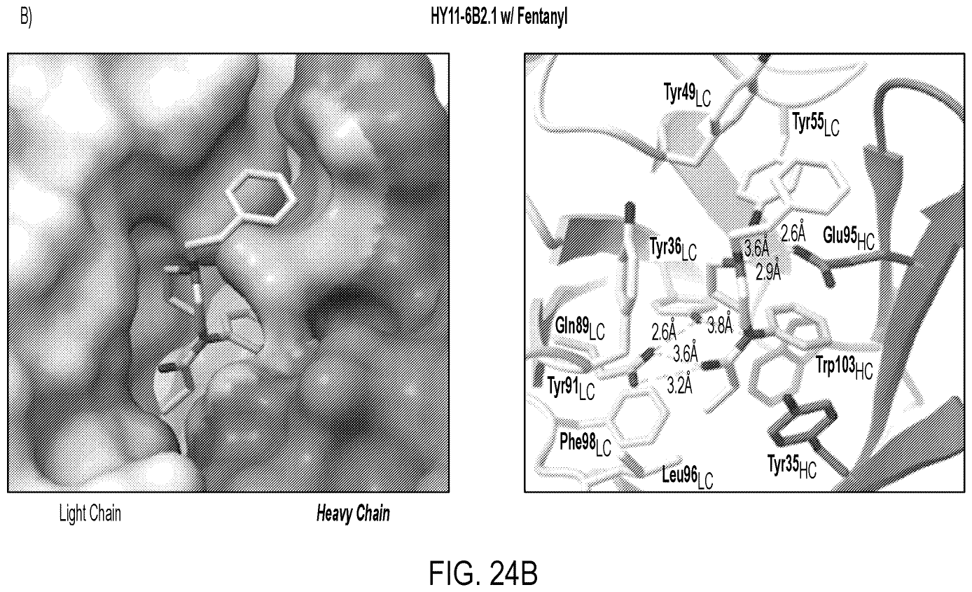

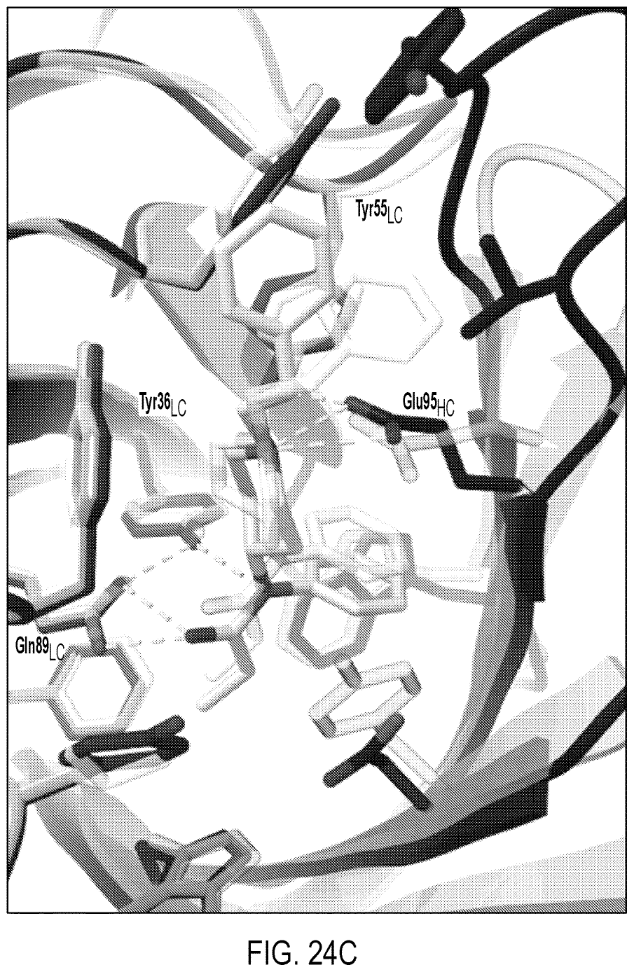

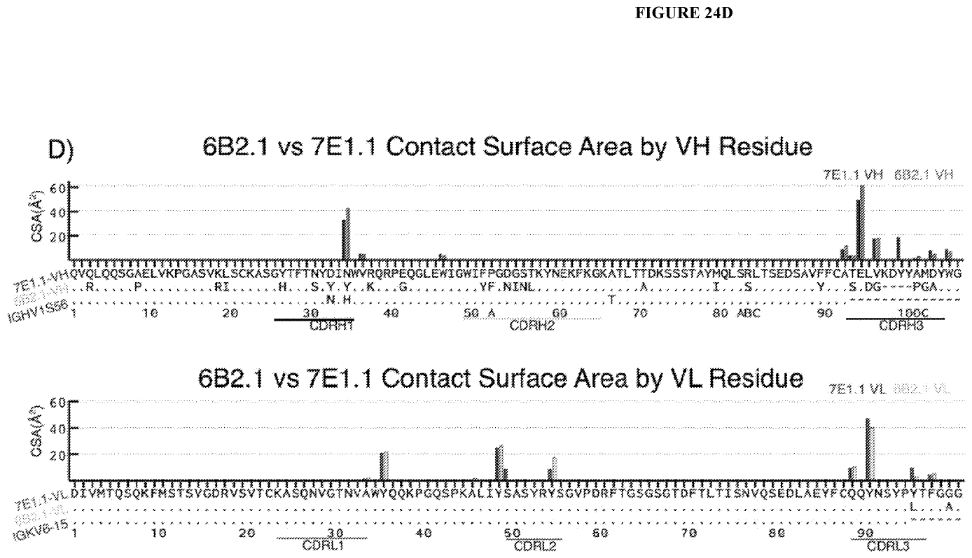

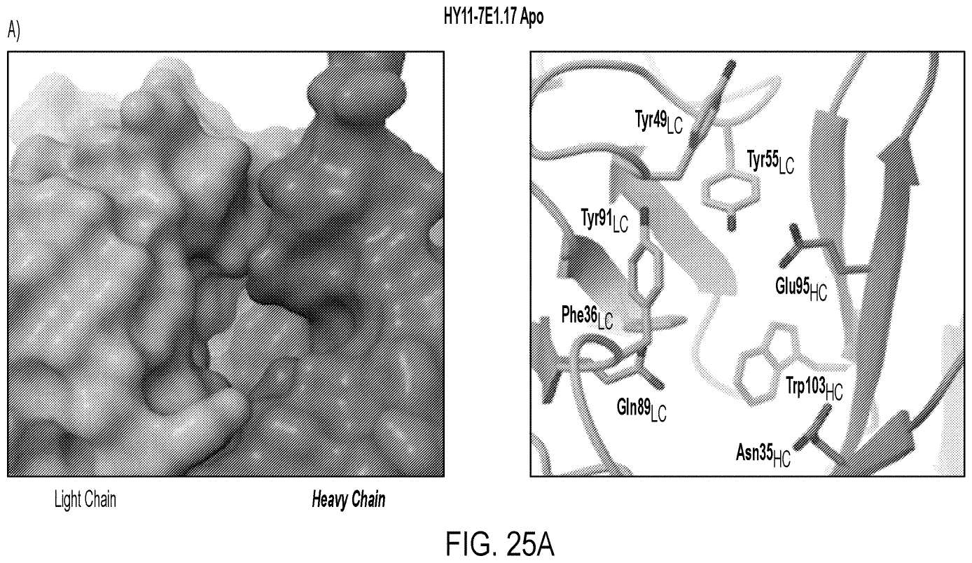

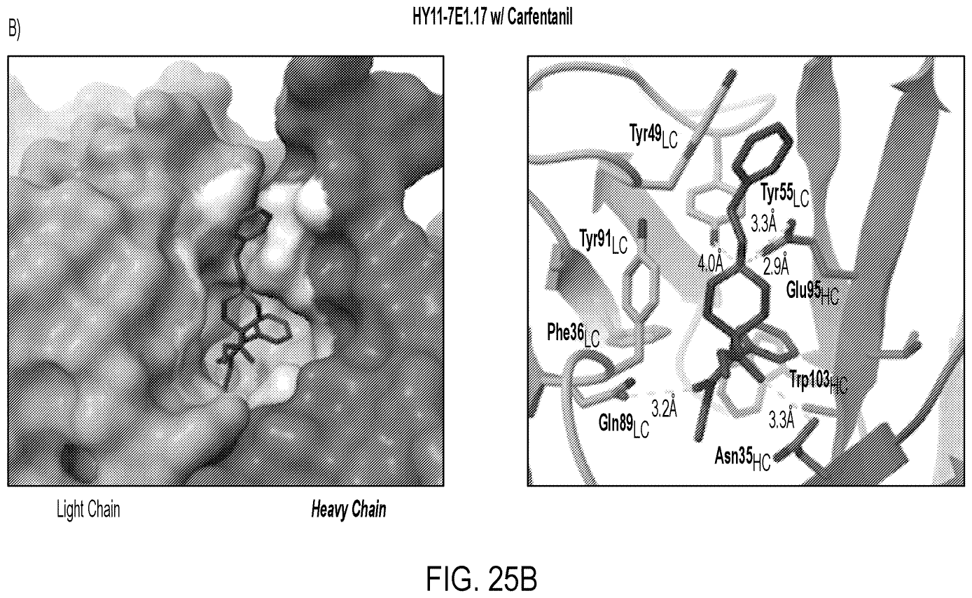

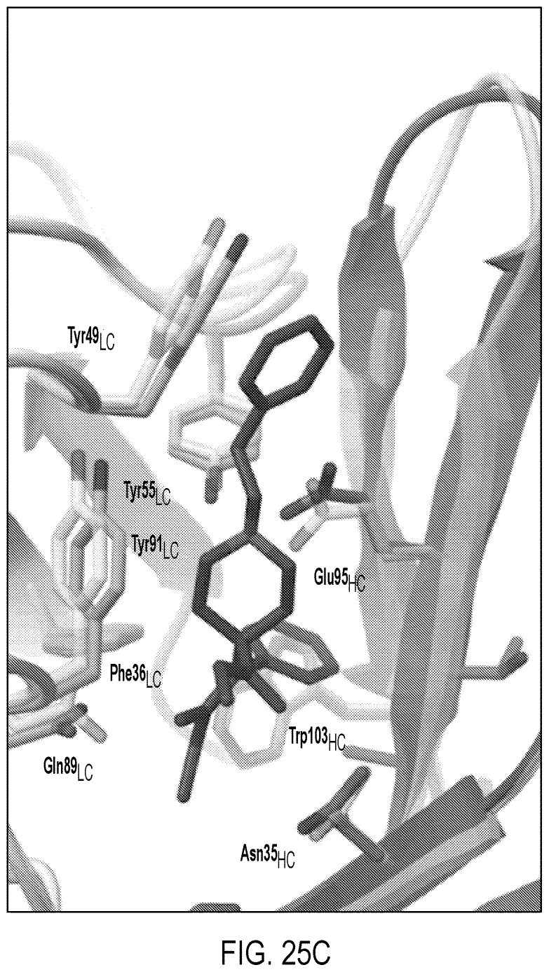

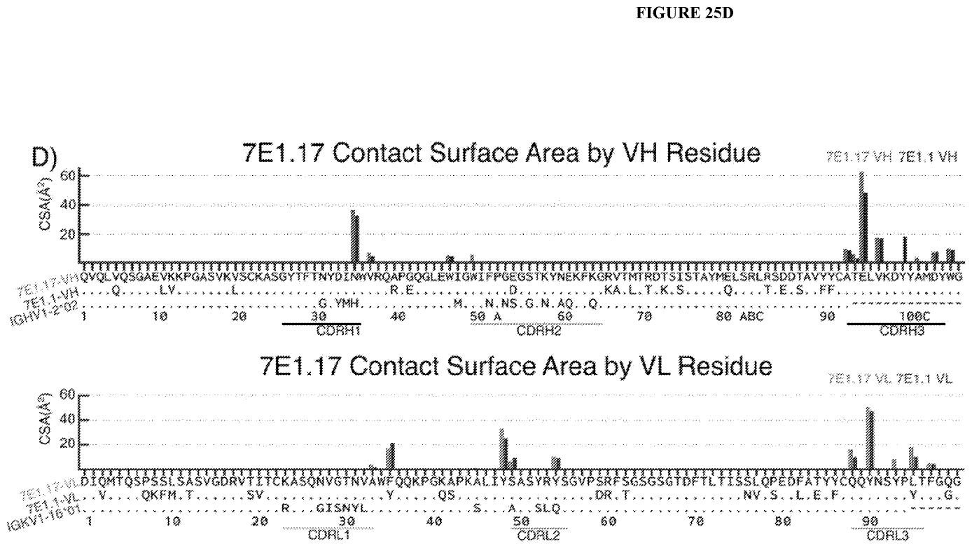

In addition, crystal data of HY11-7E1.1/HY11-6B2.1 clones binding with Fentanyl and HY11-7E1.17 clone binding with carfentanil were shown in FIG. 24 and FIG. 25 respectively.

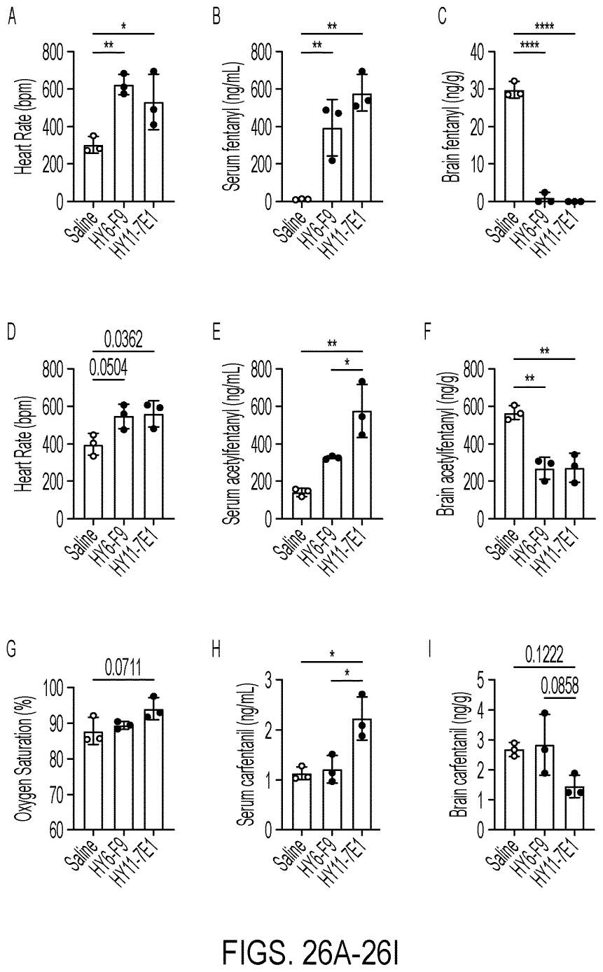

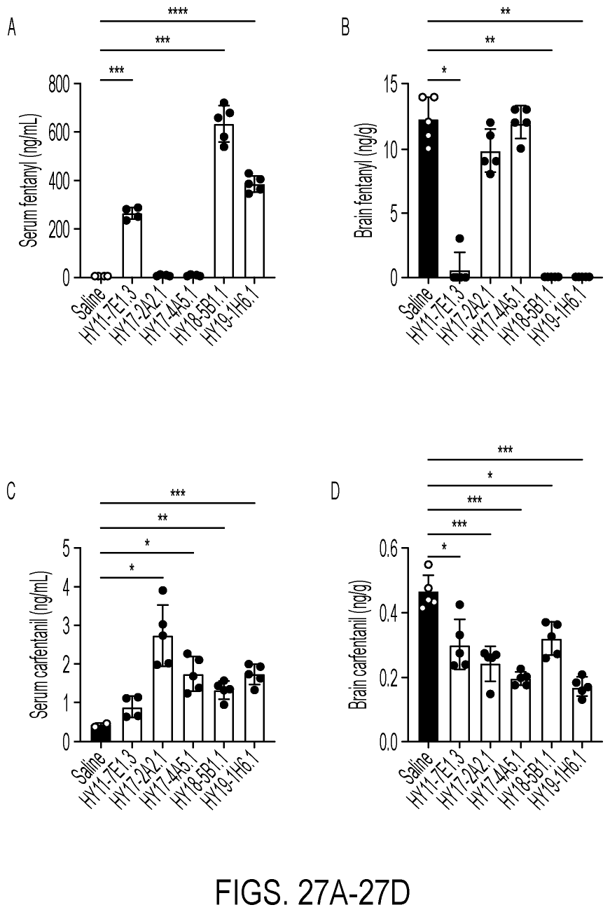

Additionally, mice studies also demonstrated efficacy of anti-fentanyl mAbs against fentanyl, acetylfentanyl, or carfentanil as shown in FIG. 26 and FIG. 27.

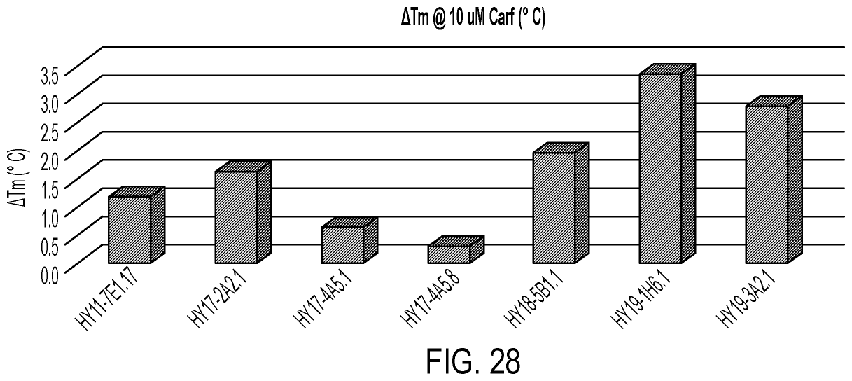

Moreover, HY11-7E1.17, HY17-2A2.1, HY17-4A5.1, HY17-4A5.8, HY18-5B1.1, HY19-1H6.1, and HY19-3A2.1 clones showed varying increases in melting temperature Tₘ upon incubation with carfentanil. Tₘ increase is indicative of a binding interaction (see FIG. 28).

All publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

1. An isolated antibody specific for fentanyl and its analog, or
fragment thereof, comprising:
(a) a heavy chain CDR1 comprising the amino acid sequence of any one of GDSITSGYWN (SEQ ID NO:62) and GDSITSGYWS (SEQ ID NO:63);
(b) a heavy chain CDR2 comprising the amino acid sequence of YISYSGSTYYNPSLKS (SEQ ID NO:64);
(c) a heavy chain CDR3 comprising the amino acid sequence of ARYYGDNYVGAOiDY (SEQ ID NO:165), wherein O₁ is M, L, Q, I, or A:
(d) a light chain CDR1 comprising the amino acid sequence of X₁SSKSLLHSX₂X₃ITYLX₄ (SEQ ID NO:66), wherein X₁ is R or K, X₂ is N, Q or D, X₃ is G, K or R, X₄ is Y, D or A;
(e) a light chain CDR2 comprising the amino acid sequence of any one of QMSNLAS (SEQ ID NO:75), QMSNRAS (SEQ ID NO:76), and QMSNRES (SEQ ID NO:77); and
(f) a light chain CDR3 comprising the amino acid sequence of AQNLELPWT (SEQ ID NO:78).

2-11. (canceled)

12. The isolated antibody of claim 1, or fragment thereof, comprising:
(a) a heavy chain CDR1 comprising the amino acid sequence of SEQ ID NO:62;
(b) a heavy chain CDR2 comprising the amino acid sequence of SEQ ID NO:64;
(c) a heavy chain CDR3 comprising the amino acid sequence of SEQ ID NO:65;
(d) a light chain CDR1 comprising the amino acid sequence of SEQ ID NO:70;
(e) a light chain CDR2 comprising the amino acid sequence of SEQ ID NO:75; and
(f) a light chain CDR3 comprising the amino acid sequence of SEQ ID NO:78.

13. The isolated antibody of claim 1, or fragment thereof, comprising a heavy chain variable region comprising an amino acid sequence that has at least 80% sequence identity to any one amino acid sequence of SEQ ID NOs:1, 3-5, and 166-169.

14. The isolated antibody of claim 1, or fragment thereof, comprising a light chain variable region comprising an amino acid sequence that has at least 80% sequence identity to any one amino acid sequence of SEQ ID NOs:2 and 6-13.

15. (canceled)

16. The isolated antibody of claim 1, or fragment thereof, comprising a heavy chain variable region comprising an amino acid sequence that has at least 90% sequence identity to the amino acid sequence of SEQ ID NO:4, and a light chain variable region comprising an amino acid sequence that has at least 90% sequence identity to the amino acid sequence of SEQ ID NO:10.

17. An isolated antibody specific for fentanyl and its analog, or fragment thereof, comprising:
(a) a heavy chain CDR1 comprising the amino acid sequence of any one of GYTFTNYDIN (SEQ ID NO:80) and GYTFTNYDMH (SEQ ID NO:81);
(b) a heavy chain CDR2 comprising the amino acid sequence of WIFPGJ₁J₂STJ₃YJ₄J₅KFJ₆G (SEQ ID NO:82), wherein J₁ is D or E, J₂ is G or V, J₃ is K or N, J₄ is N or A, J₅ is E or Q, and J₆ is K or Q;
(c) a heavy chain CDR3 comprising the amino acid sequence of ATELVKDYYAMDY (SEQ ID NO:87);
(d) a light chain CDR1 comprising the amino acid sequence of any one of KASQNVGTNVA (SEQ ID NO:89) and RASQNVGTNLA (SEQ ID NO:90);
(e) a light chain CDR2 comprising the amino acid sequence of any one of SASYRYS (SEQ ID NO:92), and SASYLQS (SEQ ID NO:93); and
(f) a light chain CDR3 comprising the amino acid sequence of any one of QQYNSYPYT (SEQ ID NO:95), QQYYNYPYT (SEQ ID NO:96), and QQYNSYPLT (SEQ ID NO:97).

18-29. (canceled)

30. The isolated antibody of claim 17, or fragment thereof, comprising:
(a) a heavy chain CDR1 comprising the amino acid sequence of SEQ ID NO: 80;
(b) a heavy chain CDR2 comprising the amino acid sequence of SEQ ID NO:85;
(c) a heavy chain CDR3 comprising the amino acid sequence of SEQ ID NO: 87;
(d) a light chain CDR1 comprising the amino acid sequence of SEQ ID NO: 89;
(e) a light chain CDR2 comprising the amino acid sequence of SEQ ID NO:92; and
(f) a light chain CDR3 comprising the amino acid sequence of SEQ ID NO:97.

31. The isolated antibody of claim 17, or fragment thereof, comprising a heavy chain variable region comprising an amino acid sequence that has at least 80% sequence identity to any one amino acid sequence of SEQ ID NOs:14, 18-20 and 25-26.

32. The isolated antibody of claim 17, or fragment thereof, comprising a light chain variable region comprising an amino acid sequence that has at least 80% sequence identity to any one amino acid sequence of SEQ ID NOs:15-17, 21-24, and 170.

33. (canceled)

34. The isolated antibody of claim 17, or fragment thereof, comprising a heavy chain variable region comprising an amino acid sequence that has at least 90% sequence identity to the amino acid sequence of SEQ ID NO:25, and a light chain variable region comprising an amino acid sequence that has at least 90% sequence identity to the amino acid sequence of SEQ ID NO:24 or 170.

35. An isolated anti-fentanyl antibody or fragment thereof, comprising:
(a) a heavy chain CDR1 comprising the amino acid sequence of any one of GYTFTESTMY (SEQ ID NO:121) and GYTFTESTMH (SEQ ID NO:182);
(b) a heavy chain CDR2 comprising the amino acid sequence of X₁INPNX₂GGTX₃Y X₄QKFX₅G (SEQ ID NO:183), wherein X₁ is H or R, X₂ is N or Q, X₃ is N, or S, X₄ is N or A, X₅ is R or Q;
(c) a heavy chain CDR3 comprising the amino acid sequence of AMELFYFDY (SEQ ID NO:123);
(d) a light chain CDR1 comprising the amino acid sequence of KASQNVGTNVA (SEQ ID NO:89);
(e) a light chain CDR2 comprising the amino acid sequence of SASYRYS (SEQ ID NO:92); and
(f) a light chain CDR3 comprising the amino acid sequence of any one of QQYNISPYT (SEQ ID NO:124), QQYQISPYT (SEQ ID NO:187), and QQYNSYPLT (SEQ ID NO:97).

36-44. (canceled)

45. The isolated anti-fentanyl antibody of claim 35, or fragment thereof, comprising one or more CDRs selected from the group consisting of:
(a) a heavy chain CDR1 comprising the amino acid sequence of SEQ ID NO:121;
(b) a heavy chain CDR2 comprising the amino acid sequence of SEQ ID NO: 122;
(c) a heavy chain CDR3 comprising the amino acid sequence of SEQ ID NO: 123;
(d) a light chain CDR1 comprising the amino acid sequence of SEQ ID NO: 89;
(e) a light chain CDR2 comprising the amino acid sequence of SEQ ID NO:92; and
(f) a light chain CDR3 comprising the amino acid sequence of SEQ ID NO:97.

46. The isolated anti-fentanyl antibody of claim 35, or fragment thereof, comprising a heavy chain variable region comprising an amino acid sequence that has at least 80% sequence identity to the amino acid sequence of any one of SEQ ID NOs:40, 176, 177, or 180.

47. The isolated anti-fentanyl antibody of claim 35, or fragment thereof, comprising a light chain variable region comprising an amino acid sequence that has at least 80% sequence identity to the amino acid sequence of any one of SEQ ID NOs:24, 41, 170, 178, or 179.

48-49. (canceled)

50. The isolated anti-fentanyl antibody of claim 35, or fragment thereof, comprising a heavy chain variable region comprising an amino acid sequence that has at least 90% sequence identity to the amino acid sequence of SEQ ID NO:177, and a light chain variable region comprising an amino acid sequence that has at least 90% sequence identity to the amino acid sequence of SEQ ID NO:170.

51-71. (canceled)

72. A pharmaceutical composition comprising an isolated antibody of claim 1, or fragment thereof, and a pharmaceutically acceptable carrier.

73. (canceled)

74. An isolated polynucleotide comprising a nucleotide sequence encoding the isolated antibody, or fragment thereof, of claim 1.

75. (canceled)

76. A cell comprising the polynucleotide of claim 74.

77. (canceled)

78. A method of inhibiting the activity of fentanyl or analog in a mammal in need of, comprising administering an isolated antibody of claim 1, or fragment thereof, to the mammal.

79-80. (canceled)

81. A method for treating fentanyl or analog related overdose, poisoning or disorder in a mammal in need of, comprising administering an effective amount of an isolated antibody of claim 1, or fragment thereof, to the mammal.

82-90. (canceled)
